(19) United States
(12) Patent Application Publication
(10) Pub. No.: US 2024/0067961 A1
(43) Pub. Date: Feb. 29, 2024

(54) SYSTEMS, METHODS, AND COMPOSITIONS FOR SITE-SPECIFIC GENETIC ENGINEERING USING PROGRAMMABLE ADDITION VIA SITE-SPECIFIC TARGETING ELEMENTS (PASTE)

(71) Applicant: Massachusetts Institute of Technology, Cambridge, MA (US)

(72) Inventors: Omar Abudayyeh, Cambridge, MA (US); Jonathan Gootenberg, Cambridge, MA (US)

(21) Appl. No.: 18/487,744

(22) Filed: Oct. 16, 2023

Related U.S. Application Data
(63) Continuation of application No. 18/066,223, filed on Dec. 14, 2022, now Pat. No. 11,827,881.
Application No. 18/066,223 is a continuation of application No. 17/649,308, filed on Jan. 28, 2022, now Pat. No. 11,572,556.
Application No. 17/649,308 is a continuation of application No. 17/451,734, filed on Oct. 21, 2021, now abandoned.
(60) Provisional application No. 63/222,550, filed on Jul. 16, 2021.
(60) Provisional application No. 63/094,803, filed on Oct. 21, 2020.

(51) Int. Cl. C12N 15/11; A61K 31/7105; C12N 9/12; C12N 9/22; C12N 15/10; C12N 15/113; C12N 15/85; C12N 15/90

(52) U.S. Cl. CPC C12N 15/111; A61K 31/7105; C12N 9/1276; C12N 9/22; C12N 15/102; C12N 15/113; C12N 15/85; C12N 15/907; C12N 2310/20; C12N 2310/3519; C12Y 207/07049

(57) ABSTRACT

This disclosure provides systems, methods, and compositions for site-specific genetic engineering using Programmable Addition via Site-Specific Targeting Elements (PASTE). PASTE comprises the addition of an integration site into a target genome followed by the insertion of one or more genes of interest or one or more nucleic acid sequences of interest at the site. PASTE combines gene editing technologies and integrase technologies to achieve unidirectional incorporation of genes in a genome for the treatment of diseases and diagnosis of disease.

### CROSS REFERENCE TO RELATED APPLICATIONS

This application claims the benefit of U.S. Provisional Patent Application Ser. No. 63/222,550, filed Jul. 16, 2021 and U.S. Provisional Patent Application Ser. No. 63/094,803, filed Oct. 21, 2020. The entire contents of the above-referenced patent applications are incorporated by reference in their entirety herein.

### FIELD OF DISCLOSURE

The subject matter disclosed herein is generally directed to systems, methods, and compositions for site-specific genetic engineering using Programmable Addition via Site-Specific Targeting Elements (PASTE) for the treatment of diseases and diagnostics.

### BACKGROUND

Editing genomes using the RNA-guided DNA targeting principle of CRISPR-Cas (Clustered Regularly Interspaced Short Palindromic Repeats-CRISPR associated proteins) immunity has been widely exploited and has become a powerful genome editing means for a wide variety of applications. The main advantage of CRISPR-Cas system lies in the minimal requirement for programmable DNA interference: an endonuclease, such as a Cas9, Cas12, or any programmable nucleases, guided by a customizable dual-RNA structure. Cas9 is a multi-domain enzyme that uses an HNH nuclease domain to cleave the target strand. The CRISPR/Cas9 protein-RNA complex is localized on the target by a guide RNA (guide RNA), then cleaved to generate a DNA double strand break (dsDNA break, DSB). After cleavage, DNA repair mechanisms are activated to repair the cleaved strand. Repair mechanisms are generally from one of two types: non-homologous end joining (NHEJ) or homologous recombination (HR). In general, NHEJ dominates the repair, and, being error prone, generates random indels (insertions or deletions) causing frame shift mutations, among others. In contrast, HR has a more precise repairing capability and is potentially capable of incorporating the exact substitution or insertion. To enhance HR, several techniques have been tried, for example: combination of fusion proteins of Cas9 nuclease with homology-directed repair (HDR) effectors to enforce their localization at DSBs, introducing an overlapping homology arm, or suppression of NHEJ. Most of these techniques rely on the host DNA repair systems.

Recently, new guided editors have been developed, such as guided prime editors (PE) PE1, PE2, and PE3, e.g., Liu, D. et al., Nature 2019, 576, 149-157. These PEs are reverse transcriptase (RT) fused with Cas 9 H 840A nickase (Cas9n (H840A)), and the genome editing is achieved using a prime-editing guide RNA (pegRNA). Despite these developments, programmable gene integration is still generally dependent on cellular pathways or repair processes.

Therefore, there is a need for more effective tools for gene editing and delivery.

### SUMMARY

The present disclosure provides a method of site-specific integration of a nucleic acid into a cell genome. The method comprises incorporating an integration site at a desired location in the cell genome by introducing into the cell a DNA binding nuclease linked to a reverse transcriptase domain, wherein the DNA binding nuclease comprises a nickase activity; and a guide RNA (gRNA) comprising a primer binding sequence linked to an integration sequence, wherein the gRNA interacts with the DNA binding nuclease and targets the desired location in the cell genome, wherein the DNA binding nuclease nicks a strand of the cell genome and the reverse transcriptase domain incorporates the integration sequence of the gRNA into the nicked site, thereby providing the integration site at the desired location of the cell genome. The method further comprises integrating the nucleic acid into the cell genome by introducing into the cell a DNA or RNA strand comprising the nucleic acid linked to a sequence that is complementary or associated to the integration site, and an integration enzyme, wherein the integration enzyme incorporates the nucleic acid into the cell genome at the integration site by integration, recombination, or reverse transcription of the sequence that is complementary or associated to the integration site, thereby introducing the nucleic acid into the desired location of the cell genome of the cell.

In some embodiments, the gRNA can be hybridized to a complementary strand of the cell genome to the genomic strand that is nicked by the DNA binding nuclease.

In some embodiments, the integration enzyme can be introduced as a peptide or a nucleic acid encoding the same.

In some embodiments, the DNA binding nuclease can be introduced as a peptide or a nucleic acid encoding the same.

In some embodiments, the DNA or RNA strand comprising the nucleic acid can be introduced into the cell as a minicircle, a plasmid, mRNA or a linear DNA.

In some embodiments, the DNA or RNA strand comprising the nucleic acid can be between 1000 bp and 10,000 bp.

In some embodiments, the DNA or RNA strand comprising the nucleic acid can be more than 10,000 bp.

In some embodiments, the DNA or RNA strand comprising the nucleic acid can be less than 1000 bp.

In some embodiments, the DNA comprising the nucleic acid can be introduced into the cell as a minicircle.

In some embodiment, the minicircle cannot comprise sequences of a bacterial origin.

In some embodiments, the DNA binding nuclease can be linked to a reverse transcriptase domain and the integration enzyme can be linked via a linker. The linker can be cleavable. The linker can be non-cleavable. The linker can be replaced by two associating binding domains of the DNA binding nuclease linked to a reverse transcriptase.

In some embodiments, the integration enzyme can be selected from the group consisting of Cre, Dre, Vika, Bxb1, φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), and Minos, and any mutants thereof.

In some embodiments, the integration enzyme can be Bxb1 or a mutant thereof.

In some embodiments, the integration site can be selected from an attB site, an attP site, an attL site, an attR site, a lox71 site a Vox site, or a FRT site.

In some embodiments, the DNA binding nuclease comprising a nickase activity can be selected from Cas9-D10A, Cas9-H840A, and Cas12a/b nickase.

In some embodiments, the reverse transcriptase domain can be selected from the group consisting of Moloney Murine Leukemia Virus (M-MLV) reverse transcriptase domain, transcription xenopolymerase (RTX), avian myeloblastosis virus reverse transcriptase (AMV-RT), and Eubacterium rectale maturase RT (MarathonRT).

In some embodiments, the reverse transcriptase domain can comprise a mutation relative to the wild-type sequence.

In some embodiments, the M-MLV reverse transcriptase domain can comprise one or more mutations selected from the group consisting of D200N, T306K, W313F, T330P and L603W.

In some embodiments, the method can further comprise introducing a second nicking guide RNA (ngRNA). The ngRNA can direct nicking at 90 bases downstream of the gRNA nick on a complementary strand.

In some embodiments, the gRNA, the nucleic acid encoding the DNA binding nuclease, the reverse transcriptase, the DNA comprising nucleic acid linked to a complementary integration site, the integration enzyme, and optionally the ngRNA can be introduced into a cell in a single reaction.

In some embodiments, the gRNA, the nucleic acid encoding the DNA binding nuclease, the reverse transcriptase, the DNA comprising nucleic acid linked to a complementary integration site, the integration enzyme, and optionally the ngRNA can be introduced using a virus, a RNP, an mRNA, a lipid, or a polymeric nanoparticle.

In some embodiments, the nucleic acid can be a reporter gene. The reporter gene can be a fluorescent protein.

In some embodiments, the cell can be a dividing cell.

In some embodiments, the cell can be a non-dividing cell.

In some embodiments, the desired location in the cell genome can be the locus of a mutated gene.

In some embodiments, the nucleic acid can be a degradation tag for programmable knockdown of proteins in the presence of small molecules.

In some embodiments, the cell can be a mammalian cell, a bacterial cell or a plant cell.

In some embodiments, nucleic acid can be a T-cell receptor (TCR), a chimeric antigen receptor (CAR), an interleukin, a cytokine, or an immune checkpoint gene for integration into a T-cell or natural killer (NK) cell. The TCR, the CAR, the interleukin, the cytokine, or the immune checkpoint gene can be incorporated into the target site of the T-cell or NK cell genome using a minicircle DNA.

In some embodiments, the nucleic acid can be a beta hemoglobin (HBB) gene and the cell can be a hematopoietic stem cell (HSC). The HBB gene can be incorporated into the target site in the HSC genome using a minicircle DNA. The nucleic acid can be a gene responsible for beta thalassemia or sickle cell anemia.

In some embodiments, the nucleic acid can be a metabolic gene. The metabolic gene can be involved in alpha-1 antitrypsin deficiency or ornithine transcarbamylase (OTC) deficiency. The metabolic gene can be a gene involved in inherited diseases.

In some embodiments, the nucleic acid can be a gene involved in an inherited disease or an inherited syndrome. The inherited disease can be cystic fibrosis, familial hypercholesterolemia, adenosine deaminase (ADA) deficiency, X-linked SCID (X-SCID), Wiskott-Aldrich syndrome (WAS), hemochromatosis, Tay-Sachs, fragile X syndrome, Huntington's disease, Marfan syndrome, phenylketonuria, or muscular dystrophy.

The present disclosure provides a vector comprising a nucleic acid encoding a DNA binding nuclease comprising a nickase activity C-terminally linked to a reverse transcriptase linked to an integration enzyme via a linker.

In some embodiments, the linker can be cleavable.

In some embodiments, the linker can be non-cleavable.

In some embodiments, the linker can comprise two associating binding domains of the DNA binding nuclease linked to a reverse transcriptase.

In some embodiments, the integration enzyme can comprise a conditional activation domain or conditional expression domain.

In some embodiments, the integration enzyme can be fused to an estrogen receptor.

In some embodiments, the DNA binding nuclease comprising a nickase activity can be selected from the group consisting of Cas9-D10A, Cas9-H840A, and Cas12a/b.

In some embodiments, the reverse transcriptase can be a M-MLV reverse transcriptase, a AMV-RT, MarathonRT, or a RTX. The reverse transcriptase can be a modified M-MLV reverse transcriptase relative to the wildtype M-MLV reverse transcriptase. The M-MLV reverse transcriptase domain can comprise one or more of the mutations selected from the group consisting of D200N, T306K, W313F, T330P and L603W.

In some embodiments, the integration enzyme can be selected from the group consisting of Cre, Dre, Vika, Bxb1, φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tcl, Tc3, Mariner (Himar 1), Mariner (mos 1), and Minos, and any mutants thereof.

In some embodiments, the recombinase or integrase can be Bxb1 or a mutant thereof.

The present disclosure provides a cell comprising a vector comprising a nucleic acid encoding a DNA binding nuclease comprising a nickase activity C-terminally linked to a reverse transcriptase linked to an integration enzyme via a linker. The cell further comprises a gRNA comprising a primer binding sequence, an integration sequence, and a guide sequence, wherein the gRNA can interact with the encoded nuclease comprising a nickase activity. The cell further comprising a DNA minicircle comprising a nucleic acid and a sequence recognized by the encoded integrase, recombinase, or reverse transcriptase. The cell further comprising a nicking guide RNA (ngRNA) capable of binding the encoded nuclease comprising a nickase activity, and wherein the ngRNA targets a sequence away from the gRNA.

In some embodiments, the minicircle cannot comprise a sequence of bacterial origin.

In some embodiments, the integration enzyme can be selected from the group consisting of Cre, Dre, Vika, Bxb1, φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tcl, Tc3, Mariner (Himar 1), Mariner (mos 1), and Minos, and any mutants thereof.

In some embodiments, the integration enzyme can be Bxb1 or a mutant thereof.

In some embodiments, the DNA binding nuclease comprising a nickase activity can be selected from the group consisting of Cas9-D10A, Cas9-H840A and Cas12a.

In some embodiments, the reverse transcriptase can be a M-MLV reverse transcriptase. The reverse transcriptase can be a modified M-MLV reverse transcriptase. The amino acid sequence of the M-MLV reverse transcriptase can comprise one or more mutations selected from the group consisting of D200N, T306K, W313F, T330P, and L603W.

In some embodiments, the cell can further comprise introducing ngRNA to the cell. The ngRNA can be a +90 ngRNA. The +90 ngRNA can direct nicking at 90 bases downstream of the gRNA nick on a complementary strand.

The present disclosure provides a polypeptide comprising a DNA binding nuclease comprising a nickase activity C-terminally linked to a reverse transcriptase linked to an integration enzyme via a linker.

In some embodiments, the linker can be cleavable.

In some embodiments, the linker can be non-cleavable.

In some embodiments, the integration enzyme can be fused to an estrogen receptor.

In some embodiments, the DNA binding nuclease comprising a nickase activity can be selected from the group consisting of Cas9-D10A, Cas9-H840A, and Cas12a/b.

In some embodiments, the reverse transcriptase can be a M-MLV reverse transcriptase, a AMV-RT, a MarathonRT, or a XRT. The reverse transcriptase can be a modified M-MLV relative to a wild-type M-MLV reverse transcriptase. The M-MLV reverse transcriptase domain can comprise one or more of mutations selected from the group consisting of D200N, T306K, W313F, T330P, and L603W.

In some embodiments, the integration enzyme can be selected from group consisting of Cre, Dre, Vika, Bxb1, φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tcl, Tc3, Mariner (Himar 1), Mariner (mos 1), and Minos, and any mutants thereof.

In some embodiments, the integration enzyme can be Bxb1 or a mutant thereof.

The present disclosure provides a gRNA that specifically binds to a DNA binding nuclease comprising nickase activity, the gRNA comprising a primer binding site, which hybridizes to a nicked DNA strand, a recognition site for an integration enzyme, and a target recognition sequence recognizing a target site in a cell genome and hybridizing to a genomic strand complementary to the strand that is nicked by the DNA binding nuclease.

In some embodiments, the DNA binding nuclease comprising a nickase activity can be selected from the group consisting of Cas9-D10A, Cas9-H840A, and Cas12a/b.

In some embodiments, the primer binding site can hybridize to the 3′ end of the nicked DNA strand.

In some embodiments, the recognition site for the integration enzyme can be selected from an attB site, an attP site, an attL site, an attR site, a lox71 site, and a FRT site.

In some embodiments, the recognition site for the integration enzyme can be a Bxb1 site.

The present disclosure provides a method of site-specific integration of two or more nucleic acids into a cell genome. The method comprises incorporating two integration sites at desired locations in the cell genome by introducing into the cell a DNA binding nuclease linked to a reverse transcriptase domain, wherein the DNA binding nuclease comprises a nickase activity, and two guide RNAs (gRNAs), each comprising, a primer binding sequence, linked to a unique integration sequence, wherein the gRNA interacts with the DNA binding nuclease and targets the desired locations in the cell genome, wherein the DNA binding nuclease nicks a strand of the cell genome and the reverse transcriptase domain incorporates each of the integration sequence of the gRNA into the nicked site, thereby providing the integration site at the desired locations of the cell genome. The method further comprises integrating the nucleic acid by introducing into the cell two or more DNA or RNA comprising the nucleic acids, wherein each DNA is flanked by orthogonal integration sites, and an integration enzyme, wherein the integration enzyme incorporates the nucleic acids into the cell genome at the integration sites by integrase, recombinase, or reverse transcriptase of the sequence that is complementary or associated to the integration site, thereby introducing the nucleic acids into the desired locations of the cell genome of the cell.

In some embodiments, each of the two different integration sites inserted into the cell genome can be attB sequences comprising different palindromic or non-palindromic central dinucleotide.

In some embodiments, each of the two different integration sites inserted into the cell genome can be attP sequences comprising different palindromic or non-palindromic central dinucleotide.

In some embodiments, the integration enzyme can enable each of the two or more DNA or RNA comprising the nucleic acids to directionally enable integration of the nucleic acids into a genome via recombination of a pair of orthogonal attB site sequence and an attP site sequence.

In some embodiments, the integration enzyme can be selected from the group consisting of Cre, Dre, Vika, Bxb1, φC31, RDF, FLP, φBT1, TP901-1, A118, φFC1, φC1, MR11, TG1, φ370.1, Wβ, BL3, SPBc, K38, Peaches, Veracruz, Rebeuca, Theia, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R1, R2, R3, R4, R5, L1, Tol2 Tc1, Tc3, Mariner (Himar 1), Mariner (mos 1), and Minos, and any mutants thereof.

In some embodiments, the integration enzyme can be Bxb1 or a mutant thereof.

In some embodiments, the DNA comprising genes can be genes involved in a cell maintenance pathway, cell-division, or a signal transduction pathway.

In some embodiments, the reverse transcriptase domain can comprise Moloney Murine Leukemia Virus (M-MLV) reverse transcriptase domain, transcription xenopolymerase (RTX), avian myeloblastosis virus reverse transcriptase (AMV-RT), or Eubacterium rectale maturase RT (MarathonRT).

In some embodiments, the DNA binding nuclease comprising a nickase activity can be selected from the group consisting of Cas9-D10A, Cas9-H840A, and Cas12a/b.

In some embodiments, the pair of an attB site sequence and an attP site sequence can be selected from the group consisting of SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34 and SEQ ID NO: 35 and SEQ ID NO: 36.

The present disclosure provides a cell comprising a vector comprising a nucleic acid encoding a DNA binding nuclease comprising a nickase activity, wherein the DNA binding nuclease is C-terminally linked to a reverse transcriptase, wherein the reverse transcriptase is linked to a recombinase or integrase via a linker. The cell further comprises two guide RNAs (gRNAs) comprising a primer binding sequence, an integration sequence and a guide sequence, wherein the gRNA can interact with the encoded DNA binding nuclease comprising a nickase activity. The cell further comprises two or more DNA or RNA strands comprising a nucleic acid and a pair of flanking attB site sequence and an attP site sequence recognized by the encoded integrase or recombinase. The cell optionally further comprises a nicking guide RNA (ngRNA) capable of binding the encoded nuclease comprising a nickase activity, and wherein the ngRNA targets a sequence away from the gRNA.

The present disclosure provides a cell comprising a modified genome, wherein the modification comprises incorporation of two orthogonal integration sites within the cell genome by introducing into the cell a: vector comprising a nucleic acid encoding a DNA binding nuclease comprising a nickase activity, wherein the DNA binding nuclease is C-terminally linked to a reverse transcriptase; two guide RNAs (gRNAs), each comprising a primer binding sequence, a genomic integration sequence, and a guide sequence, wherein the gRNA can interact with the encoded nuclease comprising a nickase activity; and optionally a nicking guide RNA (ngRNA) capable of binding the encoded nuclease comprising a nickase activity, and wherein the ngRNA targets a sequence away from the gRNA.

The present disclosure provides a method of integrating two or more nucleic acids into the cell genome of cell of claim 90, the method comprising introducing into the cell: two or more DNA, each comprising a nucleic acid and a pair of flanking orthogonal integration site sequences; an integration enzyme that can recognize the integration site sequence enabling directional linking of the two or more DNA comprising nucleic acid; and enabling incorporation of the nucleic acids into the cell genome by integrating the 5′ orthogonal integration sequence of the first DNA with the first genomic integration sequence and 3′ orthogonal integration sequence of the last DNA with the last genomic integration sequence, thereby incorporating the two or more nucleic acids into the cell genome.

The present disclosure provides a cell comprising a modified genome, wherein the modification comprises incorporation of two orthogonal integration sites within the cell genome by introducing into the cell: a vector comprising a nucleic acid encoding a DNA binding nuclease comprising a nickase activity, wherein the DNA binding nuclease is C-terminally linked to a reverse transcriptase; two guide RNAs (gRNAs), each comprising a primer binding sequence, a genomic integration sequence, and a guide sequence, wherein the gRNA can interact with the encoded nuclease comprising a nickase activity; and optionally a nicking guide RNA (ngRNA) capable of binding the encoded nuclease comprising a nickase activity, and wherein the ngRNA targets a sequence away from the gRNA; two or more DNA or RNA comprising the nucleic acids, wherein each DNA is flanked by orthogonal integration sites; and an integration enzyme, wherein the integration enzyme incorporates the nucleic acids into the cell genome at the integration sites.

### BRIEF DESCRIPTION OF THE DRAWINGS

Aspects, features, benefits and advantages of the embodiments described herein will be apparent with regard to the following description, appended claims, and accompanying drawings where:

FIG. 1 shows a schematic diagram of a concept of Programmable Addition via Site-Specific Targeting Elements (PASTE) according to embodiments of the present teachings;

FIG. 2 shows a schematic diagram of a prime editing process according to embodiments of the present teachings;

FIG. 3 shows the percent integration of green fluorescent protein (GFP) in the lentiviral integrated lox71 site in HEK293FT cell line in the presence of various plasmids according to embodiments of the present teachings;

FIG. 4 shows the percent editing of the HEK293FT genome for incorporation of various lengths of lox71 or lox66 according to embodiments of the present teachings;

FIG. 5A shows the percent editing of lox71 site with different PE/Cre vectors according to embodiments of the present teachings;

FIG. 5B shows the percent integration of GFP at the lox71 site in HEK293FT cell genome according to embodiments of the present teachings;

FIG. 6 shows a schematic representation of using Bxb1 to integrate a nucleic acid into the genome according to embodiments of the present teachings;

FIG. 7 shows the percent integration of GFP or Gluc into the attB locus using Bxb1 Programmable Addition via Site-Specific Targeting Elements (PASTE) according to embodiments of the present teachings;

FIG. 8 shows the percent editing of various HEK3 targeting pegRNA Programmable Addition via Site-Specific Targeting Elements (PASTE) according to embodiments of the present teachings;

FIG. 9A shows a fluorescent image of cells wherein the SUPT16H marker is tagged with EGFP using PASTE according to embodiments of the present teachings;

FIG. 9B shows a fluorescent image of cells wherein the SRRM2 marker is tagged with EGFP using Programmable Addition via Site-Specific Targeting Elements (PASTE) according to embodiments of the present teachings;

FIG. 9C shows a fluorescent image of cells wherein the LAMNB1 marker is tagged with EGFP using Programmable Addition via Site-Specific Targeting Elements (PASTE) according to embodiments of the present teachings;

FIG. 9D shows a fluorescent image of cells wherein the NOLC1 marker is tagged with EGFP using Programmable Addition via Site-Specific Targeting Elements (PASTE) according to embodiments of the present teachings;

FIG. 9E shows a fluorescent image of cells wherein the NOLC1 marker is tagged with EGFP using Programmable Addition via Site-Specific Targeting Elements (PASTE) according to embodiments of the present teachings;

FIG. 9F shows a fluorescent image of cells wherein the NOLC1 marker is tagged with EGFP using Programmable Addition via Site-Specific Targeting Elements (PASTE) according to embodiments of the present teachings;

FIG. 9G shows a fluorescent image of cells wherein the DEPDC4 marker is tagged with EGFP using Programmable Addition via Site-Specific Targeting Elements (PASTE) according to embodiments of the present teachings;

FIG. 10A shows comparisons of lipofectamine aided transfection in blue with electroporation aided transfection in red for the addition of the Bxb1 attB site at the ACTB N-terminal site in the genome using PASTE according to embodiments of the present teachings;

FIG. 10B shows comparisons of lipofectamine aided transfection in blue with electroporation aided transfection in red for EGFP integration at the ACTB N-terminal site in the genome using PASTE according to embodiments of the present teachings;

FIG. 11 shows a diagram of the integration of EGFP and Gluc with various HEK3 targeting pegRNAs according to embodiments of the present teachings;

FIG. 12 shows a schematic diagram of the using φC31 as the integration enzyme, according to embodiments of the present teachings;

FIG. 13 shows a schematic diagram of multiplexing involving inserting multiple genes of interest in multiple loci using unique guide RNAs that incorporated exterior flanking attB sites according to embodiments of the present teachings;

FIG. 14A shows a diagram of the orthogonal editing with the right GT-EGFP according to embodiments of the present teachings;

FIG. 14B shows a diagram of the orthogonal editing with the right GA-mCherry according to embodiments of the present teachings;

FIG. 15A shows a fluorescent image of a multiplexing of ACTB-EGFP and NOLC1-mCherry according to embodiments of the present teachings

FIG. 15B shows a fluorescent image of a multiplexing of ACTB-EGFP and LAMNB1-mCherry according to embodiments of the present teachings;

FIG. 16A shows next generation sequencing results of 9×9 attP and attB central dinucleotide variants and their edit percentage wherein the orthogonality of attB/attP combinations for potential multiplexing applications is shown according to embodiments of the present teachings;

FIG. 16B shows an heatmap of 9×9 attP and attB central dinucleotide variants and their edit percentage according to embodiments of the present teachings;

FIG. 17 shows integration of SERPINA and CPS1 into Albumin loci using Albumin guide-pegRNA in HEK293FT cells according to embodiments of the present teachings;

FIG. 18 shows schematics for different nucleic acids for engineering T-cells according to embodiments of the present teachings;

FIG. 19 shows the editing efficiency for EGFP integration at the ACTB locus in primary T-cells according to embodiments of the present teachings;

FIG. 20 shows editing in TRAC locus in HEK293FT with different pegRNA according to embodiments of the present teachings;

FIG. 21A shows the attB integration at the ACTB locus using nicking guides 1 and 2 according to embodiments of the present teachings;

FIG. 21B shows the EGFP integration at the ACTB locus using nicking guides 1 and 2 according to embodiments of the present teachings;

FIG. 21C shows the EGFP integration at an ACTB site according to embodiments of the present teachings;

FIG. 22A shows PASTE editing in liver hepatocellular carcinoma cell line HEPG2 according to embodiments of the present teachings;

FIG. 22B shows PASTE editing of chronic myelogenous leukemia cell line K562 according to embodiments of the present teachings;

FIG. 23A shows the attB addition with targeting and non-targeting guides according to embodiments of the present teachings;

FIG. 23B shows the EGFP integration with targeting and non-targeting guides according to embodiments of the present teachings;

FIG. 23C shows the EGFP integration for mutagenized Bxb1 according to embodiments of the present teachings;

FIG. 24A shows a schematic of the design parameters for the pegRNA according to embodiments of the present teachings;

FIG. 24B shows a schematic of the design parameters for nicking guide RNA according to embodiments of the present teachings;

FIG. 25A shows the integration of EGFP at the ACTD locus with different PBS and RT lengths according to embodiments of the present teachings;

FIG. 25B shows the integration of EGFP at the LMNB1 loci with different PBS and RT lengths according to embodiments of the present teachings;

FIG. 25C shows the integration of EGFP at the NOLC1 loci with different PBS and RT lengths according to embodiments of the present teachings;

FIG. 25D shows the integration of EGFP at the GRSF1 locus with different PBS and RT lengths and different nicking guides according to embodiments of the present teachings;

FIG. 25E shows EGFP integration with mutant attP sites according to embodiments of the present teachings;

FIG. 25F shows the PASTE editing of an expanded panel of genes according to embodiments of the present teachings;

FIG. 26A shows the PASTE EGPF editing at the ACTB locus according to embodiments of the present teachings;

FIG. 26B shows the HITI EGPF editing at the ACTB locus according to embodiments of the present teachings;

FIG. 26C shows the comparison between the PASTE and HITI editing a panel of 14 genes according to embodiments of the present teachings;

FIG. 26D shows PASTE Bxb1 off-target integrations according to embodiments of the present teachings;

FIG. 26E shows PASTE Cas9 off-target integrations according to embodiments of the present teachings;

FIG. 26F shows the EGFP integration for gene inserts of different sizes according to embodiments of the present teachings;

FIG. 27A shows the orthogonality between selected sets of attB and attP sites according to embodiments of the present teachings;

FIG. 27B shows the orthogonality between selected sets of attB and attP sites according to embodiments of the present teachings;

FIG. 27C shows a schematic for the orthogonal PASTE editing using engineered di-nucleotide combinations according to embodiments of the present teachings;

FIG. 28A shows fluorescent images of the GFP tagging of ACTB and SUPT16H genes with PASTE according to embodiments of the present teachings;

FIG. 28B shows fluorescent images of the GFP tagging of NOLC1 and SRRM2 genes with PASTE according to embodiments of the present teachings;

FIG. 28C shows fluorescent images of the GFP tagging of LMNB1 and DEPDC4 genes with PASTE according to embodiments of the present teachings;

FIG. 28D shows the orthogonal gene integration at three endogenous sites with PASTE according to embodiments of the present teachings;

FIG. 28E shows the multiplexed insertion via one-plex, two-plex, and three-plex gene insertion at three endogenous sites via PASTE according to embodiments of the present teachings;

FIG. 28F shows fluorescent images of two single cells with multiplexed gene tagging of ACTB (EGFP) and NOLC1 (mCherry) using PASTE according to embodiments of the present teachings;

FIG. 28G shows fluorescent images two single cells with multiplexed gene tagging of ACTB (EGFP) and LMNB1 (mCherry) using PASTE according to embodiments of the present teachings;

FIG. 29A shows the prime editing efficiency of Bxb1 attB site insertion at the ACTB locus according to embodiments of the present teachings;

FIG. 29B shows the prime editing efficiency at inserting Bxb1 attB sites of different lengths at the ACTB locus according to embodiments of the present teachings;

FIG. 29C shows the prime editing efficiency of inserting attB sequences from different integrases, wherein both orientations of landing sites are profiled (F, forward; and R, reverse) according to embodiments of the present teachings;

FIG. 29D shows the prime editing efficiency of inserting attB sequences from Bxb1 integrase and Cre recombinase, wherein both orientations of landing sites are profiled (F, forward; and R, reverse) according to embodiments of the present teachings;

FIG. 29E shows a schematic of PASTE insertion at the ACTB locus showing guide and target sequences according to embodiments of the present teachings. FIG. 29E discloses SEQ ID NOS 428-431, respectively, in order of appearance;

FIG. 29F shows a comparison of PASTE integration efficiency of GFP with a panel of integrases targeting the 5′ end of the ACTB locus, wherein both orientations of landing sites are profiled (F, forward; and R, reverse) according to embodiments of the present teachings;

FIG. 29G shows a comparison of GFP cargo integration efficiency between Bxb1 integrases and Cre recombinase according to embodiments of the present teachings;

FIG. 29H shows the dependence of PASTE editing activity on different prime and integrase components according to embodiments of the present teachings;

FIG. 29I shows a titration of a single vector PASTE system (SpCas9-RT-P2A-Bxb1) on integrase efficiency according to embodiments of the present teachings;

FIG. 29J shows the effect of cargo size on PASTE insertion efficiency at the endogenous ACTB target according to embodiments of the present teachings;

FIG. 29K shows a gel electrophoresis showing complete insertion by PASTE for multiple cargo sizes according to embodiments of the present teachings;

FIG. 30A shows a schematic of PASTE integration, including resulting attR and attL sites that are generated and PCR primers for assaying the integration junctions according to embodiments of the present teachings;

FIG. 30B shows a PCR and gel electrophoresis readout of left integration junction from PASTE insertion of GFP at the ACTB locus, wherein the insertion is analyzed for in-frame and out-of-frame GFP integration experiments as well as for a no prime control and expected sizes of the PCR fragments are shown using the primers shown in the schematic in subpanel FIG. 30A according to embodiments of the present teachings;

FIG. 30C shows a PCR and gel electrophoresis readout of right integration junction from PASTE insertion of GFP at the ACTB locus, wherein the insertion is analyzed for in-frame and out-of-frame GFP integration experiments as well as for a no prime control and the expected sizes of the PCR fragments are shown using the primers shown in the schematic in subpanel FIG. 30A according to embodiments of the present teachings;

FIG. 30D shows a Sanger sequencing shown for the right integration junction for an in-frame fusion of GFP via PASTE to the N-terminus of ACTB according to embodiments of the present teachings;

FIG. 30E shows a Sanger sequencing shown for the left integration junction for an in-frame fusion of GFP via PASTE to the N-terminus of ACTB according to embodiments of the present teachings;

FIG. 31A shows a schematic of various parameters that affect PASTE integration of ˜1 kb GFP insert, wherein on the pegRNA, the PBS, RT, and attB lengths can alter the efficiency of attB insertion, and nicking guide selection also affects overall gene integration efficiency according to embodiments of the present teachings;

FIG. 31B shows the impact of PBS and RT length on PASTE integration of GFP at the ACTB locus according to embodiments of the present teachings;

FIG. 31C shows the impact of PBS and RT length on PASTE integration of GFP at the LMNB1 locus according to embodiments of the present teachings;

FIG. 31D shows the impact of attB length on PASTE integration of GFP at the ACTB locus according to embodiments of the present teachings;

FIG. 31E shows the impact of attB length on PASTE integration of GFP at the LMNB1 locus according to embodiments of the present teachings;

FIG. 31F shows the impact of attB length on PASTE integration of GFP at the NOLC1 locus according to embodiments of the present teachings;

FIG. 31G shows the impact of minimal PBS, RT, and attB lengths on PASTE integration efficiency of GFP at the ACTB locus according to embodiments of the present teachings;

FIG. 31H shows the impact of minimal PBS, RT, and attB lengths on PASTE integration efficiency of GFP at the LMNB1 locus according to embodiments of the present teachings;

FIG. 31I shows the PASTE integration of GFP at the LMNB1 locus in the presence and absence of nicking guide, prime, and Bxb1 with a minimally compact pegRNA containing a 38 bp attB compared to a longer pegRNA design according to embodiments of the present teachings;

FIG. 32A shows the PASTE insertion efficiency at ACTB and LMNB1 loci with two different nicking guide designs according to embodiments of the present teachings;

FIG. 32B shows the PASTE editing efficiency at ACTB and LMNB1 with target and non-targeting spacers and matched pegRNAs with and without Bxb1 expression according to embodiments of the present teachings;

FIG. 33A shows the PASTE integration of GFP at the ACTB locus with different Bxb1 catalytic mutants according to embodiments of the present teachings;

FIG. 33B shows the PASTE integration of GFP at the ACTB locus with different RT catalytic mutants according to embodiments of the present teachings;

FIG. 34A shows the GFP integration by PASTE at a panel of endogenous genomic loci according to embodiments of the present teachings;

FIG. 34B shows the integration of a panel of different gene cargo at ACTB locus via PASTE according to embodiments of the present teachings;

FIG. 34C shows the integration efficiency of therapeutically relevant genes at the ACTB locus according to embodiments of the present teachings;

FIG. 34D shows the endogenous protein tagging with GFP via PASTE by in-frame endogenous gene tagging at the ACTB loci and SRRM2 loci according to embodiments of the present teachings;

FIG. 34E shows the endogenous protein tagging with GFP via PASTE by in-frame endogenous gene tagging at the NOLC1 loci and LMNB1 loci according to embodiments of the present teachings;

FIG. 35 shows the integration of a panel of different gene cargo at LMNB1 locus via PASTE according to embodiments of the present teachings;

FIG. 36A shows the PASTE integration efficiency for all 16 central dinucleotide attB/attP sequence pairs with a 5 kb GFP template at the ACTB locus according to embodiments of the present teachings;

FIG. 36B shows a schematic of the pooled attB/attP dinucleotide orthogonality assay, wherein each attB dinucleotide sequence is co-transfected with a barcoded pool of all 16 attP dinucleotide sequences and Bxb1 integrase, relative integration efficiencies are determined by next generation sequencing of barcodes, and all 16 attB dinucleotides are profiled in an arrayed format with attP pools according to embodiments of the present teachings;

FIG. 36C shows the relative insertion preferences for all possible attB/attP dinucleotide pairs determined by the pooled orthogonality assay according to embodiments of the present teachings;

FIG. 36D shows the orthogonality of top 4 attB/attP dinucleotide pairs evaluated for GFP integration with PASTE at the ACTB locus according to embodiments of the present teachings;

FIG. 37 shows the orthogonality of Bxb1 dinucleotides as measured by a pooled reporter assay, wherein each web logo motif shows the relative integration of different attP sequences in a pool at a denoted attB sequence with the listed dinucleotide according to embodiments of the present teachings;

FIG. 38A shows a schematic of multiplexed integration of different cargo sets at specific genomic loci, wherein three fluorescent cargos (GFP, mCherry, and YFP) are inserted orthogonally at three different loci (ACTB, LMNB1, NOLC1) for in-frame gene tagging according to embodiments of the present teachings;

FIG. 38B shows the efficiency of multiplexed PASTE insertion of combinations of fluorophores at ACTB, LMNB1, and NOLC1 loci according to embodiments of the present teachings;

FIG. 39A shows the GFP integration efficiency at a panel of genomic loci by PASTE compared to insertion rates by homology-independent targeted integration (HITI) according to embodiments of the present teachings;

FIG. 39B shows a comparison of unintended indel generation by PASTE and HITI at the ACTB and LMNB1 target sites, wherein the on-target EGFP integration rate observed compared to unintended indels is shown according to embodiments of the present teachings;

FIG. 39C shows the integration of a GFP template by PASTE at the ACTB locus compared to homology-directed repair (HDR) at the same target, wherein the quantification is by single-cell clone counting, wherein targeting and non-targeting guides were used for HDR insertion, and wherein for PASTE targeting and non-targeting refers to the presence or absence of the SpCas9-RT protein respectively according to embodiments of the present teachings;

FIG. 39D shows the comparison of unintended indel generation by PASTE and HDR based EGFP insertion at the ACTB target site, wherein the average indel rate measured across all single-cell clones generated is showed according to embodiments of the present teachings;

FIG. 39E shows a schematic for Bxb1 and Cas9 off-target identification and a detection assay according to embodiments of the present teachings;

FIG. 39F shows the GFP integration activity at predicted Bxb1 off-target sites in the human genome according to embodiments of the present teachings;

FIG. 39G shows the GFP integrations activity at predicted PASTE ACTB Cas9 guide off target sites according to embodiments of the present teachings;

FIG. 39H shows the GFP integration activity at predicted HITI ACTB Cas9 guide off-target sites according to embodiments of the present teachings;

FIG. 39I shows a schematic of next-generation sequencing method to assay genome-wide off-target integration sites by PASTE according to embodiments of the present teachings;

FIG. 39J shows the alignment of reads at the on-target ACTB site using a genome-wide integration assay, wherein expected on-target integration outcomes are shown according to embodiments of the present teachings;

FIG. 39K shows the analysis of on-target and off-target integration events across 3 single-cell clones for PASTE and 3 single-cell clones for no prime condition according to embodiments of the present teachings;

FIG. 39L shows a Manhattan plot of integration events for a representative single-cell clone with PASTE editing, wherein the on-target site is at the ACTB gene on chromosome 7 according to embodiments of the present teachings;

FIG. 40A shows a comparison of indel rates generated by PASTE and HITI mediated insertion of EGFP at the ACTB and LMNB1 loci in HepG2 cells according to embodiments of the present teachings;

FIG. 40B shows the validation of ddPCR assays for detecting editing at predicted Bxb1 offtarget sites using synthetic amplicons according to embodiments of the present teachings;

FIG. 40C shows the validation of ddPCR assays for detecting editing at predicted PASTE ACTB Cas9 guide off-target sites using synthetic amplicons according to embodiments of the present teachings;

FIG. 40D shows the validation of ddPCR assays for detecting editing at predicted HITI ACTB Cas9 guide off-target sites using synthetic amplicons according to embodiments of the present teachings;

FIG. 41A shows a number of significant differentially regulated genes in HEK293FT cells expressing Bxb1 integrase, PASTE targeting ACTB integration of EGFP, or Prime editing targeting ACTB for EGFP insertion without Bxb1 expression according to embodiments of the present teachings;

FIG. 41B shows Volcano plots depicting the fold expression change of sequenced mRNAs versus significance (p-value), wherein each dot represents a unique mRNA transcript and significant transcripts are shaded according to either upregulation (red) or downregulation (blue), and wherein fold expression change is measured against ACTB-targeting guide-only expression (including cargo) according to embodiments of the present teachings;

FIG. 41C shows top significantly upregulated and downregulated genes for Bxb1-only conditions, wherein genes are shown with their corresponding Z-scores of counts per million (cpm) for Bxb1 only expression, GFP-only expression, PASTE targeting ACTB for EGFP insertion, Prime targeting ACTB for EGFP expression without Bxb1, and guide/cargo only according to embodiments of the present teachings;

FIG. 42A shows a schematic of PASTE performance in the presence of cell cycle inhibition, wherein cells are transfected with plasmids for insertion with PASTE or Cas9-induced HDR and treated with aphidicolin to arrest cell division, and wherein the efficiency of PASTE and HDR are read out with ddPCR or amplicon sequencing respectively according to embodiments of the present teachings;

FIG. 42B shows the editing efficiency of single mutations by HDR at EMX1 locus with two Cas9 guides in the presence or absence of cell division read out with amplicon sequencing according to embodiments of the present teachings;

FIG. 42C shows the integration efficiency of various sized GFP inserts up to 13.3 kb at the ACTB locus with PASTE in the presence or absence of cell division according to embodiments of the present teachings;

FIG. 42D shows the PASTE editing efficiency with two vector (PE2 and Bxb1) and single vector (PE2-P2A-Bxb1) designs in K562 cells according to embodiments of the present teachings;

FIG. 42E shows the PASTE editing efficiency with single vector (PE2-P2A-Bxb1) designs in primary human T cells according to embodiments of the present teachings;

FIG. 42F shows the integration efficiency of therapeutically relevant genes at the ACTB locus according to embodiments of the present teachings;

FIG. 42G shows a schematic of protein production assay for PASTE-integrated transgene, wherein SERPINA1 and CPS1 transgenes are tagged with HIBIT luciferase for readout with both ddPCR and luminescence according to embodiments of the present teachings;

FIG. 42H shows the integration efficiency of SERPINA1 and CPS1 transgenes in HEK293FT cells at the ACTB locus according to embodiments of the present teachings;

FIG. 42I shows the integration efficiency of SERPINA1 and CPS1 transgenes in HepG2 cells at the ACTB locus according to embodiments of the present teachings;

FIG. 42J shows the intracellular levels of SERPINA1-HIBIT and CPS1-HIBIT in HepG2 cells according to embodiments of the present teachings;

FIG. 42K shows the secreted levels of SERPINA1-HIBIT and CPS1-HIBIT in HepG2 cells according to embodiments of the present teachings;

FIG. 43A shows the HDR mediated editing of the EMX1 locus that is significantly diminished in non-dividing HEK293FT cells blocked by 5 μM aphidicolin treatment according to embodiments of the present teachings;

FIG. 43B shows the effect of insert minicircle DNA amount on PASTE-mediated insertion at the ACTB locus in dividing and nondividing HEK293FT cells blocked by 5 μM aphidicolin treatment according to embodiments of the present teachings;

FIG. 43C shows the PASTE integration of GFP at the ACTB locus with the GFP template delivered via AAV, showing dose dependence of integration efficiency according to embodiments of the present teachings;

FIG. 44A shows the PASTE integration activity at three endogenous loci comparing the normal PASTE SV40 NLS to a c-Myc NLS/variable bi-partite SV40 NLS design according to embodiments of the present teachings;

FIG. 44B shows the PASTE integration activity at the ACTB locus with different GFP minicircle template amounts comparing the normal PASTE SV40 NLS to a c-Myc NLS/variable bi-partite SV40 NLS design according to embodiments of the present teachings;

FIG. 45 shows the improvement of the PASTE editing activity using a puromycin growth selection marker according to embodiments of the present teachings;

FIG. 46A shows the integration of SERPINA1 and CPS1 genes that are HIBIT tagged as measured by a protein expression luciferase assay according to embodiments of the present teachings;

FIG. 46B shows the integration of SERPINA1 and CPS1 genes that are HIBIT tagged as measured by a protein expression luciferase assay normalized to a standardized HIBIT ladder, enabling accurate quantification of protein levels according to embodiments of the present teachings;

FIG. 47A shows optimization of PASTE constructs with a panel of linkers and reverse transcriptase (RT) modifications for EGFP integration at the ACTB locus, according to embodiments of the present teachings;

FIG. 47B shows the effect of cargo size on PASTE insertion efficiency at the endogenous ACTB target. Cargos were transfected with fixed molar amounts, according to embodiments of the present teachings;

FIG. 48A shows prime editing efficiency for the insertion of different length BxbINT AttB sites at ACTB, according to embodiments of the present teachings;

FIG. 48B shows prime editing efficiency for the insertion of a BxbINT AttB site at ACTB with targeting and non-targeting guides, according to embodiments of the present teachings;

FIG. 48C shows prime editing efficiency for the insertion of different integrases' (Bxb1, Tp9, and Bt1) AttB sites at ACTB. Both orientations of landing sites are profiled (F, forward; R, reverse), according to embodiments of the present teachings;

FIG. 48D shows PASTE editing efficiency for the insertion of EGFP at ACTB with and without a nicking guide, according to embodiments of the present teachings; and

FIG. 49A shows optimization of PASTE editing by dosage titration and protein optimization. PASTE integration efficiency of EGFP at ACTB measured with different doses of a single-vector delivery of components.

FIG. 49B PASTE integration efficiency of EGFP at ACTB measured with different ratios of a single-vector delivery of components to the EGFP template vector.

FIG. 49C PASTE integration efficiency of EGFP at ACTB with different RT domain fusions.

FIG. 49D PASTE integration efficiency of EGFP at ACTB with different RT domain fusions and linkers.

FIG. 49E PASTE integration efficiency of EGFP at ACTB with mutant RT domains.

FIG. 49F PASTE integration efficiency of EGFP at ACTB with mutated BxbINT domains.

FIG. 50A Insertion templates delivered via AAV transduction. PASTE editing machinery was delivered via transfection, and templates were co-delivered via AAV dosing at levels indicated.

FIG. 50B Schematic of AdV delivery of the complete PASTE system with three viral vectors.

FIG. 50C Integration efficiency of AdV delivery of integrase, guides, and cargo in HEK293FT and HepG2 cells. BxbINT and guide RNAs or cargo were delivered either via plasmid transfection (P1), AdV transduction (AdV), or omitted (−). SpCas9-RT was only delivered as plasmid or omitted.

FIG. 50D AdV delivery of all PASTE components in HEK293FT and HepG2 cells.

FIG. 50E Schematic of mRNA and synthetic guide delivery of PASTE components.

FIG. 50F Delivery of PASTE system components with mRNA and synthetic guides, paired with either AdV or plasmid cargo.

FIG. 50G Delivery of circular mRNA with synthetic guides and either AdV or plasmid cargo.

FIG. 50H PASTE editing efficiency with single vector designs in primary human T cells.

FIG. 50I PASTE editing efficiency with single vector designs in primary human hepatocytes.

FIG. 51A PASTE editing efficiency at the LMNB1 locus with 130 bp and 385 bp deletions of the first exon of LMNB1 with combined insertion of an attB sequence.

FIG. 51B PASTE editing efficiency with a 130 bp deletion of the first exon of LMNB1 with a combined insertion of a 967 bp cargo using the PASTE system.

### DETAILED DESCRIPTION

It will be appreciated that for clarity, the following discussion will describe various aspects of embodiments of the applicant's teachings. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular feature, structures or characteristics may be combined in any suitable manner, as would be apparent to a person skilled in the art from this disclosure, in one or more embodiments.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an,” and “the” include both singular and plural referents unless the context clearly dictates otherwise. Thus, for example, reference to “a cell” includes a plurality of such cells.

As used herein, the term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, the term “about” or “approximately” refers to a measurable value such as a parameter, an amount, a temporal duration, and the like, are meant to encompass variations of and from the specified value, such as variations of +/−10% or less, +/−5% or less, +/−1% or less, +1-0.5% or less, and +1-0.1% or less of and from the specified value, insofar such variations are appropriate to perform in the disclosure. It is to be understood that the value to which the modifier “about” or “approximately” refers is itself also specifically, and preferably, disclosed.

It is noted that all publications and references cited herein are expressly incorporated herein by reference in their entirety. The publications discussed herein are provided solely for their disclosure prior to the filing date of the present application. Nothing herein is to be construed as an admission that the present disclosure is not entitled to antedate such publication. Further, the dates of publication provided may be different from the actual publication dates which may need to be independently confirmed.

### Overview

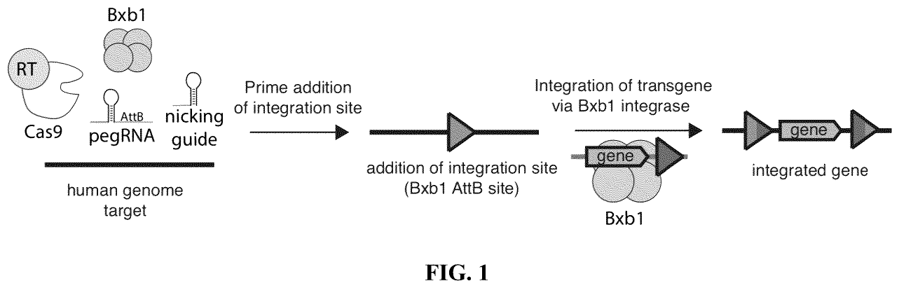

The embodiments disclosed herein provide non-naturally occurring or engineered systems, methods, and compositions for site-specific genetic engineering using Programmable Addition via Site-Specific Targeting Elements (PASTE). A schematic diagram illustrating the concept of PASTE is shown in FIG. 1. As discussed in more details below, PASTE comprises the addition of an integration site into a target genome followed by the insertion of one or more genes of interest or one or more nucleic acid sequences of interest at the site. This process can be done as one or more reactions in a cell. The addition of the integration site into the target genome is done using gene editing technologies that include for example, without limitation, prime editing, recombinant adeno-associated virus (rAAV)-mediated nucleic acid integration, transcription activator-like effector nucleases (TALENS), and zinc finger nucleases (ZFNs). The integration of the transgene at the integration site is done using integrase technologies that include for example, without limitation, integrases, recombinases and reverse transcriptases. The necessary components for the site-specific genetic engineering disclosed herein comprise at least one or more nucleases, one or more gRNA, one or more integration enzymes, and one or more sequences that are complementary or associated to the integration site and linked to the one or more genes of interest or one or more nucleic acid sequences of interest to be inserted into the cell genome.

An advantage of the non-naturally occurring or engineered systems, methods, and compositions for site-specific genetic engineering disclosed herein is programmable insertion of large elements without reliance on DNA damage responses.

Another advantage of the non-naturally occurring or engineered systems, methods, and compositions for site-specific genetic engineering disclosed herein is facile multiplexing, enabling programmable insertion at multiple sites.

Another advantage of the non-naturally occurring or engineered systems, methods, and compositions for site-specific genetic engineering disclosed herein is scalable production and delivery through minicircle templates.

### Prime Editing

The present disclosure provides non-naturally occurring or engineered systems, methods, and compositions for site-specific genetic engineering using gene editing technologies, such as prime editing, to add an integration site into a target genome. Prime editing will be discussed in more details below.

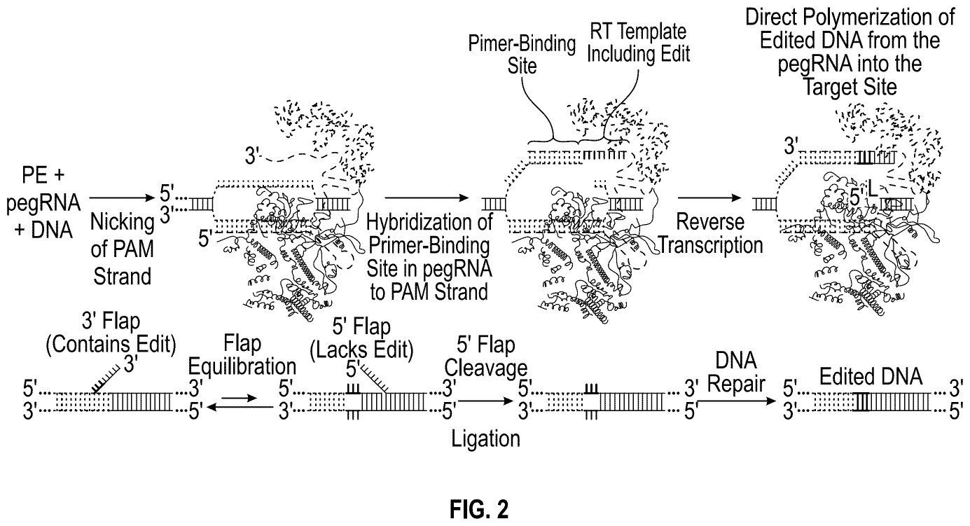

Prime editing is a versatile and precise genome editing method that directly writes new genetic information into a specified DNA site. A schematic diagram illustrating the concept of prime editing is shown in FIG. 2. See, Anzalone, A. V., et al. “Search-and-replace genome editing without double-strand breaks or donor DNA,” Nature 576, 149-157 (2019). Prime editing uses a catalytically-impaired Cas9 endonuclease that is fused to an engineered reverse transcriptase (RT) and programmed with a prime-editing guide RNA (pegRNA). The skilled person in the art would appreciate that the pegRNA both specifies the target site and encodes the desired edit. The catalytically-impaired Cas9 endonuclease also comprises a Cas9 nickase that is fused to the reverse transcriptase. During genetic editing, the Cas9 nickase part of the protein is guided to the DNA target site by the pegRNA. The reverse transcriptase domain then uses the pegRNA to template reverse transcription of the desired edit, directly polymerizing DNA onto the nicked target DNA strand. The edited DNA strand replaces the original DNA strand, creating a heteroduplex containing one edited strand and one unedited strand. Afterward, the prime editor (PE) guides resolution of the heteroduplex to favor copying the edit onto the unedited strand, completing the process.

The prime editors refer to a Moloney Murine Leukemia Virus (M-MLV) reverse transcriptase (RT) fused to a Cas9 H840A nickase. Fusing the RT to the C-terminus of the Cas9 nickase may result in higher editing efficiency. Such a complex is called PE1. The Cas9(H840A) can also be linked to a non-M-MLV reverse transcriptase such as a AMV-RT or XRT (Cas9(H840A)-AMV-RT or XRT). In some embodiments, Cas 9(H840A) can be replaced with Cas12a/b or Cas9(D10A). A Cas9 (wild type), Cas9(H840A), Cas9(D10A) or Cas 12a/b nickase fused to a pentamutant of M-MLV RT (D200N/L603W/T330P/T306K/20 W313F), having up to about 45-fold higher efficiency is called PE2. In some embodiments, the M-MLV RT comprise one or more of the mutations: Y8H, P51L, S56A, S67R, E69K, V129P, L139P, T197A, H204R, V223H, T246E, N249D, E286R, Q2911, E302K, E302R, F309N, M320L, P330E, L435G, L435R, N454K, D524A, D524G, D524N, E562Q, D583N, H594Q, E607K, D653N, and L671P. In some embodiments, the reverse transcriptase can also be a wild-type or modified transcription xenopolymerase (RTX), avian myeloblastosis virus reverse transcriptase (AMV-RT), Feline Immunodeficiency Virus reverse transcriptase (FIV-RT), FeLV-RT (Feline leukemia virus reverse transcriptase), HIV-RT (Human Immunodeficiency Virus reverse transcriptase), or Eubacterium rectale maturase RT (MarathonRT). PE3 involves nicking the non-edited strand, potentially causing the cell to remake that strand using the edited strand as the template to induce HR. The nicking of the non-edited strand can involve the use of a nicking guide RNA (ngRNA).

Nicking the non-edited strand can increase editing efficiency. For example, nicking the non-edited strand can increase editing efficiency by about 1.1 fold, about 1.3 fold, about 1.5 fold, about 1.7 fold, about 1.9 fold, about 2.1 fold, about 2.3 fold, about 2.5 fold, about 2.7 fold, about 2.9 fold, about 3.1 fold, about 3.3 fold, about 3.5 fold, about 3.7 fold, about 3.9 fold, 4.1 fold, about 4.3 fold, about 4.5 fold, about 4.7 fold, about 4.9 fold, or any range that is formed from any two of those values as endpoints.

Although the optimal nicking position varies depending on the genomic site, nicks positioned 3′ of the edit about 40-90 bp from the pegRNA-induced nick can generally increase editing efficiency without excess indel formation. The prime editing practice allows starting with non-edited strand nicks about 50 bp from the pegRNA-mediated nick, and testing alternative nick locations if indel frequencies exceed acceptable levels.

As used herein, the term “guide RNA” (gRNA) and the like refer to a RNA that guide the insertion or deletion of one or more genes of interest or one or more nucleic acid sequences of interest into a target genome. The gRNA can also refer to a prime editing guide RNA (pegRNA), a nicking guide RNA (ngRNA), and a single guide RNA (sgRNA). In some embodiments, the term “gRNA molecule” refers to a nucleic acid encoding a gRNA. In some embodiments, the gRNA molecule is naturally occurring. In some embodiments, a gRNA molecule is non-naturally occurring. In some embodiments, a gRNA molecule is a synthetic gRNA molecule. A gRNA can target a nuclease or a nickase such as Cas9, Cas 12a/b, Cas9 (H840A) or Cas9 (D10A) molecule to a target nucleic acid or sequence in a genome. In some embodiments, the gRNA can bind to a DNA nickase bound to a reverse transcriptase domain. A “modified gRNA,” as used herein, refers to a gRNA molecule that has an improved half-life after being introduced into a cell as compared to a non-modified gRNA molecule after being introduced into a cell. In some embodiments, the guide RNA can facilitate the addition of the insertion site sequence for recognition by integrases, transposases, or recombinases.

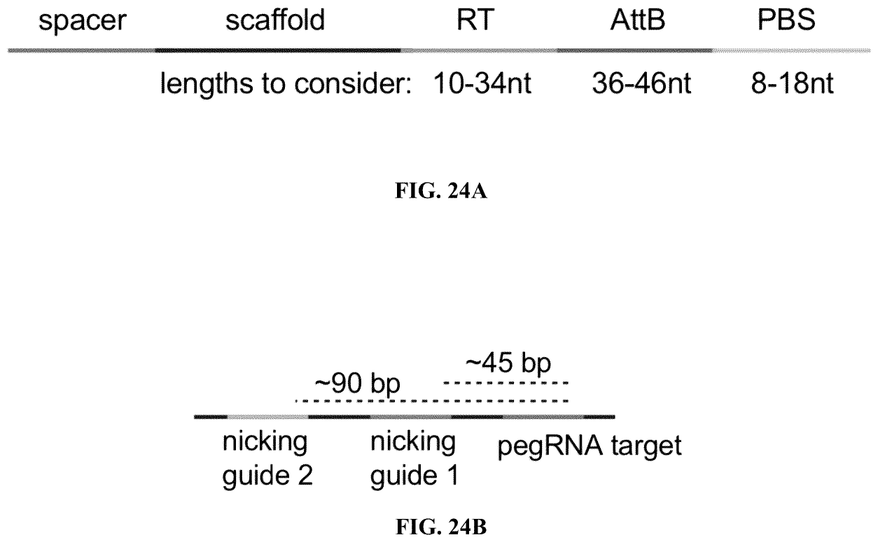

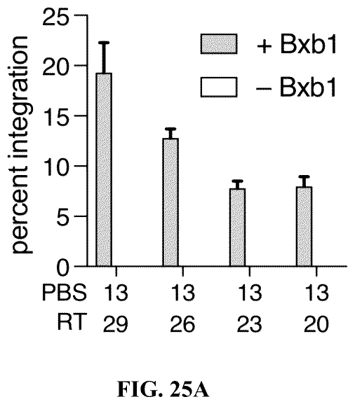

As used herein, the term “prime-editing guide RNA” (pegRNA) and the like refer to an extended single guide RNA (sgRNA) comprising a primer binding site (PBS), a reverse transcriptase (RT) template sequence, and an integration site sequence that can be recognized by recombinases, integrases, or transposases. Exemplary design parameters for pegRNA are shown in FIG. 24A. For example, the PBS can have a length of at least about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, or more nt. For example, the PBS can have a length of about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, or any range that is formed from any two of those values as endpoints. For example, the RT template sequence can have a length of at least about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, 50 nt, or more nt. For example, the RT template sequence can have a length of about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, 50 nt, or any range that is formed from any two of those values as endpoints.

During genome editing, the primer binding site allows the 3′ end of the nicked DNA strand to hybridize to the pegRNA, while the RT template serves as a template for the synthesis of edited genetic information. The pegRNA is capable for instance, without limitation, of (i) identifying the target nucleotide sequence to be edited and (ii) encoding new genetic information that replaces the targeted sequence. In some embodiments, the pegRNA is capable of (i) identifying the target nucleotide sequence to be edited and (ii) encoding an integration site that replaces the targeted sequence.

As used herein, the term “nicking guide RNA” (ngRNA) and the like refer to an RNA sequence that can nick a strand such as an edited strand and a non-edited strand. Exemplary design parameters for ngRNA are shown in FIG. 24B. The ngRNA can induce nicks at about 1 or more nt away from the site of the gRNA-induced nick. For example, the ngRNA can nick at least at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, or more nt away from the site of the gRNA induced nick. In some embodiments, the ngRNA comprises SEQ ID NO: 75 with guide sequence SEQ ID NO: 74. As used herein, the terms “reverse transcriptase” and “reverse transcriptase domain” refer to an enzyme or an enzymatically active domain that can reverse a RNA transcribe into a complementary DNA. The reverse transcriptase or reverse transcriptase domain is a RNA dependent DNA polymerase. Such reverse transcriptase domains encompass, but are not limited, to a M-MLV reverse transcriptase, or a modified reverse transcriptase such as, without limitation, Superscript® reverse transcriptase (Invitrogen; Carlsbad, California), Superscript® VILO™ cDNA synthesis (Invitrogen; Carlsbad, California), RTX, AMV-RT, and Quantiscript Reverse Transcriptase (Qiagen, Hilden, Germany).

The pegRNA-PE complex disclosed herein recognizes the target site in the genome and the Cas9 for example nicks a protospacer adjacent motif (PAM) strand. The primer binding site (PBS) in the pegRNA hybridizes to the PAM strand. The RT template operably linked to the PBS, containing the edit sequence, directs the reverse transcription of the RT template to DNA into the target site. Equilibration between the edited 3′ flap and the unedited 5′ flap, cellular 5′ flap cleavage and ligation, and DNA repair results in stably edited DNA. To optimize base editing, a Cas9 nickase can be used to nick the non-edited strand, thereby directing DNA repair to that strand, using the edited strand as a template.

### Integrase Technologies

The present disclosure provides non-naturally occurring or engineered systems, methods, and compositions for site-specific genetic engineering using integrase technologies. Integrase technologies will be discussed in more details below.

The integrase technologies used herein comprise proteins or nucleic acids encoding the proteins that direct integration of a gene of interest or nucleic acid sequence of interest into an integration site via a nuclease such as a prime editing nuclease. The protein directing the integration can be an enzyme such as integration enzyme. The integration enzyme can be an integrase that incorporates the genome or nucleic acid of interest into the cell genome at the integration site by integration. The integration enzyme can be a recombinase that incorporates the genome or nucleic acid of interest into the cell genome at the integration site by recombination. The integration enzyme can be a reverse transcriptase that incorporates the genome or nucleic acid of interest into the cell genome at the integration site by reverse transcription. The integration enzyme can be a retrotransposase that incorporates the genome or nucleic acid of interest into the cell genome at the integration site by retrotransposition.

As used herein, the term “integration enzyme” refers to an enzyme or protein used to integrate a gene of interest or nucleic acid sequence of interest into a desired location or at the integration site, in the genome of a cell, in a single reaction or multiple reactions. Example of integration enzymes include for example, without limitation, Cre, Dre, Vika, Bxb1, φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, and retrotransposases encoded by R2, L1, Tol2 Tcl, Tc3, Mariner (Himar 1), Mariner (mos 1), and Minos. In some embodiments, the term “integration enzyme” refers to a nucleic acid (DNA or RNA) encoding the above-mentioned enzymes. In some embodiments, the Cre recombinase is expressed from a Cre recombinase expression plasmid (SEQ ID NO: 71).

Mammalian expression plasmids can be found in Table 1 below.

TABLE 1

| Name | Full Description | SEQ ID NOS: |
|---|---|---|
| PE2-Bxb1 Single | pCMV-PE2- | (SEQ ID NO: 381) |
| Vector | P2A-Bxb1 | |
| PE2 prime editor | pCMV-PE2/ | (SEQ ID NO: 382) |
| | Addgene | |
| | #132775 | |
| PE2*-Bxb1 Single | New NLS | (SEQ ID NO: 383) |
| Vector | pCMV-PE2- | |
| | P2A-Bxb1 | |
| PASTEv3 | pCMV-SpCas9- | (SEQ ID NO: 384) |
| | XTEN-RT | |
| | (1-478)-Sto7d- | |
| | GGGGS- | |
| | BxbINT | |
| ACTB pegRNA | ACTB | (SEQ ID NO: 385) |
| | N-term PBS 13 | |
| | RT 29 attB 46 | |
| | pegRNA | |
| ACTB Nicking +48 | ACTB | (SEQ ID NO: 386) |
| | tN-erm Nicking | |
| | guide 1 +48 | |
| | guide | |
| Bxb1 integrase | pCAG-NLS- | (SEQ ID NO: 387) |
| | HA- | |
| | Bxblintegrase/ | |
| | Addgene | |
| | #51271 | |
| TP901-1 Integrase | TP901-1 | (SEQ ID NO: 388) |
| | Integrase | |
| PhiBT Integrase | PhiBT Integrase | (SEQ ID NO: 389) |
| HDR sgRNA guide | Minicircle U6- | (SEQ ID NO: 390) |
| | sgRNA EFS- | |
| | SpCas9 | |
| HDR EGFP cargo | Cas9 HDR | (SEQ ID NO: 391) |
| | template site | |
| | with EGFP | |
| AAV helper | PDF6 AAV | (SEQ ID NO: 392) |
| plasmid | helper plasmid | |
| AAV EGFP donor | GFP AAV donor | (SEQ ID NO: 393) |
| | plasmid | |
| AAV2/8 | AAV2/8 capsid | (SEQ ID NO: 394) |
| | protein | |

Minicircle cargo gene maps can be found in Table 2 below.

TABLE 2

| | Full | |
|---|---|---|
| Name | Description | SEQ ID NOS: |
| Cargo EGFP | Parent | (SEQ ID NO: 76) |
| | minicircle | |
| | plasmid - | |
| | Cargo EGFP | |
| | with attP Bxb1 | |
| | site | |
| Cargo | Cargo EGFP | (SEQ ID NO: 395) |
| EGFP | with attP Bxb1 | |
| post | site - post | |
| cleavage | minicircle | |
| | cleavage | |
| Cargo | Parent | (SEQ ID NO: 396) |
| EGFP | minicircle | |
| for | plasmid - | |
| fusion | Cargo EGFP | |
| | with attP | |
| | Bxbl site for | |
| | fusion | |
| mCherry | Cargo | (SEQ ID NO: 397) |
| Cargo post | mCherry | |
| cleavage | with attP | |
| | Bxb1 site - | |
| | post | |
| | minicircle | |
| | cleavage | |
| YFP | Cargo YFP | (SEQ ID NO: 398) |
| Cargo | with attP Bxb1 | |
| post | site - post | |
| cleavage | minicircle | |
| | cleavage | |
| SERPINA1 | Cargo | (SEQ ID NO: 399) |
| Cargo | SERPINA1 | |
| post | with attP | |
| cleavage | Bxbl site - | |
| | post | |
| | minicircle | |
| | cleavage | |
| CPS1 | Cargo CPS1 | (SEQ ID NO: 400) |
| Cargo | with attP Bxb1 | |
| post | site - post | |
| cleavage | minicircle | |
| | cleavage | |
| CFTR Cargo | Parent | (SEQ ID NO: 401) |
| | minicircle | |
| | plasmid - | |
| | Cargo CFTR | |
| | with attP Bxb1 | |
| | site | |
| NYESO | Cargo | (SEQ ID NO: 402) |
| TCR Cargo | NYESO | |
| post | TCR with | |
| cleavage | attP Bxb1 | |
| | site - post | |
| | minicircle | |
| | cleavage | |

In some embodiments, the serine integrase φC31 from φC31 phage is use as integration enzyme. The integrase φC31 in combination with a pegRNA can be used to insert the pseudo attP integration site (SEQ ID NO: 78). A DNA minicircle containing a gene or nucleic acid of interest and attB (SEQ ID NO: 3) site can be used to integrate the gene or nucleic acid of interest into the genome of a cell. This integration can be aided by a co-transfection of an expression vector having the φC31 integrase.

As used herein, the term “integrase” refers to a bacteriophage derived integrase, including wild-type integrase and any of a variety of mutant or modified integrases. As used herein, the term “integrase complex” may refer to a complex comprising integrase and integration host factor (IF). As used herein, the term “integrase complex” and the like may also refer to a complex comprising an integrase, an integration host factor, and a bacteriophage X-derived excisionase (Xis).

As used herein, the term “recombinase” and the like refer to a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3, β-six, CinH, ParA, γδ, Bxb1, φC31, TP901, TG1, φBT1, R1, R2, R3, R4, R5, φRV1, φFC1, MR11, A118, U153, and gp29. Examples of serine recombinases also include, without limitation, recombinases Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, and BxZ2 from Mycobacterial phages. Examples of tyrosine recombinases include, without limitation, Cre, FLP, R, Lambda, HK101, HK022, and pSAM2. The serine and tyrosine recombinase names stem from the conserved nucleophilic amino acid residue that the recombinase uses to attack the DNA and which becomes covalently linked to the DNA during strand exchange.

Recombinases have numerous applications, including the creation of gene knockouts/knock-ins and gene therapy applications. See, e.g., Brown et al., “Serine recombinases as tools for genome engineering.”Methods, 2011; 53(4):372-9; Hirano et al., “Site-specific recombinases as tools for heterologous gene integration.” Appl. Microbiol. Biotechnol. 2011; 92(2):227-39; Chavez and Calos, “Therapeutic applications of the ΦC31 integrase system.” Curr. Gene Ther. 2011; 11(5):375-81; Turan and Bode, “Site-specific recombinases: from tag-and-target- to tag-and-exchange-based genomic modifications.” FASEB J. 2011; 25(12):4088-107; Venken and Bellen, “Genome-wide manipulations of Drosophila melanogaster with transposons, Flp recombinase, and ΦC31 integrase.”Methods Mol. Biol. 2012; 859:203-28; Murphy, “Phage recombinases and their applications.”Adv. Virus Res. 2012; 83:367-414; Zhang et al., “Conditional gene manipulation: Creating a new biological era.” J. Zhejiang Univ. Sci. B. 2012; 13(7):511-24; Karpenshif and Bernstein, “From yeast to mammals: recent advances in genetic control of homologous recombination.” DNA Repair (Amst). 2012; 1; 11(10):781-8; the entire contents of each are hereby incorporated by reference in their entirety.

The recombinases provided herein are not meant to be exclusive examples of recombinases that can be used in embodiments of the disclosure. The methods and compositions of the disclosure can be expanded by mining databases for new orthogonal recombinases or designing synthetic recombinases with defined DNA specificities (See, e.g., Groth et al., “Phage integrases: biology and applications.” J. Mol. Biol. 2004; 335, 667-678; Gordley et al., “Synthesis of programmable integrases.” Proc. Natl. Acad. Sci. USA. 2009; 106, 5053-5058; the entire contents of each are hereby incorporated by reference in their entirety).

Other examples of recombinases that are useful in the systems, methods, and compositions described herein are known to those of skill in the art, and any new recombinase that is discovered or generated is expected to be able to be used in the different embodiments of the disclosure.

As used herein, the term “retrotransposase” and the like refer to an enzyme, or combination of one or more enzymes, wherein at least one enzyme has a reverse transcriptase domain. Retrotransposases are capable of inserting long sequences (e.g., over 3000 nucleotides) of heterologous nucleic acid into a genome. Examples of retrotransposases include for example, without limitation, retrotransposases encoded by elements such as R2, L1, Tol2 Tc1, Tc3, Mariner (Himar 1), Mariner (mos 1), Minos, and any mutants thereof.

In some embodiments, the one or more genes of interest or one or more nucleic acid sequences of interest are inserted into a desired location in a genome using a RNA fragment, such as a retrotransposon, encoding the nucleic acid linked to a complementary or associated integration site. The insertion of the nucleic acid of interest into a location in the desired location in the genome using a retrotransposon is aided by a retrotransposase.

The gene and nucleic acid sequence of interest disclosed herein can be any gene and nucleic acid sequence that are known in the art. The gene and nucleic acid sequence of interest can be for therapeutic and/or diagnostic uses. Examples of genes of interest include, without limitation, GBA, BTK, ADA, CNGB3, CNGA3, ATF6, GNAT2, ABCA1, ABCA7, APOE, CETP, LIPC, MMP9, PLTP, VTN, ABCA4, MFSD8, TLR3, TLR4, ERCC6, HMCN1, HTRA1, MCDR4, MCDR5, ARMS2, C2, C3, CFB, CFH, JAG1, NOTCH2, CACNA1F, SERPINA1, TTR, GSN, B2M, APOA2, APOA1, OSMR, ELP4, PAX6, ARG, ASL, PITX2, FOXC1, BBS1, BBS10, BBS2, BBS9, MKKS, MKS1, BBS4, BBS7, TTC8, ARL6, BBS5, BBS12, TRIM32, CEP290, ADIPOR1, BBIP1, CEP19, IFT27, LZTFL1, DMD, BEST1, HBB, CYP4V2, AMACR, CYP7B1, HSD3B7, AKR1D1, OPN1SW, NR2F1, RLBP1, RGS9, RGS9BP, PROM1, PRPH2, GUCY2D, CACD, CHM, ALAD, ASS1, SLC25A13, OTC, ACADVL, ETFDH, TMEM67, CC2D2A, RPGRIP1L, KCNV2, CRX, GUCA1A, CERKL, CDHR1, PDE6C, TTLL5, RPGR, CEP78, C21orf2, C8ORF37, RPGRIP1, ADAMS, POC1B, PITPNM3, RAB28, CACNA2D4, AIPL1, UNC119, PDE6H, OPN1LW, RIMS1, CNNM4, IFT81, RAX2, RDH5, SEMA4A, CORD17, PDE6B, GRK1, SAG, RHO, CABP4, GNB3, SLC24A1, GNAT1, GRM6, TRPM1, LRIT3, TGFBI, TACSTD2, KRT12, OVOL2, CPS1, UGT1A1, UGT1A9, UGT1A8, UGT1A7, UGT1A6, UGT1A5, UGT1A4, CFTR, DLD, EFEMP1, ABCC2, ZNF408, LRP5, FZD4, TSPAN12, EVR3, APOB, SLC2A2, LOC106627981, GBA1, NR2E3, OAT, SLC40A1, F8, F9, UROD, CPDX, HFE, JH, LDLR, EPHX1, TJP2, BAAT, NBAS, LARS1, HAMP, HJV, RS1, ADAMTS18, LRAT, RPE65, LCA5, MERTK, GDF6, RD3, CCT2, CLUAP1, DTHD1, NMNAT1, SPATA7, IFT140, IMPDH1, OTX2, RDH12, TULP1, CRB1, MT-ND4, MT-ND1, MT-ND6, BCKDHA, BCKDHB, DBT, MMAB, ARSB, GUSB, NAGS, NPC1, NPC2, NDP, OPA1, OPA3, OPA4, OPA5, RTN4IP1, TMEM126A, OPA6, OPA8, ACO2, PAH, PRKCSH, SEC63, GAA, UROS, PPDX, HPX, HMOX1, HMBS, MIR223, CYP1B1, LTBP2, AGXT, ATP8B1, ABCB11, ABCB4, FECH, ALAS2, PRPF31, RP1, EYS, TOPORS, USH2A, CNGA1, C2ORF71, RP2, KLHL7, ORF1, RP6, RP24, RP34, ROM1, ADGRA3, AGBL5, AHR, ARHGEF18, CA4, CLCC1, DHDDS, EMC1, FAM161A, HGSNAT, HK1, IDH3B, KIAA1549, KIZ, MAK, NEUROD1, NRL, PDE6A, PDE6G, PRCD, PRPF3, PRPF4, PRPF6, PRPF8, RBP3, REEP6, SAMD11, SLC7A14, SNRNP200, SPP2, ZNF513, NEK2, NEK4, NXNL1, OFD1, RP1L1, RP22, RP29, RP32, RP63, RP9, RGR, POMGNT1, DHX38, ARL3, COL2A1, SLCO1B1, SLCO1B3, KCNJ13, TIMP3, ELOVL4, TFR2, FAH, HPD, MYO7A, CDH23, PCDH15, DFNB31, GPR98, USH1C, USH1G, CIB2, CLRN1, HARS, ABHD12, ADGRV1, ARSG, CEP250, IMPG1, IMPG2, VCAN, G6PC1, ATP7B and any derivatives thereof.

As used here, the terms “retrotransposons,” “jumping genes,” “jumping nucleic acids,” and the like refer to cellular movable genetic elements dependent on reverse transcription. The retrotransposons are of non-replication competent cellular origin, and are capable of carrying a foreign nucleic acid sequence. The retrotransposons can act as parasites of retroviruses, retaining certain classical hallmarks, such as long terminal repeats (LTR), retroviral primer binding sites, and the like. However, the naturally occurring retrotransposons usually do not contain functional retroviral structure genes, which would normally be capable of recombining to yield replication competent viruses. Some retrotransposons are examples of so-called “selfish DNA”, or genetic information, which encodes nothing except the ability to replicate itself. The retrotransposon may do so by utilizing the occasional presence of a retrovirus or a retrotransposase within the host cell, efficiently packaging itself within the viral particle, which transports it to the new host genome, where it is expressed again as RNA. The information encoded within that RNA is potentially transported with the jumping gene. A retrotransposon can be a DNA transposon or a retrotransposon, including a LTR retrotransposon or a non-LTR retrotransposon.

Non-long terminal repeat (LTR) retrotransposons are a type of mobile genetic elements that are widespread in eukaryotic genomes. They include two classes: the apurinic/apyrimidinic endonuclease (APE)-type and the restriction enzyme-like endonuclease (RLE)-type. The APE class retrotransposons are comprised of two functional domains: an endonuclease/DNA binding domain, and a reverse transcriptase domain. The RLE class are comprised of three functional domains: a DNA binding domain, a reverse transcription domain, and an endonuclease domain. The reverse transcriptase domain of non-LTR retrotransposon functions by binding an RNA sequence template and reverse transcribing it into the host genome's target DNA. The RNA sequence template has a 3′ untranslated region which is specifically bound to the transposase, and a variable 5′ region generally having Open Reading Frame(s) (“ORF”) encoding transposase proteins. The RNA sequence template may also comprise a 5′ untranslated region which specifically binds the retrotransposase. In some embodiments, a non-LTR transposons can include a LINE retrotransposon, such as L1, and a SINE retrotransposon, such as an Alu sequence. Other examples include for example, without limitation, R1, R2, R3, R4, and R5 retro-transposons (Moss, W. N. et al., RNA Biol. 2011, 8(5), 714-718; and Burke, W. D. et al., Molecular Biology and Evolution 2003, 20(8), 1260-1270). The transposon can be autonomous or non-autonomous.

LTR retrotransposons, which include retroviruses, make up a significant fraction of the typical mammalian genome, comprising about 8% of the human genome and 10% of the mouse genome. Lander et al., 2001, Nature 409, 860-921; Waterson et al., 2002, Nature 420, 520-562. LTR elements include retrotransposons, endogenous retroviruses (ERVs), and repeat elements with HERV origins, such as SINE-R. LTR retrotransposons include two LTR sequences that flank a region encoding two enzymes: integrase and retrotransposase.

ERVs include human endogenous retroviruses (HERVs), the remnants of ancient germ-cell infections. While most HERV proviruses have undergone extensive deletions and mutations, some have retained ORFS coding for functional proteins, including the glycosylated env protein. The env gene confers the potential for LTR elements to spread between cells and individuals. Indeed, all three open reading frames (pol, gag, and env) have been identified in humans, and evidence suggests that ERVs are active in the germline. See, e.g., Wang et al., 2010, Genome Res. 20, 19-27. Moreover, a few families, including the HERV-K (HML-2) group, have been shown to form viral particles, and an apparently intact provirus has recently been discovered in a small fraction of the human population. See, e.g., Bannert and Kurth, 2006, Proc. Natl. Acad. USA 101, 14572-14579.

LTR retrotransposons insert into new sites in the genome using the same steps of DNA cleavage and DNA strand-transfer observed in DNA transposons. In contrast to DNA transposons, however, recombination of LTR retrotransposons involves an RNA intermediate. LTR retrotransposons make up about 8% of the human genome. See, e.g., Lander et al., 2001, Nature 409, 860-921; Hua-Van et al., 2011, Biol. Dir. 6, 19.

### Integration Site

The present disclosure provides non-naturally occurring or engineered systems, methods, and compositions for site-specific genetic engineering via the addition of an integration site into a target genome. The integration site will be discussed in more details below.

As used herein, the term “integration site” refers to the site within the target genome where one or more genes of interest or one or more nucleic acid sequences of interest are inserted. Examples of integration sites include for example, without limitation, a lox71 site (SEQ ID NO: 1), attB sites (SEQ ID NO: 3 and SEQ ID NO: 43), attP sites (SEQ ID NO: 4 and SEQ ID NO: 44), an attL site (SEQ ID NO: 67), an attR site (SEQ ID NO: 68), a Vox site (SEQ ID NO: 69), a FRT site (SEQ ID NO: 70), or a pseudo attP site (SEQ ID NO: 78). The integration site can be inserted into the genome or a fragment thereof of a cell using a nuclease, a gRNA, and/or an integration enzyme. The integration site can be inserted into the genome of a cell using a prime editor such as, without limitation, PE1, PE2, and PE3, wherein the integration site is carried on a pegRNA. The pegRNA can target any site that is known in the art. Examples of cites targeted by the pegRNA include, without limitation, ACTB, SUPT16H, SRRM2, NOLC1, DEPDC4, NES, LMNB1, AAVS1 locus, CC10, CFTR, SERPINA1, ABCA4, and any derivatives thereof. The complementary integration site may be operably linked to a gene of interest or nucleic acid sequence of interest in an exogenous DNA or RNA. In some embodiments, one integration site is added to a target genome. In some embodiments, more than one integration sites are added to a target genome.

To insert multiple genes or nucleic acids of interest, two or more integration sites are added to a desired location. Multiple DNA comprising nucleic acid sequences of interest are flanked orthogonal to the integration sequences, such as, without limitation, attB and attP. An integration site is “orthogonal” when it does not significantly recognize the recognition site or nucleotide sequence of a recombinase. Thus, one attB site of a recombinase can be orthogonal to an attB site of a different recombinase. In addition, one pair of attB and attP sites of a recombinase can be orthogonal to another pair of attB and attP sites recognized by the same recombinase. A pair of recombinases are considered orthogonal to each other, as defined herein, when there is recognition of each other's attB or attP site sequences.

The lack of recognition of integration sites or pairs of sites by the same recombinase or a different recombinase can be less than about 30%. In some embodiments, the lack of recognition of integration sites or pairs of sites by the same recombinase or a different recombinase can be less than about 30%, less than about 28%, less than about 26%, less than about 24%, less than about 22%, less than about 20%, less than about 18%, less than about 16%, less than about 14%, less than about 12%, less than about 10%, less than about 8%, less than about 6%, less than about 4%, less than about 2%, about 1%, or any range that is formed from any two of those values as endpoints. The crosstalk can be less than about 30%. In some embodiments, the crosstalk is less than about 30%, less than about 28%, less than about 26%, less than about 24%, less than about 22%, less than about 20%, less than about 18%, less than about 16%, less than about 14%, less than about 12%, less than about 10%, less than about 8%, less than about 6%, less than about 4%, less than about 2%, less than about 1%, or any range that is formed from any two of those values as endpoints.

In some embodiments, the attB and/or attP site sequences comprise a central dinucleotide sequence. It has been shown that, for example, the central dinucleotide can be changed to GA from GT and that only GA containing attB/attP sites interact and will not cross react with GT containing sequences. In some embodiments, the central dinucleotide is selected from the group consisting of AG, AC, TG, TC, CA, CT, GA, AA, TT, CC, GG, AT, TA, GC, CG and GT.

As used herein, the term “pair of an attB and attP site sequences” and the like refer to attB and attP site sequences that share the same central dinucleotide and can recombine. This means that in the presence of one serine integrase as many as six pairs of these orthogonal att sites can recombine (attPTT will specifically recombine with attBTT, attPTC will specifically recombine with attBTC, and so on).

In some embodiments, the central dinucleotide is nonpalindromic. In some embodiments, the central dinucleotide is palindromic. In some embodiments, a pair of an attB site sequence and an attP site sequence are used in different DNA encoding genes of interest or nucleic acid sequences of interest for inducing directional integration of two or more different nucleic acids.

The Table 3 below shows examples of pairs of attB site sequence and attP site sequence with different central dinucleotide (CD).

TABLE 3

| Pair | attB | attP | CD |
|---|---|---|---|
| 1 | SEQ ID NO: 5 | SEQ ID NO: 6 | TT |
| 2 | SEQ ID NO: 7 | SEQ ID NO: 8 | AA |
| 3 | SEQ ID NO: 9 | SEQ ID NO: 10 | CC |
| 4 | SEQ ID NO: 11 | SEQ ID NO: 12 | GG |
| 5 | SEQ ID NO: 13 | SEQ ID NO: 14 | TG |
| 6 | SEQ ID NO: 15 | SEQ ID NO: 16 | GT |
| 7 | SEQ ID NO: 17 | SEQ ID NO: 18 | CT |
| 8 | SEQ ID NO: 19 | SEQ ID NO: 20 | CA |
| 9 | SEQ ID NO: 21 | SEQ ID NO: 22 | TC |
| 10 | SEQ ID NO: 23 | SEQ ID NO: 24 | GA |
| 11 | SEQ ID NO: 25 | SEQ ID NO: 26 | AG |
| 12 | SEQ ID NO: 27 | SEQ ID NO: 28 | AC |
| 13 | SEQ ID NO: 29 | SEQ ID NO: 30 | AT |
| 14 | SEQ ID NO: 31 | SEQ ID NO: 32 | GC |
| 15 | SEQ ID NO: 33 | SEQ ID NO: 34 | CG |
| 16 | SEQ ID NO: 35 | SEQ ID NO: 36 | TA |

### Paste

The present disclosure provides non-naturally occurring or engineered systems, methods, and compositions for site-specific genetic engineering using PASTE. PASTE will be discussed in more details below.

The site-specific genetic engineering disclosed herein is for the insertion of one or more genes of interest or one or more nucleic acid sequences of interest into a genome of a cell. In some embodiments, the gene of interest is a mutated gene implicated in a genetic disease such as, without limitation, a metabolic disease, cystic fibrosis, muscular dystrophy, hemochromatosis, Tay-Sachs, Huntington disease, Congenital Deafness, Sickle cell anemia, Familial hypercholesterolemia, adenosine deaminase (ADA) deficiency, X-linked SCID (X-SCID), and Wiskott-Aldrich syndrome (WAS). In some embodiments, the gene of interest or nucleic acid sequence of interest can be a reporter gene upstream or downstream of a gene for genetic analyses such as, without limitation, for determining the expression of a gene. In some embodiments, the reporter gene is a GFP template (SEQ ID NO: 76) or a Gaussia Luciferase (G-Luciferase) template (SEQ ID NO: 77) In some embodiments, the gene of interest or nucleic acid sequence of interest can be used in plant genetics to insert genes to enhance drought tolerance, weather hardiness, and increased yield and herbicide resistance in plants. In some embodiments, the gene of interest or nucleic acid sequence of interest can be used for site-specific insertion of a protein (e.g., a lysosomal enzyme), a blood factor (e.g., Factor I, II, V, VII, X, XI, XII or XIII), a membrane protein, an exon, an intracellular protein (e.g., a cytoplasmic protein, a nuclear protein, an organellar protein such as a mitochondrial protein or lysosomal protein), an extracellular protein, a structural protein, a signaling protein, a regulatory protein, a transport protein, a sensory protein, a motor protein, a defense protein, or a storage protein, an anti-inflammatory signaling molecules into cells for treatment of immune diseases, including but not limited to arthritis, psoriasis, lupus, coeliac disease, glomerulonephritis, hepatitis, and inflammatory bowel disease.

The size of the inserted gene or nucleic acid can vary from about 1 bp to about 50,000 bp. In some embodiments, the size of the inserted gene or nucleic acid can be about 1 bp, 10 bp, 50 bp, 100 bp, 150 bp, 200 bp, 250 bp, 300 bp, 350 bp, 400 bp, 600 bp, 800 bp, 1000 bp, 1200 bp, 1400 bp, 1600 bp, 1800 bp, 2000 bp, 2200 bp, 2400 bp, 2600 bp, 2800 bp, 3000 bp, 3200 bp, 3400 bp, 3600 bp, 3800 bp, 4000 bp, 4200 bp, 4400 bp, 4600 bp, 4800 bp, 5000 bp, 5200 bp, 5400 bp, 5600 bp, 5800 bp, 6000 bp, 6200, 6400 bp, 6600 bp, 6800 bp, 7000 bp, 7200 bp, 7400 bp, 7600 bp, 7800 bp, 8000 bp, 8200 bp, 8400 bp, 8600 bp, 8800 bp, 9000 bp, 9200 bp, 9400 bp, 9600 bp, 9800 bp, 10,000 bp, 10,200 bp, 10,400 bp, 10,600 bp, 10,800 bp, 11,000 bp, 11,200 bp, 11,400 bp, 11,600 bp, 11,800 bp, 12,000 bp, 14,000 bp, 16,000 bp, 18,000 bp, 20,000 bp, 30,000 bp, 40,000 bp, 50,000 bp, or any range that is formed from any two of those values as endpoints.

In some embodiments, the site-specific engineering using the gene of interest or nucleic acid sequence of interest disclosed herein is for the engineering of T cells and NKs for tumor targeting or allogeneic generation. These can involve the use of receptor or CAR for tumor specificity, anti-PD1 antibody, cytokines like IFN-gamma, TNF-alpha, IL-15, IL-12, IL-18, IL-21, and IL-10, and immune escape genes.

In the present disclosure, the site-specific insertion of the gene of interest or nucleic acid of interest is performed through Programmable Addition via Site-Specific Targeting Elements (PASTE). Components for inserting a gene of interest or a nucleic acid of interest using PASTE are for example, without limitation, a nuclease, a gRNA adding the integration site, a DNA or RNA strand comprising the gene or nucleic acid linked to a sequence that is complementary or associated to the integration site, and an integration enzyme. Components for inserting a gene of interest or a nucleic acid of interest using PASTE are for example, without limitation, a prime editor expression, pegRNA adding the integration site, nicking guide RNA, integration enzyme (Cre or serine recombinase), transgene vector comprising the gene of interest or nucleic acid sequence of interest with gene and integration signal. The nuclease and prime editor integrate the integration site into the genome. The integration enzyme integrates the gene of interest into the integration site. In some embodiments, the transgene vector comprising the gene or nucleic acid sequence of interest with gene and integration signal is a DNA minicircle devoid of bacterial DNA sequences. In some embodiments, the transgenic vector is a eukaryotic or prokaryotic vector.

As used herein, the term “vector” or “transgene vector” refers to a recombinant DNA molecule containing a desired coding sequence and appropriate nucleic acid sequences necessary for the expression of the operably linked coding sequence in a host organism. Nucleic acid sequences necessary for expression in prokaryotes usually include for example, without limitation, a promoter, an operator (optional), a ribosome binding site, and/or other sequences. Eukaryotic cells are generally known to utilize promoters (constitutive, inducible or tissue specific), enhancers, and termination and polyadenylation signals, although some elements may be deleted and other elements added without sacrificing the necessary expression. The transgenic vector may encode the PE and the integration enzyme, linked to each other via a linker. The linker can be a cleavable linker. For example, transgenic vector encoding the PE and the integration enzyme, linked to each other via a linker is pCMV PE2 P2A Cre comprises SEQ ID NO: 73. In some embodiments, the linker can be a non-cleavable linker. In some embodiments the nuclease, prime editor, and/or integration enzyme can be encoded in different vectors.

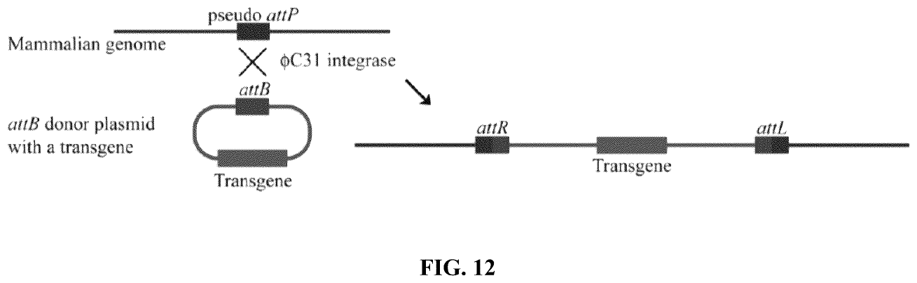

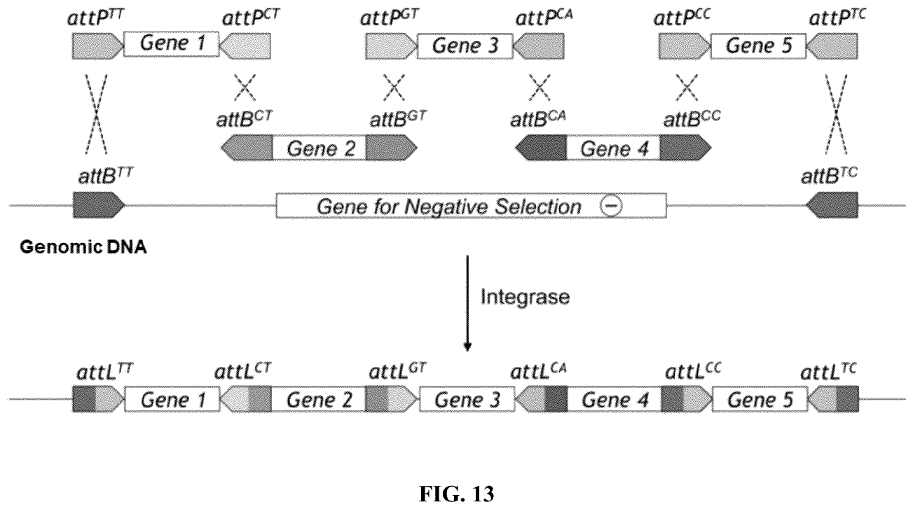

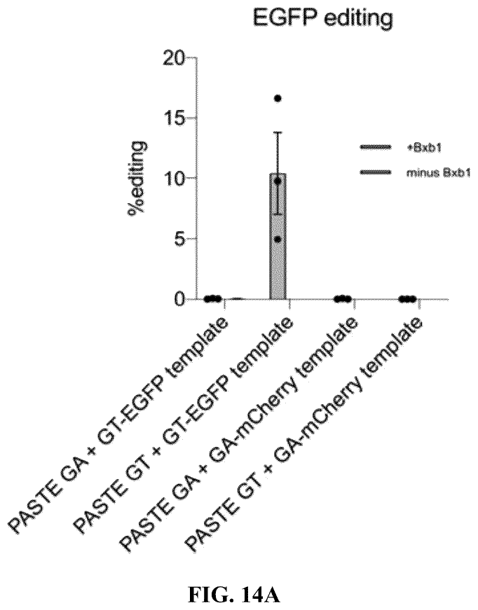

A method of inserting multiple genes or nucleic acid sequences of interest into a single site according to embodiments of the present disclosure is illustrated in FIG. 12. In some embodiments, multiplexing involves inserting multiple genes of interest in multiple loci using unique pegRNA as illustrated in FIG. 13 (Merrick, C. A. et al., ACS Synth. Biol. 2018, 7, 299-310). The insertion of multiple genes of interest or nucleic acids of interest into a cell genome, referred herein as “multiplexing,” is facilitated by incorporation of the complementary 5′ integration site to the 5′ end of the DNA or RNA comprising the first nucleic acid and 3′ integration site to the 3′ end of the DNA or RNA comprising the last nucleic acid. In some embodiments, the number of genome of interest or amino acid sequences of interest that are inserted into a cell genome using multiplexing can be about 1, 2, 3, 4, 5, 6, 7, 8, 9 10, 11, 12, 13, 14, 15, 16, 17, 18, 19, 20, 21, 22, 23, 24, 25, or any range that is formed from any two of those values as endpoints.

In some embodiments, multiplexing allows integration of for example, signaling cascade, over-expression of a protein of interest with its cofactor, insertion of multiple genes mutated in a neoplastic condition, or insertion of multiple CARs for treatment of cancer.

In some embodiments, the integration sites may be inserted into the genome using non-prime editing methods such as rAAV mediated nucleic acid integration, TALENS and ZFNs. A number of unique properties make AAV a promising vector for human gene therapy (Muzyczka, CURRENT TOPICS IN MICROBIOLOGY AND IMMUNOLOGY, 158:97-129 (1992)). Unlike other viral vectors, AAVs have not been shown to be associated with any known human disease and are generally not considered pathogenic. Wild type AAV is capable of integrating into host chromosomes in a site-specific manner M. Kotin et al., PROC. NATL. ACAD. SCI, USA, 87:2211-2215 (1990); R. J. Samulski, EMBO 10(12):3941-3950 (1991)). Instead of creating a double-stranded DNA break, AAV stimulates endogenous homologous recombination to achieve the DNA modification. Further, transcription activator-like effector nucleases (TALENs) and Zinc-finger nucleases (ZFNs) for genome editing and introducing targeted DSBs. The specificity of TALENs arises from two polymorphic amino acids, the so-called repeat variable diresidues (RVDs) located at positions 12 and 13 of a repeated unit. TALENS are linked to FokI nucleases, which cleaves the DNA at the desired locations. ZFNs are artificial restriction enzymes for custom site-specific genome editing. Zinc fingers themselves are transcription factors, where each finger recognizes 3-4 bases. By mixing and matching these finger modules, researchers can customize which sequence to target.

As used herein, the terms “administration,” “introducing,” or “delivery” into a cell, a tissue, or an organ of a plasmid, nucleic acids, or proteins for modification of the host genome refers to the transport for such administration, introduction, or delivery that can occur in vivo, in vitro, or ex vivo. Plasmids, DNA, or RNA for genetic modification can be introduced into cells by transfection, which is typically accomplished by chemical means (e.g., calcium phosphate transfection, polyethyleneimine (PEI) Or lipofection), physical means (electroporation or microinjection), infection (this typically means the introduction of an infectious agent such as a virus (e.g., a baculovirus expressing the AAV Rep gene)), transduction (in microbiology, this refers to the stable infection of cells by viruses, or the transfer of genetic material from one microorganism to another by viral factors (e.g., bacteriophages)). Vectors for the expression of a recombinant polypeptide, protein or oligonucleotide may be obtained by physical means (e.g., calcium phosphate transfection, electroporation, microinjection, or lipofection) in a cell, a tissue, an organ or a subject. The vector can be delivered by preparing the vector in a pharmaceutically acceptable carrier for the in vitro, ex vivo, or in vivo delivery to the carrier.

As used herein, the term “transfection” refers to the uptake of an exogenous nucleic acid molecule by a cell. A cell is “transfected” when an exogenous nucleic acid has been introduced into the cell membrane. The transfection can be a single transfection, co-transfection, or multiple transfection. Numerous transfection techniques are generally known in the art. See, for example, Graham et al. (1973) Virology, 52: 456. Such techniques can be used to introduce one or more exogenous nucleic acid molecules into a suitable host cell.

In some embodiments, the exogenous nucleic acid molecule and/or other components for gene editing are combined and delivered in a single transfection. In other embodiments, the exogenous nucleic acid molecule and/or other components for gene editing are not combined and delivered in a single transfection. In some embodiments, exogenous nucleic acid molecule and/or other components for gene editing are combined and delivered in a single transfection to comprise for example, without limitation, a prime editing vector, a landing site such as a landing site containing pegRNA, a nicking guide such as a nicking guide for stimulating prime editing, an expression vector such as an expression vector for a corresponding integrase or recombinase, a minicircle DNA cargo such as a minicircle DNA cargo encoding for green fluorescent protein (GFP), any derivatives thereof, and any combinations thereof. In some embodiments, the gene of interest or amino acid sequence of interest can be introduced using liposomes. In some embodiments, the gene of interest or amino acid sequence of interest can be delivered using suitable vectors for instance, without limitation, plasmids and viral vectors. Examples of viral vectors include, without limitation, adeno-associated viruses (AAV), lentiviruses, adenoviruses, other viral vectors, derivatives thereof, or combinations thereof. The proteins and one or more guide RNAs can be packaged into one or more vectors, e.g., plasmids or viral vectors. In some embodiments, the delivery is via nanoparticles or exosomes. For example, exosomes can be particularly useful in delivery RNA.

In some embodiments, the prime editing inserts the landing site with efficiencies of at least about 1%, at least about 5%, at least about 10%, at least about 15%, at least about, at least about 20%, at least about 25%, at least about 30%, at least about 35%, at least about 40%, at least about 45%, or at least about 50%. In some embodiments, the prime editing inserts the landing site(s) with efficiencies of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, or any range that is formed from any two of those values as endpoints.

### Sequences

Sequences of enzymes, guides, integration sites, and plasmids can be found in Table 4 below.

TABLE 4

| SEQ ID NO/ | |
|---|---|
| DESCRIPTION/ | |
| SOURCE | SEQUENCE |
| SEQ ID NO: 1 | ATAACTTCGTATAATGTATGCTATACGAACGGTA |
| Lox71 | |
| (Artificial sequence) | |
| SEQ ID NO: 2 | TACCGTTCGTATAATGTATGCTATACGAAGTTAT |
| Lox66 | |
| (Artificial sequence) | |
| SEQ ID NO: 3 | GGCCGGCTTGTCGACGACGGCGGTCTCCGTCGTCAGGATCATCCG |
| attB | G |
| (Artificial sequence) | |
| SEQ ID NO: 4 | CCGGATGATCCTGACGACGGAGACCGCCGTCGTCGACAAGCCGGC |
| attP | C |
| (Artificial Sequence) | |
| SEQ ID NO: 5 | GGCTTGTCGACGACGGCGTTCTCCGTCGTCAGGATCAT |
| attB-TT | |
| (Artificial Sequence) | |
| SEQ ID NO: 6 | GTGGTTTGTCTGGTCAACCACCGCGTTCTCAGTGGTGTACGGTACA |
| attP-TT | AACCCA |
| (Artificial Sequence) | |
| SEQ ID NO: 7 | GGCTTGTCGACGACGGCGAACTCCGTCGTCAGGATCAT |
| attB-AA | |
| (Artificial Sequence) | |
| SEQ ID NO: 8 | GTGGTTTGTCTGGTCAACCACCGCGAACTCAGTGGTGTACGGTAC |
| attP-AA | AAACCCA |
| (Artificial Sequence) | |
| SEQ ID NO: 9 | GGCTTGTCGACGACGGCGCCCTCCGTCGTCAGGATCAT |
| attB-CC | |
| (Artificial Sequence) | |
| SEQ ID NO: 10 | GTGGTTTGTCTGGTCAACCACCGCGCCCTCAGTGGTGTACGGTACA |
| attP-CC | AACCCA |
| (Artificial Sequence) | |
| SEQ ID NO: 11 | GGCTTGTCGACGACGGCGGGCTCCGTCGTCAGGATCAT |
| attB-GG | |
| (Artificial Sequence) | |
| SEQ ID NO: 12 | GTGGTTTGTCTGGTCAACCACCGCGGGCTCAGTGGTGTACGGTAC |
| attP-GG | AAACCCA |
| (Artificial Sequence) | |
| SEQ ID NO: 13 | GGCTTGTCGACGACGGCGTGCTCCGTCGTCAGGATCAT |
| attB-TG | |
| (Artificial Sequence) | |
| SEQ ID NO: 14 | GTGGTTTGTCTGGTCAACCACCGCGTGCTCAGTGGTGTACGGTACA |
| attP-TG | AACCCA |
| (Artificial Sequence) | |
| SEQ ID NO: 15 | GGCTTGTCGACGACGGCGGTCTCCGTCGTCAGGATCAT |
| attB-GT | |
| (Artificial Sequence) | |
| SEQ ID NO: 16 | GTGGTTTGTCTGGTCAACCACCGCGGTCTCAGTGGTGTACGGTACA |
| attP-GT | AACCCA |
| (Artificial Sequence) | |
| SEQ ID NO: 17 | GGCTTGTCGACGACGGCGCTCTCCGTCGTCAGGATCAT |
| attB-CT | |
| (Artificial Sequence) | |
| SEQ ID NO: 18 | GTGGTTTGTCTGGTCAACCACCGCGCTCTCAGTGGTGTACGGTACA |
| attP-CT | AACCCA |
| (Artificial Sequence) | |
| SEQ ID NO: 19 | GGCTTGTCGACGACGGCGCACTCCGTCGTCAGGATCAT |
| attB-CA | |
| (Artificial Sequence) | |
| SEQ ID NO: 20 | GTGGTTTGTCTGGTCAACCACCGCGCACTCAGTGGTGTACGGTACA |
| attP-CA | AACCCA |
| (Artificial Sequence) | |
| SEQ ID NO: 21 | GGCTTGTCGACGACGGCGTCCTCCGTCGTCAGGATCAT |
| attB-TC | |
| (Artificial Sequence) | |
| SEQ ID NO: 22 | GTGGTTTGTCTGGTCAACCACCGCGTCCTCAGTGGTGTACGGTACA |
| attP-TC | AACCCA |
| (Artificial Sequence) | |
| SEQ ID NO: 23 | GGCTTGTCGACGACGGCGGACTCCGTCGTCAGGATCAT |
| attB-GA | |
| (Artificial Sequence) | |
| SEQ ID NO: 24 | GTGGTTTGTCTGGTCAACCACCGCGGACTCAGTGGTGTACGGTAC |
| attP-GA | AAACCCA |
| (Artificial Sequence) | |
| SEQ ID NO: 25 | GGCTTGTCGACGACGGCGAGCTCCGTCGTCAGGATCAT |
| attB-AG | |
| (Artificial Sequence) | |
| SEQ ID NO: 26 | GTGGTTTGTCTGGTCAACCACCGCGAGCTCAGTGGTGTACGGTAC |
| attP-AG | AAACCCA |
| (Artificial Sequence) | |
| SEQ ID NO: 27 | GGCTTGTCGACGACGGCGACCTCCGTCGTCAGGATCAT |
| attB-AC | |
| (Artificial Sequence) | |
| SEQ ID NO: 28 | GTGGTTTGTCTGGTCAACCACCGCGACCTCAGTGGTGTACGGTACA |
| attP-AC | AACCCA |
| (Artificial Sequence) | |
| SEQ ID NO: 29 | GGCTTGTCGACGACGGCGATCTCCGTCGTCAGGATCAT |
| attB-AT | |
| (Artificial Sequence) | |
| SEQ ID NO: 30 | GTGGTTTGTCTGGTCAACCACCGCGATCTCAGTGGTGTACGGTACA |
| attP-AT | AACCCA |
| (Artificial Sequence) | |
| SEQ ID NO: 31 | GGCTTGTCGACGACGGCGGCCTCCGTCGTCAGGATCAT |
| attB-GC | |
| (Artificial Sequence | |
| SEQ ID NO: 32 | GTGGTTTGTCTGGTCAACCACCGCGGCCTCAGTGGTGTACGGTACA |
| attP-GC | AACCCA |
| (Artificial Sequence) | |
| SEQ ID NO: 33 | GGCTTGTCGACGACGGCGCGCTCCGTCGTCAGGATCAT |
| attB-CG | |
| (Artificial Sequence) | |
| SEQ ID NO: 34 | GTGGTTTGTCTGGTCAACCACCGCGCGCTCAGTGGTGTACGGTACA |
| attP-CG | AACCCA |
| (Artificial Sequence) | |
| SEQ ID NO: 35 | GGCTTGTCGACGACGGCGTACTCCGTCGTCAGGATCAT |
| attB-TA | |
| (Artificial Sequence) | |
| SEQ ID NO: 36 | GTGGTTTGTCTGGTCAACCACCGCGTACTCAGTGGTGTACGGTACA |
| attP-TA | AACCCA |
| (Artificial Sequence) | |
| SEQ ID NO: 37 | TGCGGGTGCCAGGGCGTGCCCTTGGGCTCCCCGGGCGCGTACTCC |
| C31-attB | |
| (Artificial Sequence) | |
| SEQ ID NO: 38 | GTGCCCCAACTGGGGTAACCTTTGAGTTCTCTCAGTTGGGGG |
| C31-attP | |
| (Artificial Sequence) | |
| SEQ ID NO: 39 | GCGCCCAAGTTGCCCATGACCATGCCGAAGCAGTGGTAGAAGGGC |
| R4-attB | ACCGGCAGACAC |
| (Artificial Sequence) | |
| SEQ ID NO: 40 | AGGCATGTTCCCCAAAGCGATACCACTTGAAGCAGTGGTACTGCT |
| R4-attP | TGTGGGTACACTCTGCGGGTGATGA |
| (Artificial Sequence) | |
| SEQ ID NO: 41 | GTCCTTGACCAGGTTTTTGACGAAAGTGATCCAGATGATCCAGCTC |
| BT1-attB | CACACCCCGAACGC |
| (Artificial Sequence) | |
| SEQ ID NO: 42 | GGTGCTGGGTTGTTGTCTCTGGACAGTGATCCATGGGAAACTACTC |
| BT1-attP | AGCACCACCAATGTTCC |
| (Artificial Sequence) | |
| SEQ ID NO: 43 | TCGGCCGGCTTGTCGACGACGGCGGTCTCCGTCGTCAGGATCATCC |
| Bxb-attB | GGGC |
| (Artificial Sequence) | |
| SEQ ID NO: 44 | GTCGTGGTTTGTCTGGTCAACCACCGCGGTCTCAGTGGTGTACGGT |
| Bxb-attP | ACAAACCCCGAC |
| (Artificial Sequence) | |
| SEQ ID NO: 45 | GATCAGCTCCGCGGGCAAGACCTTCTCCTTCACGGGGTGGAAGGT |
| TG1-attB | C |
| (Artificial Sequence) | |
| SEQ ID NO: 46 | TCAACCCCGTTCCAGCCCAACAGTGTTAGTCTTTGCTCTTACCCAG |
| TG1-attP | TTGGGCGGGATAGCCTGCCCG |
| (Artificial Sequence) | |
| SEQ ID NO: 47 | AACGATTTTCAAAGGATCACTGAATCAAAAGTATTGCTCATCCAC |
| C1-attB | GCGAAATTTTTC |
| (Artificial Sequence) | |
| SEQ ID NO: 48 | AATATTTTAGGTATATGATTTTGTTTATTAGTGTAAATAACACTAT |
| C1-attP | GTACCTAAAAT |
| (Artificial Sequence) | |
| SEQ ID NO: 49 | TGTAAAGGAGACTGATAATGGCATGTACAACTATACTCGTCGGTA |
| C370-attB | AAAAGGCA |
| (Artificial Sequence) | |
| SEQ ID NO: 50 | TAAAAAAATACAGCGTTTTTCATGTACAACTATACTAGTTGTAGTG |
| C370-attP | CCTAAA |
| (Artificial Sequence) | |
| SEQ ID NO: 51 | GAGCGCCGGATCAGGGAGTGGACGGCCTGGGAGCGCTACACGCT |
| K38-attB | GTGGCTGCGGTC |
| (Artificial Sequence) | |
| SEQ ID NO: 52 | CCCTAATACGCAAGTCGATAACTCTCCTGGGAGCGTTGACAACTT |
| K38-attP | GCGCACCCTGA |
| (Artificial Sequence) | |
| SEQ ID NO: 53 | TCTCGTGGTGGTGGAAGGTGTTGGTGCGGGGTTGGCCGTGGTCGA |
| RB-attB | GGTGGGGTGGTGGTAGCCATTCG |
| (Artificial Sequence) | |
| SEQ ID NO: 54 | GCACAGGTGTAGTGTATCTCACAGGTCCACGGTTGGCCGTGGACT |
| RV-attP | GCTGAAGAACATTCCACGCCAGGA |
| (Artificial Sequence) | |
| SEQ ID NO: 55 | AGTGCAGCATGTCATTAATATCAGTACAGATAAAGCTGTATCTCCT |
| SPBC-attB | GTGAACACAATGGGTGCCA |
| (Artificial Sequence) | |
| SEQ ID NO: 56 | AAAGTAGTAAGTATCTTAAAAAACAGATAAAGCTGTATATTAAGA |
| SPBC-attP | TACTTACTAC |
| (Artificial Sequence) | |
| SEQ ID NO: 57 | TGATAATTGCCAACACAATTAACATCTCAATCAAGGTAAATGCTTT |
| TP901-attB | TTCGTTTT |
| (Artificial Sequence) | |
| SEQ ID NO: 58 | AATTGCGAGTTTTTATTTCGTTTATTTCAATTAAGGTAACTAAAAA |
| TP901-attP | ACTCCTTT |
| (Artificial Sequence) | |
| SEQ ID NO: 59 | AAGGTAGCGTCAACGATAGGTGTAACTGTCGTGTTTGTAACGGTA |
| Wβ-attB | CTTCCAACAGCTGGCGTTTCAGT |
| (Artificial Sequence) | |
| SEQ ID NO: 60 | TAGTTTTAAAGTTGGTTATTAGTTACTGTGATATTTATCACGGTAC |
| Wβ-attP | CCAATAACCAATGAATATTTGA |
| (Artificial Sequence) | |
| SEQ ID NO: 61 | TGTAACTTTTTCGGATCAAGCTATGAAGGACGCAAAGAGGGAACT |
| A118-attB | AAACACTTAATT |
| (Artificial Sequence) | |
| SEQ ID NO: 62 | TTGTTTAGTTCCTCGTTTTCTCTCGTTGGAAGAAGAAGAAACGAGA |
| A118-attP | AACTAAAATTA |
| (Artificial Sequence) | |
| SEQ ID NO: 63 | CAACCTGTTGACATGTTTCCACAGACAACTCACGTGGAGGTAGTC |
| BL3-attB | ACGGCTTTTACGTTAGTT |
| (Artificial Sequence) | |
| SEQ ID NO: 64 | GAGAATACTGTTGAACAATGAAAAACTAGGCATGTAGAAGTTGTT |
| BL3-attP | TGTGCACTAACTTTAA |
| (Artificial Sequence) | |
| SEQ ID NO: 65 | ACAGGTCAACACATCGCAGTTATCGAACAATCTTCGAAAATGTAT |
| MR11-attB | GGAGGCACTTGTATCAATATAGGATGTATACCTTCGAAGACACTT |
| (Artificial Sequence) | GTACATGATGGATTAGAAGGCAAATCCTTT |
| SEQ ID NO: 66 | CAAAATAAAAAACATTGATTTTTATTAACTTCTTTTGTGCGGAACT |
| MR11-attP | ACGAACAGTTCATTAATACGAAGTGTACAAACTTCCATACAAAAA |
| (Artificial Sequence) | TAACCACGACAATTAAGACGTGGTTTCTA |
| SEQ ID NO: 67 | ATTATTTCTCACCCTGA |
| attL | |
| (Artificial Sequence) | |
| SEQ ID NO: 68 | ATCATCTCCCACCCGGA |
| attR | |
| (Artificial Sequence) | |
| SEQ ID NO: 69 | AATAGGTCTG AGAACGCCCA TTCTCAGACG TATT |
| Vox | |
| (Artificial Sequence) | |
| SEQ ID NO: 70 | GAAGTTCCTATAC TTTCTAGA GAATAGGAACTTC |
| FRT | |
| (Artificial Sequence) | |
| SEQ ID NO: 71 | GGTCGACATTGATTATTGACTAGTTATTAATAGTAATCAATTACGG |
| Cre recombinase | GGTCATTAGTTCATAGCCCATATATGGAGTTCCGCGTTACATAACT |
| expression plasmid | TACGGTAAATGGCCCGCCTGGCTGACCGCCCAACGACCCCCGCCC |
| (Artificial Sequence) | ATTGACGTCAATAATGACGTATGTTCCCATAGTAACGCCAATAGG |
| | GACTTTCCATTGACGTCAATGGGTGGAGTATTTACGGTAAACTGCC |
| | CACTTGGCAGTACATCAAGTGTATCATATGCCAAGTACGCCCCCTA |
| | TTGACGTCAATGACGGTAAATGGCCCGCCTGGCATTATGCCCAGT |
| | ACATGACCTTATGGGACTTTCCTACTTGGCAGTACATCTACGTATT |
| | AGTCATCGCTATTACCATGGTCGAGGTGAGCCCCACGTTCTGCTTC |
| | ACTCTCCCCATCTCCCCCCCCTCCCCACCCCCAATTTTGTATTTATT |
| | TATTTTTTAATTATTTTGTGCAGCGATGGGGGCGGGGGGGGGGGG |
| | GGCGCGCGCCAGGCGGGGGGGGGGGGGGGGGGGGGGGGGGGGG |
| | GGGGGGGCGGGGGGGGGGGGCGGCAGCCAATCAGAGCGGCGCGC |
| | TCCGAAAGTTTCCTTTTATGGCGAGGCGGCGGCGGCGGCGGCCCT |
| | ATAAAAAGCGAAGCGCGCGGCGGGCGGGAGTCGCTGCGCGCTGC |
| | CTTCGCCCCGTGCCCCGCTCCGCCGCCGCCTCGCGCCGCCCGCCCC |
| | GGCTCTGACTGACCGCGTTACTCCCACAGGTGAGCGGGCGGGACG |
| | GCCCTTCTCCTCCGGGCTGTAATTAGCGCTTGGTTTAATGACGGCT |
| | TGTTTCTTTTCTGTGGCTGCGTGAAAGCCTTGAGGGGCTCCGGGAG |
| | GGCCCTTTGTGCGGGGGGAGCGGCTCGGGGGGTGCGTGCGTGTGT |
| | GTGTGCGTGGGGAGCGCCGCGTGCGGCTCCGCGCTGCCCGGCGGC |
| | TGTGAGCGCTGCGGGCGCGGCGCGGGGCTTTGTGCGCTCCGCAGT |
| | GTGCGCGAGGGGAGCGCGGCCGGGGGCGGTGCCCCGCGGTGCGG |
| | GGGGGGCTGCGAGGGGAACAAAGGCTGCGTGCGGGGTGTGTGCG |
| | TGGGGGGGTGAGCAGGGGGTGTGGGCGCGTCGGTCGGGCTGCAA |
| | CCCCCCCTGCACCCCCCTCCCCGAGTTGCTGAGCACGGCCCGGCTT |
| | CGGGTGCGGGGCTCCGTACGGGGCGTGGCGCGGGGCTCGCCGTGC |
| | CGGGCGGGGGGTGGCGGCAGGTGGGGGTGCCGGGCGGGGCGGGG |
| | CCGCCTCGGGCCGGGGAGGGCTCGGGGGAGGGGCGCGGCGGCCC |
| | CCGGAGCGCCGGCGGCTGTCGAGGCGCGGCGAGCCGCAGCCATTG |
| | CCTTTTATGGTAATCGTGCGAGAGGGCGCAGGGACTTCCTTTGTCC |
| | CAAATCTGTGCGGAGCCGAAATCTGGGAGGCGCCGCCGCACCCCC |
| | TCTAGCGGGCGCGGGGCGAAGCGGTGCGGCGCCGGCAGGAAGGA |
| | AATGGGCGGGGAGGGCCTTCGTGCGTCGCCGCGCCGCCGTCCCCT |
| | TCTCCCTCTCCAGCCTCGGGGCTGTCCGCGGGGGGACGGCTGCCTT |
| | CGGGGGGGACGGGGCAGGGCGGGGTTCGGCTTCTGGCGTGTGACC |
| | GGCGGCTCTAGAGCCTCTGCTAACCATGTTCATGCCTTCTTCTTTTT |
| | CCTACAGCTCCTGGGCAACGTGCTGGTTATTGTGCTGTCTCATCAT |
| | TTTGGCAAAGAATTCTGAGCCGCCACCATGGCCAATTTACTGACC |
| | GTACACCAAAATTTGCCTGCATTACCGGTCGATGCAACGAGTGAT |
| | GAGGTTCGCAAGAACCTGATGGACATGTTCAGGGATCGCCAGGCG |
| | TTTTCTGAGCATACCTGGAAAATGCTTCTGTCCGTTTGCCGGTCGT |
| | GGGCGGCATGGTGCAAGTTGAATAACCGGAAATGGTTTCCCGCAG |
| | AACCTGAAGATGTTCGCGATTATCTTCTATATCTTCAGGCGCGCGG |
| | TCTGGCAGTAAAAACTATCCAGCAACATTTGGGCCAGCTAAACAT |
| | GCTTCATCGTCGGTCCGGGCTGCCACGACCAAGTGACAGCAATGC |
| | TGTTTCACTGGTTATGCGGCGGATCCGAAAAGAAAACGTTGATGC |
| | CGGTGAACGTGCAAAACAGGCTCTAGCGTTCGAACGCACTGATTT |
| | CGACCAGGTTCGTTCACTCATGGAAAATAGCGATCGCTGCCAGGA |
| | TATACGTAATCTGGCATTTCTGGGGATTGCTTATAACACCCTGTTA |
| | CGTATAGCCGAAATTGCCAGGATCAGGGTTAAAGATATCTCACGT |
| | ACTGACGGTGGGAGAATGTTAATCCATATTGGCAGAACGAAAACG |
| | CTGGTTAGCACCGCAGGTGTAGAGAAGGCACTTAGCCTGGGGGTA |
| | ACTAAACTGGTCGAGCGATGGATTTCCGTCTCTGGTGTAGCTGATG |
| | ATCCGAATAACTACCTGTTTTGCCGGGTCAGAAAAAATGGTGTTG |
| | CCGCGCCATCTGCCACCAGCCAGCTATCAACTCGCGCCCTGGAAG |
| | GGATTTTTGAAGCAACTCATCGATTGATTTACGGCGCTAAGGATG |
| | ACTCTGGTCAGAGATACCTGGCCTGGTCTGGACACAGTGCCCGTG |
| | TCGGAGCCGCGCGAGATATGGCCCGCGCTGGAGTTTCAATACCGG |
| | AGATCATGCAAGCTGGTGGCTGGACCAATGTAAATATTGTCATGA |
| | ACTATATCCGTAACCTGGATAGTGAAACAGGGGCAATGGTGCGCC |
| | TGCTGGAAGATGGCGATGGACCGGTGGAACAAAAACTTATTTCTG |
| | AAGAAGATCTGTGATAGCGGCCGCACTCCTCAGGTGCAGGCTGCC |
| | TATCAGAAGGTGGTGGCTGGTGTGGCCAATGCCCTGGCTCACAAA |
| | TACCACTGAGATCTTTTTCCCTCTGCCAAAAATTATGGGGACATCA |
| | TGAAGCCCCTTGAGCATCTGACTTCTGGCTAATAAAGGAAATTTAT |
| | TTTCATTGCAATAGTGTGTTGGAATTTTTTGTGTCTCTCACTCGGAA |
| | GGACATATGGGAGGGCAAATCATTTAAAACATCAGAATGAGTATT |
| | TGGTTTAGAGTTTGGCAACATATGCCCATATGCTGGCTGCCATGAA |
| | CAAAGGTTGGCTATAAAGAGGTCATCAGTATATGAAACAGCCCCC |
| | TGCTGTCCATTCCTTATTCCATAGAAAAGCCTTGACTTGAGGTTAG |
| | ATTTTTTTTATATTTTGTTTTGTGTTATTTTTTTCTTTAACATCCCTA |
| | AAATTTTCCTTACATGTTTTACTAGCCAGATTTTTCCTCCTCTCCTG |
| | ACTACTCCCAGTCATAGCTGTCCCTCTTCTCTTATGGAGATCCCTC |
| | GACCTGCAGCCCAAGCTTGGCGTAATCATGGTCATAGCTGTTTCCT |
| | GTGTGAAATTGTTATCCGCTCACAATTCCACACAACATACGAGCC |
| | GGAAGCATAAAGTGTAAAGCCTGGGGTGCCTAATGAGTGAGCTAA |
| | CTCACATTAATTGCGTTGCGCTCACTGCCCGCTTTCCAGTCGGGAA |
| | ACCTGTCGTGCCAGCGGATCCGCATCTCAATTAGTCAGCAACCAT |
| | AGTCCCGCCCCTAACTCCGCCCATCCCGCCCCTAACTCCGCCCAGT |
| | TCCGCCCATTCTCCGCCCCATGGCTGACTAATTTTTTTTATTTATGC |
| | AGAGGCCGAGGCCGCCTCGGCCTCTGAGCTATTCCAGAAGTAGTG |
| | AGGAGGCTTTTTTGGAGGCCTAGGCTTTTGCAAAAAGCTAACTTGT |
| | TTATTGCAGCTTATAATGGTTACAAATAAAGCAATAGCATCACAA |
| | ATTTCACAAATAAAGCATTTTTTTCACTGCATTCTAGTTGTGGTTTG |
| | TCCAAACTCATCAATGTATCTTATCATGTCTGGATCCGCTGCATTA |
| | ATGAATCGGCCAACGCGCGGGGAGAGGCGGTTTGCGTATTGGGCG |
| | CTCTTCCGCTTCCTCGCTCACTGACTCGCTGCGCTCGGTCGTTCGG |
| | CTGCGGCGAGCGGTATCAGCTCACTCAAAGGCGGTAATACGGTTA |
| | TCCACAGAATCAGGGGATAACGCAGGAAAGAACATGTGAGCAAA |
| | AGGCCAGCAAAAGGCCAGGAACCGTAAAAAGGCCGCGTTGCTGG |
| | CGTTTTTCCATAGGCTCCGCCCCCCTGACGAGCATCACAAAAATCG |
| | ACGCTCAAGTCAGAGGTGGCGAAACCCGACAGGACTATAAAGAT |
| | ACCAGGCGTTTCCCCCTGGAAGCTCCCTCGTGCGCTCTCCTGTTCC |
| | GACCCTGCCGCTTACCGGATACCTGTCCGCCTTTCTCCCTTCGGGA |
| | AGCGTGGCGCTTTCTCATAGCTCACGCTGTAGGTATCTCAGTTCGG |
| | TGTAGGTCGTTCGCTCCAAGCTGGGCTGTGTGCACGAACCCCCCGT |
| | TCAGCCCGACCGCTGCGCCTTATCCGGTAACTATCGTCTTGAGTCC |
| | AACCCGGTAAGACACGACTTATCGCCACTGGCAGCAGCCACTGGT |
| | AACAGGATTAGCAGAGCGAGGTATGTAGGCGGTGCTACAGAGTTC |
| | TTGAAGTGGTGGCCTAACTACGGCTACACTAGAAGAACAGTATTT |
| | GGTATCTGCGCTCTGCTGAAGCCAGTTACCTTCGGAAAAAGAGTT |
| | GGTAGCTCTTGATCCGGCAAACAAACCACCGCTGGTAGCGGTGGT |
| | TTTTTTGTTTGCAAGCAGCAGATTACGCGCAGAAAAAAAGGATCT |
| | CAAGAAGATCCTTTGATCTTTTCTACGGGGTCTGACGCTCAGTGGA |
| | ACGAAAACTCACGTTAAGGGATTTTGGTCATGAGATTATCAAAAA |
| | GGATCTTCACCTAGATCCTTTTAAATTAAAAATGAAGTTTTAAATC |
| | AATCTAAAGTATATATGAGTAAACTTGGTCTGACAGTTACCAATG |
| | CTTAATCAGTGAGGCACCTATCTCAGCGATCTGTCTATTTCGTTCA |
| | TCCATAGTTGCCTGACTCCCCGTCGTGTAGATAACTACGATACGGG |
| | AGGGCTTACCATCTGGCCCCAGTGCTGCAATGATACCGCGAGACC |
| | CACGCTCACCGGCTCCAGATTTATCAGCAATAAACCAGCCAGCCG |
| | GAAGGGCCGAGCGCAGAAGTGGTCCTGCAACTTTATCCGCCTCCA |
| | TCCAGTCTATTAATTGTTGCCGGGAAGCTAGAGTAAGTAGTTCGCC |
| | AGTTAATAGTTTGCGCAACGTTGTTGCCATTGCTACAGGCATCGTG |
| | GTGTCACGCTCGTCGTTTGGTATGGCTTCATTCAGCTCCGGTTCCC |
| | AACGATCAAGGCGAGTTACATGATCCCCCATGTTGTGCAAAAAAG |
| | CGGTTAGCTCCTTCGGTCCTCCGATCGTTGTCAGAAGTAAGTTGGC |
| | CGCAGTGTTATCACTCATGGTTATGGCAGCACTGCATAATTCTCTT |
| | ACTGTCATGCCATCCGTAAGATGCTTTTCTGTGACTGGTGAGTACT |
| | CAACCAAGTCATTCTGAGAATAGTGTATGCGGCGACCGAGTTGCT |
| | CTTGCCCGGCGTCAATACGGGATAATACCGCGCCACATAGCAGAA |
| | CTTTAAAAGTGCTCATCATTGGAAAACGTTCTTCGGGGCGAAAAC |
| | TCTCAAGGATCTTACCGCTGTTGAGATCCAGTTCGATGTAACCCAC |
| | TCGTGCACCCAACTGATCTTCAGCATCTTTTACTTTCACCAGCGTTT |
| | CTGGGTGAGCAAAAACAGGAAGGCAAAATGCCGCAAAAAAGGGA |
| | ATAAGGGCGACACGGAAATGTTGAATACTCATACTCTTCCTTTTTC |
| | AATATTATTGAAGCATTTATCAGGGTTATTGTCTCATGAGCGGATA |
| | CATATTTGAATGTATTTAGAAAAATAAACAAATAGGGGTTCCGCG |
| | CACATTTCCCCGAAAAGTGCCACCTG |
| SEQ ID NO: 72 | AGCTCTGATCAAGAGACAGGATGAGGATCGTTTCGCATGATTGAA |
| GFP-Lox66 Cre | CAAGATGGATTGCACGCAGGTTCTCCGGCCGCTTGGGTGGAGAGG |
| expression plasmid | CTATTCGGCTATGACTGGGCACAACAGACAATCGGCTGCTCTGAT |
| (Artificial Sequence) | GCCGCCGTGTTCCGGCTGTCAGCGCAGGGGCGCCCGGTTCTTTTTG |
| | TCAAGACCGACCTGTCCGGTGCCCTGAATGAACTGCAAGACGAGG |
| | CAGCGCGGCTATCGTGGCTGGCCACGACGGGCGTTCCTTGCGCAG |
| | CTGTGCTCGACGTTGTCACTGAAGCGGGAAGGGACTGGCTGCTAT |
| | TGGGCGAAGTGCCGGGGCAGGATCTCCATGTCATCTACACCTTGC |
| | TCCTGCCGAGAAAGTATCCATCATGGCTGATGCAATGCGGCGGCT |
| | GCATACGCTTGATCCGGCTACCTGCCCATTCGACCACCAAGCGAA |
| | ACATCGCATCGAGCGAGCACGTACTCGGATGGAAGCCGGTCTTGT |
| | CGATCAGGATGATCTGGACGAAGAGCATCAGGGGCTCGCGCCAGC |
| | CGAACTGTTCGCCAGGCTCAAGGCGAGCATGCCCGACGGCGAGGA |
| | TCTCGTCGTGACCCATGGCGATGCCTGCTTGCCGAATATCATGGTG |
| | GAAAATGGCCGCTTTTCTGGATTCATCGACTGTGGCCGGCTGGGTG |
| | TGGCGGACCGCTATCAGGACATAGCGTTGGCTACCCGTGATATTG |
| | CTGAAGAGCTTGGCGGCGAATGGGCTGACCGCTTCCTCGTGCTTTA |
| | CGGTATCGCCGCTCCCGATTCGCAGCGCATCGCCTTCTATCGCCTT |
| | CTTGACGAGTTCTTCTGAATTATTAACTCGAGATCCACTAGAGTGT |
| | GGCGGCCGCATTCTTATAATCAGCATCATGATGTGGTACCACATCA |
| | TGATGCTGATTACCCCCAACTGAGAGAACTCAAAGGTTACCCCAG |
| | TTGGGGGGGGCCCACAAATAAAGCAATAGCATCACAAATTTCACA |
| | AATAAAGCATTTTTTTCACTGCATTCTAGTTGTGGTTTGTCCAAAC |
| | TCATCGAGCTCGAGATCTGGCGAAGGCGATGGGGGTCTTGAAGGC |
| | GTGCTGGTACTCCACGATGCCCAGCTCGGTGTTGCTGTGCAGCTCC |
| | TCCACGCGGCGGAAGGCGAACATGGGGCCCCCGTTCTGCAGGATG |
| | CTGGGGTGGATGGCGCTCTTGAAGTGCATGTGGCTGTCCACCACG |
| | AAGCTGTAGTAGCCGCCGTCGCGCAGGCTGAAGGTGCGGGCGAAG |
| | CTGCCCACCAGCACGTTATCGCCCATGGGGTGCAGGTGCTCCACG |
| | GTGGCGTTGCTGCGGATGATCTTGTCGGTGAAGATCACGCTGTCCT |
| | CGGGGAAGCCGGTGCCCACCACCTTGAAGTCGCCGATCACGCGGC |
| | CGGCCTCGTAGCGGTAGCTGAAGCTCACGTGCAGCACGCCGCCGT |
| | CCTCGTACTTCTCGATGCGGGTGTTGGTGTAGCCGCCGTTGTTGAT |
| | GGCGTGCAGGAAGGGGTTCTCGTAGCCGCTGGGGTAGGTGCCGAA |
| | GTGGTAGAAGCCGTAGCCCATCACGTGGCTCAGCAGGTAGGGGCT |
| | GAAGGTCAGGGCGCCTTTGGTGCTCTTCATCTTGTTGGTCATGCGG |
| | CCCTGCTCGGGGGTGCCCTCTCCGCCGCCCACCAGCTCGAACTCCA |
| | CGCCGTTCAGGGTGCCGGTGATGCGGCACTCGATCTTCATGGCGG |
| | GCATGGTGGCGACCGGTAGCGCTAGCGGCTTCGGATAACTTCGTA |
| | TAGCATACATTATACGAACGGTAAGCGCTACCGCCGGCATACCCA |
| | AGTGAAGTTGCTCGCAGCTTATAGTCGCGCCCGGGGAGCCCAAGG |
| | GCACGCCCTGGCACCGCGGCCGCTGAGTCTCGACCATCATCATCA |
| | TCATCATTGAGTTTATCTGGGATAACAGGGTAATGTCATCTAGGGA |
| | TAACAGGGTATGTCATCTGGGATAACAGGGTAATGTATCTAGGGA |
| | TAACAGGGTAATGTCATCTGGGATAACAGGGTAATGTCATCTAGG |
| | GATAACAGGGTATGTCATCTGGGATAACAGGGTAATGTATCTAGG |
| | GATAACAGGGTAATGTCATCTGGGATAACAGGGTAATGTCATCTA |
| | GGGATAACAGGGTATGTCATCTGGGATAACAGGGTAATGTATCTA |
| | GGGATAACAGGGTAATGTCATCTGGGATAACAGGGTAATGTCATC |
| | TAGGGATAACAGGGTATGTCATCTGGGATAACAGGGTAATGTATC |
| | TAGGGATAACAGGGTAATGTCATCTGGGATAACAGGGTAATGTCA |
| | TCTAGGGATAACAGGGTATGTCATCTGGGATAACAGGGTAATGTA |
| | TCTAGGGATAACAGGGTAATGTCATCTGGGATAACAGGGTAATGT |
| | CATCTAGGGATAACAGGGTATGTCATCTGGGATAACAGGGTAATG |
| | TATCTAGGGATAACAGGGTAATGTCATCTGGGATAACAGGGTAAT |
| | GTCATCTAGGGATAACAGGGTAAATGTCATCTAGGGATAACAGGG |
| | TAATGTCATCTAGGGATAACAGGGTAATGTCATCTGGGATAACAG |
| | GGTAATGTCATCTAGGGATAACAGGGTAATGTATCGCCAGCGTCG |
| | CACAGCATGTTTGCTTGTCGCCGTCGCGTCTGTCACATCTTTTCCG |
| | CCAGCAGTTAGGGATTAGCGTCTTAAGCTGGCGCGAGGACCAACG |
| | TATCAGCCAGGCGAAGCTGCTTTTGAGCACCACCCGGATGCCTAT |
| | CGCCACCGTCGGTCGCAATGTTGGTTTTGACGATCAACTCTATTTC |
| | TCGCGGGTATTTAAAAAATGCACCGGGGCCAGCCCGAGCGAGTTC |
| | CGTGCCGGTTGTGAAGAAAAAGTGAATGATGTAGCCGTCAAGTTG |
| | TCATAATTGGTAACGAATCAGACAATTGACGGCTTGACGGAGTAG |
| | CATAGGGTTTGCAGAATCCCTGCTTCGTCCATTTGACAGGCACATT |
| | ATGCATGCCGCTTCGCCTTCGCGCGCGAATTGATCTGCTGCCTCGC |
| | GCGTTTCGGTGATGACGGTGAAAACCTCTGACACATGCAGCTCCC |
| | GGAGACGGTCACAGCTTGTCTGTAAGCGGATGCCGGGAGCAGACA |
| | AGCCCGTCAGGGCGCGTCAGCGGGTGTTGGCGGGTGTCGGGGCGC |
| | AGCCATGACCCAGTCACGTAGCGATAGCGGAGTGTATACTGGCTT |
| | AACTATGCGGCATCAGAGCAGATTGTACTGAGAGTGCACCATATG |
| | CGGTGTGAAATACCGCACAGATGCGTAAGGAGAAAATACCGCATC |
| | AGGCGCTCTTCCGCTTCCTCGCTCACTGACTCGCTGCGCTCGGTCG |
| | TTCGGCTGCGGCGAGCGGTATCAGCTCACTCAAAGGCGGTAATAC |
| | GGTTATCCACAGAATCAGGGGATAACGCAGGAAAGAACATGTGA |
| | GCAAAAGGCCAGCAAAAGGCCAGGAACCGTAAAAAGGCCGCGTT |
| | GCTGGCGTTTTTCCATAGGCTCCGCCCCCCTGACGAGCATCACAAA |
| | AATCGACGCTCAAGTCAGAGGTGGCGAAACCCGACAGGACTATAA |
| | AGATACCAGGCGTTTCCCCCTGGAAGCTCCCTCGTGCGCTCTCCTG |
| | TTCCGACCCTGCCGCTTACCGGATACCTGTCCGCCTTTCTCCCTTCG |
| | GGAAGCGTGGCGCTTTCTCATAGCTCACGCTGTAGGTATCTCAGTT |
| | CGGTGTAGGTCGTTCGCTCCAAGCTGGGCTGTGTGCACGAACCCC |
| | CCGTTCAGCCCGACCGCTGCGCCTTATCCGGTAACTATCGTCTTGA |
| | GTCCAACCCGGTAAGACACGACTTATCGCCACTGGCAGCAGCCAC |
| | TGGTAACAGGATTAGCAGAGCGAGGTATGTAGGCGGTGCTACAGA |
| | GTTCTTGAAGTGGTGGCCTAACTACGGCTACACTAGAAGGACAGT |
| | ATTTGGTATCTGCGCTCTGCTGAAGCCAGTTACCTTCGGAAAAAGA |
| | GTTGGTAGCTCTTGATCCGGCAAACAAACCACCGCTGGTAGCGGT |
| | GGTTTTTTTGTTTGCAAGCAGCAGATTACGCGCAGAAAAAAAGGA |
| | TCTCAAGAAGATCCTTTGATCTTTTCTACGGGGTCTGACGCTCAGT |
| | GGAACGAAAACTCACGTTAAGGGATTTTGGTCATGAGCGGATACA |
| | TATTTGAATGTATTTAGAAAAATAAACAAAAGAGTTTGTAGAAAC |
| | GCAAAAAGGCCATCCGTCAGGATGGCCTTCTGCTTAATTTGATGCC |
| | TGGCAGTTTATGGCGGGCGTCCTGCCCGCCACCCTCCGGGCCGTTG |
| | CTTCGCAACGTTCAAATCCGCTCCCGGCGGATTTGTCCTACTCAGG |
| | AGAGCGTTCACCGACAAACAACAGATAAAACGAAAGGCCCAGTC |
| | TTTCGACTGAGCCTTTCGTTTTATTTGATGCCTGGCAGTTCCCTACT |
| | CTCGCATGGGGAGACCCCACACTACCATCGGCGCTACGGCGTTTC |
| | ACTTCTGAGTTCGGCATGGGGTCAGGTGGGACCACCGCGCTACTG |
| | CCGCCAGGCAAATTCTGTTTTATCAGACCGCTTCTGCGTTCTGATT |
| | TAATCTGTATCAGGCTGAAAATCTTCTCTCATCCGCCAAAACAGCC |
| | AAGCTGGAGACCGTTTGGCCCCCCTCGAGCACGTAGAAAGCCAGT |
| | CCGCAGAAACGGTGCTGACCCCGGATGAATGTCAGCTACTGGGCT |
| | ATCTGGACAAGGGAAAACGCAAGCGCAAAGAGAAAGCAGGTAGC |
| | TTGCAGTGGGCTTACATGGCGATAGCTAGACTGGGCGGTTTTATG |
| | GACAGCAAGCGAACCGGAATTGCCAGCTGGGGCGCCCTCTGGTAA |
| | GGTTGGGAAGCCCTGCAAAGTAAACTGGATGGCTTTCTCGCCGCC |
| | AAGGATCTGATGGCGCAGGGGATCA |
| SEQ ID NO: 73 | ACGCGTTGACATTGATTATTGACTAGTTATTAATAGTAATCAATTA |
| pCMV PE2 P2A Cre | CGGGGTCATTAGTTCATAGCCCATATATGGAGTTCCGCGTTACATA |
| plasmid | ACTTACGGTAAATGGCCCGCCTGGCTGACCGCCCAACGACCCCCG |
| (Artificial Sequence) | CCCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCCC |
| | AGTACATGACCTTATGGGACTTTCCTACTTGGCAGTACATCTACGT |
| | ATTAGTCATCGCTATTACCATGGTGATGCGGTTTTGGCAGTACATC |
| | AATGGGCGTGGATAGCGGTTTGACTCACGGGGATTTCCAAGTCTC |
| | CACCCCATTGACGTCAATGGGAGTTTGTTTTGGCACCAAAATCAAC |
| | GGGACTTTCCAAAATGTCGTAACAACTCCGCCCCATTGACGCAAA |
| | TGGGCGGTAGGCGTGTACGGTGGGAGGTCTATATAAGCAGAGCTG |
| | GTTTAGTGAACCGTCAGATCCGCTAGAGATCCGCGGCCGCTAATA |
| | CGACTCACTATAGGGAGAGCCGCCACCATGAAACGGACAGCCGAC |
| | GGAAGCGAGTTCGAGTCACCAAAGAAGAAGCGGAAAGTCGACAA |
| | GAAGTACAGCATCGGCCTGGACATCGGCACCAACTCTGTGGGCTG |
| | GGCCGTGATCACCGACGAGTACAAGGTGCCCAGCAAGAAATTCAA |
| | GGTGCTGGGCAACACCGACCGGCACAGCATCAAGAAGAACCTGAT |
| | CGGAGCCCTGCTGTTCGACAGCGGCGAAACAGCCGAGGCCACCCG |
| | GCTGAAGAGAACCGCCAGAAGAAGATACACCAGACGGAAGAACC |
| | GGATCTGCTATCTGCAAGAGATCTTCAGCAACGAGATGGCCAAGG |
| | TGGACGACAGCTTCTTCCACAGACTGGAAGAGTCCTTCCTGGTGG |
| | AAGAGGATAAGAAGCACGAGCGGCACCCCATCTTCGGCAACATCG |
| | TGGACGAGGTGGCCTACCACGAGAAGTACCCCACCATCTACCACC |
| | TGAGAAAGAAACTGGTGGACAGCACCGACAAGGCCGACCTGCGG |
| | CTGATCTATCTGGCCCTGGCCCACATGATCAAGTTCCGGGGCCACT |
| | TCCTGATCGAGGGCGACCTGAACCCCGACAACAGCGACGTGGACA |
| | AGCTGTTCATCCAGCTGGTGCAGACCTACAACCAGCTGTTCGAGG |
| | AAAACCCCATCAACGCCAGCGGCGTGGACGCCAAGGCCATCCTGT |
| | CTGCCAGACTGAGCAAGAGCAGACGGCTGGAAAATCTGATCGCCC |
| | AGCTGCCCGGCGAGAAGAAGAATGGCCTGTTCGGAAACCTGATTG |
| | CCCTGAGCCTGGGCCTGACCCCCAACTTCAAGAGCAACTTCGACC |
| | TGGCCGAGGATGCCAAACTGCAGCTGAGCAAGGACACCTACGACG |
| | ACGACCTGGACAACCTGCTGGCCCAGATCGGCGACCAGTACGCCG |
| | ACCTGTTTCTGGCCGCCAAGAACCTGTCCGACGCCATCCTGCTGAG |
| | CGACATCCTGAGAGTGAACACCGAGATCACCAAGGCCCCCCTGAG |
| | CGCCTCTATGATCAAGAGATACGACGAGCACCACCAGGACCTGAC |
| | CCTGCTGAAAGCTCTCGTGCGGCAGCAGCTGCCTGAGAAGTACAA |
| | AGAGATTTTCTTCGACCAGAGCAAGAACGGCTACGCCGGCTACAT |
| | TGACGGCGGAGCCAGCCAGGAAGAGTTCTACAAGTTCATCAAGCC |
| | CATCCTGGAAAAGATGGACGGCACCGAGGAACTGCTCGTGAAGCT |
| | GAACAGAGAGGACCTGCTGCGGAAGCAGCGGACCTTCGACAACG |
| | GCAGCATCCCCCACCAGATCCACCTGGGAGAGCTGCACGCCATTC |
| | TGCGGCGGCAGGAAGATTTTTACCCATTCCTGAAGGACAACCGGG |
| | AAAAGATCGAGAAGATCCTGACCTTCCGCATCCCCTACTACGTGG |
| | GCCCTCTGGCCAGGGGAAACAGCAGATTCGCCTGGATGACCAGAA |
| | AGAGCGAGGAAACCATCACCCCCTGGAACTTCGAGGAAGTGGTGG |
| | ACAAGGGCGCTTCCGCCCAGAGCTTCATCGAGCGGATGACCAACT |
| | TCGATAAGAACCTGCCCAACGAGAAGGTGCTGCCCAAGCACAGCC |
| | TGCTGTACGAGTACTTCACCGTGTATAACGAGCTGACCAAAGTGA |
| | AATACGTGACCGAGGGAATGAGAAAGCCCGCCTTCCTGAGCGGCG |
| | AGCAGAAAAAGGCCATCGTGGACCTGCTGTTCAAGACCAACCGGA |
| | AAGTGACCGTGAAGCAGCTGAAAGAGGACTACTTCAAGAAAATC |
| | GAGTGCTTCGACTCCGTGGAAATCTCCGGCGTGGAAGATCGGTTC |
| | AACGCCTCCCTGGGCACATACCACGATCTGCTGAAAATTATCAAG |
| | GACAAGGACTTCCTGGACAATGAGGAAAACGAGGACATTCTGGA |
| | AGATATCGTGCTGACCCTGACACTGTTTGAGGACAGAGAGATGAT |
| | CGAGGAACGGCTGAAAACCTATGCCCACCTGTTCGACGACAAAGT |
| | GATGAAGCAGCTGAAGCGGCGGAGATACACCGGCTGGGGCAGGC |
| | TGAGCCGGAAGCTGATCAACGGCATCCGGGACAAGCAGTCCGGCA |
| | AGACAATCCTGGATTTCCTGAAGTCCGACGGCTTCGCCAACAGAA |
| | ACTTCATGCAGCTGATCCACGACGACAGCCTGACCTTTAAAGAGG |
| | ACATCCAGAAAGCCCAGGTGTCCGGCCAGGGCGATAGCCTGCACG |
| | AGCACATTGCCAATCTGGCCGGCAGCCCCGCCATTAAGAAGGGCA |
| | TCCTGCAGACAGTGAAGGTGGTGGACGAGCTCGTGAAAGTGATGG |
| | GCCGGCACAAGCCCGAGAACATCGTGATCGAAATGGCCAGAGAG |
| | AACCAGACCACCCAGAAGGGACAGAAGAACAGCCGCGAGAGAAT |
| | GAAGCGGATCGAAGAGGGCATCAAAGAGCTGGGCAGCCAGATCC |
| | TGAAAGAACACCCCGTGGAAAACACCCAGCTGCAGAACGAGAAG |
| | CTGTACCTGTACTACCTGCAGAATGGGCGGGATATGTACGTGGAC |
| | CAGGAACTGGACATCAACCGGCTGTCCGACTACGATGTGGACGCT |
| | ATCGTGCCTCAGAGCTTTCTGAAGGACGACTCCATCGACAACAAG |
| | GTGCTGACCAGAAGCGACAAGAACCGGGGCAAGAGCGACAACGT |
| | GCCCTCCGAAGAGGTCGTGAAGAAGATGAAGAACTACTGGCGGC |
| | AGCTGCTGAACGCCAAGCTGATTACCCAGAGAAAGTTCGACAATC |
| | TGACCAAGGCCGAGAGAGGCGGCCTGAGCGAACTGGATAAGGCC |
| | GGCTTCATCAAGAGACAGCTGGTGGAAACCCGGCAGATCACAAAG |
| | CACGTGGCACAGATCCTGGACTCCCGGATGAACACTAAGTACGAC |
| | GAGAATGACAAGCTGATCCGGGAAGTGAAAGTGATCACCCTGAA |
| | GTCCAAGCTGGTGTCCGATTTCCGGAAGGATTTCCAGTTTTACAAA |
| | GTGCGCGAGATCAACAACTACCACCACGCCCACGACGCCTACCTG |
| | AACGCCGTCGTGGGAACCGCCCTGATCAAAAAGTACCCTAAGCTG |
| | GAAAGCGAGTTCGTGTACGGCGACTACAAGGTGTACGACGTGCGG |
| | AAGATGATCGCCAAGAGCGAGCAGGAAATCGGCAAGGCTACCGC |
| | CAAGTACTTCTTCTACAGCAACATCATGAACTTTTTCAAGACCGAG |
| | ATTACCCTGGCCAACGGCGAGATCCGGAAGCGGCCTCTGATCGAG |
| | ACAAACGGCGAAACCGGGGAGATCGTGTGGGATAAGGGCCGGGA |
| | TTTTGCCACCGTGCGGAAAGTGCTGAGCATGCCCCAAGTGAATAT |
| | CGTGAAAAAGACCGAGGTGCAGACAGGCGGCTTCAGCAAAGAGT |
| | CTATCCTGCCCAAGAGGAACAGCGATAAGCTGATCGCCAGAAAGA |
| | AGGACTGGGACCCTAAGAAGTACGGCGGCTTCGACAGCCCCACCG |
| | TGGCCTATTCTGTGCTGGTGGTGGCCAAAGTGGAAAAGGGCAAGT |
| | CCAAGAAACTGAAGAGTGTGAAAGAGCTGCTGGGGATCACCATCA |
| | TGGAAAGAAGCAGCTTCGAGAAGAATCCCATCGACTTTCTGGAAG |
| | CCAAGGGCTACAAAGAAGTGAAAAAGGACCTGATCATCAAGCTG |
| | CCTAAGTACTCCCTGTTCGAGCTGGAAAACGGCCGGAAGAGAATG |
| | CTGGCCTCTGCCGGCGAACTGCAGAAGGGAAACGAACTGGCCCTG |
| | CCCTCCAAATATGTGAACTTCCTGTACCTGGCCAGCCACTATGAGA |
| | AGCTGAAGGGCTCCCCCGAGGATAATGAGCAGAAACAGCTGTTTG |
| | TGGAACAGCACAAGCACTACCTGGACGAGATCATCGAGCAGATCA |
| | GCGAGTTCTCCAAGAGAGTGATCCTGGCCGACGCTAATCTGGACA |
| | AAGTGCTGTCCGCCTACAACAAGCACCGGGATAAGCCCATCAGAG |
| | AGCAGGCCGAGAATATCATCCACCTGTTTACCCTGACCAATCTGG |
| | GAGCCCCTGCCGCCTTCAAGTACTTTGACACCACCATCGACCGGA |
| | AGAGGTACACCAGCACCAAAGAGGTGCTGGACGCCACCCTGATCC |
| | ACCAGAGCATCACCGGCCTGTACGAGACACGGATCGACCTGTCTC |
| | AGCTGGGAGGTGACTCTGGAGGATCTAGCGGAGGATCCTCTGGCA |
| | GCGAGACACCAGGAACAAGCGAGTCAGCAACACCAGAGAGCAGT |
| | GGCGGCAGCAGCGGCGGCAGCAGCACCCTAAATATAGAAGATGA |
| | GTATCGGCTACATGAGACCTCAAAAGAGCCAGATGTTTCTCTAGG |
| | GTCCACATGGCTGTCTGATTTTCCTCAGGCCTGGGCGGAAACCGG |
| | GGGCATGGGACTGGCAGTTCGCCAAGCTCCTCTGATCATACCTCTG |
| | AAAGCAACCTCTACCCCCGTGTCCATAAAACAATACCCCATGTCA |
| | CAAGAAGCCAGACTGGGGATCAAGCCCCACATACAGAGACTGTTG |
| | GACCAGGGAATACTGGTACCCTGCCAGTCCCCCTGGAACACGCCC |
| | CTGCTACCCGTTAAGAAACCAGGGACTAATGATTATAGGCCTGTC |
| | CAGGATCTGAGAGAAGTCAACAAGCGGGTGGAAGACATCCACCC |
| | CACCGTGCCCAACCCTTACAACCTCTTGAGCGGGCTCCCACCGTCC |
| | CACCAGTGGTACACTGTGCTTGATTTAAAGGATGCCTTTTTCTGCC |
| | TGAGACTCCACCCCACCAGTCAGCCTCTCTTCGCCTTTGAGTGGAG |
| | AGATCCAGAGATGGGAATCTCAGGACAATTGACCTGGACCAGACT |
| | CCCACAGGGTTTCAAAAACAGTCCCACCCTGTTTAATGAGGCACT |
| | GCACAGAGACCTAGCAGACTTCCGGATCCAGCACCCAGACTTGAT |
| | CCTGCTACAGTACGTGGATGACTTACTGCTGGCCGCCACTTCTGAG |
| | CTAGACTGCCAACAAGGTACTCGGGCCCTGTTACAAACCCTAGGG |
| | AACCTCGGGTATCGGGCCTCGGCCAAGAAAGCCCAAATTTGCCAG |
| | AAACAGGTCAAGTATCTGGGGTATCTTCTAAAAGAGGGTCAGAGA |
| | TGGCTGACTGAGGCCAGAAAAGAGACTGTGATGGGGCAGCCTACT |
| | CCGAAGACCCCTCGACAACTAAGGGAGTTCCTAGGGAAGGCAGGC |
| | TTCTGTCGCCTCTTCATCCCTGGGTTTGCAGAAATGGCAGCCCCCC |
| | TGTACCCTCTCACCAAACCGGGGACTCTGTTTAATTGGGGCCCAGA |
| | CCAACAAAAGGCCTATCAAGAAATCAAGCAAGCTCTTCTAACTGC |
| | CCCAGCCCTGGGGTTGCCAGATTTGACTAAGCCCTTTGAACTCTTT |
| | GTCGACGAGAAGCAGGGCTACGCCAAAGGTGTCCTAACGCAAAA |
| | ACTGGGACCTTGGCGTCGGCCGGTGGCCTACCTGTCCAAAAAGCT |
| | AGACCCAGTAGCAGCTGGGTGGCCCCCTTGCCTACGGATGGTAGC |
| | AGCCATTGCCGTACTGACAAAGGATGCAGGCAAGCTAACCATGGG |
| | ACAGCCACTAGTCATTCTGGCCCCCCATGCAGTAGAGGCACTAGT |
| | CAAACAACCCCCCGACCGCTGGCTTTCCAACGCCCGGATGACTCA |
| | CTATCAGGCCTTGCTTTTGGACACGGACCGGGTCCAGTTCGGACCG |
| | GTGGTAGCCCTGAACCCGGCTACGCTGCTCCCACTGCCTGAGGAA |
| | GGGCTGCAACACAACTGCCTTGATATCCTGGCCGAAGCCCACGGA |
| | ACCCGACCCGACCTAACGGACCAGCCGCTCCCAGACGCCGACCAC |
| | ACCTGGTACACGGATGGAAGCAGTCTCTTACAAGAGGGACAGCGT |
| | AAGGCGGGAGCTGCGGTGACCACCGAGACCGAGGTAATCTGGGCT |
| | AAAGCCCTGCCAGCCGGGACATCCGCTCAGCGGGCTGAACTGATA |
| | GCACTCACCCAGGCCCTAAAGATGGCAGAAGGTAAGAAGCTAAAT |
| | GTTTATACTGATAGCCGTTATGCTTTTGCTACTGCCCATATCCATG |
| | GAGAAATATACAGAAGGCGTGGGTGGCTCACATCAGAAGGCAAA |
| | GAGATCAAAAATAAAGACGAGATCTTGGCCCTACTAAAAGCCCTC |
| | TTTCTGCCCAAAAGACTTAGCATAATCCATTGTCCAGGACATCAAA |
| | AGGGACACAGCGCCGAGGCTAGAGGCAACCGGATGGCTGACCAA |
| | GCGGCCCGAAAGGCAGCCATCACAGAGACTCCAGACACCTCTACC |
| | CTCCTCATAGAAAATTCATCACCCTCTGGCGGCTCAAAAAGAACC |
| | GCCGACGGCAGCGAATTCGAGCCCAAGAAGAAGAGGAAAGTCGG |
| | AAGCGGAGCTACTAACTTCAGCCTGCTGAAGCAGGCTGGCGACGT |
| | GGAGGAGAACCCTGGACCTAATTTACTGACCGTACACCAAAATTT |
| | GCCTGCATTACCGGTCGATGCAACGAGTGATGAGGTTCGCAAGAA |
| | CCTGATGGACATGTTCAGGGATCGCCAGGCGTTTTCTGAGCATACC |
| | TGGAAAATGCTTCTGTCCGTTTGCCGGTCGTGGGCGGCATGGTGCA |
| | AGTTGAATAACCGGAAATGGTTTCCCGCAGAACCTGAAGATGTTC |
| | GCGATTATCTTCTATATCTTCAGGCGCGCGGTCTGGCAGTAAAAAC |
| | TATCCAGCAACATTTGGGCCAGCTAAACATGCTTCATCGTCGGTCC |
| | GGGCTGCCACGACCAAGTGACAGCAATGCTGTTTCACTGGTTATG |
| | CGGCGGATCCGAAAAGAAAACGTTGATGCCGGTGAACGTGCAAA |
| | ACAGGCTCTAGCGTTCGAACGCACTGATTTCGACCAGGTTCGTTCA |
| | CTCATGGAAAATAGCGATCGCTGCCAGGATATACGTAATCTGGCA |
| | TTTCTGGGGATTGCTTATAACACCCTGTTACGTATAGCCGAAATTG |
| | CCAGGATCAGGGTTAAAGATATCTCACGTACTGACGGTGGGAGAA |
| | TGTTAATCCATATTGGCAGAACGAAAACGCTGGTTAGCACCGCAG |
| | GTGTAGAGAAGGCACTTAGCCTGGGGGTAACTAAACTGGTCGAGC |
| | GATGGATTTCCGTCTCTGGTGTAGCTGATGATCCGAATAACTACCT |
| | GTTTTGCCGGGTCAGAAAAAATGGTGTTGCCGCGCCATCTGCCAC |
| | CAGCCAGCTATCAACTCGCGCCCTGGAAGGGATTTTTGAAGCAAC |
| | TCATCGATTGATTTACGGCGCTAAGGATGACTCTGGTCAGAGATA |
| | CCTGGCCTGGTCTGGACACAGTGCCCGTGTCGGAGCCGCGCGAGA |
| | TATGGCCCGCGCTGGAGTTTCAATACCGGAGATCATGCAAGCTGG |
| | TGGCTGGACCAATGTAAATATTGTCATGAACTATATCCGTAACCTG |
| | GATAGTGAAACAGGGGCAATGGTGCGCCTGCTGGAAGATGGCGAT |
| | TAATTTAAACCCGCTGATCAGCCTCGACTGTGCCTTCTAGTTGCCA |
| | GCCATCTGTTGTTTGCCCCTCCCCCGTGCCTTCCTTGACCCTGGAA |
| | GGTGCCACTCCCACTGTCCTTTCCTAATAAAATGAGAAAATTGCAT |
| | CGCATTGTCTGAGTAGGTGTCATTCTATTCTGGGGGGTGGGGTGGG |
| | GCAGGACAGCAAGGGGGAGGATTGGGAAGACAATAGCAGGCATG |
| | CTGGGGATGCGGTGGGCTCTATGGCTTCTGAGGCGGAAAGAACCA |
| | GCTGGGGCTCGATACCGTCGACCTCTAGCTAGAGCTTGGCGTAAT |
| | CATGGTCATAGCTGTTTCCTGTGTGAAATTGTTATCCGCTCACAAT |
| | TCCACACAACATACGAGCCGGAAGCATAAAGTGTAAAGCCTAGGG |
| | TGCCTAATGAGTGAGCTAACTCACATTAATTGCGTTGCGCTCACTG |
| | CCCGCTTTCCAGTCGGGAAACCTGTCGTGCCAGCTGCATTAATGAA |
| | TCGGCCAACGCGCGGGGAGAGGCGGTTTGCGTATTGGGCGCTCTT |
| | CCGCTTCCTCGCTCACTGACTCGCTGCGCTCGGTCGTTCGGCTGCG |
| | GCGAGCGGTATCAGCTCACTCAAAGGCGGTAATACGGTTATCCAC |
| | AGAATCAGGGGATAACGCAGGAAAGAACATGTGAGCAAAAGGCC |
| | AGCAAAAGGCCAGGAACCGTAAAAAGGCCGCGTTGCTGGCGTTTT |
| | TCCATAGGCTCCGCCCCCCTGACGAGCATCACAAAAATCGACGCT |
| | CAAGTCAGAGGTGGCGAAACCCGACAGGACTATAAAGATACCAG |
| | GCGTTTCCCCCTGGAAGCTCCCTCGTGCGCTCTCCTGTTCCGACCC |
| | TGCCGCTTACCGGATACCTGTCCGCCTTTCTCCCTTCGGGAAGCGT |
| | GGCGCTTTCTCATAGCTCACGCTGTAGGTATCTCAGTTCGGTGTAG |
| | GTCGTTCGCTCCAAGCTGGGCTGTGTGCACGAACCCCCCGTTCAGC |
| | CCGACCGCTGCGCCTTATCCGGTAACTATCGTCTTGAGTCCAACCC |
| | GGTAAGACACGACTTATCGCCACTGGCAGCAGCCACTGGTAACAG |
| | GATTAGCAGAGCGAGGTATGTAGGCGGTGCTACAGAGTTCTTGAA |
| | GTGGTGGCCTAACTACGGCTACACTAGAAGAACAGTATTTGGTAT |
| | CTGCGCTCTGCTGAAGCCAGTTACCTTCGGAAAAAGAGTTGGTAG |
| | CTCTTGATCCGGCAAACAAACCACCGCTGGTAGCGGTGGTTTTTTT |
| | GTTTGCAAGCAGCAGATTACGCGCAGAAAAAAAGGATCTCAAGA |
| | AGATCCTTTGATCTTTTCTACGGGGTCTGACGCTCAGTGGAACGAA |
| | AACTCACGTTAAGGGATTTTGGTCATGAGATTATCAAAAAGGATC |
| | TTCACCTAGATCCTTTTAAATTAAAAATGAAGTTTTAAATCAATCT |
| | AAAGTATATATGAGTAAACTTGGTCTGACAGTTACCAATGCTTAAT |
| | CAGTGAGGCACCTATCTCAGCGATCTGTCTATTTCGTTCATCCATA |
| | GTTGCCTGACTCCCCGTCGTGTAGATAACTACGATACGGGAGGGC |
| | TTACCATCTGGCCCCAGTGCTGCAATGATACCGCGAGACCCACGC |
| | TCACCGGCTCCAGATTTATCAGCAATAAACCAGCCAGCCGGAAGG |
| | GCCGAGCGCAGAAGTGGTCCTGCAACTTTATCCGCCTCCATCCAGT |
| | CTATTAATTGTTGCCGGGAAGCTAGAGTAAGTAGTTCGCCAGTTA |
| | ATAGTTTGCGCAACGTTGTTGCCATTGCTACAGGCATCGTGGTGTC |
| | ACGCTCGTCGTTTGGTATGGCTTCATTCAGCTCCGGTTCCCAACGA |
| | TCAAGGCGAGTTACATGATCCCCCATGTTGTGCAAAAAAGCGGTT |
| | AGCTCCTTCGGTCCTCCGATCGTTGTCAGAAGTAAGTTGGCCGCAG |
| | TGTTATCACTCATGGTTATGGCAGCACTGCATAATTCTCTTACTGT |
| | CATGCCATCCGTAAGATGCTTTTCTGTGACTGGTGAGTACTCAACC |
| | AAGTCATTCTGAGAATAGTGTATGCGGCGACCGAGTTGCTCTTGCC |
| | CGGCGTCAATACGGGATAATACCGCGCCACATAGCAGAACTTTAA |
| | AAGTGCTCATCATTGGAAAACGTTCTTCGGGGCGAAAACTCTCAA |
| | GGATCTTACCGCTGTTGAGATCCAGTTCGATGTAACCCACTCGTGC |
| | ACCCAACTGATCTTCAGCATCTTTTACTTTCACCAGCGTTTCTGGG |
| | TGAGCAAAAACAGGAAGGCAAAATGCCGCAAAAAAGGGAATAAG |
| | GGCGACACGGAAATGTTGAATACTCATACTCTTCCTTTTTCAATAT |
| | TATTGAAGCATTTATCAGGGTTATTGTCTCATGAGCGGATACATAT |
| | TTGAATGTATTTAGAAAAATAAACAAATAGGGGTTCCGCGCACAT |
| | TTCCCCGAAAAGTGCCACCTGACGTCGACGGATCGGGAGATCGAT |
| | CTCCCGATCCCCTAGGGTCGACTCTCAGTACAATCTGCTCTGATGC |
| | CGCATAGTTAAGCCAGTATCTGCTCCCTGCTTGTGTGTTGGAGGTC |
| | GCTGAGTAGTGCGCGAGCAAAATTTAAGCTACAACAAGGCAAGGC |
| | TTGACCGACAATTGCATGAAGAATCTGCTTAGGGTTAGGCGTTTTG |
| | CGCTGCTTCGCGATGTACGGGCCAGATAT |
| SEQ ID NO: 74 | GTCAACCAGTATCCCGGTGC |
| +90 ngRNA guide | |
| sequence | |
| (Artificial Sequence) | |
| SEQ ID NO: 75 | GTCAACCAGTATCCCGGTGCGTTTTAGAGCTAGAAATAGCAAGTT |
| +90 ngRNA | AAAATAAGGCTAGTCCGTTATCAACTTGAAAAAGTGGCACCGAGT |
| (Artificial Sequence) | CGGTGC |
| SEQ ID NO: 76 | TGATCCCCTGCGCCATCAGATCCTTGGCGGCGAGAAAGCCATCCA |
| GFP minicircle | GTTTACTTTGCAGGGCTTCCCAACCTTACCAGAGGGCGCCCCAGCT |
| template (before | GGCAATTCCGGTTCGCTTGCTGTCCATAAAACCGCCCAGTCTAGCT |
| cleavage into a | ATCGCCATGTAAGCCCACTGCAAGCTACCTGCTTTCTCTTTGCGCT |
| minicircle) | TGCGTTTTCCCTTGTCCAGATAGCCCAGTAGCTGACATTCATCCGG |
| (Artificial Sequence) | GGTCAGCACCGTTTCTGCGGACTGGCTTTCTACGTGCTCGAGGGGG |
| | GCCAAACGGTCTCCAGCTTGGCTGTTTTGGCGGATGAGAGAAGAT |
| | TTTCAGCCTGATACAGATTAAATCAGAACGCAGAAGCGGTCTGAT |
| | AAAACAGAATTTGCCTGGCGGCAGTAGCGCGGTGGTCCCACCTGA |
| | CCCCATGCCGAACTCAGAAGTGAAACGCCGTAGCGCCGATGGTAG |
| | TGTGGGGTCTCCCCATGCGAGAGTAGGGAACTGCCAGGCATCAAA |
| | TAAAACGAAAGGCTCAGTCGAAAGACTGGGCCTTTCGTTTTATCT |
| | GTTGTTTGTCGGTGAACGCTCTCCTGAGTAGGACAAATCCGCCGG |
| | GAGCGGATTTGAACGTTGCGAAGCAACGGCCCGGAGGGTGGCGG |
| | GCAGGACGCCCGCCATAAACTGCCAGGCATCAAATTAAGCAGAAG |
| | GCCATCCTGACGGATGGCCTTTTTGCGTTTCTACAAACTCTTTTGTT |
| | TATTTTTCTAAATACATTCAAATATGTATCCGCTCATGACCAAAAT |
| | CCCTTAACGTGAGTTTTCGTTCCACTGAGCGTCAGACCCCGTAGAA |
| | AAGATCAAAGGATCTTCTTGAGATCCTTTTTTTCTGCGCGTAATCT |
| | GCTGCTTGCAAACAAAAAAACCACCGCTACCAGCGGTGGTTTGTT |
| | TGCCGGATCAAGAGCTACCAACTCTTTTTCCGAAGGTAACTGGCTT |
| | CAGCAGAGCGCAGATACCAAATACTGTCCTTCTAGTGTAGCCGTA |
| | GTTAGGCCACCACTTCAAGAACTCTGTAGCACCGCCTACATACCTC |
| | GCTCTGCTAATCCTGTTACCAGTGGCTGCTGCCAGTGGCGATAAGT |
| | CGTGTCTTACCGGGTTGGACTCAAGACGATAGTTACCGGATAAGG |
| | CGCAGCGGTCGGGCTGAACGGGGGGTTCGTGCACACAGCCCAGCT |
| | TGGAGCGAACGACCTACACCGAACTGAGATACCTACAGCGTGAGC |
| | TATGAGAAAGCGCCACGCTTCCCGAAGGGAGAAAGGCGGACAGG |
| | TATCCGGTAAGCGGCAGGGTCGGAACAGGAGAGCGCACGAGGGA |
| | GCTTCCAGGGGGAAACGCCTGGTATCTTTATAGTCCTGTCGGGTTT |
| | CGCCACCTCTGACTTGAGCGTCGATTTTTGTGATGCTCGTCAGGGG |
| | GGCGGAGCCTATGGAAAAACGCCAGCAACGCGGCCTTTTTACGGT |
| | TCCTGGCCTTTTGCTGGCCTTTTGCTCACATGTTCTTTCCTGCGTTA |
| | TCCCCTGATTCTGTGGATAACCGTATTACCGCCTTTGAGTGAGCTG |
| | ATACCGCTCGCCGCAGCCGAACGACCGAGCGCAGCGAGTCAGTGA |
| | GCGAGGAAGCGGAAGAGCGCCTGATGCGGTATTTTCTCCTTACGC |
| | ATCTGTGCGGTATTTCACACCGCATATGGTGCACTCTCAGTACAAT |
| | CTGCTCTGATGCCGCATAGTTAAGCCAGTATACACTCCGCTATCGC |
| | TACGTGACTGGGTCATGGCTGCGCCCCGACACCCGCCAACACCCG |
| | CTGACGCGCCCTGACGGGCTTGTCTGCTCCCGGCATCCGCTTACAG |
| | ACAAGCTGTGACCGTCTCCGGGAGCTGCATGTGTCAGAGGTTTTC |
| | ACCGTCATCACCGAAACGCGCGAGGCAGCAGATCAATTCGCGCGC |
| | GAAGGCGAAGCGGCATGCATAATGTGCCTGTCAAATGGACGAAGC |
| | AGGGATTCTGCAAACCCTATGCTACTCCGTCAAGCCGTCAATTGTC |
| | TGATTCGTTACCAATTATGACAACTTGACGGCTACATCATTCACTT |
| | TTTCTTCACAACCGGCACGGAACTCGCTCGGGCTGGCCCCGGTGC |
| | ATTTTTTAAATACCCGCGAGAAATAGAGTTGATCGTCAAAACCAA |
| | CATTGCGACCGACGGTGGCGATAGGCATCCGGGTGGTGCTCAAAA |
| | GCAGCTTCGCCTGGCTGATACGTTGGTCCTCGCGCCAGCTTAAGAC |
| | GCTAATCCCTAACTGCTGGCGGAAAAGATGTGACAGACGCGACGG |
| | CGACAAGCAAACATGCTGTGCGACGCTGGCGATACATTACCCTGT |
| | TATCCCTAGATGACATTACCCTGTTATCCCAGATGACATTACCCTG |
| | TTATCCCTAGATGACATTACCCTGTTATCCCTAGATGACATTTACC |
| | CTGTTATCCCTAGATGACATTACCCTGTTATCCCAGATGACATTAC |
| | CCTGTTATCCCTAGATACATTACCCTGTTATCCCAGATGACATACC |
| | CTGTTATCCCTAGATGACATTACCCTGTTATCCCAGATGACATTAC |
| | CCTGTTATCCCTAGATACATTACCCTGTTATCCCAGATGACATACC |
| | CTGTTATCCCTAGATGACATTACCCTGTTATCCCAGATGACATTAC |
| | CCTGTTATCCCTAGATACATTACCCTGTTATCCCAGATGACATACC |
| | CTGTTATCCCTAGATGACATTACCCTGTTATCCCAGATGACATTAC |
| | CCTGTTATCCCTAGATACATTACCCTGTTATCCCAGATGACATACC |
| | CTGTTATCCCTAGATGACATTACCCTGTTATCCCAGATGACATTAC |
| | CCTGTTATCCCTAGATACATTACCCTGTTATCCCAGATGACATACC |
| | CTGTTATCCCTAGATGACATTACCCTGTTATCCCAGATGACATTAC |
| | CCTGTTATCCCTAGATACATTACCCTGTTATCCCAGATGACATACC |
| | CTGTTATCCCTAGATGACATTACCCTGTTATCCCAGATAAACTCAA |
| | TGATGATGATGATGATGGTCGAGACTCAGCGGCCGCGGTGCCAGG |
| | GCGTGCCCTTGGGCTCCCCGGGCGCGACTATAAGCTGCGAGCAAC |
| | TTCACTTGGGTATGCCGGCGGTAGCGCTTACCGTTCGTATAATGTA |
| | TGCTATACGAAGTTATCCGAAGCCGCTAGCGGTGGTTTGTCTGGTC |
| | AACCACCGCGGTCTCAGTGGTGTACGGTACAAACCCAGCTACCGG |
| | TCGCCACCATGCCCGCCATGAAGATCGAGTGCCGCATCACCGGCA |
| | CCCTGAACGGCGTGGAGTTCGAGCTGGTGGGCGGCGGAGAGGGC |
| | ACCCCCGAGCAGGGCCGCATGACCAACAAGATGAAGAGCACCAA |
| | AGGCGCCCTGACCTTCAGCCCCTACCTGCTGAGCCACGTGATGGG |
| | CTACGGCTTCTACCACTTCGGCACCTACCCCAGCGGCTACGAGAA |
| | CCCCTTCCTGCACGCCATCAACAACGGCGGCTACACCAACACCCG |
| | CATCGAGAAGTACGAGGACGGCGGCGTGCTGCACGTGAGCTTCAG |
| | CTACCGCTACGAGGCCGGCCGCGTGATCGGCGACTTCAAGGTGGT |
| | GGGCACCGGCTTCCCCGAGGACAGCGTGATCTTCACCGACAAGAT |
| | CATCCGCAGCAACGCCACCGTGGAGCACCTGCACCCCATGGGCGA |
| | TAACGTGCTGGTGGGCAGCTTCGCCCGCACCTTCAGCCTGCGCGA |
| | CGGCGGCTACTACAGCTTCGTGGTGGACAGCCACATGCACTTCAA |
| | GAGCGCCATCCACCCCAGCATCCTGCAGAACGGGGGCCCCATGTT |
| | CGCCTTCCGCCGCGTGGAGGAGCTGCACAGCAACACCGAGCTGGG |
| | CATCGTGGAGTACCAGCACGCCTTCAAGACCCCCATCGCCTTCGCC |
| | AGATCTCGAGCTCGATGAGTTTGGACAAACCACAACTAGAATGCA |
| | GTGAAAAAAATGCTTTATTTGTGAAATTTGTGATGCTATTGCTTTA |
| | TTTGTGGGCCCGCCCCAACTGGGGTAACCTTTGAGTTCTCTCAGTT |
| | GGGGGTAATCAGCATCATGATGTGGTACCACATCATGATGCTGAT |
| | TATAAGAATGCGGCCGCCACACTCTAGTGGATCTCGAGTTAATAA |
| | TTCAGAAGAACTCGTCAAGAAGGCGATAGAAGGCGATGCGCTGCG |
| | AATCGGGAGCGGCGATACCGTAAAGCACGAGGAAGCGGTCAGCC |
| | CATTCGCCGCCAAGCTCTTCAGCAATATCACGGGTAGCCAACGCT |
| | ATGTCCTGATAGCGGTCCGCCACACCCAGCCGGCCACAGTCGATG |
| | AATCCAGAAAAGCGGCCATTTTCCACCATGATATTCGGCAAGCAG |
| | GCATCGCCATGGGTCACGACGAGATCCTCGCCGTCGGGCATGCTC |
| | GCCTTGAGCCTGGCGAACAGTTCGGCTGGCGCGAGCCCCTGATGC |
| | TCTTCGTCCAGATCATCCTGATCGACAAGACCGGCTTCCATCCGAG |
| | TACGTGCTCGCTCGATGCGATGTTTCGCTTGGTGGTCGAATGGGCA |
| | GGTAGCCGGATCAAGCGTATGCAGCCGCCGCATTGCATCAGCCAT |
| | GATGGATACTTTCTCGGCAGGAGCAAGGTGTAGATGACATGGAGA |
| | TCCTGCCCCGGCACTTCGCCCAATAGCAGCCAGTCCCTTCCCGCTT |
| | CAGTGACAACGTCGAGCACAGCTGCGCAAGGAACGCCCGTCGTGG |
| | CCAGCCACGATAGCCGCGCTGCCTCGTCTTGCAGTTCATTCAGGGC |
| | ACCGGACAGGTCGGTCTTGACAAAAAGAACCGGGCGCCCCTGCGC |
| | TGACAGCCGGAACACGGCGGCATCAGAGCAGCCGATTGTCTGTTG |
| | TGCCCAGTCATAGCCGAATAGCCTCTCCACCCAAGCGGCCGGAGA |
| | ACCTGCGTGCAATCCATCTTGTTCAATCATGCGAAACGATCCTCAT |
| | CCTGTCTCTTGATCAGAGCT |
| SEQ ID NO: 77 | TGATCCCCTGCGCCATCAGATCCTTGGCGGCGAGAAAGCCATCCA |
| Gaussia Luciferase | GTTTACTTTGCAGGGCTTCCCAACCTTACCAGAGGGCGCCCCAGCT |
| minicircle template | GGCAATTCCGGTTCGCTTGCTGTCCATAAAACCGCCCAGTCTAGCT |
| (Artificial Sequence) | ATCGCCATGTAAGCCCACTGCAAGCTACCTGCTTTCTCTTTGCGCT |
| | TGCGTTTTCCCTTGTCCAGATAGCCCAGTAGCTGACATTCATCCGG |
| | GGTCAGCACCGTTTCTGCGGACTGGCTTTCTACGTGCTCGAGGGGG |
| | GCCAAACGGTCTCCAGCTTGGCTGTTTTGGCGGATGAGAGAAGAT |
| | TTTCAGCCTGATACAGATTAAATCAGAACGCAGAAGCGGTCTGAT |
| | AAAACAGAATTTGCCTGGCGGCAGTAGCGCGGTGGTCCCACCTGA |
| | CCCCATGCCGAACTCAGAAGTGAAACGCCGTAGCGCCGATGGTAG |
| | TGTGGGGTCTCCCCATGCGAGAGTAGGGAACTGCCAGGCATCAAA |
| | TAAAACGAAAGGCTCAGTCGAAAGACTGGGCCTTTCGTTTTATCT |
| | GTTGTTTGTCGGTGAACGCTCTCCTGAGTAGGACAAATCCGCCGG |
| | GAGCGGATTTGAACGTTGCGAAGCAACGGCCCGGAGGGTGGCGG |
| | GCAGGACGCCCGCCATAAACTGCCAGGCATCAAATTAAGCAGAAG |
| | GCCATCCTGACGGATGGCCTTTTTGCGTTTCTACAAACTCTTTTGTT |
| | TATTTTTCTAAATACATTCAAATATGTATCCGCTCATGACCAAAAT |
| | CCCTTAACGTGAGTTTTCGTTCCACTGAGCGTCAGACCCCGTAGAA |
| | AAGATCAAAGGATCTTCTTGAGATCCTTTTTTTCTGCGCGTAATCT |
| | GCTGCTTGCAAACAAAAAAACCACCGCTACCAGCGGTGGTTTGTT |
| | TGCCGGATCAAGAGCTACCAACTCTTTTTCCGAAGGTAACTGGCTT |
| | CAGCAGAGCGCAGATACCAAATACTGTCCTTCTAGTGTAGCCGTA |
| | GTTAGGCCACCACTTCAAGAACTCTGTAGCACCGCCTACATACCTC |
| | GCTCTGCTAATCCTGTTACCAGTGGCTGCTGCCAGTGGCGATAAGT |
| | CGTGTCTTACCGGGTTGGACTCAAGACGATAGTTACCGGATAAGG |
| | CGCAGCGGTCGGGCTGAACGGGGGGTTCGTGCACACAGCCCAGCT |
| | TGGAGCGAACGACCTACACCGAACTGAGATACCTACAGCGTGAGC |
| | TATGAGAAAGCGCCACGCTTCCCGAAGGGAGAAAGGCGGACAGG |
| | TATCCGGTAAGCGGCAGGGTCGGAACAGGAGAGCGCACGAGGGA |
| | GCTTCCAGGGGGAAACGCCTGGTATCTTTATAGTCCTGTCGGGTTT |
| | CGCCACCTCTGACTTGAGCGTCGATTTTTGTGATGCTCGTCAGGGG |
| | GGCGGAGCCTATGGAAAAACGCCAGCAACGCGGCCTTTTTACGGT |
| | TCCTGGCCTTTTGCTGGCCTTTTGCTCACATGTTCTTTCCTGCGTTA |
| | TCCCCTGATTCTGTGGATAACCGTATTACCGCCTTTGAGTGAGCTG |
| | ATACCGCTCGCCGCAGCCGAACGACCGAGCGCAGCGAGTCAGTGA |
| | GCGAGGAAGCGGAAGAGCGCCTGATGCGGTATTTTCTCCTTACGC |
| | ATCTGTGCGGTATTTCACACCGCATATGGTGCACTCTCAGTACAAT |
| | CTGCTCTGATGCCGCATAGTTAAGCCAGTATACACTCCGCTATCGC |
| | TACGTGACTGGGTCATGGCTGCGCCCCGACACCCGCCAACACCCG |
| | CTGACGCGCCCTGACGGGCTTGTCTGCTCCCGGCATCCGCTTACAG |
| | ACAAGCTGTGACCGTCTCCGGGAGCTGCATGTGTCAGAGGTTTTC |
| | ACCGTCATCACCGAAACGCGCGAGGCAGCAGATCAATTCGCGCGC |
| | GAAGGCGAAGCGGCATGCATAATGTGCCTGTCAAATGGACGAAGC |
| | AGGGATTCTGCAAACCCTATGCTACTCCGTCAAGCCGTCAATTGTC |
| | TGATTCGTTACCAATTATGACAACTTGACGGCTACATCATTCACTT |
| | TTTCTTCACAACCGGCACGGAACTCGCTCGGGCTGGCCCCGGTGC |
| | ATTTTTTAAATACCCGCGAGAAATAGAGTTGATCGTCAAAACCAA |
| | CATTGCGACCGACGGTGGCGATAGGCATCCGGGTGGTGCTCAAAA |
| | GCAGCTTCGCCTGGCTGATACGTTGGTCCTCGCGCCAGCTTAAGAC |
| | GCTAATCCCTAACTGCTGGCGGAAAAGATGTGACAGACGCGACGG |
| | CGACAAGCAAACATGCTGTGCGACGCTGGCGATACATTACCCTGT |
| | TATCCCTAGATGACATTACCCTGTTATCCCAGATGACATTACCCTG |
| | TTATCCCTAGATGACATTACCCTGTTATCCCTAGATGACATTTACC |
| | CTGTTATCCCTAGATGACATTACCCTGTTATCCCAGATGACATTAC |
| | CCTGTTATCCCTAGATACATTACCCTGTTATCCCAGATGACATACC |
| | CTGTTATCCCTAGATGACATTACCCTGTTATCCCAGATGACATTAC |
| | CCTGTTATCCCTAGATACATTACCCTGTTATCCCAGATGACATACC |
| | CTGTTATCCCTAGATGACATTACCCTGTTATCCCAGATGACATTAC |
| | CCTGTTATCCCTAGATACATTACCCTGTTATCCCAGATGACATACC |
| | CTGTTATCCCTAGATGACATTACCCTGTTATCCCAGATGACATTAC |
| | CCTGTTATCCCTAGATACATTACCCTGTTATCCCAGATGACATACC |
| | CTGTTATCCCTAGATGACATTACCCTGTTATCCCAGATGACATTAC |
| | CCTGTTATCCCTAGATACATTACCCTGTTATCCCAGATGACATACC |
| | CTGTTATCCCTAGATGACATTACCCTGTTATCCCAGATGACATTAC |
| | CCTGTTATCCCTAGATACATTACCCTGTTATCCCAGATGACATACC |
| | CTGTTATCCCTAGATGACATTACCCTGTTATCCCAGATAAACTCAA |
| | TGATGATGATGATGATGGTCGAGACTCAGCGGCCGCGGTGCCAGG |
| | GCGTGCCCTTGGGCTCCCCGGGCGCGACTATAAGCTGCGAGCAAC |
| | TTCACTTGGGTATGCCGGCGGTAGCGCTTACCGTTCGTATAATGTA |
| | TGCTATACGAAGTTATCCGAAGCCGCTAGCGGTGGTTTGTCTGGTC |
| | AACCACCGCGGTCTCAGTGGTGTACGGTACAAACCCACTACCGGT |
| | CGCCACCATGGGAGTCAAAGTTCTGTTTGCCCTGATCTGCATCGCT |
| | GTGGCCGAGGCCAAGCCCACCGAGAACAACGAAGACTTCAACATC |
| | GTGGCCGTGGCCAGCAACTTCGCGACCACGGATCTCGATGCTGAC |
| | CGCGGGAAGTTGCCCGGCAAGAAGCTGCCGCTGGAGGTGCTCAAA |
| | GAGATGGAAGCCAATGCCCGGAAAGCTGGCTGCACCAGGGGCTGT |
| | CTGATCTGCCTGTCCCACATCAAGTGCACGCCCAAGATGAAGAAG |
| | TTCATCCCAGGACGCTGCCACACCTACGAAGGCGACAAAGAGTCC |
| | GCACAGGGCGGCATAGGCGAGGCGATCGTCGACATTCCTGAGATT |
| | CCTGGGTTCAAGGACTTGGAGCCCATGGAGCAGTTCATCGCACAG |
| | GTCGATCTGTGTGTGGACTGCACAACTGGCTGCCTCAAAGGGCTT |
| | GCCAACGTGCAGTGTTCTGACCTGCTCAAGAAGTGGCTGCCGCAA |
| | CGCTGTGCGACCTTTGCCAGCAAGATCCAGGGCCAGGTGGACAAG |
| | ATCAAGGGGGCCGGTGGTGACTAAGCGGAGCTCGATGAGTTTGGA |
| | CAAACCACAACTAGAATGCAGTGAAAAAAATGCTTTATTTGTGAA |
| | ATTTGTGATGCTATTGCTTTATTTGTGGGCCCGCCCCAACTGGGGT |
| | AACCTTTGAGTTCTCTCAGTTGGGGGTAATCAGCATCATGATGTGG |
| | TACCACATCATGATGCTGATTATAAGAATGCGGCCGCCACACTCT |
| | AGTGGATCTCGAGTTAATAATTCAGAAGAACTCGTCAAGAAGGCG |
| | ATAGAAGGCGATGCGCTGCGAATCGGGAGCGGCGATACCGTAAA |
| | GCACGAGGAAGCGGTCAGCCCATTCGCCGCCAAGCTCTTCAGCAA |
| | TATCACGGGTAGCCAACGCTATGTCCTGATAGCGGTCCGCCACAC |
| | CCAGCCGGCCACAGTCGATGAATCCAGAAAAGCGGCCATTTTCCA |
| | CCATGATATTCGGCAAGCAGGCATCGCCATGGGTCACGACGAGAT |
| | CCTCGCCGTCGGGCATGCTCGCCTTGAGCCTGGCGAACAGTTCGG |
| | CTGGCGCGAGCCCCTGATGCTCTTCGTCCAGATCATCCTGATCGAC |
| | AAGACCGGCTTCCATCCGAGTACGTGCTCGCTCGATGCGATGTTTC |
| | GCTTGGTGGTCGAATGGGCAGGTAGCCGGATCAAGCGTATGCAGC |
| | CGCCGCATTGCATCAGCCATGATGGATACTTTCTCGGCAGGAGCA |
| | AGGTGTAGATGACATGGAGATCCTGCCCCGGCACTTCGCCCAATA |
| | GCAGCCAGTCCCTTCCCGCTTCAGTGACAACGTCGAGCACAGCTG |
| | CGCAAGGAACGCCCGTCGTGGCCAGCCACGATAGCCGCGCTGCCT |
| | CGTCTTGCAGTTCATTCAGGGCACCGGACAGGTCGGTCTTGACAA |
| | AAAGAACCGGGCGCCCCTGCGCTGACAGCCGGAACACGGCGGCA |
| | TCAGAGCAGCCGATTGTCTGTTGTGCCCAGTCATAGCCGAATAGC |
| | CTCTCCACCCAAGCGGCCGGAGAACCTGCGTGCAATCCATCTTGTT |
| | CAATCATGCGAAACGATCCTCATCCTGTCTCTTGATCAGAGCT |
| SEQ ID NO: 78 | CCCCAACTGGGGTAACCTTTGAGTTCTCTCAGTTGGGG |
| pseudo attP site | |
| (Artificial sequence) | |
| SEQ ID NO: 79 | GACTGAAACTTCACAGAATAGTTTTAGAGCTAGAAATAGCAAGTT |
| Albumin-pegRNA- | AAAATAAGGCTAGTCCGTTATCAACTTGAAAAAGTGGCACCGAGT |
| SERPIN | CGGTGCTTGGGATAGTTATGAATTCAATCTTCAACCCTATCCGGAT |
| (Artificial Sequence) | GATCCTGACGACGGAGACCGCCGTCGTCGACAAGCCGGCCTCTGT |
| | GAAGTTTCAGTCA |
| SEQ ID NO: 80 | GACTGAAACTTCACAGAATAGTTTTAGAGCTAGAAATAGCAAGTT |
| Albumin-pegRNA- | AAAATAAGGCTAGTCCGTTATCAACTTGAAAAAGTGGCACCGAGT |
| CPS1 | CGGTGCTTGGGATAGTTATGAATTCAATCTTCAACCCTATCCGGAT |
| (Artificial Sequence) | GATCCTGACGACGGAGACCGCCGTCGTCGACAAGCCGGCCTCTGT |
| | GAAGTTTC |
| SEQ ID NO: 81 | GGCCCAGACTGAGCACGTGAGTTTTAGAGCTAGAAATAGCAAGTT |
| 34 bp lox71 pegRNA | AAAATAAGGCTAGTCCGTTATCAACTTGAAAAAGTGGCACCGAGT |
| (Artificial Sequence) | CGGTGCTGGAGGAAGCAGGGCTTCCTTTCCTCTGCCATCATACCGT |
| | TCGTATAGCATACATTATACGAAGTTATCGTGCTCAGTCTG |
| SEQ ID NO: 82 | GGCCCAGACTGAGCACGTGAGTTTTAGAGCTAGAAATAGCAAGTT |
| 34 bp lox66 pegRNA | AAAATAAGGCTAGTCCGTTATCAACTTGAAAAAGTGGCACCGAGT |
| (Artificial Sequence) | CGGTGCTGGAGGAAGCAGGGCTTCCTTTCCTCTGCCATCAATAACT |
| | TCGTATAGCATACATTATACGAACGGTACGTGCTCAGTCTG |
| SEQ ID NO: 83 | GGCCCAGACTGAGCACGTGA |
| gRNA | |
| (Artificial Sequence) | |
| SEQ ID NO: 84 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 46 | GGTGCGACGAGCGCGGCGATATCATCATCCATGGCCGGATGATCC |
| (original length) | TGACGACGGAGACCGCCGTCGTCGACAAGCCGGCCTGAGCTGCGA |
| pegRNA | GAA |
| (Artificial Sequence) | |
| SEQ ID NO: 85 | GCTATTCTCGCAGCTCACCAGTTTTAGAGCTAGAAATAGCAAGTT |
| ACTB N-term | AAAATAAGGCTAGTCCGTTATCAACTTGAAAAAGTGGCACCGAG |
| PBS_13_RT_29_with | TCGGTGCGAGTCGGTGCGACGAGCGCGGCGATATCATCATCCAT |
| TP901-1 minimal | GGCACAATTAACATCTCAATCAAGGTAAATGCTTGAGCTGCGAG |
| attB f pegRNA | AA |
| (Artificial Sequence) | |
| SEQ ID NO: 86 | GCTATTCTCGCAGCTCACCAGTTTTAGAGCTAGAAATAGCAAGTT |
| ACTB N-term | AAAATAAGGCTAGTCCGTTATCAACTTGAAAAAGTGGCACCGAG |
| PBS_13_RT_29_with | TCGGTGCGAGTCGGTGCGACGAGCGCGGCGATATCATCATCCAT |
| TP901-1 minimal | GGAGCATTTACCTTGATTGAGATGTTAATTGTGTGAGCTGCGAGA |
| attB rc pegRNA | A |
| (Artificial Sequence) | |
| SEQ ID NO: 87 | GCTATTCTCGCAGCTCACCAGTTTTAGAGCTAGAAATAGCAAGTT |
| ACTB N-term | AAAATAAGGCTAGTCCGTTATCAACTTGAAAAAGTGGCACCGAG |
| PBS_13_RT_29_with | TCGGTGCGAGTCGGTGCGACGAGCGCGGCGATATCATCATCCAT |
| PhiBT1 minimal | GGCAGGTTTTTGACGAAAGTGATCCAGATGATCCAGTGAGCTGC |
| attB f pegRNA | GAGAA |
| (Artificial Sequence) | |
| SEQ ID NO: 88 | GCTATTCTCGCAGCTCACCAGTTTTAGAGCTAGAAATAGCAAGTT |
| ACTB N-term | AAAATAAGGCTAGTCCGTTATCAACTTGAAAAAGTGGCACCGAG |
| PBS_13_RT_29_with | TCGGTGCGAGTCGGTGCGACGAGCGCGGCGATATCATCATCCAT |
| PhiBT1 minimal | GGCTGGATCATCTGGATCACTTTCGTCAAAAACCTGTGAGCTGCG |
| attB rc pegRNA | AGAA |
| (Artificial Sequence) | |
| SEQ ID NO: 89 | GAAGCCGGCCTTGCACATGCGTTTTAGAGCTAGAAATAGCAAGT |
| ACTB N-term | TAAAATAAGGCTAGTCCGTTATCAACTTGAAAAAGTGGCACCGA |
| Nicking guide 1 +48 | GTCGGTGC |
| guide | |
| (Artificial Sequence) | |
| SEQ ID NO: 90 | GAAGCCGGCCTTGCACATGCGTTTTAGAGCTAGAAATAGCAAGT |
| ACTB N-term | TAAAATAAGGCTAGTCCGTTATCAACTTGAAAAAGTGGCACCGA |
| PBS_18_RT_16_with_ | GTCGGTGCATATCATCATCCATGGTACCGTTCGTATAGCATACAT |
| Lox71_Cre | TATACGAAGTTATTGAGCTGCGAGAATAGCC |
| pegRNA | |
| (Artificial Sequence) | |
| SEQ ID NO: 91 | GAAGCCGGCCTTGCACATGCGTTTTAGAGCTAGAAATAGCAAGT |
| ACTB N-term | TAAAATAAGGCTAGTCCGTTATCAACTTGAAAAAGTGGCACCGA |
| PBS_13_RT_29_with_ | GTCGGTGCGACGAGCGCGGCGATATCATCATCCATGGTACCGTT |
| Lox71_Cre | CGTATAGCATACATTATACGAAGTTATTGAGCTGCGAGAA |
| pegRNA | |
| (Artificial Sequence) | |
| SEQ ID NO: 92 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 34 pegRNA | GGTGCTCGACGACGAGCGCGGCGATATCATCATCCATGGCCGGAT |
| (Artificial Sequence) | GATCCTGACGACGGAGACCGCCGTCGTCGACAAGCCGGCCTGAGC |
| | TGCGAGAA |
| SEQ ID NO: 93 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 26 pegRNA | GGTGCGAGCGCGGCGATATCATCATCCATGGCCGGATGATCCTGA |
| (Artificial Sequence) | CGACGGAGACCGCCGTCGTCGACAAGCCGGCCTGAGCTGCGAGAA |
| SEQ ID NO: 94 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 23 pegRNA | GGTGCCGCGGCGATATCATCATCCATGGCCGGATGATCCTGACGAC |
| (Artificial Sequence) | GGAGACCGCCGTCGTCGACAAGCCGGCCTGAGCTGCGAGAA |
| SEQ ID NO: 95 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 20 pegRNA | GGTGCGGCGATATCATCATCCATGGCCGGATGATCCTGACGACGG |
| (Artificial Sequence) | AGACCGCCGTCGTCGACAAGCCGGCCTGAGCTGCGAGAA |
| SEQ ID NO: 96 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 16 pegRNA | GGTGCATATCATCATCCATGGCCGGATGATCCTGACGACGGAGAC |
| (Artificial Sequence) | CGCCGTCGTCGACAAGCCGGCCTGAGCTGCGAGAA |
| SEQ ID NO: 97 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 18 RT 34 pegRNA | GGTGCTCGACGACGAGCGCGGCGATATCATCATCCATGGCCGGAT |
| (Artificial Sequence) | GATCCTGACGACGGAGACCGCCGTCGTCGACAAGCCGGCCTGAGC |
| | TGCGAGAATAGCC |
| SEQ ID NO: 98 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 18 RT 29 pegRNA | GGTGCGACGAGCGCGGCGATATCATCATCCATGGCCGGATGATCC |
| (Artificial Sequence) | TGACGACGGAGACCGCCGTCGTCGACAAGCCGGCCTGAGCTGCGA |
| | GAATAGCC |
| SEQ ID NO: 99 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 18 RT 16 pegRNA | GGTGCATATCATCATCCATGGCCGGATGATCCTGACGACGGAGAC |
| (Artificial Sequence) | CGCCGTCGTCGACAAGCCGGCCTGAGCTGCGAGAATAGCC |
| SEQ ID NO: 100 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAG |
| 13 RT 39 pegRNA | TCGGTGCCTGCCCATCCGCGGCGGCACGGGGGTCGCAGTCGCCA |
| (Artificial Sequence) | TGCCGGATGATCCTGACGACGGAGACCGCCGTCGTCGACAAGCC |
| | GGCCCGGGCGGCGGAGA |
| SEQ ID NO: 101 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAG |
| 13 RT 34 pegRNA | TCGGTGCCATCCGCGGCGGCACGGGGGTCGCAGTCGCCATGCCG |
| (Artificial Sequence) | GATGATCCTGACGACGGAGACCGCCGTCGTCGACAAGCCGGCCC |
| | GGGCGGCGGAGA |
| SEQ ID NO: 102 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAG |
| 13 RT 29 pegRNA | TCGGTGCGCGGCGGCACGGGGGTCGCAGTCGCCATGCCGGATGA |
| (Artificial Sequence) | TCCTGACGACGGAGACCGCCGTCGTCGACAAGCCGGCCCGGGCG |
| | GCGGAGA |
| SEQ ID NO: 103 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAG |
| 13 RT 24 pegRNA | TCGGTGCGGCACGGGGGTCGCAGTCGCCATGCCGGATGATCCTG |
| (Artificial Sequence) | ACGACGGAGACCGCCGTCGTCGACAAGCCGGCCCGGGCGGCGGA |
| | GA |
| SEQ ID NO: 104 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAG |
| 13 RT 19 pegRNA | TCGGTGCGGGGGTCGCAGTCGCCATGCCGGATGATCCTGACGAC |
| (Artificial Sequence) | GGAGACCGCCGTCGTCGACAAGCCGGCCCGGGCGGCGGAGA |
| SEQ ID NO: 105 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAG |
| 18 RT 39 pegRNA | TCGGTGCCTGCCCATCCGCGGCGGCACGGGGGTCGCAGTCGCCA |
| (Artificial Sequence) | TGCCGGATGATCCTGACGACGGAGACCGCCGTCGTCGACAAGCC |
| | GGCCCGGGCGGCGGAGACAGCG |
| SEQ ID NO: 106 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAG |
| 18 RT 34 pegRNA | TCGGTGCCATCCGCGGCGGCACGGGGGTCGCAGTCGCCATGCCG |
| (Artificial Sequence) | GATGATCCTGACGACGGAGACCGCCGTCGTCGACAAGCCGGCCC |
| | GGGCGGCGGAGACAGCG |
| SEQ ID NO: 107 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAGT |
| 18 RT 29 pegRNA | CGGTGCGCGGCGGCACGGGGGTCGCAGTCGCCATGCCGGATGATC |
| (Artificial Sequence) | CTGACGACGGAGACCGCCGTCGTCGACAAGCCGGCCCGGGCGGCG |
| | GAGACAGCG |
| SEQ ID NO: 108 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAG |
| 18 RT 24 pegRNA | TCGGTGCGGCACGGGGGTCGCAGTCGCCATGCCGGATGATCCTG |
| (Artificial Sequence) | ACGACGGAGACCGCCGTCGTCGACAAGCCGGCCCGGGCGGCGGA |
| | GACAGCG |
| SEQ ID NO: 109 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAG |
| 18 RT 19 pegRNA | TCGGTGCGGGGGTCGCAGTCGCCATGCCGGATGATCCTGACGAC |
| (Artificial Sequence) | GGAGACCGCCGTCGTCGACAAGCCGGCCCGGGCGGCGGAGACAG |
| | CG |
| SEQ ID NO: 110 | GCGTGGTGGGGCCGCCAGCGGTTTTAGAGCTAGAAATAGCAAGT |
| LMNB1 N-term | TAAAATAAGGCTAGTCCGTTATCAACTTGAAAAAGTGGCACCGA |
| Nicking guide 1 +46 | GTCGGTGC |
| (Artificial Sequence) | |
| SEQ ID NO: 111 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 42 | GGTGCGACGAGCGCGGCGATATCATCATCCATGGGGATGATCCTG |
| pegRNA | ACGACGGAGACCGCCGTCGTCGACAAGCCGGTGAGCTGCGAGAA |
| (Artificial Sequence) | |
| SEQ ID NO: 112 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 40 | GGTGCGACGAGCGCGGCGATATCATCATCCATGGGATGATCCTGA |
| pegRNA | CGACGGAGACCGCCGTCGTCGACAAGCCGTGAGCTGCGAGAA |
| (Artificial Sequence) | |
| SEQ ID NO: 113 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 38 | GGTGCGACGAGCGCGGCGATATCATCATCCATGGATGATCCTGAC |
| pegRNA | GACGGAGACCGCCGTCGTCGACAAGCCTGAGCTGCGAGAA |
| (Artificial Sequence) | |
| SEQ ID NO: 114 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 36 | GGTGCGACGAGCGCGGCGATATCATCATCCATGGTGATCCTGACG |
| pegRNA | ACGGAGACCGCCGTCGTCGACAAGCTGAGCTGCGAGAA |
| (Artificial Sequence) | |
| SEQ ID NO: 115 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAGT |
| 13 RT 29 attB 44 | CGGTGCGCGGCGGCACGGGGGTCGCAGTCGCCATGCGGATGATCC |
| pegRNA v2 | TGACGACGGAGACCGCCGTCGTCGACAAGCCGGCCGGGCGGCGG |
| (Artificial Sequence) | AGA |
| SEQ ID NO: 116 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAGT |
| 13 RT 29 attB 42 | CGGTGCGCGGCGGCACGGGGGTCGCAGTCGCCATGGGATGATCCT |
| pegRNA v2 | GACGACGGAGACCGCCGTCGTCGACAAGCCGGCGGGCGGCGGAG |
| (Artificial Sequence) | A |
| SEQ ID NO: 117 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAGT |
| 13 RT 29 attB 40 | CGGTGCGCGGCGGCACGGGGGTCGCAGTCGCCATGGATGATCCTG |
| pegRNA v2 | ACGACGGAGACCGCCGTCGTCGACAAGCCGCGGGCGGCGGAGA |
| (Artificial Sequence) | |
| SEQ ID NO: 118 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAGT |
| 13 RT 29 attB 38 | CGGTGCGCGGCGGCACGGGGGTCGCAGTCGCCATGATGATCCTGA |
| pegRNA v2 | CGACGGAGACCGCCGTCGTCGACAAGCCCGGGCGGCGGAGA |
| (Artificial Sequence) | |
| SEQ ID NO: 119 | GCGTATTGCCTGGAGGATGGGTTTTAGAGCTAGAAATAGCAAGT |
| NOLC1 N-term PBS | TAAAATAAGGCTAGTCCGTTATCAACTTGAAAAAGTGGCACCGA |
| 18 RT 29 attB 46 | GTCGGTGCGAACCACGCGGCGAATGCCGGCGTCCGCCCCGGATG |
| pegRNA | ATCCTGACGACGGAGACCGCCGTCGTCGACAAGCCGGCCTCCTC |
| (Artificial Sequence) | CAGGCAATACGCG |
| SEQ ID NO: 120 | GCGTATTGCCTGGAGGATGGGTTTTAGAGCTAGAAATAGCAAGTT |
| NOLC1 N-term PBS | AAAATAAGGCTAGTCCGTTATCAACTTGAAAAAGTGGCACCGAGT |
| 13 RT 29 attB 46 | CGGTGCGAACCACGCGGCGAATGCCGGCGTCCGCCCCGGATGATC |
| pegRNA | CTGACGACGGAGACCGCCGTCGTCGACAAGCCGGCCTCCTCCAGG |
| (Artificial Sequence) | CAAT |
| SEQ ID NO: 121 | GCGTATTGCCTGGAGGATGGGTTTTAGAGCTAGAAATAGCAAGT |
| NOLC1 N-term PBS | TAAAATAAGGCTAGTCCGTTATCAACTTGAAAAAGTGGCACCGA |
| 13 RT 29 attB 44 | GTCGGTGCGAACCACGCGGCGAATGCCGGCGTCCGCCCGGATGA |
| pegRNA | TCCTGACGACGGAGACCGCCGTCGTCGACAAGCCGGCTCCTCCA |
| (Artificial Sequence) | GGCAAT |
| SEQ ID NO: 122 | GCGTATTGCCTGGAGGATGGGTTTTAGAGCTAGAAATAGCAAGT |
| NOLC1 N-term PBS | TAAAATAAGGCTAGTCCGTTATCAACTTGAAAAAGTGGCACCGA |
| 13 RT 29 attB 42 | GTCGGTGCGAACCACGCGGCGAATGCCGGCGTCCGCCGGATGAT |
| pegRNA | CCTGACGACGGAGACCGCCGTCGTCGACAAGCCGGTCCTCCAGG |
| (Artificial Sequence) | CAAT |
| SEQ ID NO: 123 | GCGTATTGCCTGGAGGATGGGTTTTAGAGCTAGAAATAGCAAGT |
| NOLC1 N-term PBS | TAAAATAAGGCTAGTCCGTTATCAACTTGAAAAAGTGGCACCGA |
| 13 RT 29 attB 40 | GTCGGTGCGAACCACGCGGCGAATGCCGGCGTCCGCCGATGATC |
| pegRNA | CTGACGACGGAGACCGCCGTCGTCGACAAGCCGTCCTCCAGGCA |
| (Artificial Sequence) | AT |
| SEQ ID NO: 124 | GCGTATTGCCTGGAGGATGGGTTTTAGAGCTAGAAATAGCAAGTT |
| NOLC1 N-term PBS | AAAATAAGGCTAGTCCGTTATCAACTTGAAAAAGTGGCACCGAG |
| 13 RT 29 attB 38 | TCGGTGCGAACCACGCGGCGAATGCCGGCGTCCGCCATGATCCT |
| pegRNA | GACGACGGAGACCGCCGTCGTCGACAAGCCTCCTCCAGGCAAT |
| (Artificial Sequence) | |
| SEQ ID NO: 125 | GAGCCGAGCACGAGGGGATACGTTTTAGAGCTAGAAATAGCAAGT |
| NOLC1 nicking | TAAAATAAGGCTAGTCCGTTATCAACTTGAAAAAGTGGCACCGAG |
| guide-43 | TCGGTGC |
| (Artificial Sequence) | |
| SEQ ID NO: 126 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 20 attB 38 | GGTGCGGCGATATCATCATCCATGGATGATCCTGACGACGGAGAC |
| pegRNA | CGCCGTCGTCGACAAGCCTGAGCTGCGAGAA |
| (Artificial Sequence) | |
| SEQ ID NO: 127 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 15 attB 38 | GGTGCTATCATCATCCATGGATGATCCTGACGACGGAGACCGCCG |
| pegRNA | TCGTCGACAAGCCTGAGCTGCGAGAA |
| (Artificial Sequence) | |
| SEQ ID NO: 128 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 10 attB 38 | GGTGCTCATCCATGGATGATCCTGACGACGGAGACCGCCGTCGTC |
| pegRNA | GACAAGCCTGAGCTGCGAGAA |
| (Artificial Sequence) | |
| SEQ ID NO: 129 | GCTATTCTCGCAGCTCACCAGTTTTAGAGCTAGAAATAGCAAGTT |
| ACTB N-term PBS 9 | AAAATAAGGCTAGTCCGTTATCAACTTGAAAAAGTGGCACCGAG |
| RT 20 attB 38 | TCGGTGCGGCGATATCATCATCCATGGATGATCCTGACGACGGAG |
| pegRNA | ACCGCCGTCGTCGACAAGCCTGAGCTGCG |
| (Artificial Sequence) | |
| SEQ ID NO: 130 | GCTATTCTCGCAGCTCACCAGTTTTAGAGCTAGAAATAGCAAGTTA |
| ACTB N-term PBS 9 | AAATAAGGCTAGTCCGTTATCAACTTGAAAAAGTGGCACCGAGTC |
| RT 15 attB 38 | GGTGCTATCATCATCCATGGATGATCCTGACGACGGAGACCGCCG |
| pegRNA | TCGTCGACAAGCCTGAGCTGCG |
| (Artificial Sequence) | |
| SEQ ID NO: 131 | GCTATTCTCGCAGCTCACCAGTTTTAGAGCTAGAAATAGCAAGTTA |
| ACTB N-term PBS 9 | AAATAAGGCTAGTCCGTTATCAACTTGAAAAAGTGGCACCGAGTC |
| RT 10 attB 38 | GGTGCTCATCCATGGATGATCCTGACGACGGAGACCGCCGTCGTC |
| pegRNA | GACAAGCCTGAGCTGCG |
| (Artificial Sequence) | |
| SEQ ID NO: 132 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAG |
| 13 RT 20 attB 38 | TCGGTGCCGGGGGTCGCAGTCGCCATGATGATCCTGACGACGGA |
| pegRNA | GACCGCCGTCGTCGACAAGCCCGGGCGGCGGAGA |
| (Artificial Sequence) | |
| SEQ ID NO: 133 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAG |
| 13 RT 15 attB 38 | TCGGTGCGTCGCAGTCGCCATGATGATCCTGACGACGGAGACCG |
| pegRNA | CCGTCGTCGACAAGCCCGGGCGGCGGAGA |
| (Artificial Sequence) | |
| SEQ ID NO: 134 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAG |
| 13 RT 10 attB 38 | TCGGTGCAGTCGCCATGATGATCCTGACGACGGAGACCGCCGTC |
| pegRNA | GTCGACAAGCCCGGGCGGCGGAGA |
| (Artificial Sequence) | |
| SEQ ID NO: 135 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAGT |
| 9 RT 20 attB 38 | CGGTGCCGGGGGTCGCAGTCGCCATGATGATCCTGACGACGGAGA |
| pegRNA | CCGCCGTCGTCGACAAGCCCGGGCGGCG |
| (Artificial Sequence) | |
| SEQ ID NO: 136 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAG |
| 9 RT 15 attB 38 | TCGGTGCGTCGCAGTCGCCATGATGATCCTGACGACGGAGACCG |
| pegRNA | CCGTCGTCGACAAGCCCGGGCGGCG |
| (Artificial Sequence) | |
| SEQ ID NO: 137 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAGT |
| 9 RT 10 attB 38 | CGGTGCAGTCGCCATGATGATCCTGACGACGGAGACCGCCGTCGT |
| pegRNA | CGACAAGCCCGGGCGGCG |
| (Artificial Sequence) | |
| SEQ ID NO: 138 | GAGAAGCGGCGTCCGGGGCTAGTTTTAGAGCTAGAAATAGCAAGT |
| SUPT16H N-term | TAAAATAAGGCTAGTCCGTTATCAACTTGAAAAAGTGGCACCGAG |
| PBS 13 RT 24 Bxb1- | TCGGTGCTCTTTGTCCAGAGTCACAGCCATACCGGATGATCCTGAC |
| GT_Initial length | GACGGAGACCGCCGTCGTCGACAAGCCGGCCCCCCGGACGCCGC |
| (Artificial Sequence) | |
| SEQ ID NO: 139 | GGGCACGGGGCCATGTACAAGTTTTAGAGCTAGAAATAGCAAGT |
| SRRM2 N-term PBS | TAAAATAAGGCTAGTCCGTTATCAACTTGAAAAAGTGGCACCGA |
| 13 RT 24 Bxb1 | GTCGGTGCGGCGTCGGCAGCCCGATCCCGTTGCCGGATGATCCT |
| Initial length | GACGACGGAGACCGCCGTCGTCGACAAGCCGGCCTACATGGCCC |
| (Artificial Sequence) | CGT |
| SEQ ID NO: 140 | GTGTCAGGTGGGGCGGGGCTAGTTTTAGAGCTAGAAATAGCAAG |
| DEPDC4 N-term | TTAAAATAAGGCTAGTCCGTTATCAACTTGAAAAAGTGGCACCG |
| PBS 18 RT 24 Bxb1 | AGTCGGTGCGCTGGCTCCTCCCCTGGCACCATACCGGATGATCCT |
| Initial length | GACGACGGAGACCGCCGTCGTCGACAAGCCGGCCCCCCGCCCCA |
| (Artificial Sequence) | CCTGACAC |
| SEQ ID NO: 141 | GAGTGGGTCAGACGAGCAGGAGTTTTAGAGCTAGAAATAGCAAGT |
| NES N-term PBS 13 | TAAAATAAGGCTAGTCCGTTATCAACTTGAAAAAGTGGCACCGAG |
| RT 29 Bxb1 Initial | TCGGTGCGATGGAGGGCTGCATGGGGGAGGAGTCGCCGGATGATC |
| length | CTGACGACGGAGACCGCCGTCGTCGACAAGCCGGCCTGCTCGTCT |
| (Artificial Sequence) | GACC |
| SEQ ID NO: 142 | GCAGCCACCCGCTCTCGGCCCGTTTTAGAGCTAGAAATAGCAAG |
| SUPT16H nicking | TTAAAATAAGGCTAGTCCGTTATCAACTTGAAAAAGTGGCACCG |
| guide-53 | AGTCGGTGC |
| (Artificial Sequence) | |
| SEQ ID NO: 143 | GTGTAGTCAGGCCGCTCACCCGTTTTAGAGCTAGAAATAGCAAG |
| SRRM2 N-term | TTAAAATAAGGCTAGTCCGTTATCAACTTGAAAAAGTGGCACCG |
| nicking guide 1 +87 | AGTCGGTGC |
| (Artificial Sequence) | |
| SEQ ID NO: 144 | GCTGACAAGTCTACGGAACCTGTTTTAGAGCTAGAAATAGCAAG |
| DEPDC4 N-term | TTAAAATAAGGCTAGTCCGTTATCAACTTGAAAAAGTGGCACCG |
| Nicking guide 1 +59 | AGTCGGTGC |
| (Artificial Sequence) | |
| SEQ ID NO: 145 | GCTCCTCCAGCGCCTTGACCGTTTTAGAGCTAGAAATAGCAAGTTA |
| NES N-term Nicking | AAATAAGGCTAGTCCGTTATCAACTTGAAAAAGTGGCACCGAGTC |
| guide 2 +79 | GGTGC |
| (Artificial Sequence) | |
| SEQ ID NO: 146 | GCTATTCTCGCAGCTCACCA |
| HITI_ACTB_guide | |
| (Artificial Sequence) | |
| SEQ ID NO: 147 | AGAAGCGGCGTCCGGGGCTA |
| HITI_SUPTH16_guide | |
| (Artificial Sequence) | |
| SEQ ID NO: 148 | GGGCACGGGGCCATGTACAA |
| HITI_SRRM2_guide | |
| (Artificial Sequence) | |
| SEQ ID NO: 149 | GCGTATTGCCTGGAGGATGG |
| HITI_NOLC1_guide | |
| (Artificial Sequence) | |
| SEQ ID NO: 150 | TGTCAGGTGGGGGGGGCTA |
| HITI_DEPDC4_guide | |
| (Artificial Sequence) | |
| SEQ ID NO: 151 | AGTGGGTCAGACGAGCAGGA |
| HITI_NES_guide | |
| (Artificial Sequence) | |
| SEQ ID NO: 152 | GCTGTCTCCGCCGCCCGCCA |
| HITI_LMNB1_guide | |
| (Artificial Sequence) | |
| SEQ ID NO: 153 | GCTATTCTCGCAGCTCACCAGTTTTAGAGCTAGAAATAGCAAGTT |
| HDR Cas9 ACTB guide | AAAATAAGGCTAGTCCGTTATCAACTTGAAAAAGTGGCACCGAG |
| (Artificial Sequence) | TCGGTGC |
| SEQ ID NO: 154 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB | GGTGCGACGAGCGCGGCGATATCATCATCCATGGCCGGATGATCC |
| original length | TGACGACGGAGXXCGCCGTCGTCGACAAGCCGGCCTGAGCTGCGA |
| pegRNAs for | GAA |
| dinucleotides | XX: CG, GC, AT, TA, GG, TT, GA, AG, CC, TC, CT, |
| (Artificial Sequence) | AA, TG, GT, CA, or AC |
| SEQ ID NO: 155 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 pegRNA | GGTGCGACGAGCGCGGCGATATCATCATCCATGCCGGATGATCCT |
| with attB 46 GT for | GACGACGGAGACCGCCGTCGTCGACAAGCCGGCCTGAGCTGCGAG |
| fusion | AA |
| (Artificial Sequence) | |
| SEQ ID NO: 156 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 pegRNA | GGTGCGACGAGCGCGGCGATATCATCATCCATGCCGGATGATCCT |
| with attB 46 CT for | GACGACGGAGAGCGCCGTCGTCGACAAGCCGGCCTGAGCTGCGA |
| multiplexing | GAA |
| (Artificial Sequence) | |
| SEQ ID NO: 157 | GCGTATTGCCTGGAGGATGGGTTTTAGAGCTAGAAATAGCAAGTT |
| NOLC1 N-term PBS | AAAATAAGGCTAGTCCGTTATCAACTTGAAAAAGTGGCACCGAGT |
| 18 RT 29 pegRNA | CGGTGCGAACCACGCGGCGAATGCCGGCGTCCGCCCCGGATGATC |
| with attB 46 GA for | CTGACGACGGAGTCCGCCGTCGTCGACAAGCCGGCCTCCTCCAGG |
| multiplexing | CAATACGCG |
| (Artificial Sequence) | |
| SEQ ID NO: 158 | GCTGTCTCCGCCGCCCGCCAGTTTTAGAGCTAGAAATAGCAAGTT |
| LMNB1 N-term PBS | AAAATAAGGCTAGTCCGTTATCAACTTGAAAAAGTGGCACCGAGT |
| 18 RT 29 pegRNA | CGGTGCGCGGCGGCACGGGGGTCGCAGTCGCCATGCCGGATGATC |
| with attB 46 AG for | CTGACGACGGAGCTCGCCGTCGTCGACAAGCCGGCCCGGGCGGCG |
| multiplexing | GAGACAGCG |
| (Artificial Sequence) | |
| SEQ ID NO: 159 | GTCACCTCCAATGACTAGGG |
| EMX1 Cas9 guide 1 | |
| (Artificial Sequence) | |
| SEQ ID NO: 160 | GGGCAACCACAAACCCACGA |
| EMX1 Cas9 guide 2 | |
| (Artificial Sequence) | |
| SEQ ID NO: 161 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 56 GA | GGTGCGACGAGCGCGGCGATATCATCATCCATGGCTATGCCGGAT |
| pegRNA | GATCCTGACGACGGAGTCCGCCGTCGTCGACAAGCCGGCCCTAGC |
| (Artificial Sequence) | TGAGCTGCGAGAA |
| SEQ ID NO: 162 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 51 GA | GGTGCGACGAGCGCGGCGATATCATCATCCATGGTGCCGGATGAT |
| pegRNA | CCTGACGACGGAGTCCGCCGTCGTCGACAAGCCGGCCCTATGAGC |
| (Artificial Sequence) | TGCGAGAA |
| SEQ ID NO: 163 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 46 GA | GGTGCGACGAGCGCGGCGATATCATCATCCATGGCCGGATGATCC |
| pegRNA | TGACGACGGAGTCCGCCGTCGTCGACAAGCCGGCCTGAGCTGCGA |
| (Artificial Sequence) | GAA |
| SEQ ID NO: 164 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 41 GA | GGTGCGACGAGCGCGGCGATATCATCATCCATGGGGATGATCCTG |
| pegRNA | ACGACGGAGTCCGCCGTCGTCGACAAGCCGTGAGCTGCGAGAA |
| (Artificial Sequence) | |
| SEQ ID NO: 165 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 36 GA | GGTGCGACGAGCGCGGCGATATCATCATCCATGGTGATCCTGACG |
| pegRNA | ACGGAGTCCGCCGTCGTCGACAAGCTGAGCTGCGAGAA |
| (Artificial Sequence) | |
| SEQ ID NO: 166 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 31 GA | GGTGCGACGAGCGCGGCGATATCATCATCCATGGATCCTGACGAC |
| pegRNA | GGAGTCCGCCGTCGTCGACATGAGCTGCGAGAA |
| (Artificial Sequence) | |
| SEQ ID NO: 167 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 26 GA | GGTGCGACGAGCGCGGCGATATCATCATCCATGGCCTGACGACGG |
| pegRNA | AGTCCGCCGTCGTCGTGAGCTGCGAGAA |
| (Artificial Sequence) | |
| SEQ ID NO: 168 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 21 GA | GGTGCGACGAGCGCGGCGATATCATCATCCATGGTGACGACGGAG |
| pegRNA | TCCGCCGTCGTGAGCTGCGAGAA |
| (Artificial Sequence) | |
| SEQ ID NO: 169 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 16 GA | GGTGCGACGAGCGCGGCGATATCATCATCCATGGACGACGGAGTC |
| pegRNA | CGCCGTGAGCTGCGAGAA |
| (Artificial Sequence) | |
| SEQ ID NO: 170 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 11 GA | GGTGCGACGAGCGCGGCGATATCATCATCCATGGGACGGAGTCCG |
| pegRNA | TGAGCTGCGAGAA |
| (Artificial Sequence) | |
| SEQ ID NO: 171 | GCTATTCTCGCAGCTCACCAGTTTTAGAGCTAGAAATAGCAAGTTA |
| ACTB N-term PBS | AAATAAGGCTAGTCCGTTATCAACTTGAAAAAGTGGCACCGAGTC |
| 13 RT 29 attB 6 GA | GGTGCGACGAGCGCGGCGATATCATCATCCATGGCGGAGTTGAGC |
| pegRNA | TGCGAGAA |
| (Artificial Sequence) | |
| SEQ ID NO: 172 | GAAGCCGGCCTTGCACATGCGTTTTAGAGCTAGAAATAGCAAGTT |
| ACTB N-term | AAAATAAGGCTAGTCCGTTATCAACTTGAAAAAGTGGCACCGAGT |
| PBS_18_RT_34_with_ | CGGTGCTCGACGACGAGCGCGGCGATATCATCATCCATGGTACCG |
| Lox71_Cre | TTCGTATAGCATACATTATACGAAGTTATTGAGCTGCGAGAATAG |
| pegRNA | CC |
| (Artificial Sequence) | |
| SEQ ID NO: 173 | GAAGCCGGCCTTGCACATGCGTTTTAGAGCTAGAAATAGCAAGTT |
| ACTB N-term | AAAATAAGGCTAGTCCGTTATCAACTTGAAAAAGTGGCACCGAGT |
| PBS_18_RT_29_with_ | CGGTGCGACGAGCGCGGCGATATCATCATCCATGGTACCGTTCGT |
| Lox71_Cre | ATAGCATACATTATACGAAGTTATTGAGCTGCGAGAATAGCC |
| pegRNA | |
| (Artificial Sequence) | |
| SEQ ID NO: 174 | GAAGCCGGCCTTGCACATGCGTTTTAGAGCTAGAAATAGCAAGTT |
| ACTB N-term | AAAATAAGGCTAGTCCGTTATCAACTTGAAAAAGTGGCACCGAGT |
| PBS_13_RT _34_with_ | CGGTGCTCGACGACGAGCGCGGCGATATCATCATCCATGGTACCG |
| Lox71_Cre | TTCGTATAGCATACATTATACGAAGTTATTGAGCTGCGAGAA |
| pegRNA | |
| (Artificial Sequence) | |
| SEQ ID NO: 175 | GAAGCCGGCCTTGCACATGCGTTTTAGAGCTAGAAATAGCAAGTT |
| ACTB N-term | AAAATAAGGCTAGTCCGTTATCAACTTGAAAAAGTGGCACCGAGT |
| PBS_13_RT_16_with_ | CGGTGCATATCATCATCCATGGTACCGTTCGTATAGCATACATTAT |
| Lox71_Cre | ACGAAGTTATTGAGCTGCGAGAA |
| pegRNA | |
| (Artificial Sequence) | |
| SEQ ID NO: 176 | CCCCACGATGGAGGGGAAGAGTTTTAGAGCTAGAAATAGCAAGTT |
| ACTB N-term | AAAATAAGGCTAGTCCGTTATCAACTTGAAAAAGTGGCACCGAGT |
| Nicking guide 2 +93 | CGGTGC |
| guide | |
| (Artificial Sequence) | |
| SEQ ID NO: 177 | CCTTCTCCTGGAGCCGCGACGTTTTAGAGCTAGAAATAGCAAGTT |
| LMNB1 N-term | AAAATAAGGCTAGTCCGTTATCAACTTGAAAAAGTGGCACCGAGT |
| Nicking guide 2 +87 | CGGTGC |
| guide | |
| (Artificial Sequence) | |

Sequences of insertion sites can be found in Table 4 below.

TABLE 4

| | FORWARD SEQUENCE (5′-3′) | | REVERSE SEQUENCE (5′-3′) | |
|---|---|---|---|---|
| DESCRIPTION/ | SEQ ID | | SEQ ID | |
| SOURCE | NO | Sequence | NO | Sequence |
| Bxb1_attP_GT_ | 178 | GTGGTTTGTCTGGTC | 179 | TGGGTTTGTACCGTA |
| original_site | | AACCACCGCGGTCT | | CACCACTGAGACCG |
| (Artificial | | CAGTGGTGTACGGT | | CGGTGGTTGACCAG |
| Sequence) | | ACAAACCCA | | ACAAACCAC |
| Bxb1_attP_CG_site | 180 | GTGGTTTGTCTGGTC | 181 | TGGGTTTGTACCGTA |
| (Artificial | | AACCACCGCGCGCT | | CACCACTGAGCGCG |
| Sequence) | | CAGTGGTGTACGGT | | CGGTGGTTGACCAG |
| | | ACAAACCCA | | ACAAACCAC |
| Bxb1_attP_GC_site | 182 | GTGGTTTGTCTGGTC | 183 | TGGGTTTGTACCGTA |
| (Artificial | | AACCACCGCGGCCT | | CACCACTGAGGCCG |
| Sequence) | | CAGTGGTGTACGGT | | CGGTGGTTGACCAG |
| | | ACAAACCCA | | ACAAACCAC |
| Bxb1_attP_AT_site | 184 | GTGGTTTGTCTGGTC | 185 | TGGGTTTGTACCGTA |
| (Artificial | | AACCACCGCGATCT | | CACCACTGAGATCG |
| Sequence) | | CAGTGGTGTACGGT | | CGGTGGTTGACCAG |
| | | ACAAACCCA | | ACAAACCAC |
| Bxb1_attP_TA_site | 186 | GTGGTTTGTCTGGTC | 187 | TGGGTTTGTACCGTA |
| (Artificial | | AACCACCGCGTACT | | CACCACTGAGTACG |
| Sequence) | | CAGTGGTGTACGGT | | CGGTGGTTGACCAG |
| | | ACAAACCCA | | ACAAACCAC |
| Bxb1_attP_GG_site | 188 | GTGGTTTGTCTGGTC | 189 | TGGGTTTGTACCGTA |
| (Artificial | | AACCACCGCGGGCT | | CACCACTGAGCCCG |
| Sequence) | | CAGTGGTGTACGGT | | CGGTGGTTGACCAG |
| | | ACAAACCCA | | ACAAACCAC |
| Bxb1_attP_TT_site | 190 | GTGGTTTGTCTGGTC | 191 | TGGGTTTGTACCGTA |
| (Artificial | | AACCACCGCGTTCTC | | CACCACTGAGAACG |
| Sequence) | | AGTGGTGTACGGTA | | CGGTGGTTGACCAG |
| | | CAAACCCA | | ACAAACCAC |
| Bxb1_attP_GA_site | 192 | GTGGTTTGTCTGGTC | 193 | TGGGTTTGTACCGTA |
| (Artificial | | AACCACCGCGGACT | | CACCACTGAGTCCG |
| Sequence) | | CAGTGGTGTACGGT | | CGGTGGTTGACCAG |
| | | ACAAACCCA | | ACAAACCAC |
| Bxb1_attP_AG_site | 194 | GTGGTTTGTCTGGTC | 195 | TGGGTTTGTACCGTA |
| (Artificial | | AACCACCGCGAGCT | | CACCACTGAGCTCG |
| Sequence) | | CAGTGGTGTACGGT | | CGGTGGTTGACCAG |
| | | ACAAACCCA | | ACAAACCAC |
| Bxb1_attP_CC_site | 196 | GTGGTTTGTCTGGTC | 197 | TGGGTTTGTACCGTA |
| (Artificial | | AACCACCGCGCCCT | | CACCACTGAGGGCG |
| Sequence) | | CAGTGGTGTACGGT | | CGGTGGTTGACCAG |
| | | ACAAACCCA | | ACAAACCAC |
| Bxb1_attP_TC_site | 198 | GTGGTTTGTCTGGTC | 199 | TGGGTTTGTACCGTA |
| (Artificial | | AACCACCGCGTCCTC | | CACCACTGAGGACG |
| Sequence) | | AGTGGTGTACGGTA | | CGGTGGTTGACCAG |
| | | CAAACCCA | | ACAAACCAC |
| Bxb1_attP_CT_site | 200 | GTGGTTTGTCTGGTC | 201 | TGGGTTTGTACCGTA |
| (Artificial | | AACCACCGCGCTCTC | | CACCACTGAGAGCG |
| Sequence) | | AGTGGTGTACGGTA | | CGGTGGTTGACCAG |
| | | CAAACCCA | | ACAAACCAC |
| Bxb1_attP_AA_site | 202 | GTGGTTTGTCTGGTC | 203 | TGGGTTTGTACCGTA |
| (Artificial | | AACCACCGCGAACT | | CACCACTGAGTTCGC |
| Sequence) | | CAGTGGTGTACGGT | | GGTGGTTGACCAGA |
| | | ACAAACCCA | | CAAACCAC |
| Bxb1_attP_CA_site | 204 | GTGGTTTGTCTGGTC | 205 | TGGGTTTGTACCGTA |
| (Artificial | | AACCACCGCGCACT | | CACCACTGAGTGCG |
| Sequence) | | CAGTGGTGTACGGT | | CGGTGGTTGACCAG |
| | | ACAAACCCA | | ACAAACCAC |
| Bxb1_attP_AC_site | 206 | GTGGTTTGTCTGGTC | 207 | TGGGTTTGTACCGTA |
| (Artificial | | AACCACCGCGACCT | | CACCACTGAGGTCG |
| Sequence) | | CAGTGGTGTACGGT | | CGGTGGTTGACCAG |
| | | ACAAACCCA | | ACAAACCAC |
| Bxb1_attP_TG_site | 208 | GTGGTTTGTCTGGTC | 209 | TGGGTTTGTACCGTA |
| (Artificial | | AACCACCGCGTGCT | | CACCACTGAGCACG |
| Sequence) | | CAGTGGTGTACGGT | | CGGTGGTTGACCAG |
| | | ACAAACCCA | | ACAAACCAC |
| Bxb1_attB_46_GT | 210 | GGCCGGCTTGTCGA | 211 | CCGGATGATCCTGA |
| original_site | | CGACGGCGGTCTCC | | CGACGGAGACCGCC |
| (Artificial | | GTCGTCAGGATCATC | | GTCGTCGACAAGCC |
| Sequence) | | CGG | | GGCC |
| Bxb1_attB_46_AA_site | 212 | GGCCGGCTTGTCGA | 213 | CCGGATGATCCTGA |
| (Artificial | | CGACGGCGAACTCC | | CGACGGAGTTCGCC |
| Sequence) | | GTCGTCAGGATCATC | | GTCGTCGACAAGCC |
| | | CGG | | GGCC |
| Bxb1_attB_46_GA_site | 214 | GGCCGGCTTGTCGA | 215 | CCGGATGATCCTGA |
| (Artificial | | CGACGGCGGACTCC | | CGACGGAGTCCGCC |
| Sequence) | | GTCGTCAGGATCATC | | GTCGTCGACAAGCC |
| | | CGG | | GGCC |
| Bxb1_attB_46_CA_site | 216 | GGCCGGCTTGTCGA | 217 | CCGGATGATCCTGA |
| (Artificial | | CGACGGCGCACTCC | | CGACGGAGTGCGCC |
| Sequence) | | GTCGTCAGGATCATC | | GTCGTCGACAAGCC |
| | | CGG | | GGCC |
| Bxb1_attB_46_TA_site | 218 | GGCCGGCTTGTCGA | 219 | CCGGATGATCCTGA |
| (Artificial | | CGACGGCGTACTCC | | CGACGGAGTACGCC |
| Sequence) | | GTCGTCAGGATCATC | | GTCGTCGACAAGCC |
| | | CGG | | GGCC |
| Bxb1_attB_46_AG_site | 220 | GGCCGGCTTGTCGA | 221 | CCGGATGATCCTGA |
| (Artificial | | CGACGGCGAGCTCC | | CGACGGAGCTCGCC |
| Sequence) | | GTCGTCAGGATCATC | | GTCGTCGACAAGCC |
| | | CGG | | GGCC |
| Bxb1_attB_46_GG_site | 222 | GGCCGGCTTGTCGA | 223 | CCGGATGATCCTGA |
| (Artificial | | CGACGGCGGGCTCC | | CGACGGAGCCCGCC |
| Sequence) | | GTCGTCAGGATCATC | | GTCGTCGACAAGCC |
| | | CGG | | GGCC |
| Bxb1_attB_46_CG_site | 224 | GGCCGGCTTGTCGA | 225 | CCGGATGATCCTGA |
| (Artificial | | CGACGGCGCGCTCC | | CGACGGAGCGCGCC |
| Sequence) | | GTCGTCAGGATCATC | | |
| | | CGG | | GTCGTCGACAAGCC |
| | | | | GGCC |
| Bxb1_attB_46_TG_site | 226 | GGCCGGCTTGTCGA | 227 | CCGGATGATCCTGA |
| (Artificial | | CGACGGCGTGCTCC | | CGACGGAGCACGCC |
| Sequence) | | GTCGTCAGGATCATC | | GTCGTCGACAAGCC |
| | | CGG | | GGCC |
| Bxb1_attB_46_AC_site | 228 | GGCCGGCTTGTCGA | 229 | CCGGATGATCCTGA |
| (Artificial | | CGACGGCGACCTCC | | CGACGGAGGTCGCC |
| Sequence) | | GTCGTCAGGATCATC | | |
| | | CGG | | GTCGTCGACAAGCC |
| | | | | GGCC |
| Bxb1_attB_46_GC_site | 230 | GGCCGGCTTGTCGA | 231 | CCGGATGATCCTGA |
| (Artificial | | CGACGGCGGCCTCC | | CGACGGAGGCCGCC |
| Sequence) | | GTCGTCAGGATCATC | | GTCGTCGACAAGCC |
| | | CGG | | GGCC |
| Bxb1_attB_46_CC_site | 232 | GGCCGGCTTGTCGA | 233 | CCGGATGATCCTGA |
| (Artificial | | CGACGGCGCCCTCC | | CGACGGAGGGCGCC |
| Sequence) | | GTCGTCAGGATCATC | | GTCGTCGACAAGCC |
| | | CGG | | GGCC |
| Bxb1_attB_46_TC_site | 234 | GGCCGGCTTGTCGA | 235 | CCGGATGATCCTGA |
| (Artificial | | CGACGGCGTCCTCC | | CGACGGAGGACGCC |
| Sequence) | | GTCGTCAGGATCATC | | GTCGTCGACAAGCC |
| | | CGG | | GGCC |
| Bxb1_attB_46_AT_site | 236 | GGCCGGCTTGTCGA | 237 | CCGGATGATCCTGA |
| (Artificial | | CGACGGCGATCTCC | | CGACGGAGATCGCC |
| Sequence) | | GTCGTCAGGATCATC | | GTCGTCGACAAGCC |
| | | CGG | | GGCC |
| Bxb1_attB_46_CT_site | 238 | GGCCGGCTTGTCGA | 239 | CCGGATGATCCTGA |
| (Artificial | | CGACGGCGCTCTCC | | CGACGGAGAGCGCC |
| Sequence) | | GTCGTCAGGATCATC | | GTCGTCGACAAGCC |
| | | CGG | | GGCC |
| Bxb1_attB_46_TT_site | 240 | GGCCGGCTTGTCGA | 241 | CCGGATGATCCTGA |
| (Artificial | | CGACGGCGTTCTCCG | | CGACGGAGAACGCC |
| Sequence) | | TCGTCAGGATCATCC | | GTCGTCGACAAGCC |
| | | GG | | GGCC |
| Bxb1_attB_38_GT_site | 242 | GGCTTGTCGACGAC | 243 | ATGATCCTGACGAC |
| (Artificial | | GGCGGTCTCCGTCGT | | GGAGACCGCCGTCG |
| Sequence) | | CAGGATCAT | | TCGACAAGCC |
| Bxb1_attB_38_AA_site | 244 | GGCTTGTCGACGAC | 245 | ATGATCCTGACGAC |
| (Artificial | | GGCGAACTCCGTCG | | GGAGTTCGCCGTCGT |
| Sequence) | | TCAGGATCAT | | CGACAAGCC |
| Bxb1_attB_38_GA_site | 246 | GGCTTGTCGACGAC | 247 | ATGATCCTGACGAC |
| (Artificial | | GGCGGACTCCGTCG | | GGAGTCCGCCGTCG |
| Sequence) | | TCAGGATCAT | | TCGACAAGCC |
| Bxb1_attB_38_CA_site | 248 | GGCTTGTCGACGAC | 249 | ATGATCCTGACGAC |
| (Artificial | | GGCGCACTCCGTCGT | | GGAGTGCGCCGTCG |
| Sequence) | | CAGGATCAT | | TCGACAAGCC |
| Bxb1_attB_38_TA_site | 250 | GGCTTGTCGACGAC | 251 | ATGATCCTGACGAC |
| (Artificial | | GGCGTACTCCGTCGT | | GGAGTACGCCGTCG |
| Sequence) | | CAGGATCAT | | TCGACAAGCC |
| Bxb1_attB_38_AG_site | 252 | GGCTTGTCGACGAC | 253 | ATGATCCTGACGAC |
| (Artificial | | GGCGAGCTCCGTCG | | GGAGCTCGCCGTCG |
| Sequence) | | TCAGGATCAT | | TCGACAAGCC |
| Bxb1_attB_38_GG_site | 254 | GGCTTGTCGACGAC | 255 | ATGATCCTGACGAC |
| (Artificial | | GGCGGGCTCCGTCG | | GGAGCCCGCCGTCG |
| Sequence) | | TCAGGATCAT | | TCGACAAGCC |
| Bxb1_attB_38_CG_site | 256 | GGCTTGTCGACGAC | 257 | ATGATCCTGACGAC |
| (Artificial | | GGCGCGCTCCGTCGT | | GGAGCGCGCCGTCG |
| Sequence) | | CAGGATCAT | | TCGACAAGCC |
| Bxb1_attB_38_TG_site | 258 | GGCTTGTCGACGAC | 259 | ATGATCCTGACGAC |
| (Artificial | | GGCGTGCTCCGTCGT | | GGAGCACGCCGTCG |
| Sequence) | | CAGGATCAT | | TCGACAAGCC |
| Bxb1_attB_38_AC_site | 260 | GGCTTGTCGACGAC | 261 | ATGATCCTGACGAC |
| (Artificial | | GGCGACCTCCGTCGT | | GGAGGTCGCCGTCG |
| Sequence) | | CAGGATCAT | | TCGACAAGCC |
| Bxb1_attB_38_GC_site | 262 | GGCTTGTCGACGAC | 263 | ATGATCCTGACGAC |
| (Artificial | | GGCGGCCTCCGTCGT | | GGAGGCCGCCGTCG |
| Sequence) | | CAGGATCAT | | TCGACAAGCC |
| Bxb1_attB_38_CC_site | 264 | GGCTTGTCGACGAC | 265 | ATGATCCTGACGAC |
| (Artificial | | GGCGCCCTCCGTCGT | | GGAGGGCGCCGTCG |
| Sequence) | | CAGGATCAT | | TCGACAAGCC |
| Bxb1_attB_38_TC_site | 266 | GGCTTGTCGACGAC | 267 | ATGATCCTGACGAC |
| (Artificial | | GGCGTCCTCCGTCGT | | GGAGGACGCCGTCG |
| Sequence) | | CAGGATCAT | | TCGACAAGCC |
| Bxb1_attB_38_AT_site | 268 | GGCTTGTCGACGAC | 269 | ATGATCCTGACGAC |
| (Artificial | | GGCGATCTCCGTCGT | | GGAGATCGCCGTCG |
| Sequence) | | CAGGATCAT | | TCGACAAGCC |
| Bxb1_attB_38_CT_site | 270 | GGCTTGTCGACGAC | 271 | ATGATCCTGACGAC |
| (Artificial | | GGCGCTCTCCGTCGT | | GGAGAGCGCCGTCG |
| Sequence) | | CAGGATCAT | | TCGACAAGCC |
| Bxb1_attB_38_TT_site | 272 | GGCTTGTCGACGAC | 273 | ATGATCCTGACGAC |
| (Artificial | | GGCGTTCTCCGTCGT | | GGAGAACGCCGTCG |
| Sequence) | | CAGGATCAT | | TCGACAAGCC |
| Cre Lox 66 site | 274 | TACCGTTCGTATAAT | 275 | ATAACTTCGTATAGC |
| (Artificial | | GTATGCTATACGAA | | ATACATTATACGAA |
| Sequence) | | GTTAT | | CGGTA |
| Cre Lox 71 site | 276 | ATAACTTCGTATAAT | 277 | TACCGTTCGTATAGC |
| (Artificial | | GTATGCTATACGAA | | ATACATTATACGAA |
| Sequence) | | CGGTA | | GTTAT |
| TP901-1 | 278 | TTTACCTTGATTGAG | 279 | CACAATTAACATCTC |
| minimal attB site | | ATGTTAATTGTG | | AATCAAGGTAAA |
| (Artificial | | | | |
| Sequence) | | | | |
| TP901-1 | 280 | GCGAGTTTTTATTTC | 281 | AAAGGAGTTTTTTAG |
| minimal attP site | | GTTTATTTCAATTAA | | TTACCTTAATTGAAA |
| (Artificial | | GGTAACTAAAAAAC | | TAAACGAAATAAAA |
| Sequence) | | TCCTTT | | ACTCGC |
| PhiBT1 | 282 | CTGGATCATCTGGAT | 283 | CAGGTTTTTGACGAA |
| minimal attB site | | CACTTTCGTCAAAAA | | AGTGATCCAGATGA |
| (Artificial | | CCTG | | TCCAG |
| Sequence) | | | | |
| PhiBT1 | 284 | TTCGGGTGCTGGGTT | 285 | TGGTGCTGAGTAGTT |
| minimal attP site | | GTTGTCTCTGGACAG | | TCCCATGGATCACTG |
| (Artificial | | TGATCCATGGGAAA | | TCCAGAGACAACAA |
| Sequence) | | CTACTCAGCACCA | | CCCAGCACCCGAA |

Sequences of Bxb1 and RT mutants can be found in Table 6 below.

TABLE 6

| SEQ ID NO/ | |
|---|---|
| DESCRIPTION/ | |
| SOURCE | FORWARD SEQUENCE (5′-3′) |
| SEQ ID NO: 286 | AAAAGTGTGGGCTGCAGGATCTGA |
| Bxb1_mut_V368A | |
| (Artificial Sequence) | |
| SEQ ID NO: 287 | GGAGCTGGCAGCTGTCAATGCC |
| Bxb1_mut_E379A | |
| (Artificial Sequence) | |
| SEQ ID NO: 288 | AGTCAATGCCGCTCTCGTGGA |
| Bxb1_mut_E383A | |
| (Artificial Sequence) | |
| SEQ ID NO: 403 | TTGAGCGGGCCCCCACCGT |
| RT_mut_L139P | |
| (Artificial Sequence) | |
| SEQ ID NO: 289 | CAGCGGGCTCAGCTGATAGCA |
| RT_mut_E562Q | |
| (Artificial Sequence) | |
| SEQ ID NO: 290 | CGGATGGCTAACCAAGCGGCC |
| RT_mut_D653N | |
| (Artificial Sequence) | |
| SEQ ID NO: 404 | atgactcactatcaggccttgcttttggacacggaccgggtccag |
| RT(1-478)_Sto7d | ttcggaccggtggtagccctgaacccggctacgctgctccc |
| fusion | actgcctgaggaagggctgcaacacaactgccttgatGGGACAGGTGG |
| | CGGTGGTGTCACCGTCAAGTTCAAGTACAAGGGTGAGGAACTT |
| | GAAGTTGATATTAGCAAAATCAAGAAGGTTTGGCGCGTTGGTA |
| | AAATGATATCTTTTACTTATGACGACAACGGCAAGACAGGTAG |
| | AGGGGCAGTGTCTGAGAAAGACGCCCCCAAGGAGCTGTTGCAA |
| | ATGTTGGAAAAGTCTGGGAAAAAGtctggcggctcaaaaagaa |
| | ccgccgacggcagcgaattcgagcccaagaagaagaggaaagtc |

Sequences of primers, probes and restriction enzymes used in ddPCR readout can be found in Table 7 below.

TABLE 7

| | | SEQ | Forward | SEQ | Reverse | | SEQ | Restriction |
|---|---|---|---|---|---|---|---|---|
| Locus | Cargo | ID NO: | Primer | ID NO: | Primer | Probe | ID NO: | Enzymes |
| ACTB | GFP | 291 | CCCG | 292 | GAAC | /56- | 405 | Eco91I, |
| | (pDY0186) | | GCTTC | | TCCAC | FAM/C | | HindIII |
| | | | CTTTG | | GCCG | C GGC | | |
| | | | TCC | | TTCA | TTG | | |
| | | | | | | T/ZEN/ | | |
| | | | | | | C GAC | | |
| | | | | | | GAC | | |
| | | | | | | GGC | | |
| | | | | | | G/3IAB | | |
| | | | | | | kFQ/ | | |
| ACTB | TP90-1 | 293 | CCCG | 294 | AACC | /56- | 406 | None |
| | GFP | | GCTTC | | ACAA | FAM/T | | |
| | (pDY0333) | | CTTTG | | CTAG | G CTA | | |
| | | | TCC | | AATG | TTG | | |
| | | | | | CAGT | C/ZEN/ | | |
| | | | | | GA | T TTA | | |
| | | | | | | TTT | | |
| | | | | | | GTG | | |
| | | | | | | GGC | | |
| | | | | | | CCG | | |
| | | | | | | /3IABk | | |
| | | | | | | FQ/ | | |
| ACTB | TP90-1 | 295 | CCCG | 296 | GAAC | /56- | 407 | None |
| | rc GFP | | GCTTC | | TCCAC | FAM/ | | |
| | (pDY0334) | | CTTTG | | GCCG | CC | | |
| | | | TCC | | TTCA | ATG | | |
| | | | | | | AAG | | |
| | | | | | | A/ZE | | |
| | | | | | | N/T | | |
| | | | | | | CGA | | |
| | | | | | | GTG | | |
| | | | | | | CCG | | |
| | | | | | | CAT | | |
| | | | | | | CA/3I | | |
| | | | | | | ABkF | | |
| | | | | | | Q/ | | |
| ACTB | PhiBT1 | 297 | CCCG | 298 | AACC | /56- | 406 | None |
| | GFP | | GCTTC | | ACAA | FAM/T | | |
| | (pDY0367) | | CTTTG | | CTAG | G CTA | | |
| | | | TCC | | AATG | TTG | | |
| | | | | | CAGT | C/ZEN/ | | |
| | | | | | GA | T TTA | | |
| | | | | | | TTT | | |
| | | | | | | GTG | | |
| | | | | | | GGC | | |
| | | | | | | CCG | | |
| | | | | | | /3IABk | | |
| | | | | | | FQ/ | | |
| ACTB | PhiBT1 | 299 | CCCG | 300 | GAAC | /56- | 407 | None |
| | rc GFP | | GCTTC | | TCCAC | FAM/ | | |
| | (pDY0368) | | CTTTG | | GCCG | CC | | |
| | | | TCC | | TTCA | ATG | | |
| | | | | | | AAG | | |
| | | | | | | A/ZE | | |
| | | | | | | N/T | | |
| | | | | | | CGA | | |
| | | | | | | GTG | | |
| | | | | | | CCG | | |
| | | | | | | CAT | | |
| | | | | | | CA/3I | | |
| | | | | | | ABkF | | |
| | | | | | | Q/ | | |
| LMNB1 | GFP | 301 | TCCTT | 302 | GAAC | /56- | 407 | Eco91I, |
| | (pDY0186) | | ATCA | | TCCAC | FAM/ | | HindIII |
| | | | CGGT | | GCCG | CC | | |
| | | | CCCG | | TTCA | ATG | | |
| | | | CTCG | | | AAG | | |
| | | | | | | A/ZE | | |
| | | | | | | N/T | | |
| | | | | | | CGA | | |
| | | | | | | GTG | | |
| | | | | | | CCG | | |
| | | | | | | CAT | | |
| | | | | | | CA/3I | | |
| | | | | | | ABkF | | |
| | | | | | | Q/ | | |
| NOLC1 | GFP | 303 | CGTC | 304 | GAAC | /56- | 407 | Eco91I, |
| | (pDY0186) | | GACA | | TCCAC | FAM/ | | HindIII |
| | | | ACGG | | GCCG | CC | | |
| | | | TAGT | | TTCA | ATG | | |
| | | | G | | | AAG | | |
| | | | | | | A/ZE | | |
| | | | | | | N/T | | |
| | | | | | | CGA | | |
| | | | | | | GTG | | |
| | | | | | | CCG | | |
| | | | | | | CAT | | |
| | | | | | | CA/3I | | |
| | | | | | | ABkF | | |
| | | | | | | Q/ | | |
| SUPT16 H | GFP | 305 | TCGC | 306 | GAAC | /56- | 407 | Eco91I, |
| | (pDY0186) | | GTGA | | TCCA | FAM/C | | HindIII |
| | | | TTCTC | | CGCC | C ATG | | |
| | | | GGAA | | GTTC | AAG | | |
| | | | C | | A | A/ZEN/ | | |
| | | | | | | T CGA | | |
| | | | | | | GTG | | |
| | | | | | | CCG | | |
| | | | | | | CAT | | |
| | | | | | | CA/3IA | | |
| | | | | | | BkFQ/ | | |
| SRRM2 | GFP | 307 | GGGC | 308 | GAAC | /56- | 407 | Eco91I, |
| | (pDY0186) | | GGTA | | TCCAC | FAM/ | | HindIII |
| | | | AGTG | | GCCG | CC | | |
| | | | GTTA | | TTCA | ATG | | |
| | | | GTTT | | | AAG | | |
| | | | | | | A/ZE | | |
| | | | | | | N/T | | |
| | | | | | | CGA | | |
| | | | | | | GTG | | |
| | | | | | | CCG | | |
| | | | | | | CAT | | |
| | | | | | | CA/3I | | |
| | | | | | | ABkF | | |
| | | | | | | Q/ | | |
| DEPDC4 | GFP | 309 | AAGA | 310 | GAAC | /56- | 407 | Eco91I, |
| | (pDY0186) | | GGCG | | TCCAC | FAM/ | | HindIII |
| | | | GAGC | | GCCG | CC | | |
| | | | CAGT | | TTCA | ATG | | |
| | | | A | | | AAG | | |
| | | | | | | A/ZE | | |
| | | | | | | N/T | | |
| | | | | | | CGA | | |
| | | | | | | GTG | | |
| | | | | | | CCG | | |
| | | | | | | CAT | | |
| | | | | | | CA/3I | | |
| | | | | | | ABkF | | |
| | | | | | | Q/ | | |
| NES | GFP | 311 | CTCCC | 312 | GAAC | /56- | 405 | Eco91I, |
| | (pDY0186) | | TTCTC | | TCCAC | FAM/C | | HindIII |
| | | | CCGG | | GCCG | C GGC | | |
| | | | TGCCC | | TTCA | TTG | | |
| | | | | | | T/ZEN/ | | |
| | | | | | | C GAC | | |
| | | | | | | GAC | | |
| | | | | | | GGC | | |
| | | | | | | G/3IAB | | |
| | | | | | | kFQ/ | | |
| ACTB | ACTB | 313 | CCCG | 314 | GAAC | /56- | 407 | Eco91I |
| | HITI | | GCTTC | | TCCAC | FAM/ | | |
| | template | | CTTTG | | GCCG | CC | | |
| | GFP | | TCC | | TTCA | ATG | | |
| | (pDY0219) | | | | | AAG | | |
| | | | | | | A/ZE | | |
| | | | | | | N/T | | |
| | | | | | | CGA | | |
| | | | | | | GTG | | |
| | | | | | | CCG | | |
| | | | | | | CAT | | |
| | | | | | | CA/3I | | |
| | | | | | | ABkF | | |
| | | | | | | Q | | |
| SRRM2 | SRRM2 | 315 | GGGC | 316 | GAAC | /56- | 407 | Eco91I |
| | HITI | | GGTA | | TCCAC | FAM/ | | |
| | template | | AGTG | | GCCG | CC | | |
| | GFP | | GTTA | | TTCA | ATG | | |
| | (aRY0182_A2) | | GTTT | | | AAG | | |
| | | | | | | A/ZE | | |
| | | | | | | N/T | | |
| | | | | | | CGA | | |
| | | | | | | GTG | | |
| | | | | | | CCG | | |
| | | | | | | CAT | | |
| | | | | | | CA/3I | | |
| | | | | | | ABkF | | |
| | | | | | | Q/ | | |
| NOLC1 | NOLC1 | 317 | CGTC | 318 | GAAC | /56- | 407 | Eco91I |
| | HITI | | GACA | | TCCAC | FAM/ | | |
| | template | | ACGG | | GCCG | CC | | |
| | GFP | | TAGT | | TTCA | ATG | | |
| | (aRY0182_A3) | | G | | | AAG | | |
| | | | | | | A/ZE | | |
| | | | | | | N/T | | |
| | | | | | | CGA | | |
| | | | | | | GTG | | |
| | | | | | | CCG | | |
| | | | | | | CAT | | |
| | | | | | | CA/3I | | |
| | | | | | | ABkF | | |
| | | | | | | Q/ | | |
| DEPDC4 | DEPDC4 HITI | 319 | AAGA | 320 | GAAC | /56- | 407 | Eco91I |
| | template | | GGCG | | TCCAC | FAM/ | | |
| | GFP | | GAGC | | GCCG | CC | | |
| | (aRY0182_A5) | | CAGT | | TTCA | ATG | | |
| | | | A | | | AAG | | |
| | | | | | | A/ZE | | |
| | | | | | | N/T | | |
| | | | | | | CGA | | |
| | | | | | | GTG | | |
| | | | | | | CCG | | |
| | | | | | | CAT | | |
| | | | | | | CA/3I | | |
| | | | | | | ABkF | | |
| | | | | | | Q/ | | |
| NES | NES | 321 | CTCCC | 322 | GAAC | /56- | 407 | Eco91I |
| | HITI | | TTCTC | | TCCAC | FAM/ | | |
| | template | | CCGG | | GCCG | CC | | |
| | GFP | | TGCCC | | TTCA | ATG | | |
| | (aRY0182_A7) | | | | | AAG | | |
| | | | | | | A/ZE | | |
| | | | | | | N/T | | |
| | | | | | | CGA | | |
| | | | | | | GTG | | |
| | | | | | | CCG | | |
| | | | | | | CAT | | |
| | | | | | | CA/3I | | |
| | | | | | | ABkF | | |
| | | | | | | Q/ | | |
| LMNB1 | LMNB1 HITI | 323 | TCCTT | 324 | GAAC | /56- | 407 | Eco91I |
| | template | | ATCA | | TCCAC | FAM/ | | |
| | GFP | | CGGT | | GCCG | CC | | |
| | (aRY0182_A4) | | CCCG | | TTCA | ATG | | |
| | | | CTCG | | | AAG | | |
| | | | | | | A/ZE | | |
| | | | | | | N/T | | |
| | | | | | | CGA | | |
| | | | | | | GTG | | |
| | | | | | | CCG | | |
| | | | | | | CAT | | |
| | | | | | | CA/3I | | |
| | | | | | | ABkF | | |
| | | | | | | Q/ | | |
| ACTB | SERPINA | 325 | CCCG | 326 | GGCC | /56- | 405 | EcoRI, |
| | (pDY0298) | | GCTTC | | TGCC | FAM/ | | XhoI, |
| | | | CTTTG | | AGCA | CC | | HindIII |
| | | | TCC | | GGAG | GGC | | |
| | | | | | GA | TTG | | |
| | | | | | | T/ZE | | |
| | | | | | | N/C | | |
| | | | | | | GAC | | |
| | | | | | | GAC | | |
| | | | | | | GGC | | |
| | | | | | | G/3I | | |
| | | | | | | ABkF | | |
| | | | | | | Q/ | | |
| ACTB | CPS1 | 327 | CCCG | 328 | GGTG | /56 | 408 | XhoI, |
| | (pDY299) | | GCTTC | | TGCA | FAM/ | | HindIII |
| | | | CTTTG | | GTCA | AC | | |
| | | | TCC | | CATTG | AGC | | |
| | | | | | GTAA | TTT | | |
| | | | | | AGCC | C/ZE | | |
| | | | | | | N/A | | |
| | | | | | | AAG | | |
| | | | | | | TGG | | |
| | | | | | | TGA | | |
| | | | | | | GGA | | |
| | | | | | | CAC | | |
| | | | | | | T/3IA | | |
| | | | | | | BkFQ/ | | |
| ACTB | CFTR | 329 | CCCG | 330 | GATG | /56- | 409 | Eco91I, |
| | (pDY0373) | | GCTTC | | GGTCT | FAM/ | | HindIII |
| | | | CTTTG | | AGTC | TAC | | |
| | | | TCC | | CAGC | GGT | | |
| | | | | | TAAA | ACA/ | | |
| | | | | | G | ZEN/ | | |
| | | | | | | AAC | | |
| | | | | | | CC | | |
| | | | | | | ACC | | |
| | | | | | | CGA | | |
| | | | | | | GAG | | |
| | | | | | | A/3I | | |
| | | | | | | ABkF | | |
| | | | | | | Q/ | | |
| ACTB | NYESO | 331 | CCCG | 332 | GAGA | /56- | 409 | Eco47III, |
| | TRAC | | GCTTC | | GACA | FAM/ | | HindIII |
| | (pDY0318) | | CTTTG | | AGGC | TAC | | |
| | | | TCC | | TGCA | GGT | | |
| | | | | | CA | ACA/ | | |
| | | | | | | ZEN/ | | |
| | | | | | | AAC | | |
| | | | | | | CC | | |
| | | | | | | ACC | | |
| | | | | | | CGA | | |
| | | | | | | GAG | | |
| | | | | | | A/3I | | |
| | | | | | | ABkF | | |
| | | | | | | Q/ | | |
| NC_0000 03 | GFP | 333 | CCAG | 334 | GAAC | /56- | 405 | Eco91I, |
| | (pDY0186) | | GTGA | | TCCAC | FAM/ | | HindIII |
| | | | GAGT | | GCCG | CC | | |
| | | | CAGG | | TTCA | GGC | | |
| | | | GTAG | | | TTG | | |
| | | | TGTTC | | | T/ZE | | |
| | | | A | | | N/C | | |
| | | | | | | GAC | | |
| | | | | | | GAC | | |
| | | | | | | GGC | | |
| | | | | | | G/3I | | |
| | | | | | | ABkF | | |
| | | | | | | Q/ | | |
| NC_0000 02 | GFP | 335 | AGGG | 336 | GAAC | /56- | 405 | Eco91I, |
| | (pDY0186) | | ACCTT | | TCCAC | FAM/ | | HindIII |
| | | | TGCCT | | GCCG | CC | | |
| | | | GTGT | | TTCA | GGC | | |
| | | | GAGT | | | TTG | | |
| | | | C | | | T/ZE | | |
| | | | | | | N/C | | |
| | | | | | | GAC | | |
| | | | | | | GAC | | |
| | | | | | | GGC | | |
| | | | | | | G/3I | | |
| | | | | | | ABkF | | |
| | | | | | | Q/ | | |
| NC_0000 09 | GFP | 337 | TCAG | 338 | GAAC | /56- | 405 | Eco91I, |
| | (pDY0186) | | CTCTG | | TCCAC | FAM/ | | HindIII |
| | | | TGCTG | | GCCG | CC | | |
| | | | AGGC | | TTCA | GGC | | |
| | | | GAA | | | TTG | | |
| | | | | | | T/ZE | | |
| | | | | | | N/C | | |
| | | | | | | GAC | | |
| | | | | | | GAC | | |
| | | | | | | GGC | | |
| | | | | | | G/3I | | |
| | | | | | | ABkF | | |
| | | | | | | Q/ | | |
| chr6:149045959 | GFP | 339 | AAGC | 340 | GAAC | /56- | 405 | Eco91I, |
| | (pDY0186) | | CATCT | | TCCAC | FAM/ | | HindIII |
| | | | CCCA | | GCCG | CC | | |
| | | | GAAT | | TTCA | GGC | | |
| | | | ATCTG | | | TTG | | |
| | | | CTTAG | | | T/ZE | | |
| | | | AAAT | | | N/C | | |
| | | | G | | | GAC | | |
| | | | | | | GAC | | |
| | | | | | | GGC | | |
| | | | | | | G/3I | | |
| | | | | | | ABkF | | |
| | | | | | | Q/ | | |
| chr16:18607730 | GFP | 341 | GAGA | 342 | GAAC | /56- | 405 | Eco91I, |
| | (pDY0186) | | GGAG | | TCCAC | FAM/ | | HindIII |
| | | | CAAC | | GCCG | CC | | |
| | | | AGTG | | TTCA | GGC | | |
| | | | AGCA | | | TTG | | |
| | | | TGAT | | | T/ZE | | |
| | | | G | | | N/C | | |
| | | | | | | GAC | | |
| | | | | | | GAC | | |
| | | | | | | GGC | | |
| | | | | | | G/3I | | |
| | | | | | | ABkF | | |
| | | | | | | Q/ | | |
| chr6:149045959 | ACTB | 343 | AAGC | 344 | GAAC | /56- | 405 | Eco91I |
| | HITI | | CATCT | | TCCAC | FAM/ | | |
| | template | | CCCA | | GCCG | CC | | |
| | GFP | | GAAT | | TTCA | GGC | | |
| | (pDY0219) | | ATCTG | | | TTG | | |
| | | | CTTAG | | | T/ZE | | |
| | | | AAAT | | | N/C | | |
| | | | G | | | GAC | | |
| | | | | | | GAC | | |
| | | | | | | GGC | | |
| | | | | | | G/3I | | |
| | | | | | | ABkF | | |
| | | | | | | Q/ | | |
| chr16:18607730 | ACTB | 345 | GAGA | 346 | GAAC | /56- | 405 | Eco91I |
| | HITI | | GGAG | | TCCAC | FAM/ | | |
| | template | | CAAC | | GCCG | CC | | |
| | GFP | | AGTG | | TTCA | GGC | | |
| | (pDY0219) | | AGCA | | | TTG | | |
| | | | TGAT | | | T/ZE | | |
| | | | G | | | N/C | | |
| | | | | | | GAC | | |
| | | | | | | GAC | | |
| | | | | | | GGC | | |
| | | | | | | G/3I | | |
| | | | | | | ABkF | | |
| | | | | | | Q/ | | |
| ACTB | CAG Kozak | 347 | CCCG | 348 | GGCT | /56- | 405 | Eco91I, |
| | b GH | | GCTTC | | ATGA | FAM/ | | HindIII |
| | therapeutic | | CTTTG | | ACTA | CC | | |
| | genes | | TCC | | ATGA | GGC | | |
| | generic | | | | CCCC | TTG | | |
| | minicircle | | | | GT | T/ZE | | |
| | | | | | | N/C | | |
| | | | | | | GAC | | |
| | | | | | | GAC | | |
| | | | | | | GGC | | |
| | | | | | | G/3I | | |
| | | | | | | ABkF | | |
| | | | | | | Q/ | | |
| ACTB | Hibit- | 349 | CCCG | 350 | GGCC | /56- | 405 | EcoRI, |
| | SERPINA | | GCTTC | | TGCC | FAM/ | | XhoI, |
| | (pDY0405) | | CTTTG | | AGCA | CC | | HindIII |
| | | | TCC | | GGAG | GGC | | |
| | | | | | GA | TTG | | |
| | | | | | | T/ZE | | |
| | | | | | | N/C | | |
| | | | | | | GAC | | |
| | | | | | | GAC | | |
| | | | | | | GGC | | |
| | | | | | | G/3I | | |
| | | | | | | ABkF | | |
| | | | | | | Q/ | | |
| ACTB | Hibit- | 351 | CCCG | 352 | GGTG | /56- | 408 | XhoI, |
| | CPS1 | | GCTTC | | TGCA | FAM/ | | HindIII |
| | (pDY406) | | CTTTG | | GTCA | AC | | |
| | | | TCC | | CATTG | AGC | | |
| | | | | | GTAA | TTT | | |
| | | | | | AGCC | C/ZE | | |
| | | | | | | N/A | | |
| | | | | | | AAG | | |
| | | | | | | TGG | | |
| | | | | | | TGA | | |
| | | | | | | GGA | | |
| | | | | | | CAC | | |
| | | | | | | T/3IA | | |
| | | | | | | BkFQ/ | | |

Sequences of primers used for NGS readout can be found in Table 8 below.

TABLE 8

| SEQ ID NO/ | | |
|---|---|---|
| DESCRIPTION/ | | |
| SOURCE | ID | SEQUENCE (5′-3′) |
| SEQ ID NO: 353 | PD0966 | ACACTCTTTCCCTACACGACGCTCTTCCGATCTCCGAC |
| N-term ACTB Tn5 | | CTCGGC TCACAGCG |
| readout F 1 | | |
| (Artificial Sequence) | | |
| SEQ ID NO: 354 | PD0967 | ACACTCTTTCCCTACACGACGCTCTTCCGATCTACCGA |
| N-term ACTB Tn5 | | CCTCGG CTCACAGCG |
| readout F 2 | | |
| (Artificial Sequence) | | |
| SEQ ID NO: 355 | PD0968 | ACACTCTTTCCCTACACGACGCTCTTCCGATCTGACCG |
| N-term ACTB Tn5 | | ACCTCG GCTCACAGCG |
| readout F 3 | | |
| (Artificial Sequence) | | |
| SEQ ID NO: 356 | PD0969 | ACACTCTTTCCCTACACGACGCTCTTCCGATCTTGACC |
| N-term ACTB Tn5 | | GACCTC GGCTCACAGCG |
| readout F 4 | | |
| (Artificial Sequence) | | |
| SEQ ID NO: 357 | PD0970 | ACACTCTTTCCCTACACGACGCTCTTCCGATCTCTGAC |
| N-term ACTB Tn5 | | CGACCT CGGCTCACAGCG |
| readout F 5 | | |
| (Artificial Sequence) | | |
| SEQ ID NO: 358 | PD0971 | ACACTCTTTCCCTACACGACGCTCTTCCGATCTACTGA |
| N-term ACTB Tn5 | | CCGACC TCGGCTCACAGCG |
| readout F 6 | | |
| (Artificial Sequence) | | |
| SEQ ID NO: 359 | PD0972 | ACACTCTTTCCCTACACGACGCTCTTCCGATCTTACTG |
| N-term ACTB Tn5 | | ACCGAC CTCGGCTCACAGCG |
| readout F 7 | | |
| (Artificial Sequence) | | |
| SEQ ID NO: 360 | PD0973 | ACACTCTTTCCCTACACGACGCTCTTCCGATCTGTACT |
| N-term ACTB Tn5 | | GACCGA CCTCGGCTCACAGCG |
| readout F 8 | | |
| (Artificial Sequence) | | |
| SEQ ID NO: 361 | FP0952 | GTGACTGGAGTTCAGACGTGTGCTCTTCCGATCTCCAC |
| ACTB N-term NGS | | CCAGCC AGCTCCC |
| R for Cas14 indels | | |
| (Artificial | | |
| Sequence) | | |
| SEQ ID NO: 362 | PD0313 | ACACTCTTTCCCTACACGACGCTCTTCCGATCTCCGGT |
| NGS EMX1 | | GGCGCAT TGCCAC |
| Forward 1 | | |
| (Artificial Sequence) | | |
| SEQ ID NO: 363 | PD0314 | ACACTCTTTCCCTACACGACGCTCTTCCGATCTACCGG |
| NGS EMX1 | | TGGCGCA TTGCCAC |
| Forward 2 | | |
| (Artificial Sequence) | | |
| SEQ ID NO: 364 | PD0315 | ACACTCTTTCCCTACACGACGCTCTTCCGATCTGACCG |
| NGS EMX1 | | GTGGCGC ATTGCCAC |
| Forward 3 | | |
| (Artificial Sequence) | | |
| SEQ ID NO: 365 | PD0316 | ACACTCTTTCCCTACACGACGCTCTTCCGATCTTGACC |
| NGS EMX1 | | GGTGGCG CATTGCCAC |
| Forward 4 | | |
| (Artificial Sequence) | | |
| SEQ ID NO: 366 | PD0317 | ACACTCTTTCCCTACACGACGCTCTTCCGATCTCTGAC |
| NGS EMX1 | | CGGTGGC GCATTGCCAC |
| Forward 5 | | |
| (Artificial Sequence) | | |
| SEQ ID NO: 367 | PD0318 | ACACTCTTTCCCTACACGACGCTCTTCCGATCTACTGA |
| NGS EMX1 | | CCGGTGG CGCATTGCCAC |
| Forward 6 | | |
| (Artificial Sequence) | | |
| SEQ ID NO: 368 | PD0319 | ACACTCTTTCCCTACACGACGCTCTTCCGATCTTACTG |
| NGS EMX1 | | ACCGGTG GCGCATTGCCAC |
| Forward 7 | | |
| (Artificial Sequence) | | |
| SEQ ID NO: 369 | PD0320 | ACACTCTTTCCCTACACGACGCTCTTCCGATCTGTACT |
| NGS EMX1 | | GACCGGT GGCGCATTGCCAC |
| Forward 8 | | |
| (Artificial Sequence) | | |
| SEQ ID NO: 370 | PD0321 | GTGACTGGAGTTCAGACGTGTGCTCTTCCGATCTCAGA |
| NGS EMX1 Reverse | | GTCCAGC TTGGGCCCA |
| (Artificial Sequence) | | |

Sequences of off-target sites can be found in Table 9 below.

TABLE 9

| SEQ ID NO/ | |
|---|---|
| DESCRIPTION/ | |
| SOURCE | SEQUENCE (5′-3′) |
| SEQ ID NO: 371 | GATATTTTCCCAGCTCACCA |
| Cas9_chr6:149045959 | |
| (Artificial | |
| Sequence) | |
| SEQ ID NO: 372 | TCTATTCTCCCAGCTCCCCA |
| Cas9_chr16:18607730 | |
| (Artificial | |
| Sequence) | |
| SEQ ID NO: 373 | AGCGGCTTCTGTCTCTGTGAGTGAGCTGGCGGTCTCCGTC |
| Bxb1_NC_000002 | |
| (Artificial | |
| Sequence) | |
| SEQ ID NO: 374 | GACTAGCCCACGCTCCGGTTCTGAGCCGCGACGGCGGTCTCCG |
| Bxb1_NC_000003 | |
| (Artificial | |
| Sequence) | |
| SEQ ID NO: 375 | CCCAGGGTCCCATGCGCTCCCCGGCCCTGACGGCGGTCTCC |
| Bxb1_NC_000009 | |
| (Artificial | |
| Sequence) | |

Linker sequences in Table 10 below.

TABLE 10

| Description | Sequence (5′-3′) | Amino acid sequence |
|---|---|---|
| A - P2A | GGAAGCGGAGCTACTAACTTCAGCCT | GSGATNFSLLKQAGDVEENPGP (SEQ ID |
| | GCTGAAGCAGGCTGGCGACGTGGAGG | NO: 418) |
| | AGAACCCTGGACCT (SEQ ID NO: 410) | |
| B - (GGGS)3 | GGGGGAGGAGGTTCTGGAGGCGGAGG | GGGGSGGGGSGGGGS (SEQ ID NO: 419) |
| | CTCCGGAGGCGGAGGGTCA (SEQ ID | |
| | NO: 411) | |
| C - GGGGS | GGAGGTGGCGGGAGC (SEQ ID NO: | GGGGS (SEQ ID NO: 420) |
| | 412) | |
| D - PAPAP | CCCGCACCAGCGCCT (SEQ ID NO: | PAPAP (SEQ ID NO: 421) |
| | 413) | |
| E - (EAAAK)3 | GAGGCAGCTGCCAAGGAAGCCGCT | EAAAKEAAAKEAAAK (SEQ ID NO: |
| | GCCAAGGAGGCGGCCGCAAAG | 422) |
| | (SEQ ID NO: 414) | |
| F - XTEN | AGTGGGAGCGAGACCCCTGGGACT | SGSETPGTSESATPES (SEQ ID NO: 423) |
| | AGCGAGTCAGCTACACCCGAAAGC | |
| | (SEQ ID NO: 415) | |
| G - (GGS)6 | GGGGGGTCAGGTGGATCCGGCGG | GGSGGSGGSGGSGGSGGS (SEQ ID NO |
| | AAGTGGCGGATCCGGTGGATCTGG | 424) |
| | CGGCAGT (SEQ ID NO: 416) | |
| H - EAAAK | GAAGCTGCTGCTAAG (SEQ ID NO: | EAAAK (SEQ ID NO: 425) |
| | 417) | |

Exemplary fusion sequences in Table 11 below.

| Description | Sequence |
|---|---|
| SpCas9-XTEN- | MKRTADGSEFESPKKKRKVDKKYSIGLDIGTNSVGWAVITDEYKVPS |
| RT(1-478)-Sto7d- | KKFKVLGNTDRHSIKKNLIGALLFDSGETAEATRLKRTARRRYTRR |
| GGGGS-BxbINT | KNRICYLQEIFSNEMAKVDDSFFHRLEESFLVEEDKKHERHPIFGNI |
| Amino acid | VDEVAYHEKYPTIYHLRKKLVDSTDKADLRLIYLALAHMIKFRGHF |
| SEQ ID NO: 376 | LIEGDLNPDNSDVDKLFIQLVQTYNQLFEENPINASGVDAKAILSAR |
| | LSKSRRLENLIAQLPGEKKNGLFGNLIALSLGLTPNFKSNFDLAEDA |
| | KLQLSKDTYDDDLDNLLAQIGDQYADLFLAAKNLSDAILLSDILRVN |
| | TEITKAPLSASMIKRYDEHHQDLTLLKALVRQQLPEKYKEIFFDQS |
| | KNGYAGYIDGGASQEEFYKFIKPILEKMDGTEELLVKLNREDLLRK |
| | QRTFDNGSIPHQIHLGELHAILRRQEDFYPFLKDNREKIEKILTFRIP |
| | YYVGPLARGNSRFAWMTRKSEETITPWNFEEVVDKGASAQSFIER |
| | MTNFDKNLPNEKVLPKHSLLYEYFTVYNELTKVKYVTEGMRKPAF |
| | LSGEQKKAIVDLLFKTNRKVTVKQLKEDYFKKIECFDSVEISGVED |
| | RFNASLGTYHDLLKIIKDKDFLDNEENEDILEDIVLTLTLFEDREMIE |
| | ERLKTYAHLFDDKVMKQLKRRRYTGWGRLSRKLINGIRDKQSGK |
| | TILDFLKSDGFANRNFMQLIHDDSLTFKEDIQKAQVSGQGDSLHEHI |
| | ANLAGSPAIKKGILQTVKVVDELVKVMGRHKPENIVIEMARENQTT |
| | QKGQKNSRERMKRIEEGIKELGSQILKEHPVENTQLQNEKLYLYYL |
| | QNGRDMYVDQELDINRLSDYDVDAIVPQSFLKDDSIDNKVLTRSDK |
| | NRGKSDNVPSEEVVKKMKNYWRQLLNAKLITQRKFDNLTKAERG |
| | GLSELDKAGFIKRQLVETRQITKHVAQILDSRMNTKYDENDKLIRE |
| | VKVITLKSKLVSDFRKDFQFYKVREINNYHHAHDAYLNAVVGTALI |
| | KKYPKLESEFVYGDYKVYDVRKMIAKSEQEIGKATAKYFFYSNIMN |
| | FFKTEITLANGEIRKRPLIETNGETGEIVWDKGRDFATVRKVLSMP |
| | QVNIVKKTEVQTGGFSKESILPKRNSDKLIARKKDWDPKKYGGFDS |
| | PTVAYSVLVVAKVEKGKSKKLKSVKELLGITIMERSSFEKNPIDFLE |
| | AKGYKEVKKDLIIKLPKYSLFELENGRKRMLASAGELQKGNELAL |
| | PSKYVNFLYLASHYEKLKGSPEDNEQKQLFVEQHKHYLDEIIEQISE |
| | FSKRVILADANLDKVLSAYNKHRDKPIREQAENIIHLFTLTNLGAPA |
| | AFKYFDTTIDRKRYTSTKEVLDATLIHQSITGLYETRIDLSQLGGDS |
| | GGSSGGSSGSETPGTSESATPESSGSETPGTSESATPESSGSETPGTSESAT |
| | PESSGGSSGGSSTLNIEDEYRLHETSKEPDVSLGSTWLSDFPQAWAET |
| | GGMGLAVRQAPLIIPLKATSTPVSIKQYPMSQEARLGIKPHIQRLLD |
| | QGILVPCQSPWNTPLLPVKKPGTNDYRPVQDLREVNKRVEDIHPTV |
| | PNPYNLLSGPPPSHQWYTVLDLKDAFFCLRLHPTSQPLFAFEWRDP |
| | EMGISGQLTWTRLPQGFKNSPTLFNEALHRDLADFRIQHPDLILLQ |
| | YVDDLLLAATSELDCQQGTRALLQTLGNLGYRASAKKAQICQKQV |
| | KYLGYLLKEGQRWLTEARKETVMGQPTPKTPRQLREFLGKAGFC |
| | RLFIPGFAEMAAPLYPLTKPGTLFNWGPDQQKAYQEIKQALLTAPA |
| | LGLPDLTKPFELFVDEKQGYAKGVLTQKLGPWRRPVAYLSKKLDP |
| | VAAGWPPCLRMVAAIAVLTKDAGKLTMGQPLVILAPHAVEALVK |
| | QPPDRWLSNARMTHYQALLLDTDRVQFGPVVALNPATLLPLPEEG |
| | LQHNCLDGTGGGGVTVKFKYKGEELEVDISKIKKVWRVGKMISFT |
| | YDDNGKTGRGAVSEKDAPKELLQMLEKSGKKSGGSKRTADGSEFE |
| | PKKKRKVGGGGSPKKKRKVYPYDVPDYAGSRALVVIRLSRVTDATTS |
| | PERQLESCQQLCAQRGWDVVGVAEDLDVSGAVDPFDRKRRPNLAR |
| | WLAFEEQPFDVIVAYRVDRLTRSIRHLQQLVHWAEDHKKLVVSAT |
| | EAHFDTTTPFAAVVIALMGTVAQMELEAIKERNRSAAHFNIRAGKY |
| | RGSLPPWGYLPTRVDGEWRLVPDPVQRERILEVYHRVVDNHEPLH |
| | LVAHDLNRRGVLSPKDYFAQLQGREPQGREWSATALKRSMISEAM |
| | LGYATLNGKTVRDDDGAPLVRAEPILTREQLEALRAELVKTSRAKP |
| | AVSTPSLLLRVLFCAVCGEPAYKFAGGGRKHPRYRCRSMGFPKHC |
| | GNGTVAMAEWDAFCEEQVLDLLGDAERLEKVWVAGSDSAVELAE |
| | VNAELVDLTSLIGSPAYRAGSPQREALDARIAALAARQEELEGLEAR |
| | PSGWEWRETGQRFGDWWREQDTAAKNTWLRSMNVRLTFDVRGG |
| | LTRTIDFGDLQEYEQHLRLGSVVERLHTGMS |
| SpCas9-XTEN- | ATGAAACGGACAGCCGACGGAAGCGAGTTCGAGTCACCAAAGAAG |
| RT(1-478)-Sto7d- | AAGCGGAAAGTCGACAAGAAGTACAGCATCGGCCTGGACATCGGCA |
| GGGGS-BxbINT | CCAACTCTGTGGGCTGGGCCGTGATCACCGACGAGTACAAGGTGCC |
| Nucleic acid | CAGCAAGAAATTCAAGGTGCTGGGCAACACCGACCGGCACAGCATC |
| SEQ ID NO: 377 | AAGAAGAACCTGATCGGAGCCCTGCTGTTCGACAGCGGCGAAACAG |
| | CCGAGGCCACCCGGCTGAAGAGAACCGCCAGAAGAAGATACACCA |
| | GACGGAAGAACCGGATCTGCTATCTGCAAGAGATCTTCAGCAACGA |
| | GATGGCCAAGGTGGACGACAGCTTCTTCCACAGACTGGAAGAGTCC |
| | TTCCTGGTGGAAGAGGATAAGAAGCACGAGCGGCACCCCATCTTCG |
| | GCAACATCGTGGACGAGGTGGCCTACCACGAGAAGTACCCCACCAT |
| | CTACCACCTGAGAAAGAAACTGGTGGACAGCACCGACAAGGCCGAC |
| | CTGCGGCTGATCTATCTGGCCCTGGCCCACATGATCAAGTTCCGGGG |
| | CCACTTCCTGATCGAGGGCGACCTGAACCCCGACAACAGCGACGTG |
| | GACAAGCTGTTCATCCAGCTGGTGCAGACCTACAACCAGCTGTTCGA |
| | GGAAAACCCCATCAACGCCAGCGGCGTGGACGCCAAGGCCATCCTG |
| | TCTGCCAGACTGAGCAAGAGCAGACGGCTGGAAAATCTGATCGCCC |
| | AGCTGCCCGGCGAGAAGAAGAATGGCCTGTTCGGAAACCTGATTGC |
| | CCTGAGCCTGGGCCTGACCCCCAACTTCAAGAGCAACTTCGACCTGG |
| | CCGAGGATGCCAAACTGCAGCTGAGCAAGGACACCTACGACGACGA |
| | CCTGGACAACCTGCTGGCCCAGATCGGCGACCAGTACGCCGACCTG |
| | TTTCTGGCCGCCAAGAACCTGTCCGACGCCATCCTGCTGAGCGACAT |
| | CCTGAGAGTGAACACCGAGATCACCAAGGCCCCCCTGAGCGCCTCT |
| | ATGATCAAGAGATACGACGAGCACCACCAGGACCTGACCCTGCTGA |
| | AAGCTCTCGTGCGGCAGCAGCTGCCTGAGAAGTACAAAGAGATTTT |
| | CTTCGACCAGAGCAAGAACGGCTACGCCGGCTACATTGACGGCGGA |
| | GCCAGCCAGGAAGAGTTCTACAAGTTCATCAAGCCCATCCTGGAAA |
| | AGATGGACGGCACCGAGGAACTGCTCGTGAAGCTGAACAGAGAGG |
| | ACCTGCTGCGGAAGCAGCGGACCTTCGACAACGGCAGCATCCCCCA |
| | CCAGATCCACCTGGGAGAGCTGCACGCCATTCTGCGGCGGCAGGAA |
| | GATTTTTACCCATTCCTGAAGGACAACCGGGAAAAGATCGAGAAGA |
| | TCCTGACCTTCCGCATCCCCTACTACGTGGGCCCTCTGGCCAGGGGA |
| | AACAGCAGATTCGCCTGGATGACCAGAAAGAGCGAGGAAACCATCA |
| | CCCCCTGGAACTTCGAGGAAGTGGTGGACAAGGGCGCTTCCGCCCA |
| | GAGCTTCATCGAGCGGATGACCAACTTCGATAAGAACCTGCCCAAC |
| | GAGAAGGTGCTGCCCAAGCACAGCCTGCTGTACGAGTACTTCACCG |
| | TGTATAACGAGCTGACCAAAGTGAAATACGTGACCGAGGGAATGAG |
| | AAAGCCCGCCTTCCTGAGCGGCGAGCAGAAAAAGGCCATCGTGGAC |
| | CTGCTGTTCAAGACCAACCGGAAAGTGACCGTGAAGCAGCTGAAAG |
| | AGGACTACTTCAAGAAAATCGAGTGCTTCGACTCCGTGGAAATCTCC |
| | GGCGTGGAAGATCGGTTCAACGCCTCCCTGGGCACATACCACGATC |
| | TGCTGAAAATTATCAAGGACAAGGACTTCCTGGACAATGAGGAAAA |
| | CGAGGACATTCTGGAAGATATCGTGCTGACCCTGACACTGTTTGAGG |
| | ACAGAGAGATGATCGAGGAACGGCTGAAAACCTATGCCCACCTGTT |
| | CGACGACAAAGTGATGAAGCAGCTGAAGCGGCGGAGATACACCGG |
| | CTGGGGCAGGCTGAGCCGGAAGCTGATCAACGGCATCCGGGACAAG |
| | CAGTCCGGCAAGACAATCCTGGATTTCCTGAAGTCCGACGGCTTCGC |
| | CAACAGAAACTTCATGCAGCTGATCCACGACGACAGCCTGACCTTT |
| | AAAGAGGACATCCAGAAAGCCCAGGTGTCCGGCCAGGGCGATAGCC |
| | TGCACGAGCACATTGCCAATCTGGCCGGCAGCCCCGCCATTAAGAA |
| | GGGCATCCTGCAGACAGTGAAGGTGGTGGACGAGCTCGTGAAAGTG |
| | ATGGGCCGGCACAAGCCCGAGAACATCGTGATCGAAATGGCCAGAG |
| | AGAACCAGACCACCCAGAAGGGACAGAAGAACAGCCGCGAGAGAA |
| | TGAAGCGGATCGAAGAGGGCATCAAAGAGCTGGGCAGCCAGATCCT |
| | GAAAGAACACCCCGTGGAAAACACCCAGCTGCAGAACGAGAAGCT |
| | GTACCTGTACTACCTGCAGAATGGGCGGGATATGTACGTGGACCAG |
| | GAACTGGACATCAACCGGCTGTCCGACTACGATGTGGACGCTATCG |
| | TGCCTCAGAGCTTTCTGAAGGACGACTCCATCGACAACAAGGTGCT |
| | GACCAGAAGCGACAAGAACCGGGGCAAGAGCGACAACGTGCCCTC |
| | CGAAGAGGTCGTGAAGAAGATGAAGAACTACTGGCGGCAGCTGCTG |
| | AACGCCAAGCTGATTACCCAGAGAAAGTTCGACAATCTGACCAAGG |
| | CCGAGAGAGGCGGCCTGAGCGAACTGGATAAGGCCGGCTTCATCAA |
| | GAGACAGCTGGTGGAAACCCGGCAGATCACAAAGCACGTGGCACA |
| | GATCCTGGACTCCCGGATGAACACTAAGTACGACGAGAATGACAAG |
| | CTGATCCGGGAAGTGAAAGTGATCACCCTGAAGTCCAAGCTGGTGT |
| | CCGATTTCCGGAAGGATTTCCAGTTTTACAAAGTGCGCGAGATCAAC |
| | AACTACCACCACGCCCACGACGCCTACCTGAACGCCGTCGTGGGAA |
| | CCGCCCTGATCAAAAAGTACCCTAAGCTGGAAAGCGAGTTCGTGTA |
| | CGGCGACTACAAGGTGTACGACGTGCGGAAGATGATCGCCAAGAGC |
| | GAGCAGGAAATCGGCAAGGCTACCGCCAAGTACTTCTTCTACAGCA |
| | ACATCATGAACTTTTTCAAGACCGAGATTACCCTGGCCAACGGCGA |
| | GATCCGGAAGCGGCCTCTGATCGAGACAAACGGCGAAACCGGGGA |
| | GATCGTGTGGGATAAGGGCCGGGATTTTGCCACCGTGCGGAAAGTG |
| | CTGAGCATGCCCCAAGTGAATATCGTGAAAAAGACCGAGGTGCAGA |
| | CAGGCGGCTTCAGCAAAGAGTCTATCCTGCCCAAGAGGAACAGCGA |
| | TAAGCTGATCGCCAGAAAGAAGGACTGGGACCCTAAGAAGTACGGC |
| | GGCTTCGACAGCCCCACCGTGGCCTATTCTGTGCTGGTGGTGGCCAA |
| | AGTGGAAAAGGGCAAGTCCAAGAAACTGAAGAGTGTGAAAGAGCT |
| | GCTGGGGATCACCATCATGGAAAGAAGCAGCTTCGAGAAGAATCCC |
| | ATCGACTTTCTGGAAGCCAAGGGCTACAAAGAAGTGAAAAAGGACC |
| | TGATCATCAAGCTGCCTAAGTACTCCCTGTTCGAGCTGGAAAACGGC |
| | CGGAAGAGAATGCTGGCCTCTGCCGGCGAACTGCAGAAGGGAAACG |
| | AACTGGCCCTGCCCTCCAAATATGTGAACTTCCTGTACCTGGCCAGC |
| | CACTATGAGAAGCTGAAGGGCTCCCCCGAGGATAATGAGCAGAAAC |
| | AGCTGTTTGTGGAACAGCACAAGCACTACCTGGACGAGATCATCGA |
| | GCAGATCAGCGAGTTCTCCAAGAGAGTGATCCTGGCCGACGCTAAT |
| | CTGGACAAAGTGCTGTCCGCCTACAACAAGCACCGGGATAAGCCCA |
| | TCAGAGAGCAGGCCGAGAATATCATCCACCTGTTTACCCTGACCAAT |
| | CTGGGAGCCCCTGCCGCCTTCAAGTACTTTGACACCACCATCGACCG |
| | GAAGAGGTACACCAGCACCAAAGAGGTGCTGGACGCCACCCTGATC |
| | CACCAGAGCATCACCGGCCTGTACGAGACACGGATCGACCTGTCTC |
| | AGCTGGGAGGTGACTCTGGAGGATCTAGCGGAGGATCCTCTGGCAG |
| | CGAGACACCAGGAACAAGCGAGTCAGCAACACCAGAGAGCTCTGGT |
| | AGCGAGACACCCGGTACCAGTGAAAGCGCCACGCCAGAAAGCAGT |
| | GGGAGTGAGACTCCGGGTACATCTGAATCAGCGACACCGGAATCAA |
| | GTGGCGGCAGCAGCGGCGGCAGCAGCACCCTAAATATAGAAGATGA |
| | GTATCGGCTACATGAGACCTCAAAAGAGCCAGATGTTTCTCTAGGGT |
| | CCACATGGCTGTCTGATTTTCCTCAGGCCTGGGCGGAAACCGGGGGC |
| | ATGGGACTGGCAGTTCGCCAAGCTCCTCTGATCATACCTCTGAAAGC |
| | AACCTCTACCCCCGTGTCCATAAAACAATACCCCATGTCACAAGAA |
| | GCCAGACTGGGGATCAAGCCCCACATACAGAGACTGTTGGACCAGG |
| | GAATACTGGTACCCTGCCAGTCCCCCTGGAACACGCCCCTGCTACCC |
| | GTTAAGAAACCAGGGACTAATGATTATAGGCCTGTCCAGGATCTGA |
| | GAGAAGTCAACAAGCGGGTGGAAGACATCCACCCCACCGTGCCCAA |
| | CCCTTACAACCTCTTGAGCGGGCCCCCACCGTCCCACCAGTGGTACA |
| | CTGTGCTTGATTTAAAGGATGCCTTTTTCTGCCTGAGACTCCACCCC |
| | ACCAGTCAGCCTCTCTTCGCCTTTGAGTGGAGAGATCCAGAGATGGG |
| | AATCTCAGGACAATTGACCTGGACCAGACTCCCACAGGGTTTCAAA |
| | AACAGTCCCACCCTGTTTAATGAGGCACTGCACAGAGACCTAGCAG |
| | ACTTCCGGATCCAGCACCCAGACTTGATCCTGCTACAGTACGTGGAT |
| | GACTTACTGCTGGCCGCCACTTCTGAGCTAGACTGCCAACAAGGTAC |
| | TCGGGCCCTGTTACAAACCCTAGGGAACCTCGGGTATCGGGCCTCG |
| | GCCAAGAAAGCCCAAATTTGCCAGAAACAGGTCAAGTATCTGGGGT |
| | ATCTTCTAAAAGAGGGTCAGAGATGGCTGACTGAGGCCAGAAAAGA |
| | GACTGTGATGGGGCAGCCTACTCCGAAGACCCCTCGACAACTAAGG |
| | GAGTTCCTAGGGAAGGCAGGCTTCTGTCGCCTCTTCATCCCTGGGTT |
| | TGCAGAAATGGCAGCCCCCCTGTACCCTCTCACCAAACCGGGGACT |
| | CTGTTTAATTGGGGCCCAGACCAACAAAAGGCCTATCAAGAAATCA |
| | AGCAAGCTCTTCTAACTGCCCCAGCCCTGGGGTTGCCAGATTTGACT |
| | AAGCCCTTTGAACTCTTTGTCGACGAGAAGCAGGGCTACGCCAAAG |
| | GTGTCCTAACGCAAAAACTGGGACCTTGGCGTCGGCCGGTGGCCTA |
| | CCTGTCCAAAAAGCTAGACCCAGTAGCAGCTGGGTGGCCCCCTTGC |
| | CTACGGATGGTAGCAGCCATTGCCGTACTGACAAAGGATGCAGGCA |
| | AGCTAACCATGGGACAGCCACTAGTCATTCTGGCCCCCCATGCAGTA |
| | GAGGCACTAGTCAAACAACCCCCCGACCGCTGGCTTTCCAACGCCC |
| | GGATGACTCACTATCAGGCCTTGCTTTTGGACACGGACCGGGTCCAG |
| | TTCGGACCGGTGGTAGCCCTGAACCCGGCTACGCTGCTCCCACTGCC |
| | TGAGGAAGGGCTGCAACACAACTGCCTTGATGGGACAGGTGGCGGT |
| | GGTGTCACCGTCAAGTTCAAGTACAAGGGTGAGGAACTTGAAGTTG |
| | ATATTAGCAAAATCAAGAAGGTTTGGCGCGTTGGTAAAATGATATC |
| | TTTTACTTATGACGACAACGGCAAGACAGGTAGAGGGGCAGTGTCT |
| | GAGAAAGACGCCCCCAAGGAGCTGTTGCAAATGTTGGAAAAGTCTG |
| | GGAAAAAGTCTGGCGGCTCAAAAAGAACCGCCGACGGCAGCGAATT |
| | CGAGCCCAAGAAGAAGAGGAAAGTCGGAGGTGGCGGGAGCCCAAA |
| | AAAGAAAAGAAAAGTGTATCCCTATGATGTCCCCGATTATGCCGGT |
| | TCAAGAGCCCTGGTCGTGATTAGACTGAGCCGAGTGACAGACGCCA |
| | CCACAAGTCCCGAGAGACAGCTGGAATCATGCCAGCAGCTCTGTGC |
| | TCAGCGGGGTTGGGATGTGGTCGGCGTGGCAGAGGATCTGGACGTG |
| | AGCGGGGCCGTCGATCCATTCGACAGAAAGAGGAGGCCCAACCTGG |
| | CAAGATGGCTCGCTTTCGAGGAACAGCCCTTTGATGTGATCGTCGCC |
| | TACAGAGTGGACCGGCTGACCCGCTCAATTCGACATCTCCAGCAGCT |
| | GGTGCATTGGGCTGAGGACCACAAGAAACTGGTGGTCAGCGCAACA |
| | GAAGCCCACTTCGATACTACCACACCTTTTGCCGCTGTGGTCATCGC |
| | ACTGATGGGCACTGTGGCCCAGATGGAGCTCGAAGCTATCAAGGAG |
| | CGAAACAGGAGCGCAGCCCATTTCAATATTAGGGCCGGTAAATACA |
| | GAGGCTCCCTGCCCCCTTGGGGATATCTCCCTACCAGGGTGGATGGG |
| | GAGTGGAGACTGGTGCCAGACCCCGTCCAGAGAGAGCGGATTCTGG |
| | AAGTGTACCACAGAGTGGTCGATAACCACGAACCACTCCATCTGGT |
| | GGCACACGACCTGAATAGACGCGGCGTGCTCTCTCCAAAGGATTAT |
| | TTTGCTCAGCTGCAGGGAAGAGAGCCACAGGGAAGAGAATGGAGTG |
| | CTACTGCACTGAAGAGATCTATGATCAGTGAGGCTATGCTGGGTTAC |
| | GCAACACTCAATGGCAAAACTGTCCGGGACGATGACGGAGCCCCTC |
| | TGGTGAGGGCTGAGCCTATTCTCACCAGAGAGCAGCTCGAAGCTCT |
| | GCGGGCAGAACTGGTCAAGACTAGTCGCGCCAAACCTGCCGTGAGC |
| | ACCCCAAGCCTGCTCCTGAGGGTGCTGTTCTGCGCCGTCTGTGGAGA |
| | GCCAGCATACAAGTTTGCCGGCGGAGGGCGCAAACATCCCCGCTAT |
| | CGATGCAGGAGCATGGGGTTCCCTAAGCACTGTGGAAACGGGACAG |
| | TGGCCATGGCTGAGTGGGACGCCTTTTGCGAGGAACAGGTGCTGGA |
| | TCTCCTGGGTGACGCTGAGCGGCTGGAAAAAGTGTGGGTGGCAGGA |
| | TCTGACTCCGCTGTGGAGCTGGCAGAAGTCAATGCCGAGCTCGTGG |
| | ATCTGACTTCCCTCATCGGATCTCCTGCATATAGAGCTGGGTCCCCA |
| | CAGAGAGAAGCTCTGGACGCACGAATTGCTGCACTCGCTGCTAGAC |
| | AGGAGGAACTGGAGGGCCTGGAGGCCAGGCCCTCTGGATGGGAGTG |
| | GCGAGAAACCGGACAGAGGTTTGGGGATTGGTGGAGGGAGCAGGA |
| | CACCGCAGCCAAGAACACATGGCTGAGATCCATGAATGTCCGGCTC |
| | ACATTCGACGTGCGCGGTGGCCTGACTCGAACCATCGATTTTGGCGA |
| | CCTGCAGGAGTATGAACAGCACCTGAGACTGGGGTCCGTGGTCGAA |
| | AGACTGCACACTGGGATGTCC |
| SpCas9 | DKKYSIGLDIGTNSVGWAVITDEYKVPSKKFKVLGNTDRHSIKKNLIGA |
| Amino acid | LLFDSGETAEATRLKRTARRRYTRRKNRICYLQEIFSNEMAKVDDSFFH |
| SEQ ID NO: 378 | RLEESFLVEEDKKHERHPIFGNIVDEVAYHEKYPTIYHLRKKLVDSTDK |
| | ADLRLIYLALAHMIKFRGHFLIEGDLNPDNSDVDKLFIQLVQTYNQLFE |
| | ENPINASGVDAKAILSARLSKSRRLENLIAQLPGEKKNGLFGNLIALSLG |
| | LTPNFKSNFDLAEDAKLQLSKDTYDDDLDNLLAQIGDQYADLFLAAKN |
| | LSDAILLSDILRVNTEITKAPLSASMIKRYDEHHQDLTLLKALVRQQLPE |
| | KYKEIFFDQSKNGYAGYIDGGASQEEFYKFIKPILEKMDGTEELLVKLN |
| | REDLLRKQRTFDNGSIPHQIHLGELHAILRRQEDFYPFLKDNREKIEKILT |
| | FRIPYYVGPLARGNSRFAWMTRKSEETITPWNFEEVVDKGASAQSFIER |
| | MTNFDKNLPNEKVLPKHSLLYEYFTVYNELTKVKYVTEGMRKPAFLSG |
| | EQKKAIVDLLFKTNRKVTVKQLKEDYFKKIECFDSVEISGVEDRFNASL |
| | GTYHDLLKIIKDKDFLDNEENEDILEDIVLTLTLFEDREMIEERLKTYAH |
| | LFDDKVMKQLKRRRYTGWGRLSRKLINGIRDKQSGKTILDFLKSDGFA |
| | NRNFMQLIHDDSLTFKEDIQKAQVSGQGDSLHEHIANLAGSPAIKKGIL |
| | QTVKVVDELVKVMGRHKPENIVIEMARENQTTQKGQKNSRERMKRIE |
| | EGIKELGSQILKEHPVENTQLQNEKLYLYYLQNGRDMYVDQELDINRL |
| | SDYDVDAIVPQSFLKDDSIDNKVLTRSDKNRGKSDNVPSEEVVKKMKN |
| | YWRQLLNAKLITQRKFDNLTKAERGGLSELDKAGFIKRQLVETRQITK |
| | HVAQILDSRMNTKYDENDKLIREVKVITLKSKLVSDFRKDFQFYKVREI |
| | NNYHHAHDAYLNAVVGTALIKKYPKLESEFVYGDYKVYDVRKMIAKS |
| | EQEIGKATAKYFFYSNIMNFFKTEITLANGEIRKRPLIETNGETGEIVWD |
| | KGRDFATVRKVLSMPQVNIVKKTEVQTGGFSKESILPKRNSDKLIARKK |
| | DWDPKKYGGFDSPTVAYSVLVVAKVEKGKSKKLKSVKELLGITIMERS |
| | SFEKNPIDFLEAKGYKEVKKDLIIKLPKYSLFELENGRKRMLASAGELQ |
| | KGNELALPSKYVNFLYLASHYEKLKGSPEDNEQKQLFVEQHKHYLDEII |
| | EQISEFSKRVILADANLDKVLSAYNKHRDKPIREQAENIIHLFTLTNLGA |
| | PAAFKYFDTTIDRKRYTSTKEVLDATLIHQSITGLYETRIDLSQLGGD |
| RT(1-478)-Sto7d | LNIEDEYRLHETSKEPDVSLGSTWLSDFPQAWAETGGMGLAVRQAPLII |
| Amino acid | PLKATSTPVSIKQYPMSQEARLGIKPHIQRLLDQGILVPCQSPWNTPLLP |
| SEQ ID NO: 379 | VKKPGTNDYRPVQDLREVNKRVEDIHPTVPNPYNLLSGPPPSHQWYTV |
| | LDLKDAFFCLRLHPTSQPLFAFEWRDPEMGISGQLTWTRLPQGFKNSPT |
| | LFNEALHRDLADFRIQHPDLILLQYVDDLLLAATSELDCQQGTRALLQT |
| | LGNLGYRASAKKAQICQKQVKYLGYLLKEGQRWLTEARKETVMGQPT |
| | PKTPRQLREFLGKAGFCRLFIPGFAEMAAPLYPLTKPGTLFNWGPDQQK |
| | AYQEIKQALLTAPALGLPDLTKPFELFVDEKQGYAKGVLTQKLGPWRR |
| | PVAYLSKKLDPVAAGWPPCLRMVAAIAVLTKDAGKLTMGQPLVILAP |
| | HAVEALVKQPPDRWLSNARMTHYQALLLDTDRVQFGPVVALNPATLL |
| | PLPEEGLQHNCLDGTGGGGVTVKFKYKGEELEVDISKIKKVWRVGKMI |
| | SFTYDDNGKTGRGAVSEKDAPKELLQMLEKSGKKSGGSKRTADGS |
| BxbINT | SRALVVIRLSRVTDATTSPERQLESCQQLCAQRGWDVVGVAEDLDVSG |
| Amino acid | AVDPFDRKRRPNLARWLAFEEQPFDVIVAYRVDRLTRSIRHLQQLVHW |
| SEQ ID NO: 380 | AEDHKKLVVSATEAHFDTTTPFAAVVIALMGTVAQMELEAIKERNRSA |
| | AHFNIRAGKYRGSLPPWGYLPTRVDGEWRLVPDPVQRERILEVYHRVV |
| | DNHEPLHLVAHDLNRRGVLSPKDYFAQLQGREPQGREWSATALKRSM |
| | ISEAMLGYATLNGKTVRDDDGAPLVRAEPILTREQLEALRAELVKTSRA |
| | KPAVSTPSLLLRVLFCAVCGEPAYKFAGGGRKHPRYRCRSMGFPKHCG |
| | NGTVAMAEWDAFCEEQVLDLLGDAERLEKVWVAGSDSAVELAEVNA |
| | ELVDLTSLIGSPAYRAGSPQREALDARIAALAARQEELEGLEARPSGWE |
| | WRETGQRFGDWWREQDTAAKNTWLRSMNVRLTFDVRGGLTRTIDFG |
| | DLQEYEQHLRLGSVVERLHTGMS |

### EXAMPLES

While several experimental Examples are contemplated, these Examples are intended to be non-limiting.

### Example 1

### CRE Integration Efficiency

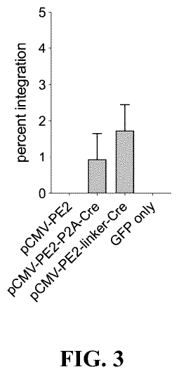

The efficiency of the CRE integration was tested. In order to test the efficacy of PASTE with GFP using lox71/lox66/Cre recombinase system, a clonal HEK293FT cell line with lox71 sequence (SEQ ID NO: 1) integrated into the genome using lentivirus was developed. The integration of GFP was tested by transfection of modified HEK293FT cell line with: (1) plus/minus SEQ ID NO: 71 comprising a Cre recombinase expression plasmid, and (2) SEQ ID NO: 72 comprising a GFP template and a lox 66 Cre site of SEQ ID NO: 2. After 72 hours, the percent integration of GFP into the lox71 site was probed. FIG. 3 shows the percent integration of GFP in the lentiviral integrated lox71 site in HEK293FT cell line in the presence of various plasmids. It was observed that pCMV PE2 P2A Cre (SEQ ID NO: 73), a mammalian expression vector with prime editing complex and Cre recombinase linked to PE2 via a cleavable linker or a non-cleavable linker, shows integration of GFP.

### Example 2

### Programmable Addition Via Site-Specific Targeting Elements (PASTE) with Cre Recombinase—Addition of Lox Site

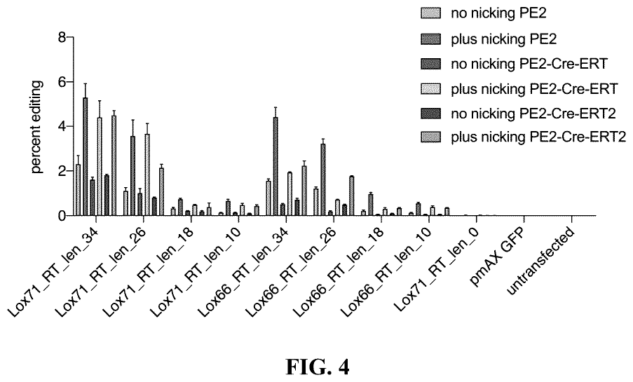

The lox71 (SEQ ID NO: 1) or lox66 (SEQ ID NO: 2) sequence was inserted into the HEK293FT cell genome using prime editing to test integration of GFP into the HEK293FT genome. In order to insert lox71 or lox66 sequence into HEK293FT cell genome, a pegRNA with PBS length of 13 base pairs operably linked to RT region of varying lengths was used. The following plasmids were used in the transfection of HEK293FT cells. The cells were transfected with (1) prime editing construct (PE2) or PE2 with conditional Cre expression, (2) Lox71 or Lox66 pegRNA targeting the HEK3 locus, and (3) plus/minus +90 HEK3 nicking second guide RNA targeting the HEK3 locus (+90 ngRNA). After 72 hours, the percent editing of the HEK293FT genome at the HEK3 locus was probed for incorporation of various lengths of lox71 or lox66 (see FIG. 4). It was observed that 34 base pair lox71 (HEK3 locus guide, SEQ ID NO: 83; and Lox71 pegRNA with RT 34 and PBS 13, SEQ ID NO: 81) with +90 ngRNA (SEQ ID NO: 75) and 34 base pair lox66 (HEK3 locus guide, SEQ ID NO: 83; and Lox66 pegRNA with RT 34 and PBS 13, SEQ ID NO: 82) with +90 ngRNA (SEQ ID NO: 75) had the highest percent editing.

### Example 3

### PASTE with Cre Recombinase—Integration of Gene

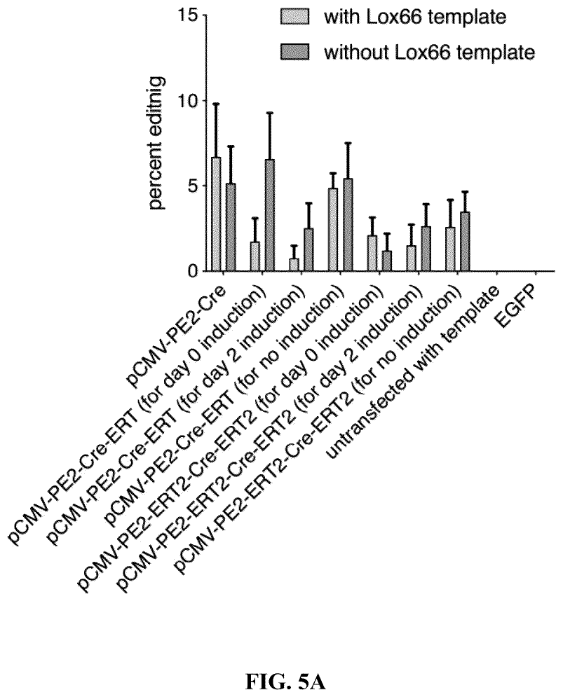

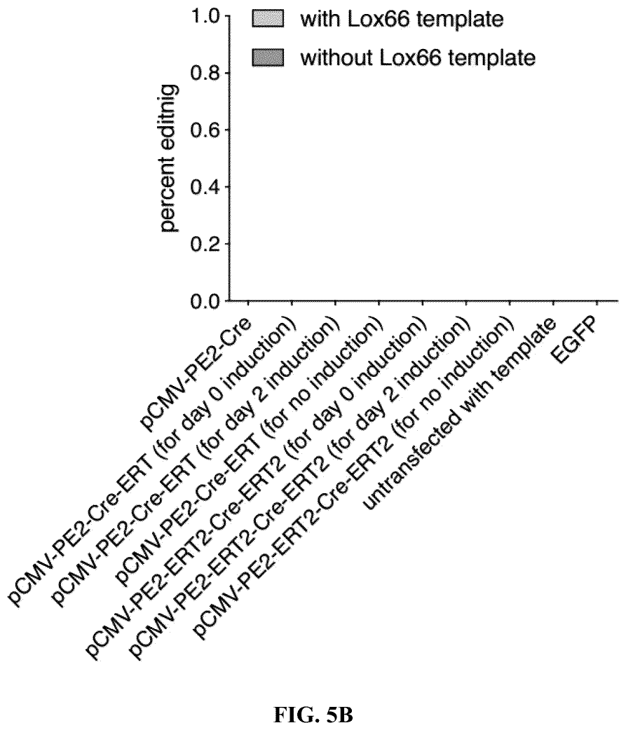

The lox71 or lox66 pegRNAs having PBS length of 13 base pairs and insert length of 34 base pairs were used to probe integration of GFP in the HEK293F genome. The PE and Cre were delivered in an inducible expression vectors and induced at day 2. The HEK293FT cells were transfected with the following plasmids: (1) prime editing construct (PE2 or PE2 with conditional Cre expression); (2) Lox71 pegRNA; (3) plus/minus +90 HEK3 nicking guide RNA; and (4) EGFP template with Lox66 site. After 72 hours, the percent editing of lox71 site and percent integration of GFP was probed with or without lox66 site in the presence of various PE/Cre constructs. FIG. 5A summarizes the percent editing of lox71 site with different PE/Cre vectors. FIG. 5B summarizes the percent integration of GFP at the lox71 site in HEK293FT cell genome. It was observed that although the lox71 site was edited in the presence of inducible or non-inducible PE/Cre expression system, there was no GFP integration.

### Example 4

### Bxb1 Integration Data Lenti Reporter

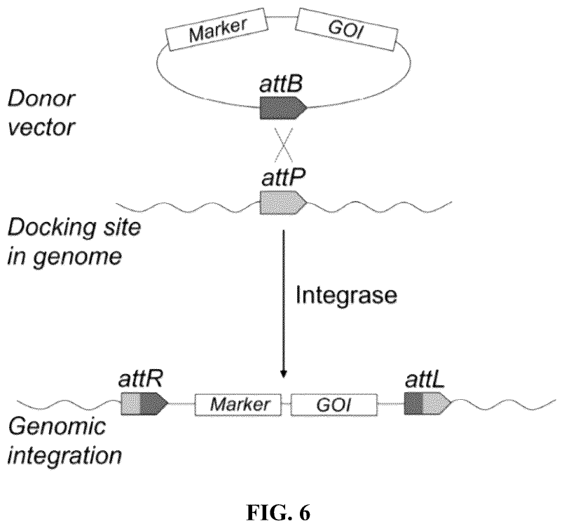

The integration system was switched to an integrase system that could result in an integration of target genes into a genome with higher efficiency. Serine integrase Bxb1 has been shown to be more active than Cre recombinase and highly efficient in bacteria and mammalian cells for irreversible integration of target genes. FIG. 6 shows a schematic of PASTE methodology using Bxb1 (Merrick, C. A. et al., ACS Synth. Biol. 2018, 7, 299-310).

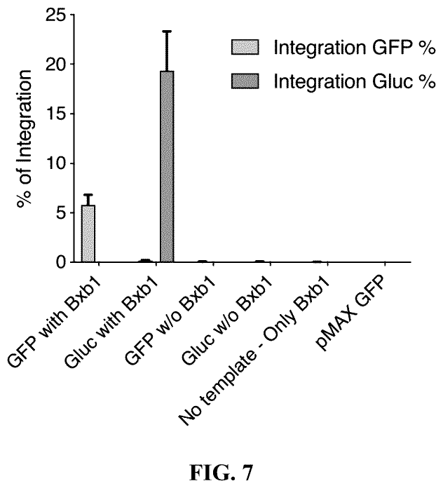

To probe the efficiency of the Bxb1 integration system, a clonal HEK293FT cell line with attB Bxb1 site (SEQ ID NO: 3) integrated using lentivirus was developed. The modified HEK293FT cell line was then transferred with the following plasmids: (1) plus/minus Bxb1 expression plasmid and (2) plus/minus GFP (SEQ ID NO: 76) or G-Luc (SEQ ID NO: 77) minicircle template with attP Bxb1 site. After 72 hours, the integration of GFP or Gluc into the attB site in the HEK293FT genome was probed. The percent integrations of GFP or Gluc into the attB locus are shown in FIG. 7. It was observed that GFP and Gluc showed efficient integration into the attB site in HEK293FT cells.

### Example 5

### Addition of Bxb1 Site to Human Genome Using PRIME

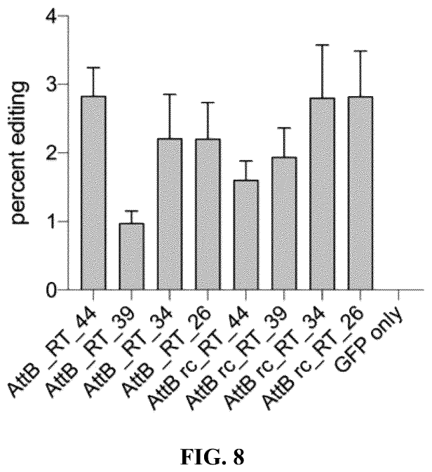

The maximum length of attB that can be integrated into a HEK293FT cell line with the best efficiency was probed. To probe the best length of attB (SEQ ID NO: 3) or its reverse complement attP (SEQ ID NO: 4) for prime editing, pegRNAs having PBS length of 13 nt with varying RT homology length were used. The following plasmids were transfected in HEK293FT: (1) prime expression plasmid; (2) HEK3 targeting pegRNA design; and (3) HEK3 +90 nicking guide. After 72 hours, the percent integration of each of the attB construct was probed. FIG. 8 shows the percent editing in each HEK3 targeting pegRNA. It was observed that attB with 44, 34 and 26 base pairs and attB reverse complement with 34 and 26 base pairs showed the highest percent editing.

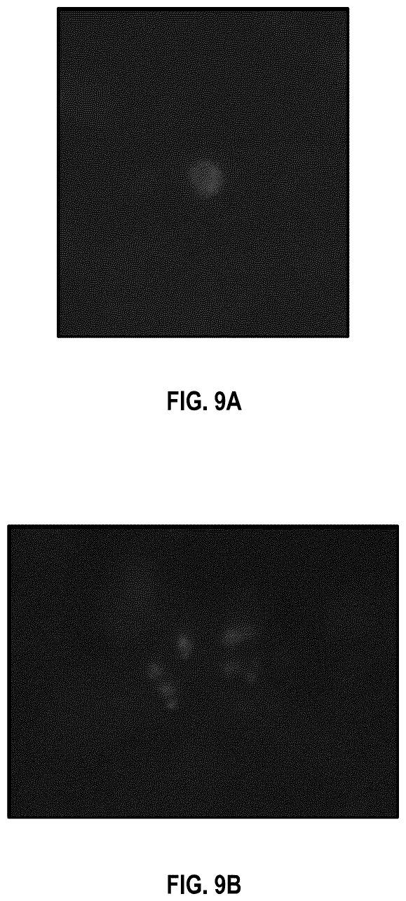

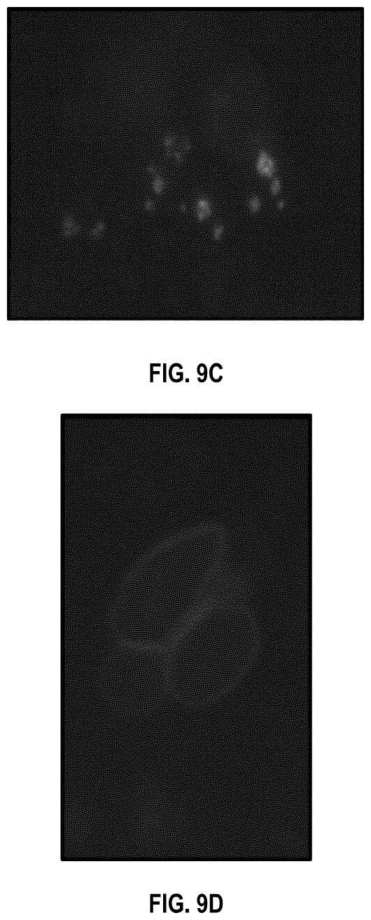

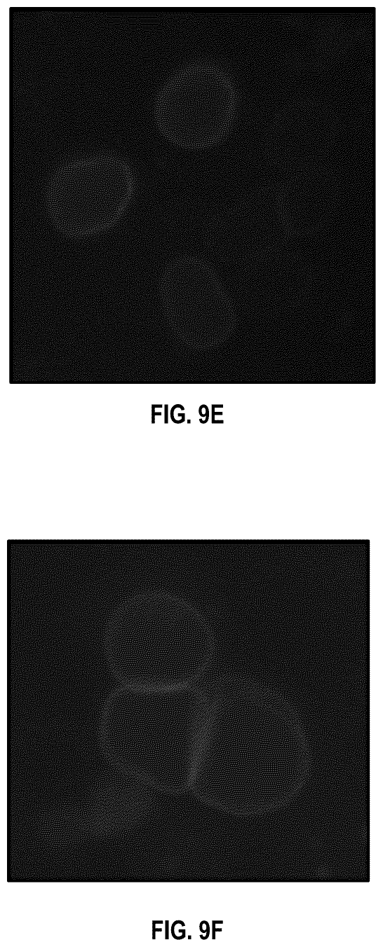

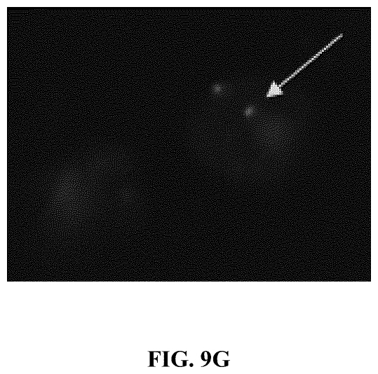

Integration PASTE was then tested with tagging cell-organelle marker proteins with GFP in HEK29FT cells. PASTE was used to tag SUPT16H, SRRM2, LAMNB1, NOLC1 and DEPDC4 with GFP in different cell-culture wells and to test the usefulness of PASTE in tracking protein localization within the cells using microscopy. FIGS. 9A-9G shows the fluorescent microscopy results for each of the organelles. SUPT16H-GFP was observed to be enriched in the nucleus, SRRM2-GFP was observed to be enriched in the nuclear speckles, LAMNB1-GFP was observed to be enriched in the nuclear membrane, NOLC1-GFP was observed to be enriched in the fibrillar center, and DEPDC4-GFP was observed to be enriched in the aggresome.

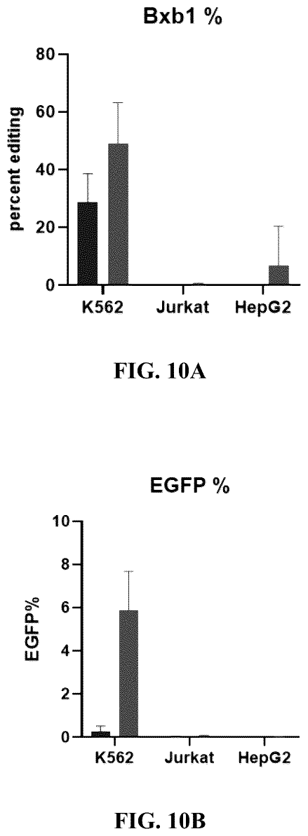

The transfection of the plasmids can be achieved using electroporation as illustrated in FIGS. 10A-10B.

### Example 6

### Programmable Integration of Genes with PASTE

The efficiency of gene integration of Gluc or EGFP with PASTE was tested. To enable gene integration with PASTE, the following HEK3 targeting pegRNAs were used: (1) 44 pegRNA: PBS of 13 nt and RT homology of 44 nt; (2) 34 pegRNA: PBS of 13 nt and RT homology of 34 nt; and (3) 26 pegRNA: PBS of 13 nt and RT homology of 26 nt.

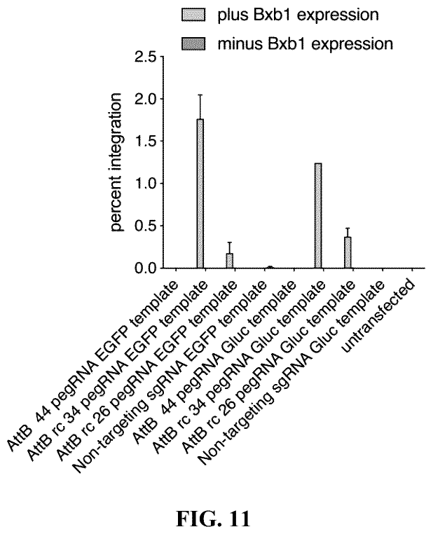

A HEK293 cell line was transfected with following plasmids HEK293FT: (1) Prime expression plasmid; (2) Bxb1 expression plasmid; (3) HEK3 targeting pegRNA design; (4) HEK3 +90 nicking guide; and (5) EGFP or Gluc minicircle. After 72 hours, the percent integration of Gluc or EGFP was observed. FIG. 11 shows integration of EGFP and Gluc with each of the tested HEK3 targeting pegRNAs. It was observed that EGFP and Gluc were efficiently integrated using PASTE.

### Example 7

### PASTE for Integration of Multiple Genes

The PASTE technique for site-specific integration of multiple genes into a cell is facilitated with the use of orthogonal attB and attP sites. Central dinucleotide can be changed to GA from GT, and only GA containing attB/attP sites can interact and do not cross react with GT containing sequences. A screen of dinucleotide combinations to find orthogonal attB/attP pairs for multiplexed PASTE editing can be performed. It has been shown that many orthogonal dinucleotide combinations can be found using a Bxb1 reporter system.

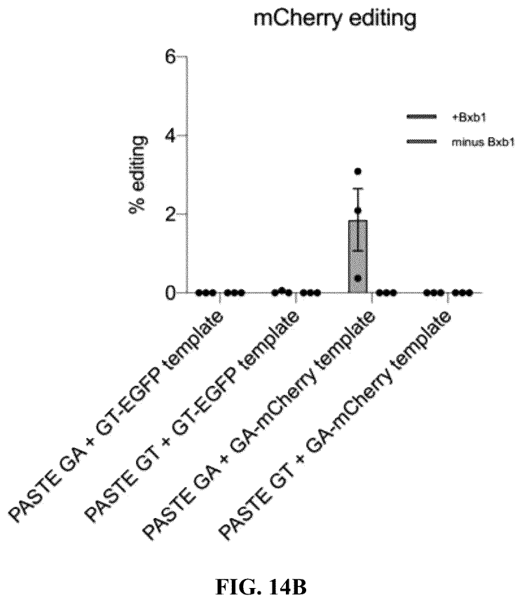

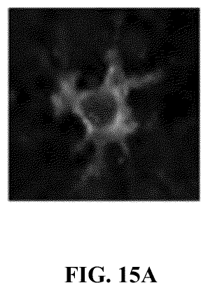

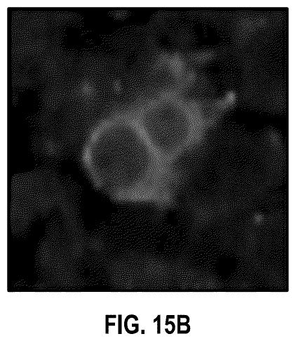

To test this, attB^{GT} and attB^{GA} dinucleotides for Bxb1 was added at a ACTB site by prime editing. A EGFP—attP^{GT} DNA minicircle and a mCherry—attP^{GA} DNA minicircle was introduced to test the percent EGFP and mCherry editing in the presence or absence of Bxb1. The results of EGFP and mCherry editing are shown in FIGS. 14A-14B.

Orthogonal editing with the right GT-EGFP and GA-mCherry pairs was achieved demonstrating the ability for multiplexed PASTE editing in cells.

Two genes were introduced in the same cell using multiplexed PASTE to tag two different genes in a single reaction. EGFP and mCherry were tagged into the loci of ACTB and NOLC1 in a x cell line, in a single reaction. Further, EGFP and mCherry were tagged into the loci of ACTB and LAMNB1. The cells were visualized using fluorescence microscopy. FIGS. 15A-15B show the results of fluorescent microscopy for multiplexed PASTE.

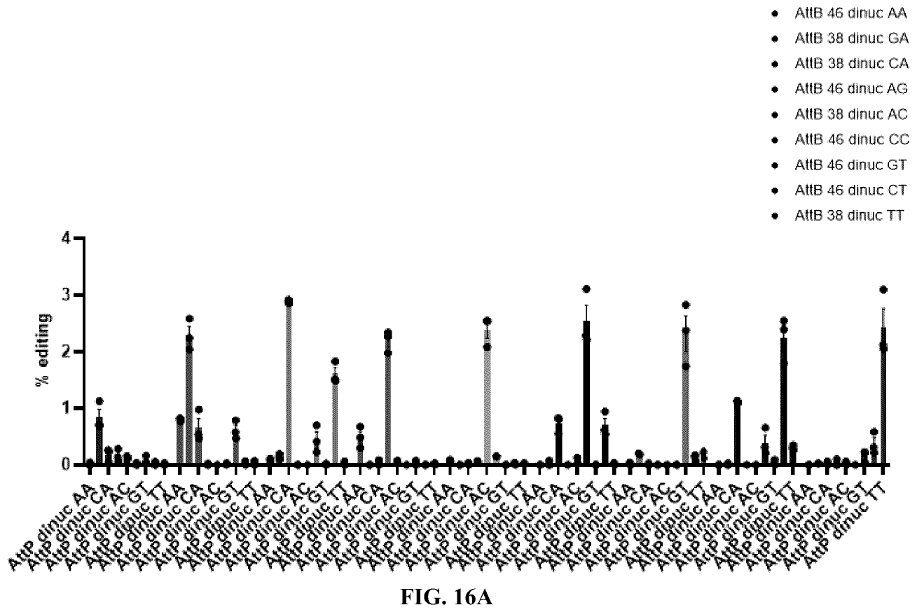

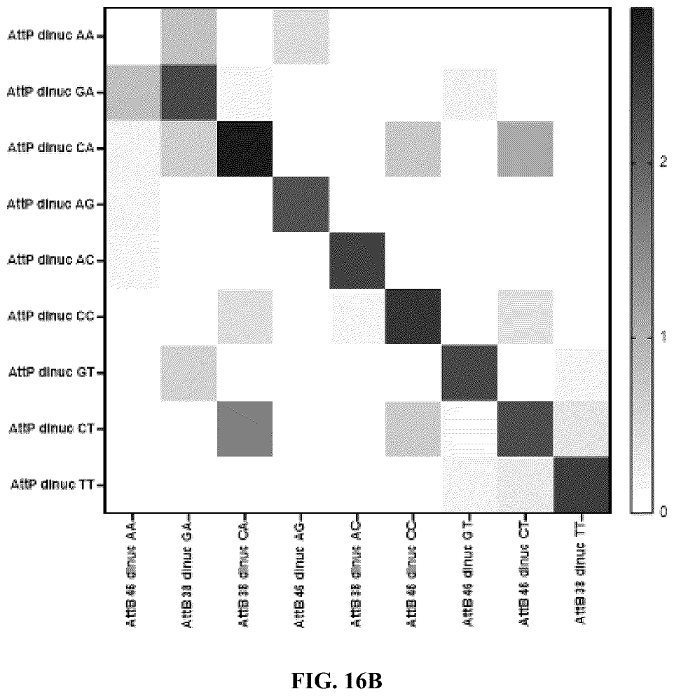

The ability of multiplexing with 9-different attB and attP central dinucleotides—AA, GA, CA, AG, AC, CC, GT, CT and TT (SEQ ID NOs: 7, 8, 23, 24, 19, 20, 25, 26, 27, 28, 9, 10, 15, 16, 17, 18, 5 and 6)—in a 9×9 cross of attB and attP was tested. The edits were probed using next-generation sequencing. The results of the 9×9 cross of attB and attP central dinucleotides—AA, GA, CA, AG, AC, CC, GT, CT and TT—are shown in FIG. 16A. Only orthogonal pairs of attB and attP show the highest edit percentage. This result is also shown in the heat-map of FIG. 16B.

### Example 8

### Integration of Albumin and CPS1 Into Albumin Locus

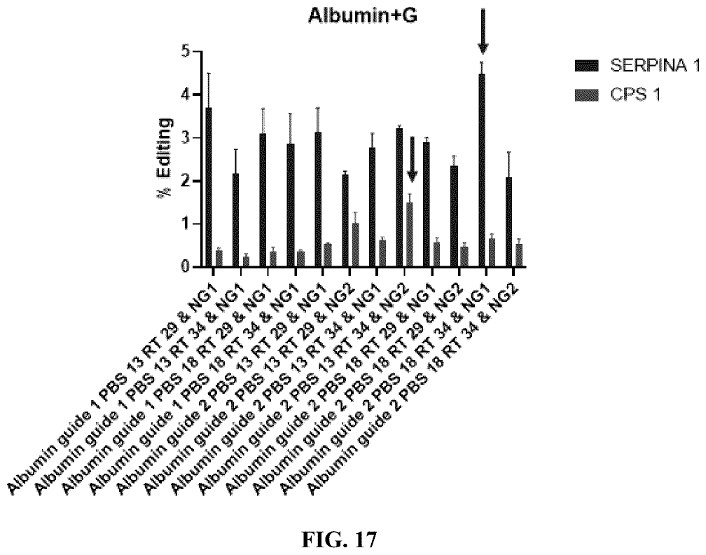

12 pegRNAs with albumin guide were linked to PBS and reverse transcriptase sequence of variable length, and different nicking guide RNAs were used to transfect HEK293FT cells. The percent editing in the albumin was probed using next-generation sequencing. The results of prime editing at the albumin locus are shown in FIG. 17. It was observed that SEQ ID NO: 79 showed the highest percent edits with SERPINA1 and SEQ ID NO: 80 showed the highest percent edits with CPS1.

### Example 9

### Engineering T-cells

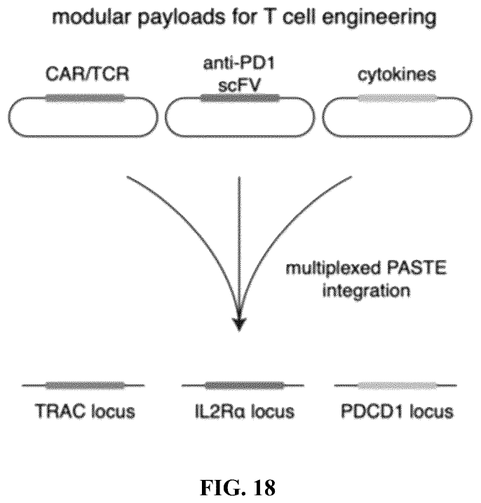

In order to engineer CD8+ T-cells, the efficiency of PASTE delivery and editing in T-cells can be evaluated (FIG. 18). ACTB targeting pegRNA can be used to insert an integration site with an EGFP insertion template. To deliver the PASTE components to CD8+ T-cells, electroporation can be used along with an optimized electroporation protocol for unstimulated T-cells. As multiple plasmids may reduce the efficiency of electroporation, the consolidated PASTE components that use fewer vectors can be applied.

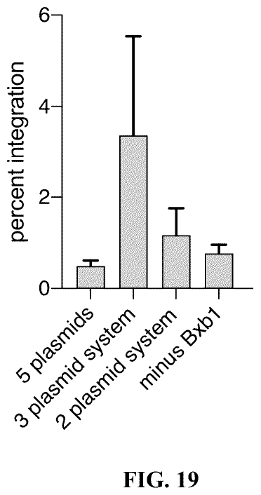

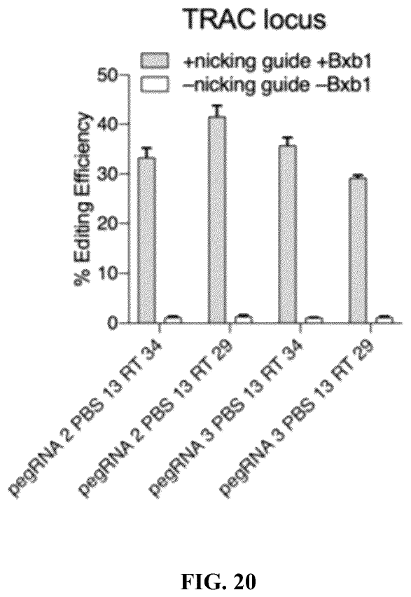

Five vectors, three vectors, and two vectors PASTE systems show that robust T-cell editing can be achieved with maximal editing using the three-vector approach (FIG. 19). Further, expanded sets of electroporation conditions, including the overall plasmid amounts, cell numbers, and voltage/amperage protocol can be tested. In addition, stimulation of T-cells may influence the efficiency of transduction and PASTE efficiency. Further, CD4+/CD8+ T cell mixtures stimulated with T-Activator CD3/CD28 ligands can have higher PASTE editing efficiency versus unstimulated cells. In order to separate efficiency of PASTE from the overall delivery rate, an mCherry expression cassette on PASTE vectors can be evaluated in order to sort successfully transfected T cells. Once optimized parameters are achieved, a panel of 10 insertion sites with PASTE in T cells, including the TRAC, IL2Rα, and PDCD1 loci, can be evaluated, using different insertions (e.g. EGFP, BFP, and YFP), both in single and multiplexed editing contexts. A tested subset of relevant sites in HEK293FT achieved greater than 40% editing for EGFP insertion (FIG. 20). The PASTE efficiency at TRAC locus with different TCR and CAR constructs can be evaluated. The T-cells can successfully be transfected to achieve insertion of CARs or TCRs.

### Example 10

### PASTE for CFTR

PASTE for the CFTR locus can be tested in HEK293FT cells to identify top performing pegRNA and nicking designs for human cells. Neuro-2A cells can also be tested to identify top performing pegRNA and nicking designs for mouse cells. The best constructs can be applied for testing in mouse air lung interface (ALI) organoids in vitro or for delivery in pre-clinical models of cystic fibrosis in mice. Table 12 shows the pegRNA, nicking guide and minicircle DNA characteristics for the CFTR gene modulation.

TABLE 12

| Variables | Characteristics |
|---|---|
| pegRNA | 38 bp shortened minimal attB and normal 46 bp attB |
| | sequence with: |
| | a. PBS of 17, 13, and 9 nt length, and |
| | b. RT of 20, 15, and 10 nt in length |
| Nicking | Nicking guide 1 +64 bp Nicking guide 2 +23 bp Nicking |
| guides | guide 3 −60 bp |
| | Nicking guide 4 −78 bp (distance is calculated from cut |
| | site of pegRNA) |
| Minicircle | A. CFTR coding sequence alone (~4,454 pb in size) |
| template | B. CFTR coding sequence plus 5′ and 3′ UTRs (~6,011 |
| | bp in size) |
| | (Both minicircles have attP site on them for integration |
| | by Bxb1 and a bGH poly A signal) |

### Example 11

### AttB and EGPF Integration Using PASTE

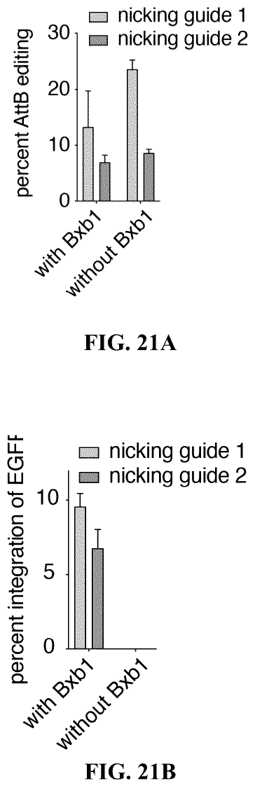

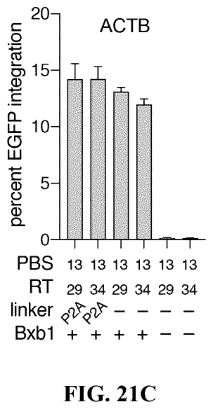

The efficiency of the integration of attB and EGPF at the ACTB locus was evaluated (FIGS. 21A-21C). To investigate whether Bxb1 can add an EGFP template into this site, a delivery approach using a 5 plasmid system expressing each of the following component was deployed: 1) pegRNA expression, 2) nicking guide expression, 3) Prime expression (Cas9-RT), 4) Bxb1 expression and 5) the insertion template (in this case EGFP). This approach was found to yield editing efficiency of the attB site up to 24% and integration of EGFP ˜10% in HEK293FT cells as measured by sequencing (FIGS. 21A-21B). Optimal activity is achieved in 3-4 days and can be performed as a single step transfection or electroporation of all components. Because the EGFP plasmid is designed as a minicircle, allowing removal of all undesired bacterial components, only the desired gene is inserted along with minimal scars from the Bxb1 recombined sites.

To make the tool simpler to use, the Bxb1 can be linked to Prime via a P2A linker to the Cas9-RT fusion, allowing for only a single plasmid to be used for PASTE protein expression rather than two. This optimization can maintain the same level of editing, making it easier to use the tool and deliver it (FIG. 21C).

### Example 12

### Programmable EGFP Integrations in Different Cell Types

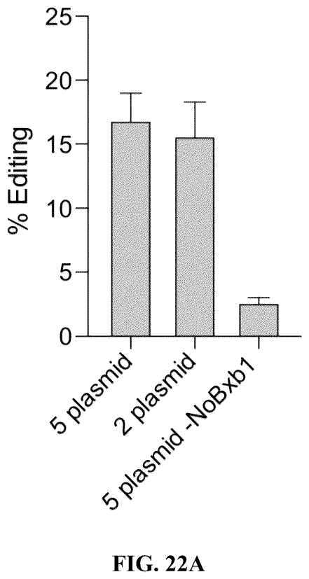

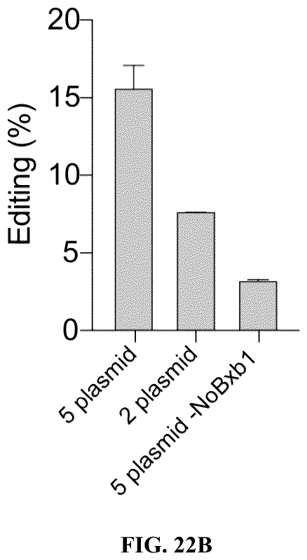

The programmable EGFP integration in liver hepatocellular carcinoma cell line HEPG2 (FIG. 22A) and chronic myelogenous leukemia cell line K562 (FIG. 22B) was evaluated. EGFP integration at the ACTB locus in K562 and HEPG2 cells of about 15% was observed, demonstrating robustness of the platform across cell types.

### Example 13

### Mutagenesis of Bxb1 for Enhanced PASTE Activity

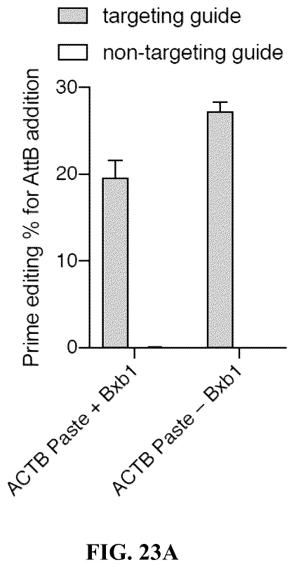

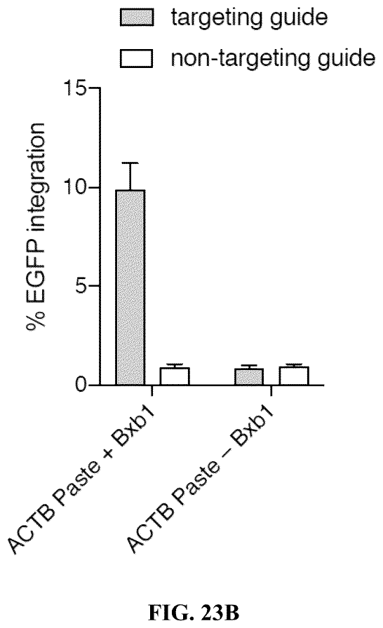

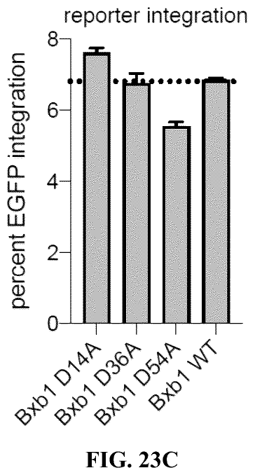

The mutagenesis of Bxb1 for enhanced PASTE activity was evaluated (FIGS. 23A-23C). Two levers for optimizing PASTE activity exist: 1) improving the activity of the integrase and 2) enhancing the Prime addition of the integration sequence. As illustrated in FIGS. 23A-23B, Bxb1 activity can be improved as only about 30% of Bxb1 attB sites that are added by PASTE are integrated into by Bxb1. This illustrates that if the Bxb1 efficiency can be improved, the PASTE can be improved. Furthermore, catalytic residues in the Bxb1 integrase were identified via conservation and structural analyses and Bxb1 mutants were generated to test as part of PASTE. As illustrated in FIG. 23B, the mutations can improve integration by about 20-30%.

### Example 14

### Effect of the pegRNA PBS and RT Lengths on the Prime Editing Integration Efficiency

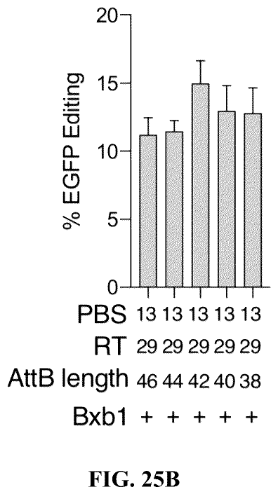

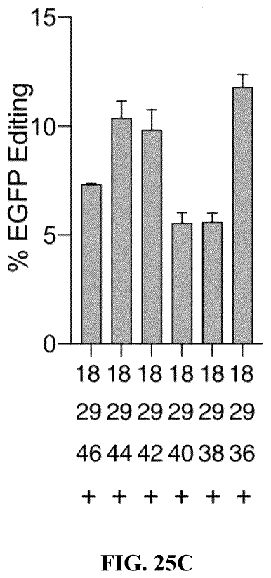

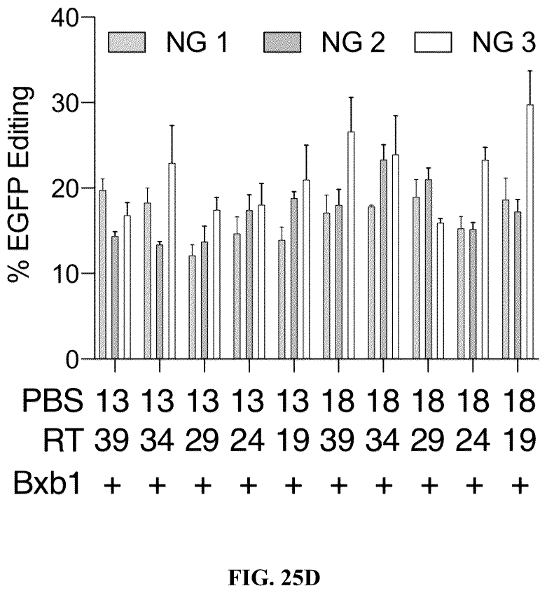

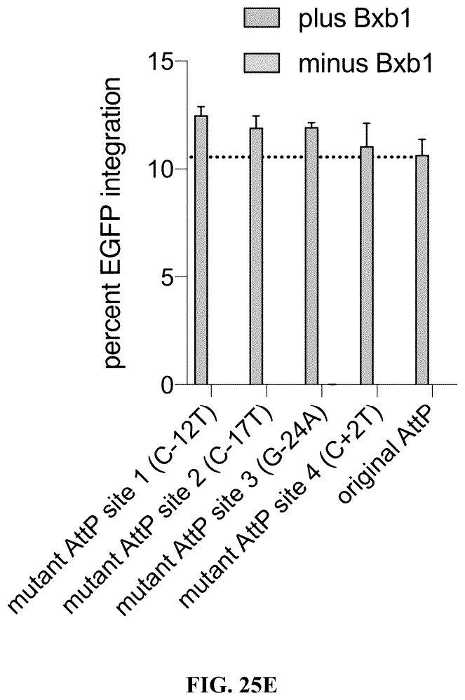

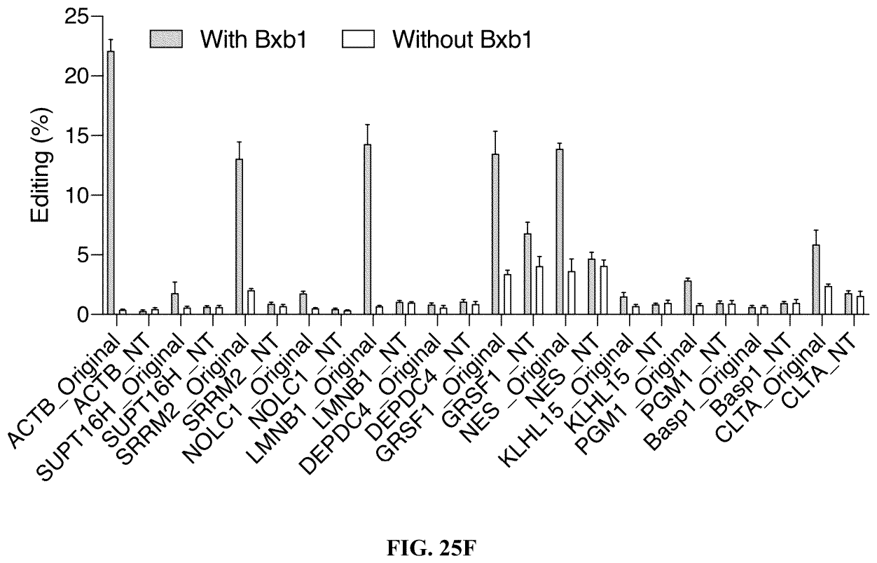

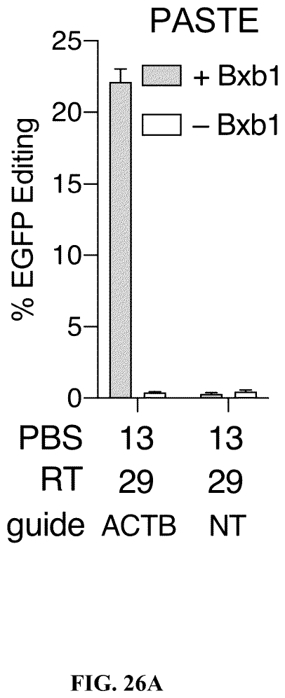

The effect of the pegRNA PBS and RT lengths on the prime editing integration efficiency was evaluated (FIGS. 25A-25F). It was found that PASTE can be optimized by tuning the PBS and RT lengths at the ACTB locus to achieve editing rates up to about 20% (FIG. 25A). It was found that shortening the attB site can help improve PASTE function as Prime is better at inserting shorter sequences. Further optimization of PBS, RT, and attB lengths showed that optimal designs can be found for insertion upstream of the LMNB1, NOLC1, and GRSF1 loci (FIGS. 25B, 25C, and 25D). Lengths as short as 36 nt for attB were found to be still functional for integration into a reporter plasmid (FIGS. 25B and 25C). It was found that the reverse complemented version of the attB sequence was better integrated via Prime editing, suggesting that the sequence of what Prime is inserting matters. EGFP integrations with attP site mutants showed that certain mutants can improve integration efficiency significantly (FIG. 25E). PASTE was also performed with a large panel of genes, inserting EGFP at the N-terminus of ACTB, LMNB1, SUPT16H, SRRM2, NOLC1, KLHL15, GRSF1, DEPDC4, NES, PGM1, CLTA, BASP1, and DNAJC18 (FIG. 25F). Editing rates that are about 5%-40% were found using digital droplet PCR (ddPCR).

### Example 15

### Comparison of PASTE and HITI On-Target and Off-Target Activities

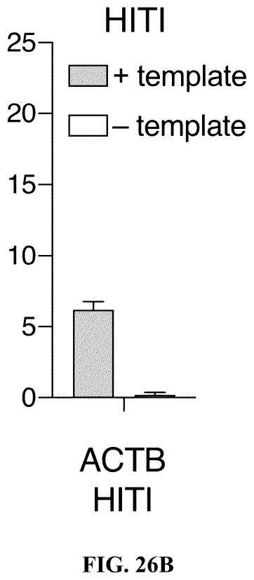

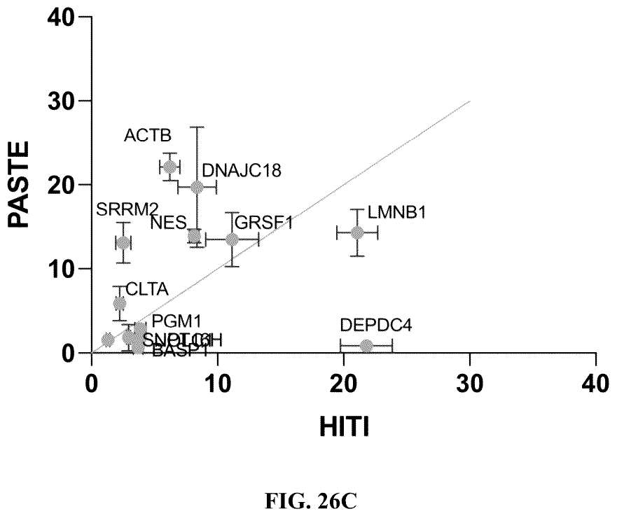

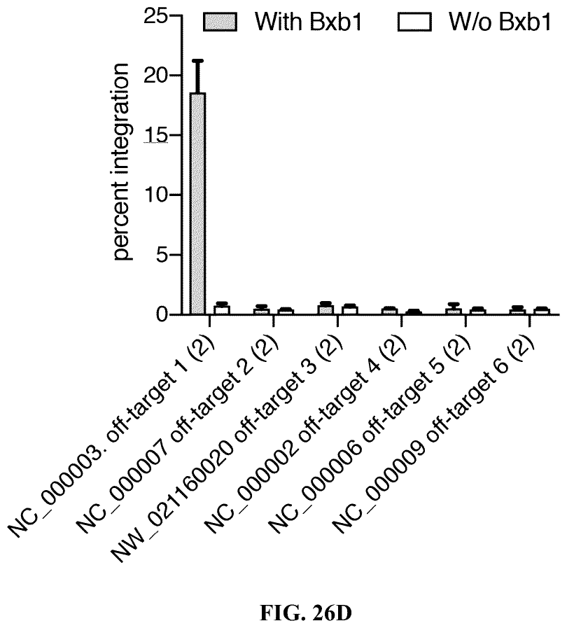

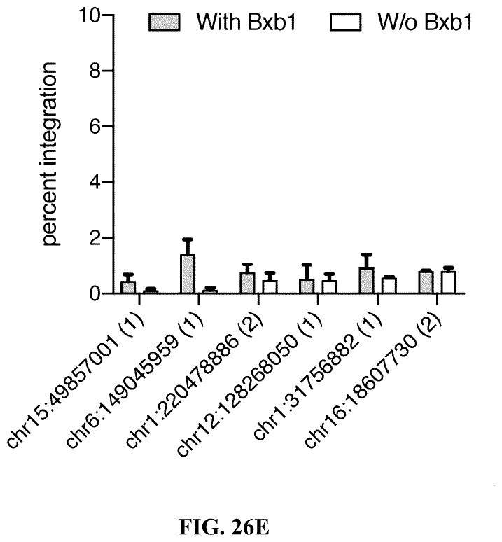

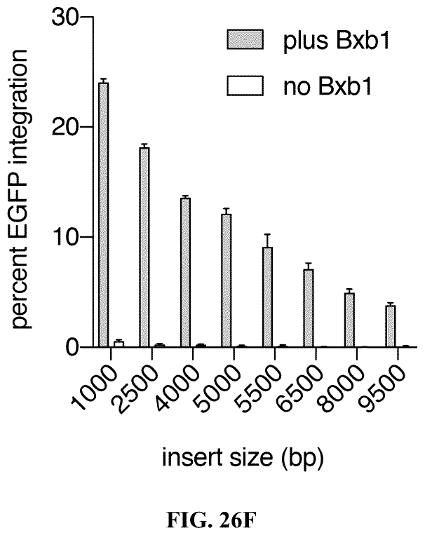

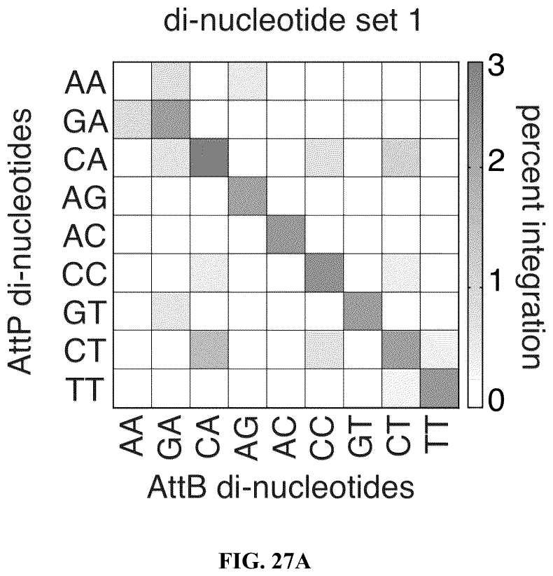

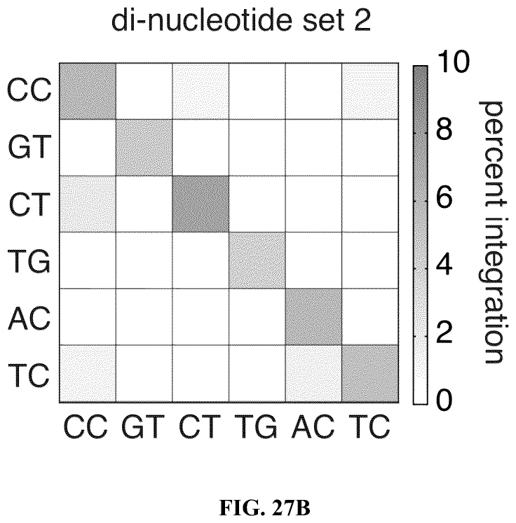

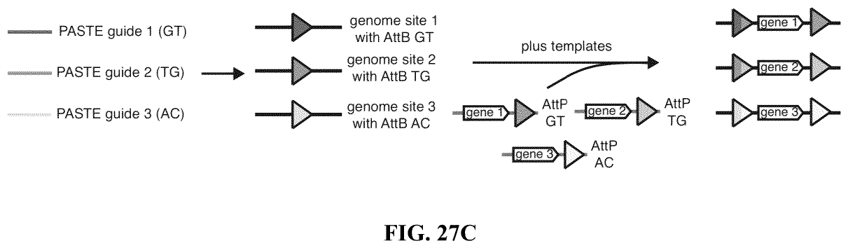

The PASTE and HITI on-target and off-target activities were compared (FIGS. 26A-26F). PASTE and HITI were found to have about 22% and 5% integration efficiencies respectively when using the same guide sequence (FIGS. 26A and 26B). PASTE was found to outperform HITI at most sites when analyzing the editing of 14 genes (FIG. 26C). Using a ddPCR based approach, it was found that PASTE was very specific with minimal off-target activity for Bxb1 off-targets integrations (FIG. 26D) and Cas9 off-targets integrations (FIG. 26E). The analysis of inserts of different sizes showed that PASTE can reliably insert sequences 1 kb-10 kb in size (FIG. 26F), revealing the wide range of sequence sizes PASTE is capable of working with. A decrease in insertion efficiency at larger sizes was also observed, which was likely due to the reduction in plasmid delivery to HEK293FT cells at larger plasmid sizes.

### Example 16

### Multiplexing with PASTE and Orthogonal Di-Nucleotide attB and attP Sites

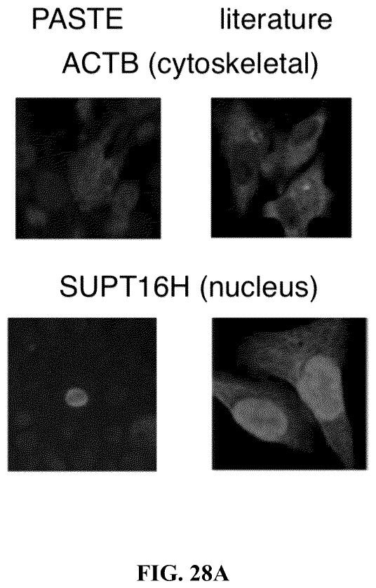

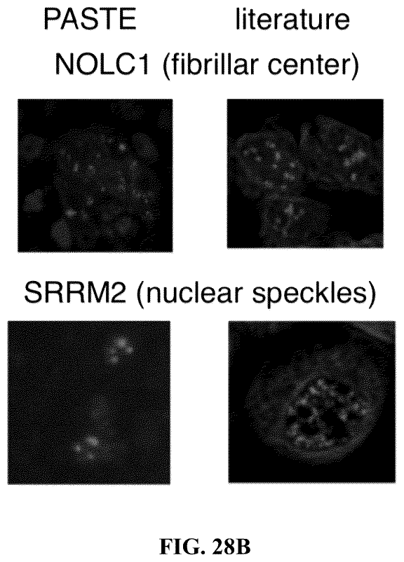

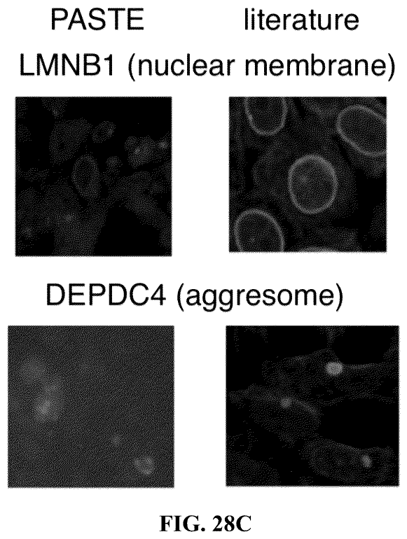

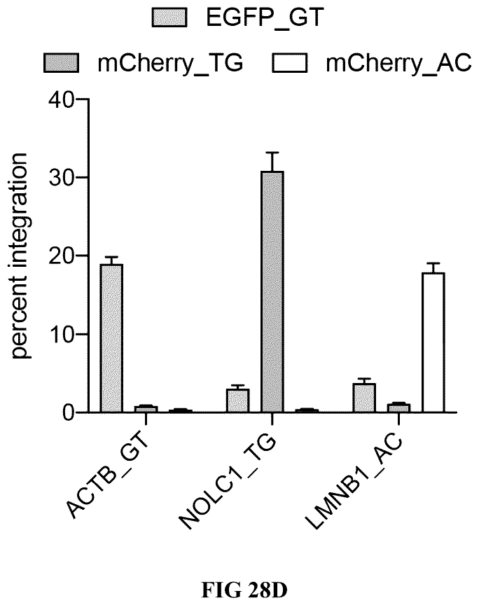

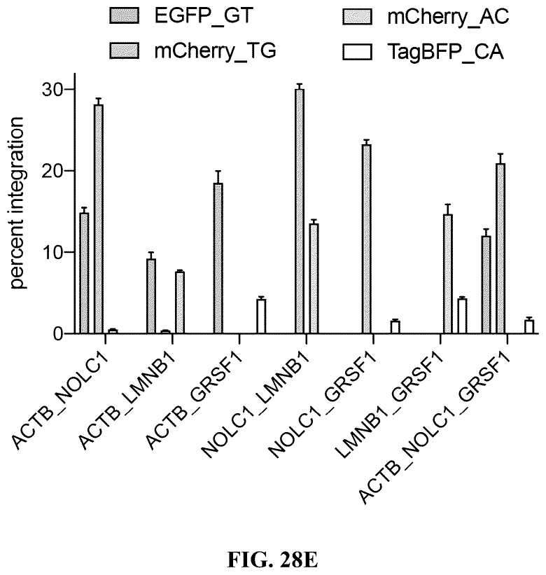

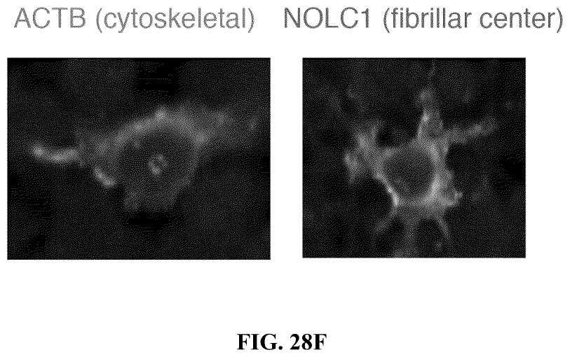

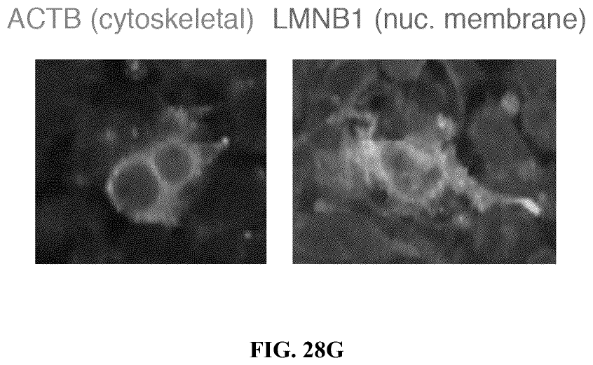

Multiplexing with PASTE and orthogonal di-nucleotide attB and attP sites was evaluated (FIGS. 28A-28C). Multiple orthogonal combinations were found for mutants of the central di-nucleotide motif (FIGS. 28A and 28B). As illustrated in FIG. 28C, programmable multiplexed gene insertion can be achieved by using these orthogonal combinations with PASTE only delivering different pegRNAs and gene inserts while keeping the protein components the same (FIG. 8C).

### Example 17

### PASTE Multiplexed Integrations at Endogenous Sites

PASTE multiplexed integrations at endogenous sites were evaluated (FIGS. 28A-28G). A reading frame for the attR scar that is left post-integration by Bxb1 that is ideal for a protein linker due to the enrichment of glycines, serines, and prolines in the sequence (GLSGQPPRSPSSGSSG (SEQ ID NO: 426)) was identified. PegRNAs were designed using this linker frame for the resolution of the attR for tagging a number of genes at the N-terminus with EGFP (ACTB, NOLC1, LMNB1, SUPT16H, SRRM2, and DEPDC4). As these genes all have distinct protein localization appearances, microscopy can be used for ascertaining proper gene tagging. PASTE was found to be capable of high-efficiency gene tagging with protein localizations that match the reference images and expected localization of the proteins in the cells (FIGS. 28A-28C). Genes were also tagged in multiplexed fashion to demonstrate the orthogonality of the engineered integration sites. ACTB, LMNB1, NOLC1, and GRSF were targeted with orthogonal pegRNAs carrying GT, TG, AC, and CA, respectively in HEK293FT in groups of single, dual-plexing, and triple-plexing (FIGS. 28D-28E). These dinucleotides were paired with templates carrying EGFP, BFP, and mCherry to allow for multicolor imaging of these labeled genes. The efficiencies of integration for these multiplexing experiments were found to range from about 5%-32%, revealing efficient multiplex integration with PASTE. Using confocal microscopy of these multiplexed integration experiments, cells were found with simultaneous labeling of these different proteins (FIGS. 28F-28G).

### Example 18

### Combination of CRISPR-Based Genome Editing and Site-Specific Integration

The combination of CRISPR-based genome editing and site-specific integration was evaluated.

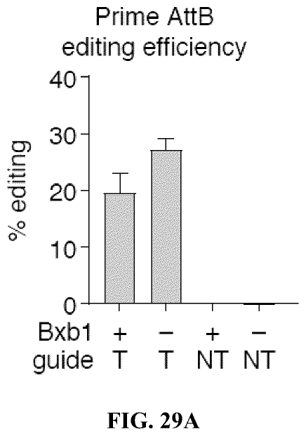

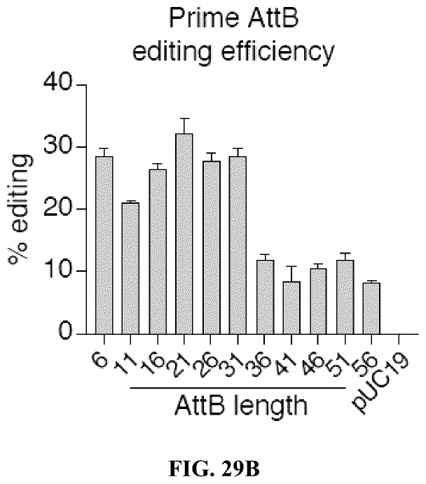

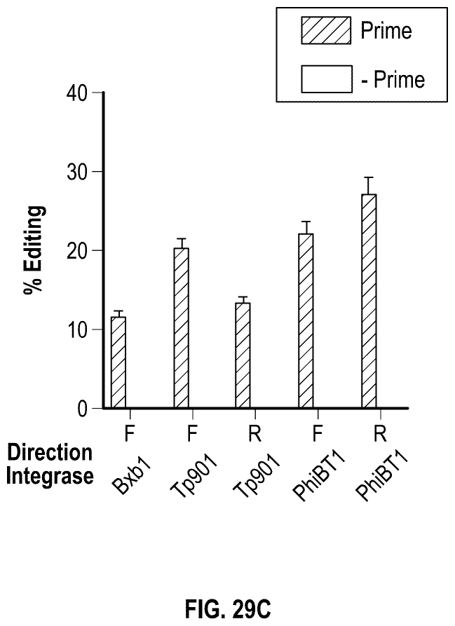

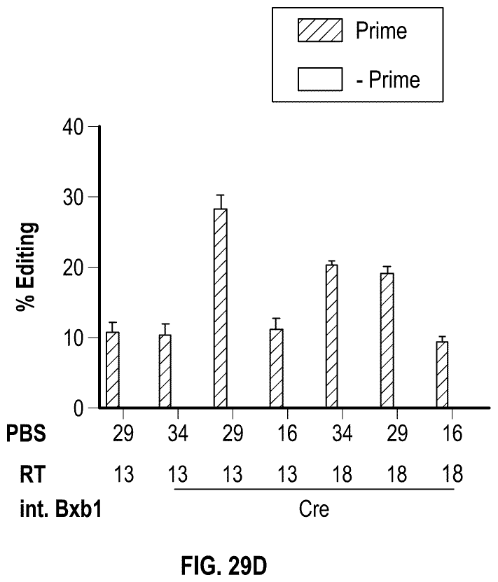

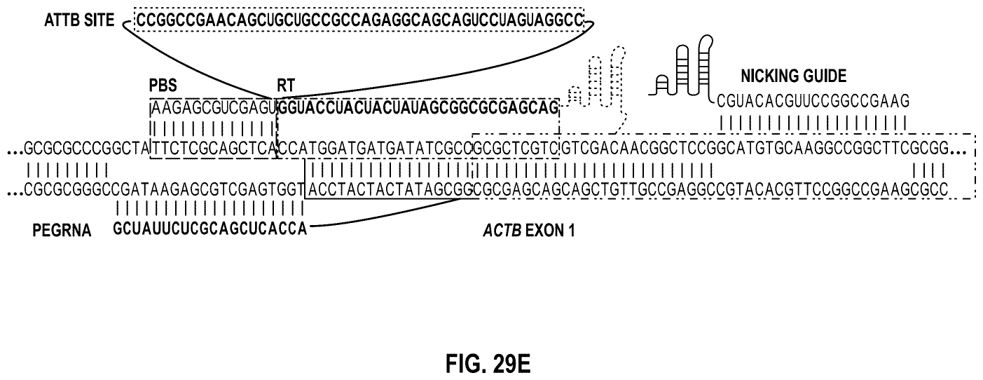

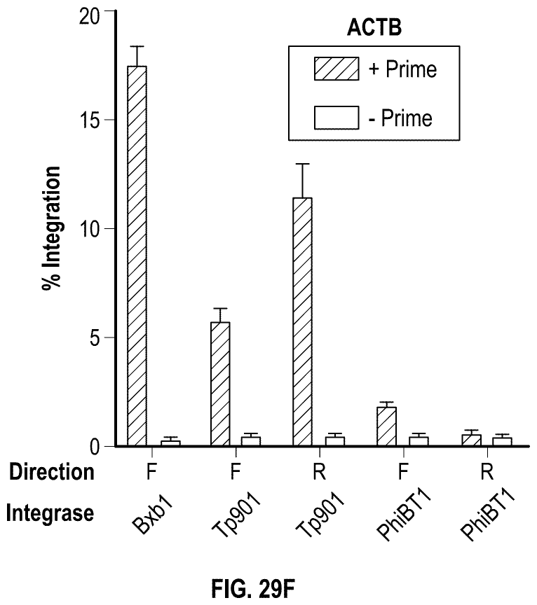

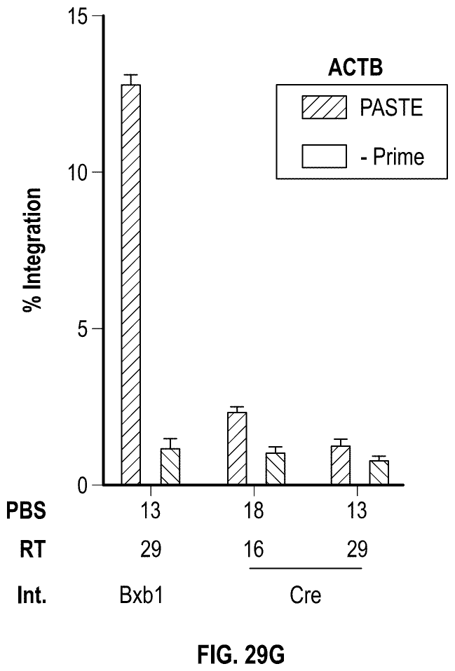

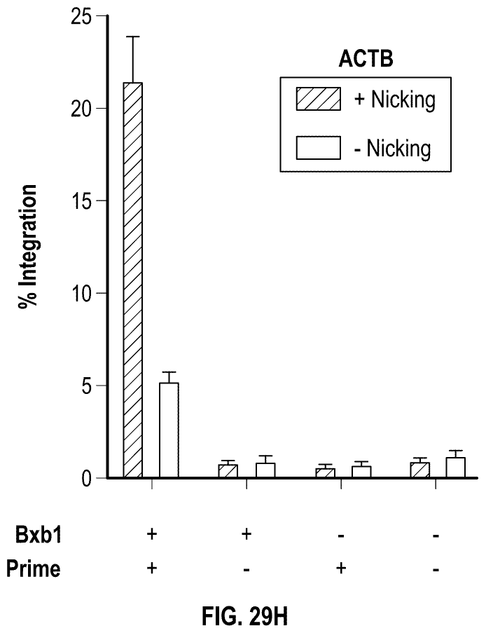

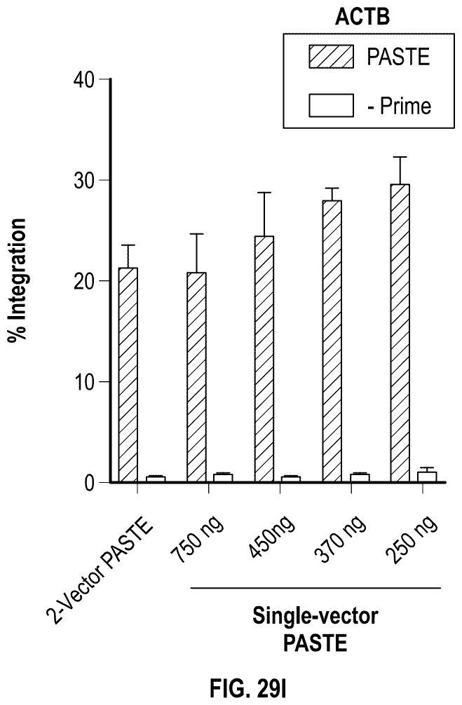

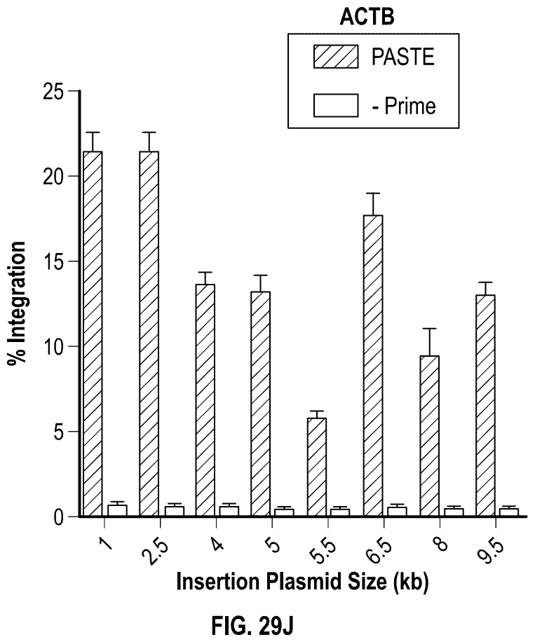

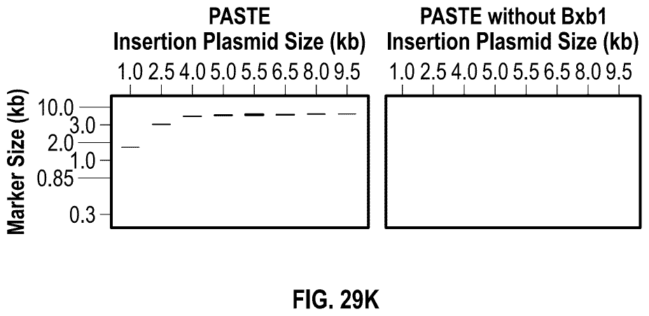

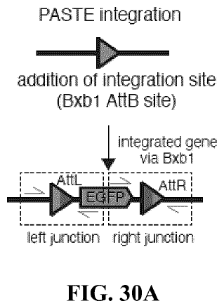

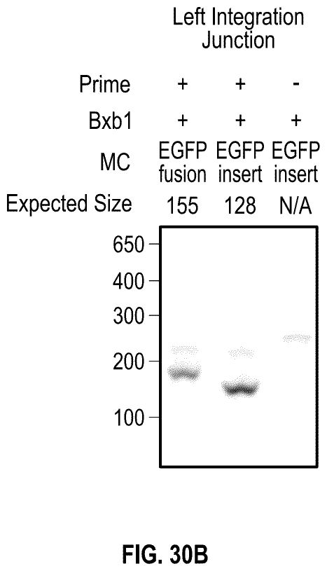

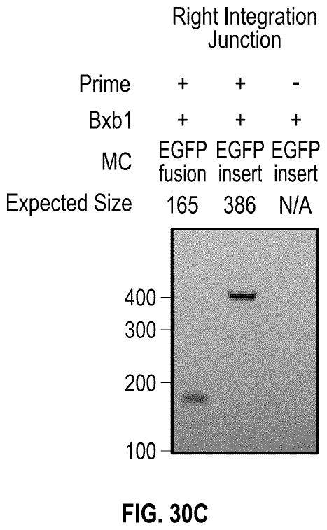

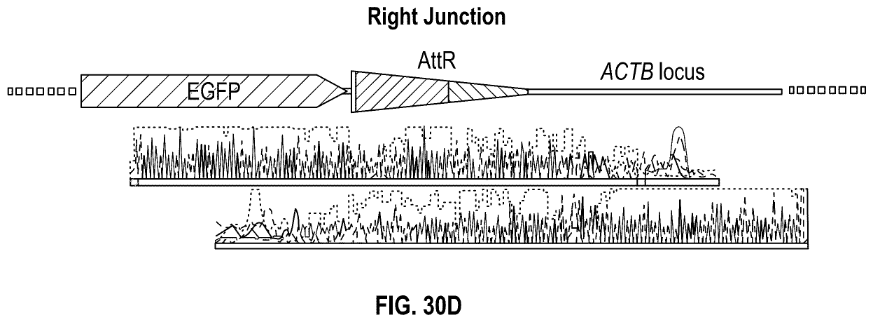

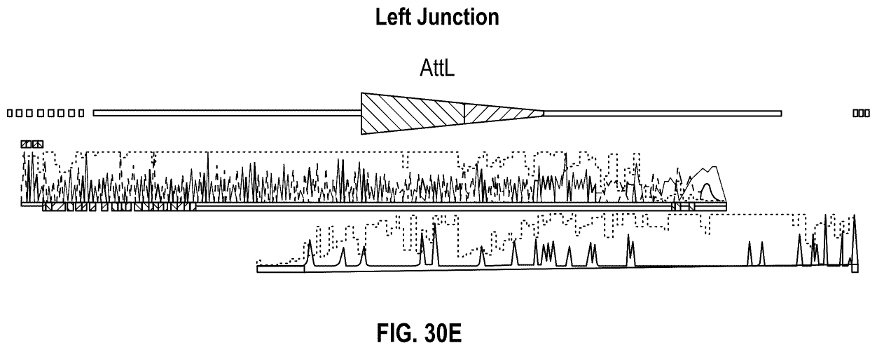

PegRNAs containing different attB length truncations were assessed (FIG. 29A). Prime editing was found to be capable of inserting sequences up to 56 bp at the beta-actin (ACTB) gene locus, with higher efficiency at lengths below 31 bp (FIGS. 29A-B) The integration of cognate landing sites was tested for multiple insertion enzymes: Bxb1, TP901, and phiBT1 phage serine integrases and Cre recombinase. Prime editing successfully inserted all landing sites tested, with efficiencies between 10-30% (FIGS. 29C-D). To test the complete system, all components were combined and delivered in a single transfection: the prime editing vector, the landing site containing pegRNA, a nicking guide for stimulating prime editing, a mammalian expression vector for the corresponding integrase or recombinase and a 969 bp minicircle DNA cargo encoding green fluorescent protein (GFP) (FIG. 29E). GFP integration rates among the four integrases and recombinases were compared and Bxb1 integrase was found to have the highest integration rate (˜20%) at the targeted ACTB locus and require the prime editing nicking guide for optimal performance (FIGS. 29F-H). Finally, to reduce the number of transfected components, Bxb1 was co-expressed with the SpCas9-M-MLV reverse transcriptase (PE2) fusion protein via a P2A protein cleavage site. This combination maintained high GFP insertion efficiency, up to 30% (FIG. 29E). The complete system, PASTE, achieved precise integration of templates as large as 9,500 bp with greater than 10% integration efficiency (FIGS. 29J-K and 26E), with complete integration of the full-length cargo confirmed by Sanger sequencing (FIG. 30A-E).

### Example 19

### Impact of Prime Editing and Integrase Parameters on PRIME Editing

The impact of prime editing and integrase parameters on the integration efficiency of PRIME editing was assessed.

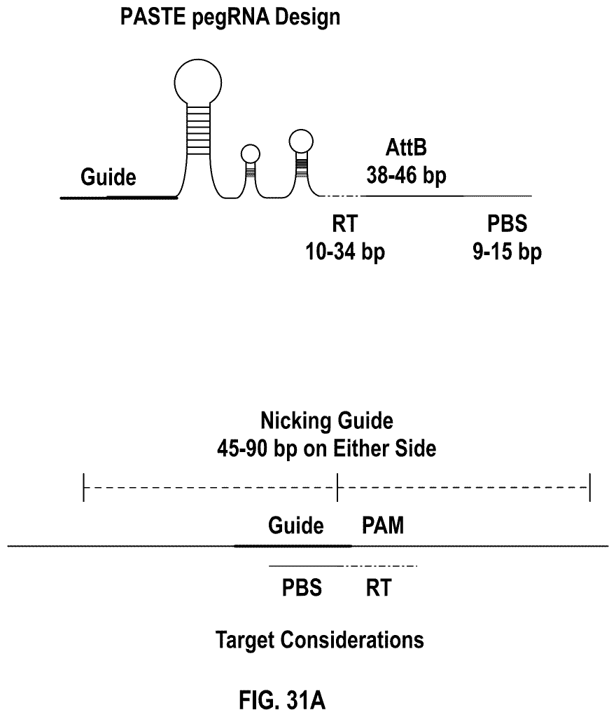

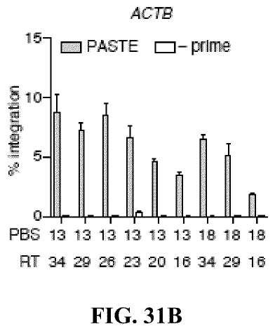

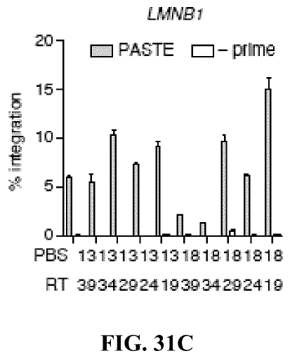

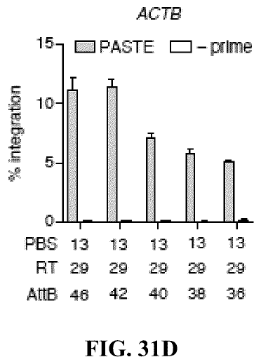

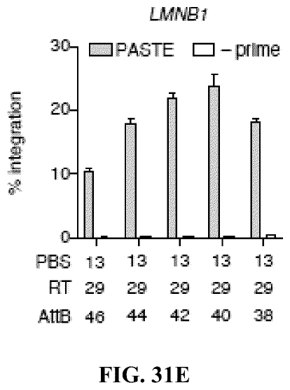

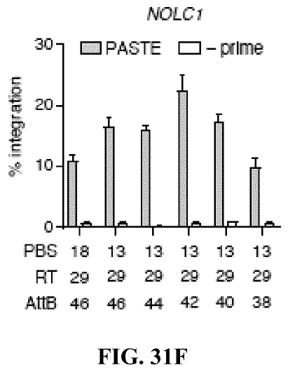

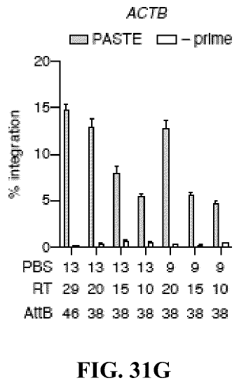

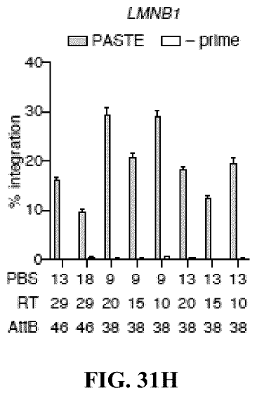

Relevant pegRNA parameters for PASTE include the primer binding site (PBS), reverse transcription template (RT), and attB site lengths, as well as the relative locations and efficacy of the pegRNA spacer and nicking guide (FIG. 31A). A range of PBS and RT lengths were tested at two loci, ACTB and lamin B1 (LMNB1), and rules governing efficiency were found to vary between loci, with shorter PBS lengths and longer RT designs having higher editing at the ACTB locus (FIG. 31B) and longer PBS and shorter RT designs performing better at LMNB1 (FIG. 31C).

The length of the attB landing site must balance two conflicting factors: the higher efficiency of prime editing for smaller inserts and reduced efficiency of Bxb1 integration at shorter attB lengths. AttB lengths were evaluated at ACTB, LMNB1, and nucleolar phosphoprotein p130 (NOLC1), and the optimal attB length was found to be locus dependent. At the ACTB locus, long attB lengths could be inserted by prime editing (FIG. 29B) and overall PASTE efficiencies for the insertion of GFP were highest for long attB lengths (FIG. 31d). In contrast, intermediate attB lengths had higher overall integration efficiencies (>20%) at LMNB1 (FIG. 31E) and NOLC1 (FIG. 31F), indicating that the increased efficiency of installing shorter attB sequences overcame the reduction of Bxb1 integration at these sites.

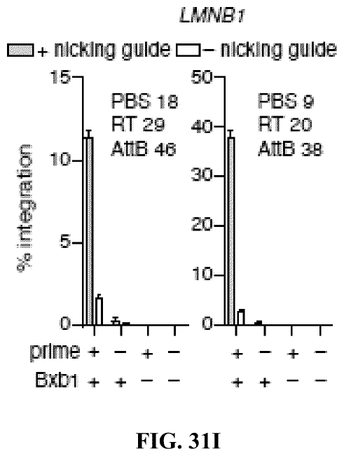

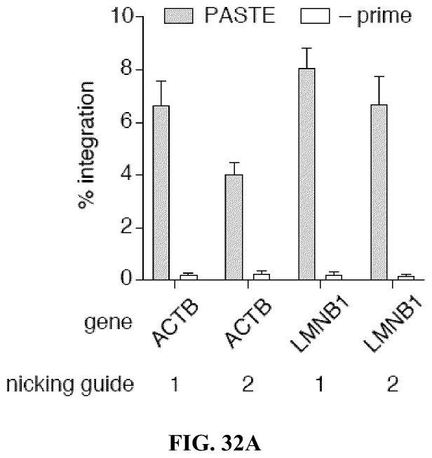

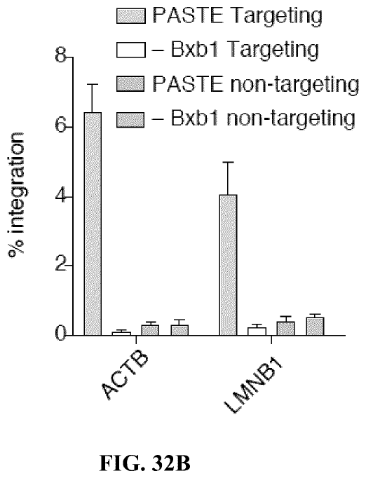

The PE3 version of prime editing combines PE2 and an additional nicking guide to bias resolution of the flap intermediate towards insertion. To test the importance of nicking guide selection on PASTE editing, editing at ACTB and LMNB1 loci was tested with two nicking guide positions. Suboptimal nicking guide positions were found to reduce the PASTE efficiency up to 30% (FIG. 32A) in agreement with the 75% reduction of PASTE efficiency in the absence of nicking guide (FIG. 29G). The pegRNA spacer sequence was found to be necessary for PASTE editing, and substitution of the spacer sequence with a non-targeting guide was found to eliminate editing (FIG. 32B).

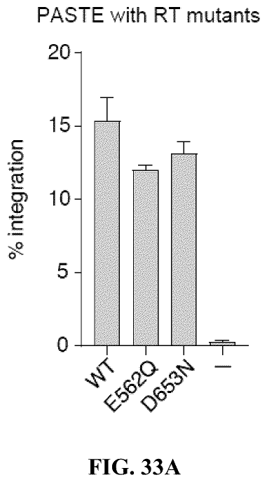

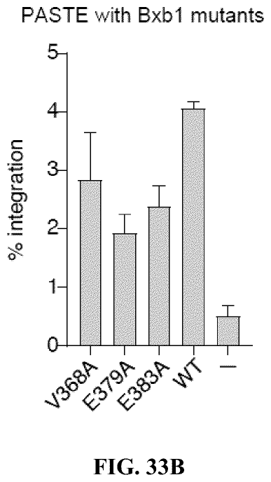

Rational mutations were also introduced in both the Bxb1 integrase and reverse transcriptase domain of the PE2 construct to optimize PASTE further. While some of these mutations were well tolerated by PASTE (FIGS. 33A-B), none of them improved PASTE editing efficiency.

Short RT and PBS lengths can offer additional improvements for editing. A panel of shorter RT and PBS guides were tested at ACTB and LMNB1 loci and while shorter RT and PBS sequences did not increase editing at ACTB (FIG. 31G), it was found that they had improved editing at LMNB1 (FIG. 31H) with best performing guides reaching GFP insertion rates of ˜40% (FIG. 31I).

### Example 20

### PASTE Tagging at Multiple Endogenous Genes

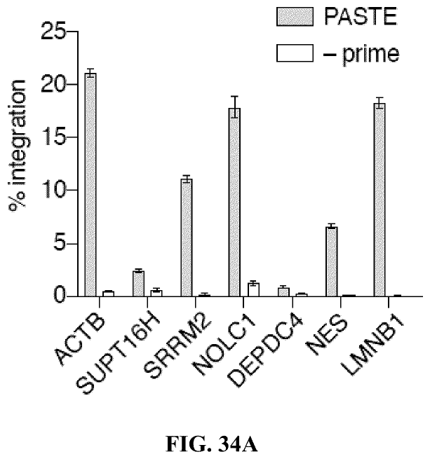

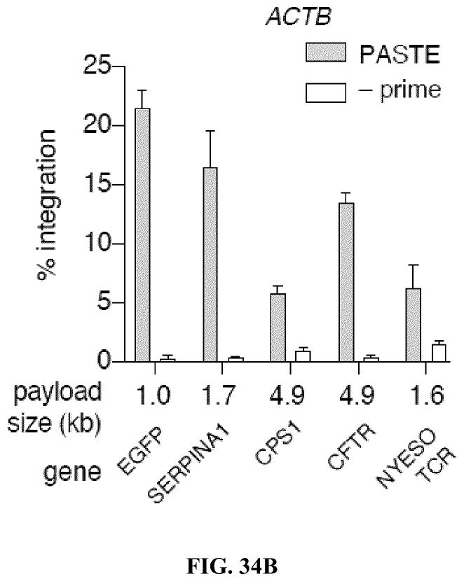

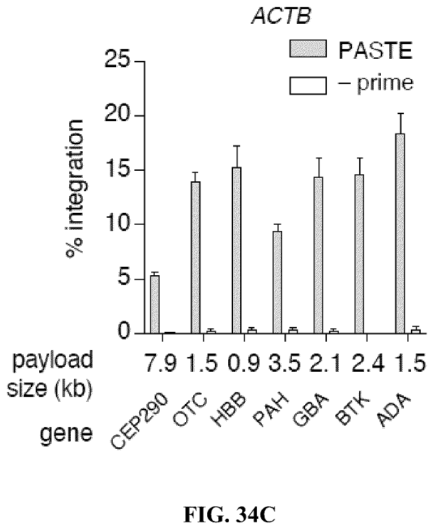

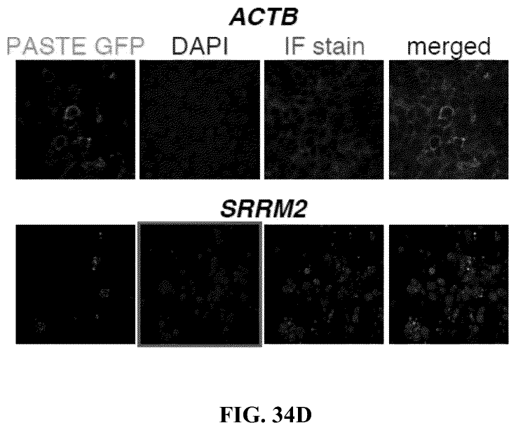

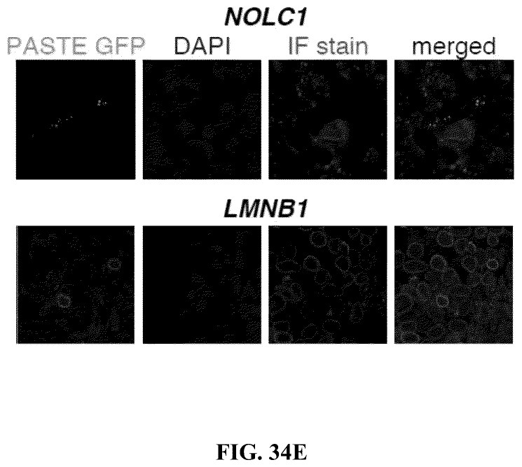

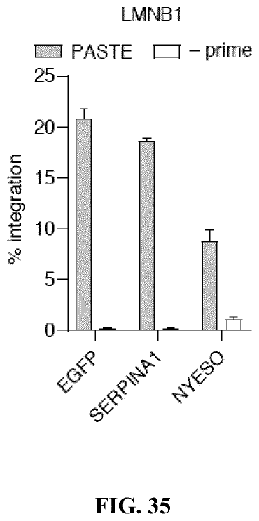

GFP insertion efficiency was measured at seven different gene loci—ACTB, SUPT16H, SRM2, NOLC1, DEPDC4, NES, and LMNB1—to test the versatility of the PASTE programming. A range of integration rates up to 22% was found (FIG. 34A). Because PASTE does not require homology or sequence similarity on cargo plasmids, integration of diverse cargo sequences is modular and easily scaled across different loci. Six different gene cargos, varying in size from 969 bp to 4906 bp, were tested for insertion at ACTB and LMNB1 loci with PASTE. Integration frequencies between 5% and 22% depending on the gene and insertion locus were found (FIGS. 34B and 35). Additionally, a panel of seven common therapeutic genes, CEP290, OTC, HBB, PAH, GBA, BTK, and ADA was evaluated for insertion at the ACTB locus, and the efficient integration of these cargos were found between 5%-20% (FIG. 34C).

The precise insertions of PASTE for in-frame protein tagging or expressing cargo without disruption of endogenous gene expression was assessed. As Bxb1 leaves residual sequences in the genome (termed attL and attR) after cargo integration, these genomic scars can serve as protein linkers. The frame of the attR sequence was positioned through strategic placement of the attP on the minicircle cargo, achieving a suitable protein linker, GGLSGQPPRSPSSGSSG (SEQ ID NO: 427). Using this linker, four genes (ACTB, SRRM2, NOLC1, and LMNB1) were tagged with GFP using PASTE. To assess correct gene tagging, the subcellular location of GFP was compared with the tagged gene product by immunofluorescence. For all four targeted loci, GFP co-localized with the tagged gene product, indicating successful tagging (FIGS. 34D-E).

### Example 21

### Orthogonal Sequence Preferences for Bxb1 Integration

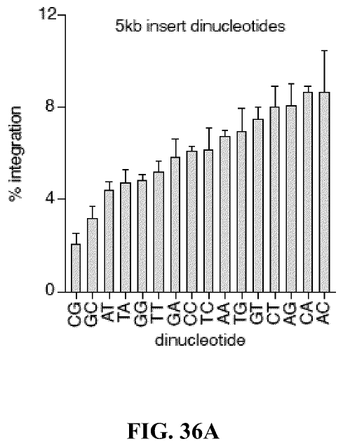

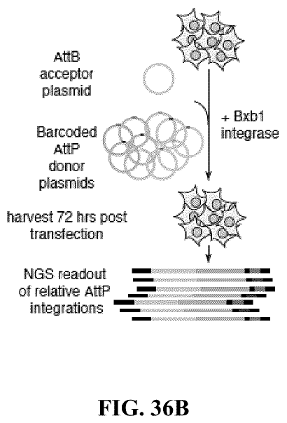

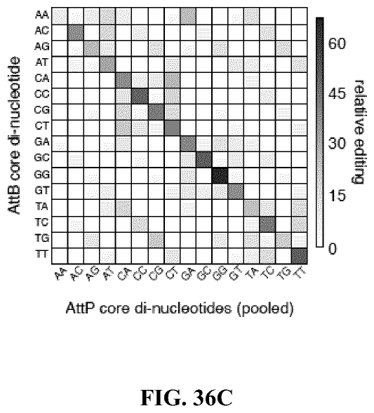

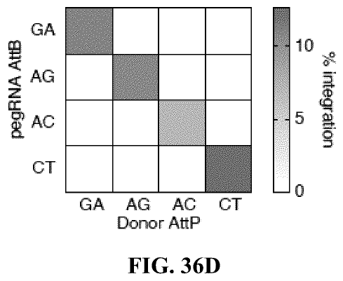

The central dinucleotide of Bxb1 is involved in the association of attB and attP sites for integration, and changing the matched central dinucleotide sequences can modify integrase activity and provide orthogonality for insertion of two genes. Expanding the set of attB/attP dinucleotides can enable multiplexed gene insertion with PASTE. The efficiency of GFP integration at the ACTB locus with PASTE across all 16 dinucleotide attB/attP sequence pairs was profiled to find optimal attB/attP dinucleotides for PASTE insertion. Several dinucleotides with integration efficiencies greater than the wild-type GT sequence were found (FIG. 36A). A majority of dinucleotides had 75% editing efficiency or greater compared to wild-type attB/attP efficiency, implying that these dinucleotides can be orthogonal channels for multiplexed gene insertion with PASTE.

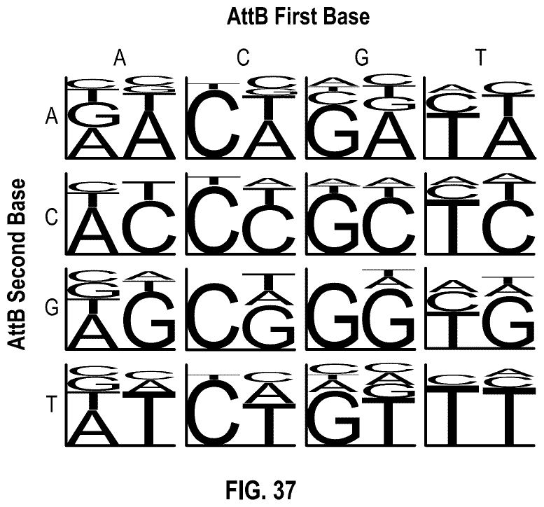

The specificity of matched and unmatched attB/attP dinucleotide interactions was then assessed. The interactions between all dinucleotide combinations in a scalable fashion using a pooled assay to compare attB/attP integration were profiled (FIG. 36B). By barcoding 16 attP dinucleotide plasmids with unique identifiers, co-transfecting this attP pool with the Bxb1 integrase expression vector and a single attB dinucleotide acceptor plasmid, and sequencing the resulting integration products, the relative integration efficiencies of all possible attB/attP pairs were measured (FIG. 36C). Dinucleotide specificity was found to vary, with some dinucleotides (GG) exhibiting strong self-interaction with negligible crosstalk, and others (AA) showing minimal self-preference. Sequence logos of attP preferences (FIG. 37) revealed that dinucleotides with C or G in the first position have stronger preferences for attB dinucleotide sequences with shared first bases, while other attP dinucleotides, especially those with an A in the first position, have reduced specificity for the first attB base.

GA, AG, AC, and CT dinucleotide pegRNAs were then tested for GFP integration at ACTB, either paired with their corresponding attP cargo or mispaired with the other three dinucleotide attP sequences. All four of the tested dinucleotides efficiently were found to integrate cargo only when paired with the corresponding attB/attP pair, with no detectable integration across mispaired combinations (FIG. 36D).

### Example 22

### Multiplex Gene Integration with PASTE

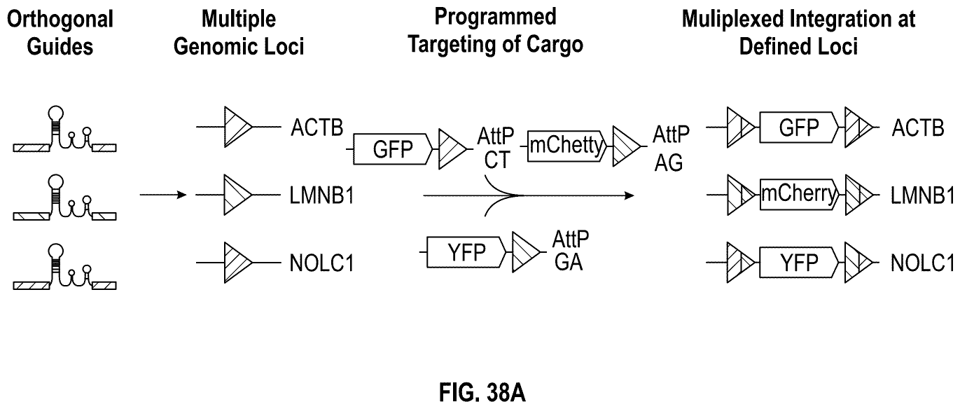

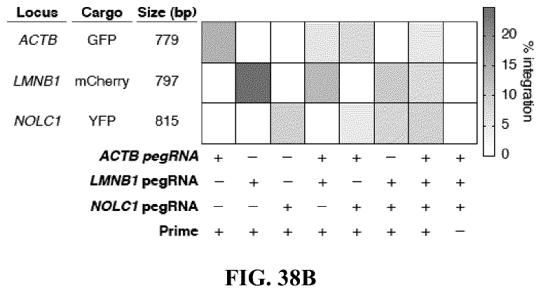

Multiplexing in cells by using orthogonal pegRNAs that direct a matched attP cargo to a specific site in the genome was assessed (FIG. 38A). Selecting the three top dinucleotide attachment site pairs (CT, AG, and GA), pegRNAs that target ACTB (CT), LMNB1 (AG), and NOLC1 (GA) and corresponding minicircle cargo containing GFP (CT), mCherry (AG), and YFP (GA) were designed. Upon co-delivering these reagents to cells, single-plex, dual-plex, and trip-plex editing of all possible combinations of these pegRNAs and cargo in the range of 5%-25% integration was found to be achieved (FIG. 38B).

An application for multiplexed gene integration is for labeling different proteins to visualize intracellular localization and interactions within the same cell. PASTE was used to simultaneously tag ACTB (GFP) and NOLC1 (mCherry) or ACTB (GFP) and LMNB1 (mCherry) in the same cell. No overlap of GFP and mCherry fluorescence was observed and tagged genes were confirmed to be visible in their appropriate cellular compartments, based on the known subcellular localizations of the ACTB, NOLC1 and LMNB1 protein products (FIGS. 15A-B).

### Example 23

### PASTE Efficiencies Compared with DSB-Based Insertion Methods

PASTE efficiencies were found to exceed comparable DSB-based insertion methods.

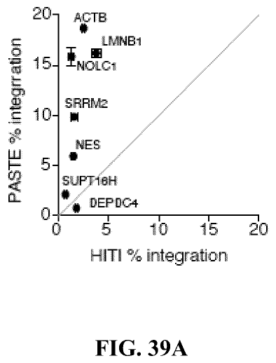

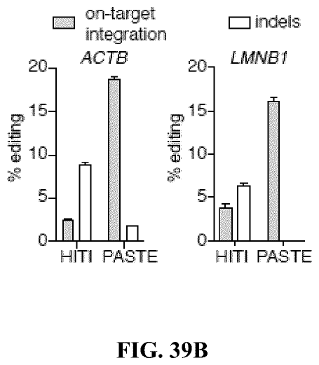

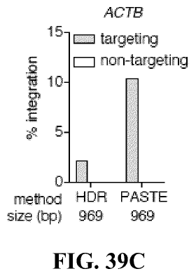

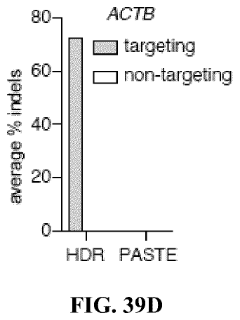

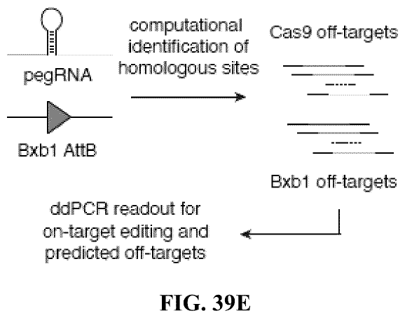

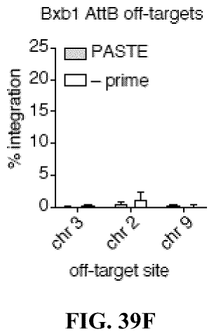

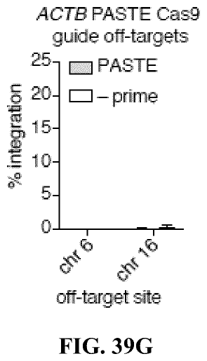

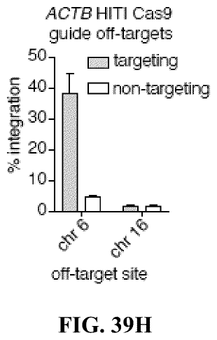

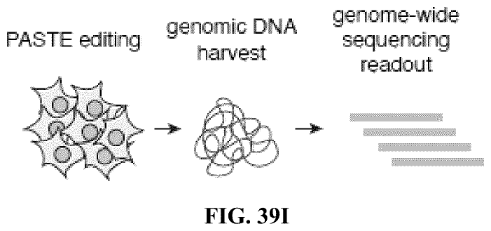

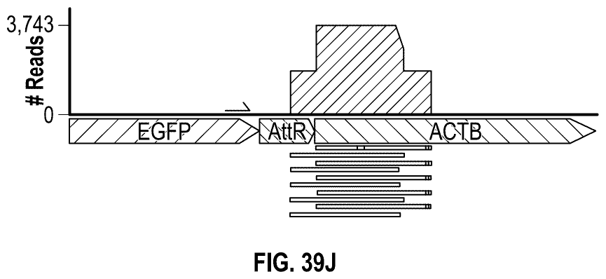

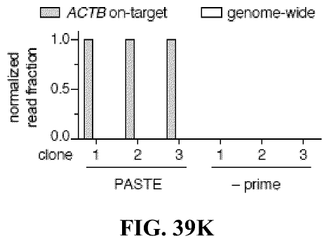

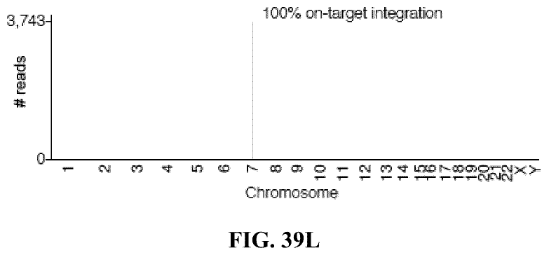

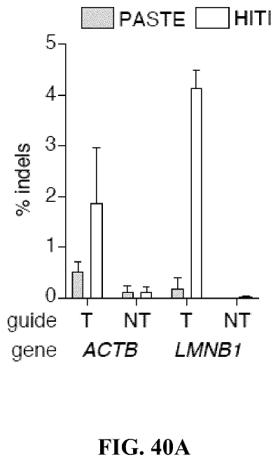

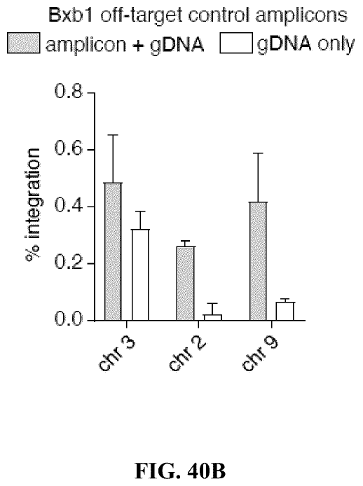

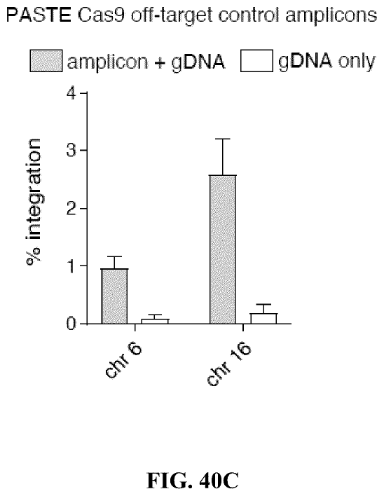

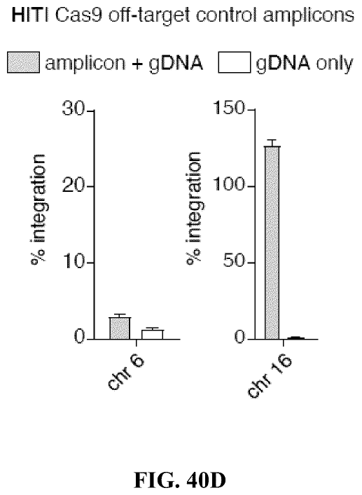

PASTE editing was assessed alongside DSB-dependent gene integration using either NHEJ (i.e., homology-independent targeted integration, HITI) or HDR pathways. PASTE had equivalent or better gene insertion efficiencies than either HITI (FIG. 39A-B) or HDR (FIGS. 39C-D). On a panel of 7 different endogenous targets, PASTE exceeded HITI editing at 6 out of 7 genes, with similar efficiency for the 7th gene (FIG. 39A). As DSB generation can lead to insertions or deletions (indels) as an alternative and undesired editing outcome, the indel frequency of all three methods was assessed by next-generation sequencing, finding significantly fewer indels generated with PASTE than either HDR or HITI in both HEK293FT and HepG2 cells (FIGS. 39B, 39D and 40A), showcasing the high purity of gene integration outcomes with PASTE.

### Example 24

### Off-Target Characterization of PASTE and HITI Gene Integration

Off-target editing can be used in genome editing technologies. The specificity of PASTE at specific sites was assessed based on off-targets generated by Bxb1 integration into pseudo-attB sites in the human genome and off-targets generated via guide- and Cas9-dependent editing in the human genome (FIG. 39E). While Bxb1 lacks documented integration into the human genome at pseudo-attachment sites, potential sites with partial similarity to the natural Bxb1 attB core sequence were computationally identified. Bxb1 integration by ddPCR across these sites was tested and no off-target activity was found (FIGS. 39F and 40B-D). To assay Cas9 off-targets for the ACTB pegRNA, two potential off-target sites were identified via computational prediction and no off-target integration for PASTE was found (FIGS. 39G and 40A-D), but substantial off-target activity by HITI at one of the sites was found (FIGS. 39H and 40A-D).

Genome-wide off-targets due to either Cas9 or Bxb1 through tagging and PCR amplification of insert-genomic junctions were additionally assessed (FIG. 39I). Single cell clones were isolated for conditions with PASTE editing and negative controls missing PE2, and deep sequencing of insert genomic junctions from these clones showed all reads aligning to the on-target ACTB site, confirming no off-target genomic insertions (FIGS. 39J-L).

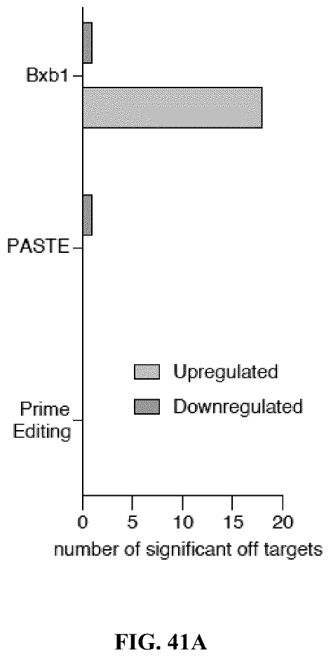

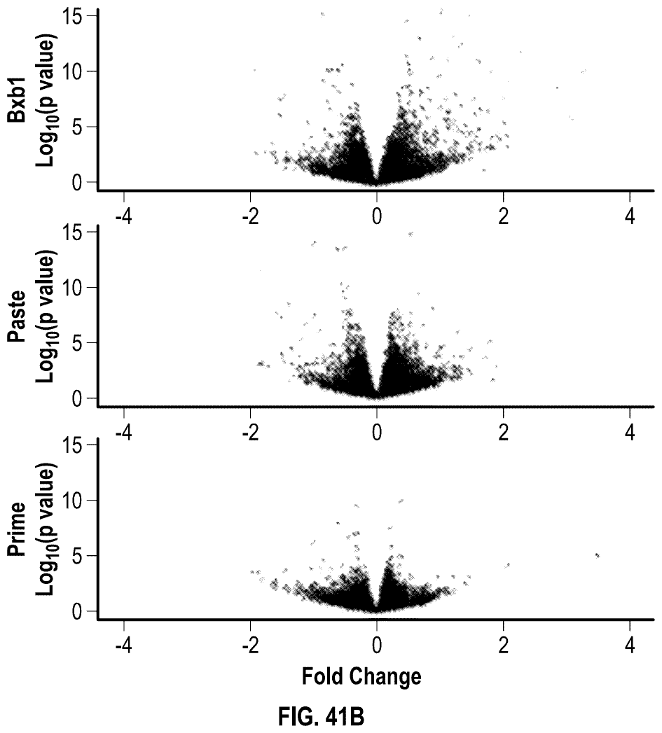

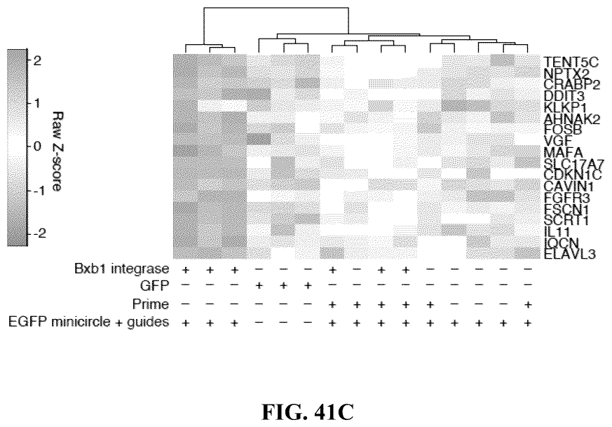

Expression of reverse transcriptases and integrases involved in PASTE can have detrimental effects on cellular health. The complete PASTE system, the corresponding guides and cargo with only PE2, and the corresponding guides and cargo with only Bxb1 were transfected and compared to both GFP control transfections and guides without protein expression via transcriptome-wide RNA sequencing to determine the extent of these effects. While Bxb1 expression in the absence of Prime editing was found to have several significant off targets, the complete PASTE system had only one differentially regulated gene with more than a 1.5-fold change (FIGS. 41A-B). Genes upregulated by Bxb1 overexpression included stress response genes, such as TENT5C and DDIT3, but these changes were not seen in the expression of the PASTE system (FIG. 41C), potentially due to the decreased expression of Bxb1 from the P2A linker on the PASTE construct.

### Example 25

### PASTE Efficiency in Non-Dividing Cell

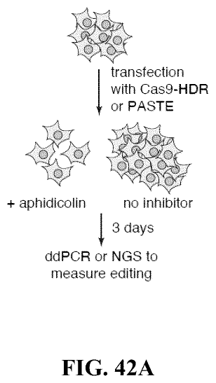

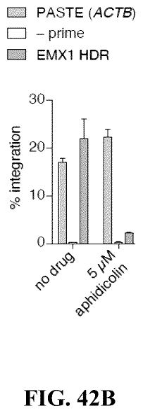

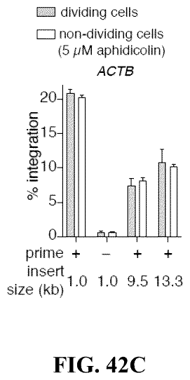

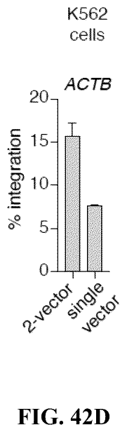

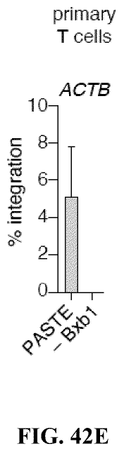

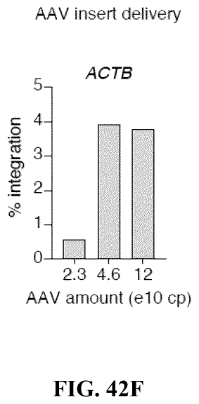

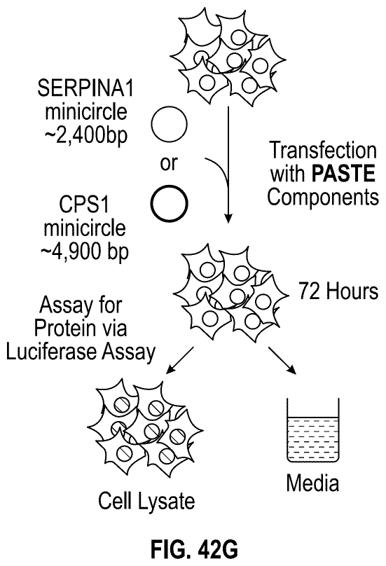

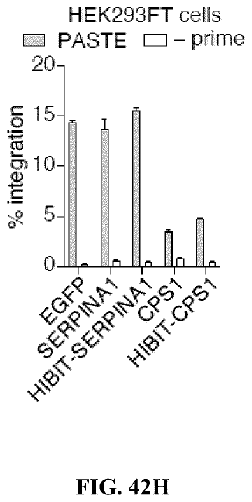

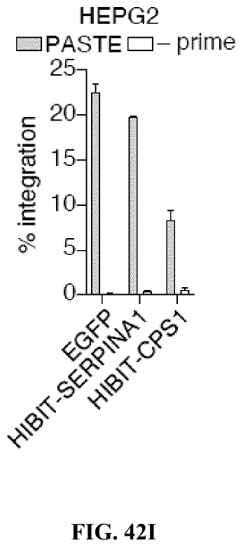

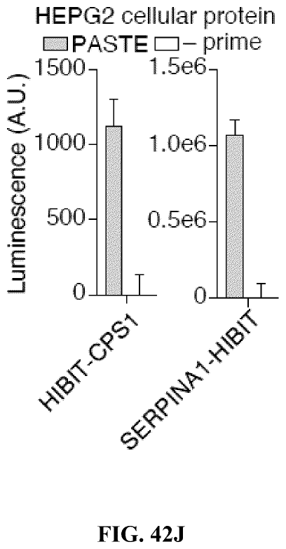

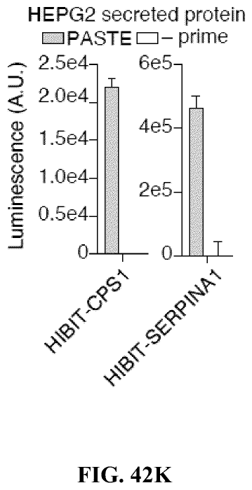

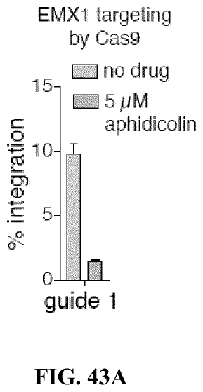

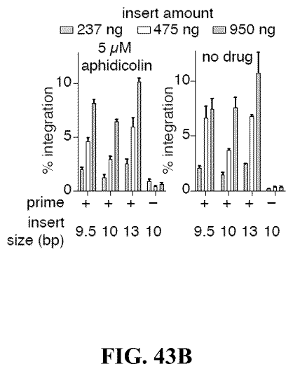

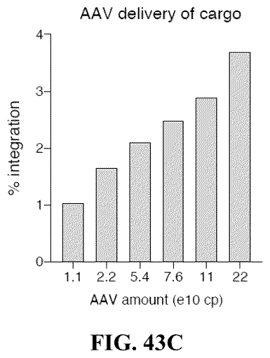

PASTE activity in non-dividing cells was assessed. Cas9 and HDR templates or PASTE were transfected into HEK293FT cells and cell division was arrested via aphidicolin treatment (FIG. 42A). In this model of blocked cell division, PASTE was found to maintain a GFP gene integration activity greater than 20% at the ACTB locus whereas HDR-mediated integration was abolished (FIGS. 42B and 43A).

### Example 26

### Production and Secretion of Therapeutic Transgene

PASTE with larger transgenes and in additional cell lines were assessed.

To evaluate the size limits for therapeutic transgenes, insertion of cargos up to 13.3 kb in length in both dividing and aphidicolin treated cells was assessed. Insertion efficiency greater than 10% was found (FIG. 42C), enabling insertion of ˜99.7% of all full-length human cDNA transgenes. To overcome reduction of large insert delivery to cells because of delivery inefficiencies, delivering larger DNA amounts of insert was found to significantly improve gene integration efficiency (FIG. 43B). PASTE editing to additional cell types such as PASTE in the K562 lymphoblast line and in primary human T cells were also assessed. Both PE2-P2A-Bxb1 (PASTE) and separate delivery of PE2 and Bxb1 were found to result in efficient editing in both cell types (FIGS. 42D-E). Lastly, as therapeutic delivery of PASTE in vivo might require viral delivery of the DNA cargo, whether AAV could deliver an attP containing payload that could be integrated into the genome via Bxb1 was evaluated. Targeting the ACTB locus, AAV was found to be capable of delivering the appropriate template for integrase mediated insertion with rates up to 4% in a dose dependent fashion (FIGS. 42F and 43C).

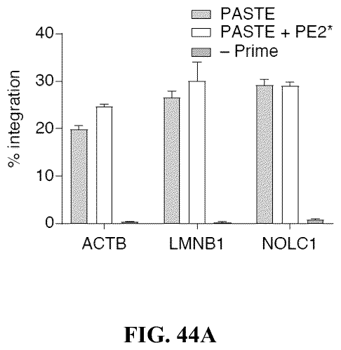

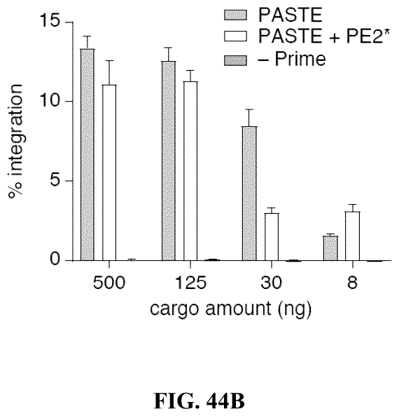

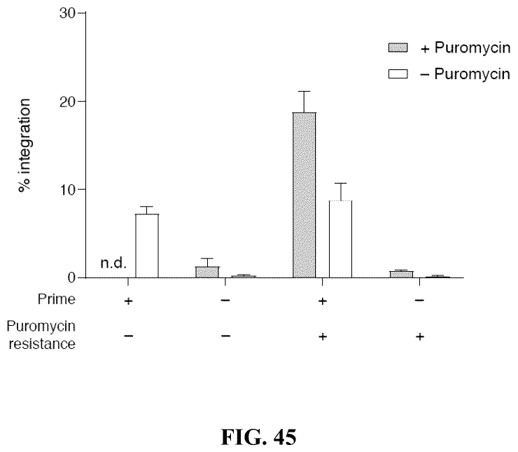

To improve the efficiency of PASTE, PE2* NLS was incorporated for prime editing and improved PASTE integration at multiple loci was found (FIG. 44A). Furthermore, PE2* resulted in more robust integration at lower titrations of cargo plasmid, demonstrating integration at amounts as low as 8 ng of plasmid (FIG. 44B). To combat reductions in PASTE efficiency due to incomplete plasmid delivery, a puromycin resistance gene was co-delivered and found to increase the PASTE efficiency in the presence of drug selection (FIG. 45).

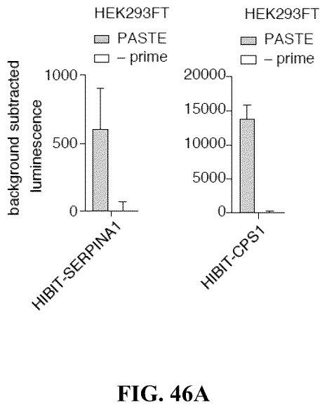

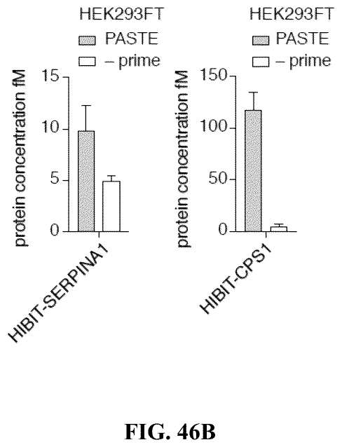

Programmable gene integration provides a modality for expression of therapeutic protein products, and protein production was assessed for therapeutically relevant proteins Alpha-1 antitrypsin (encoded by SERPINA1) and Carbamoyl phosphate synthetase I (encoded by CPS1), involved in the diseases Alpha-1 antitrypsin deficiency and CPS1 deficiency, respectively. By tagging gene products with the luminescent protein subunit HiBiT, the transgene production and secretion were assessed independently in response to PASTE treatment (FIG. 42G). PASTE was transfected with SERPINA1 or CPS1 cargo in HEK293FT cells and a human hepatocellular carcinoma cell line (HepG2) and efficient integration at the ACTB locus was found (FIG. 42H-I). This integration resulted in robust protein expression, intracellular accumulation of transgene products (FIGS. 42J and 46A-B), and secretion of proteins into the media (FIG. 42K).

### Example 27

### Optimized PASTE Constructs

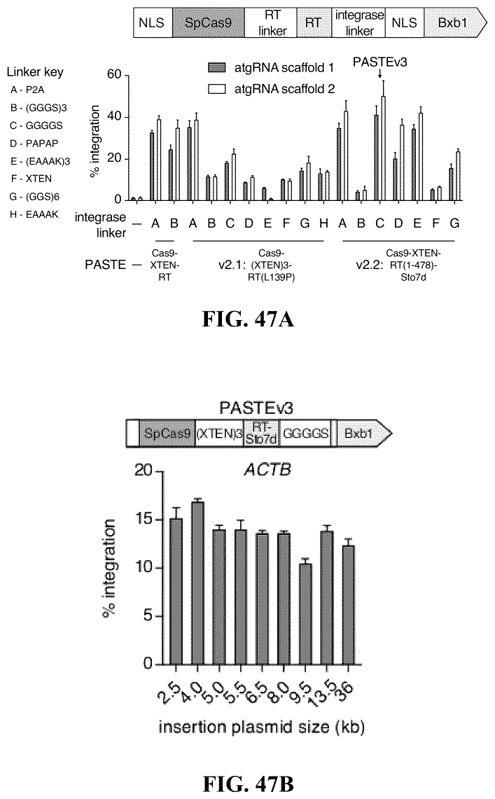

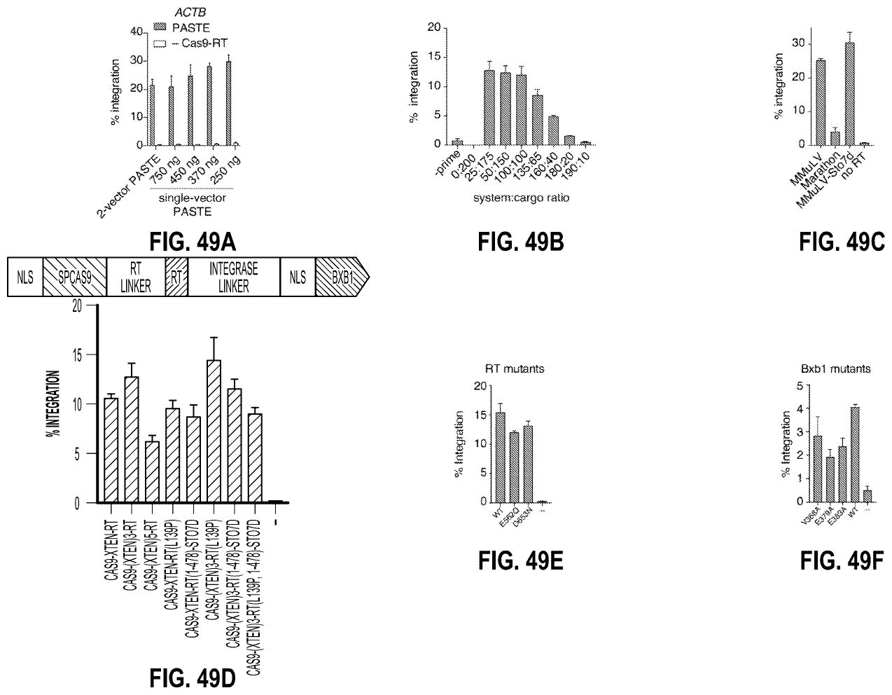

To optimize complex activity, a panel of protein modifications were screened, including alternative reverse transcriptase fusions and mutations, various linkers between the reverse transcriptase domain and integrase and between the Cas9 and reverse transcriptase domain, and reverse transcriptase and BxbINT domain mutants (FIG. 47A and FIG. 49C-FIG. 49F). A number of protein modifications, including a 48 residue XTEN linker between the Cas9 and reverse transcriptase and the fusion of MMuLV to the Sto7d DNA binding domain (Oscorbin et al. FEBS Lett. 594. 4338-4356. 2020) improved editing efficiency (FIG. 47A and FIG. 49C-FIG. 49D). When these top modifications were combined with a GGGGS linker (SEQ ID NO: 420) between the reverse transcriptase-Sto7d domain and the BxbINT, they produced ˜55% gene integration, highlighting the importance of directly recruiting the integrase to the target site (FIG. 47A). This optimized construct was referred to as SpCas9-(XTEN-48)-RT-Sto7d-(GGGGS)-BxbINT. The optimized contruct achieved precise integration of templates as large as ˜36,000 bp with ˜20% integration efficiency (FIG. 47A), with complete integration of the full-length cargo confirmed by Sanger sequencing.

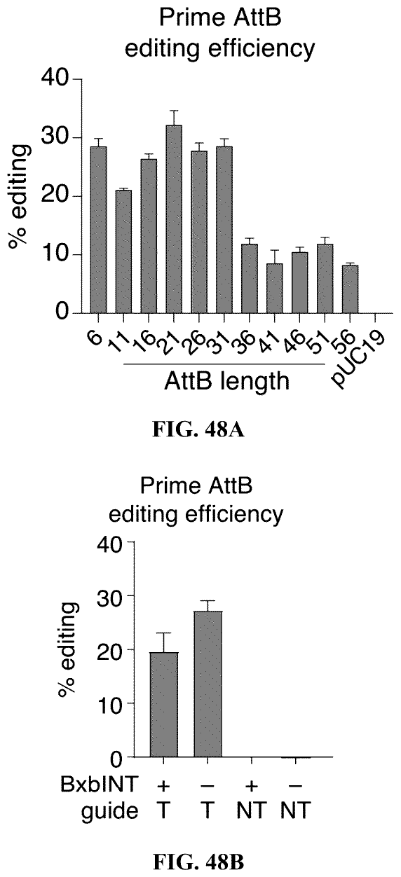

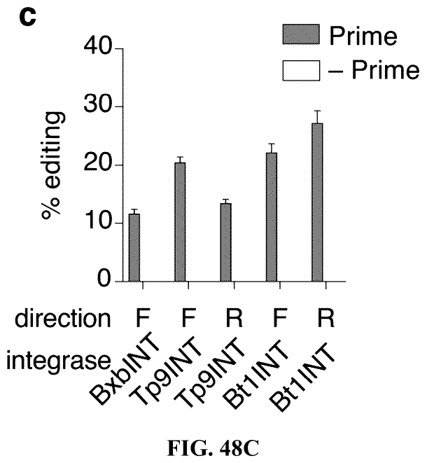

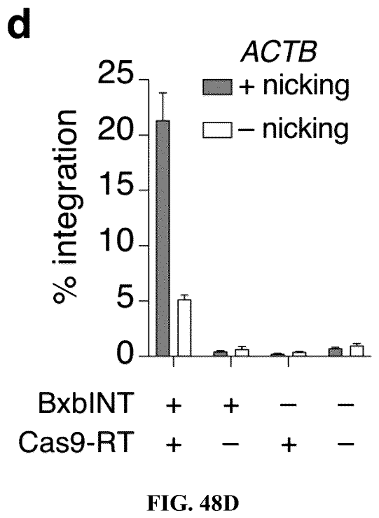

Additionally, pegRNAs containing different AttB length truncations were tested and found that prime editing was capable of inserting sequences up to 56 bp at the beta-actin (ACTB) gene locus, with higher efficiency at lengths below 31 bp (FIG. 48A-FIG. 48B). A panel of multiple enzymes was evaluated, including Bxb1 (i.e., BxbINT), TP901 (i.e., Tp9INT), and phiBT1 (i.e., Bt1INT) phage serine integrases. Prime editing successfully inserted all landing sites tested, with efficiencies between 10-30% (FIG. 48C-FIG. 48D)

### Example 28

### Viral Delivery & In Vivo Editing

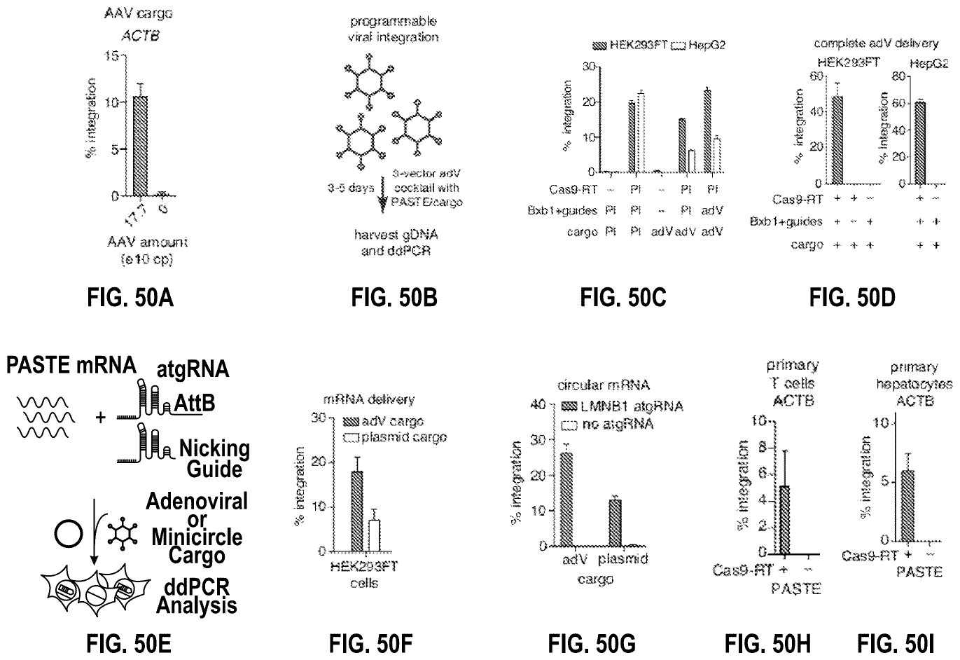

In order to package the complete PASTE system in viral vectors, an AdV vector was utilized (FIG. 50B). Adenovirus was evaluated for if it could deliver a suitable template for BxbINT-mediated insertion along with plasmids for SpCas9-RT-BxbINT and guide expression, or AdV delivery of guides and BxbINT with plasmid delivery of SpCas9-RT, finding that 10-20% integration of the ˜36 kb adenovirus genome carrying EGFP in HEK293FT and HepG2 cells was achieved (FIG. 50C). Upon packaging and delivering the cargo and PASTE system components across 3 AdV vectors, the complete PASTE system (Cas9-reverse transcriptase, integrase and guide RNAs, or cargo) could be substituted by adenoviral delivery, with integration of up to ˜50-60% with viral-only delivery in HEK293FT and HepG2 cells (FIG. 50D).

To further demonstrate PASTE would be amenable for in vivo delivery, an mRNA version of the PASTE protein components was developed as well as chemically-modified synthetic atgRNA and nicking guide against the LMNB1 target (FIG. 50E). Electroporation of the mRNA and guides along with delivery of the template via adenovirus or plasmid yielded high efficiency integration up to ˜23% (FIG. 50E-FIG. 50F). More sustained BxbINT expression could allow for integration into newly placed AttB sites in the genome, so circular mRNA expression was tested and found to boost the efficiency of integration to ˜30% (FIG. 50G-FIG. 50I).

### Example 29

### Simultaneous Deletion & Insertion With PASTE

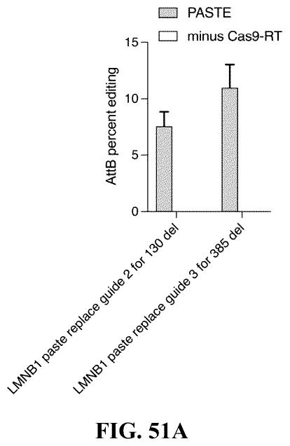

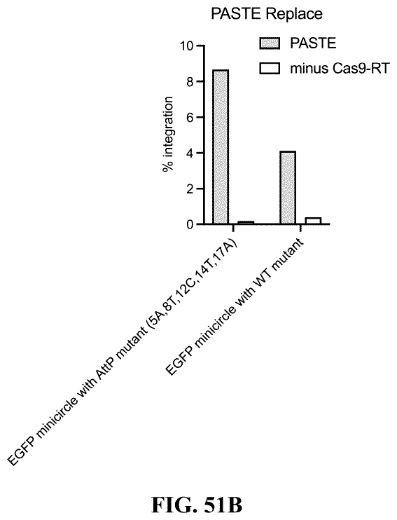

The PASTE system was used to simultaneously delete one sequence and insert another. 130 bp and 385 bp deletions of first exon of LMNB1 with combined insertion of AttB nucleic acid sequence was performed (FIG. 51A). This data shows that it is possible to replace DNA sequence using the PASTE system.

A130 bp deletion of the first exon of LMNB1 with combined insertion of a 967 bp cargo using the PASTE system was also performed.

One of two attP sequences were inserted using the mini circle template that has mutated AttP, as described above. This AttP mutants shows better integration kinetics and efficiency, especially for the shorter AttBs (38-44 bp). The LMNB1 AttB used in this experiment is 38 bp (FIG. 51B).

1.-91. (canceled)

92. A system capable of site-specifically integrating an exogenous nucleic acid into a mammalian cell genome at a desired target site, wherein the system comprises, in a single composition:
(a) a nucleic acid encoding a DNA binding nickase domain linked to a reverse transcriptase domain;
(b) a nucleic acid encoding at least two guide RNAs (gRNAs) comprising, from 3′ to 5′,
i. a primer binding sequence,
ii. a sequence complementary to one strand of an integration recognition sequence, and
iii. a target binding sequence,
wherein the gRNA is capable of guiding the linked nickase-reverse transcriptase domains to the genomic target site;
(c) nucleic acid encoding an integration enzyme; and
(d) an exogenous nucleic acid linked to a sequence that is an integration cognate of the integration recognition sequence.

93. The system of claim 92, wherein the DNA binding nickase domain is linked to the reverse transcriptase domain by in-frame fusion.

94. The system of claim 92, wherein the DNA binding nickase domain is linked to the reverse transcriptase domain by a linker.

95. The system of claim 94, wherein the linker is a peptide fused in-frame between the nickase and reverse transcriptase domains.

96. The system of claim 92, wherein the linked DNA binding nickase-reverse transcriptase domains are further linked to the integration enzyme.

97. The system of claim 92, wherein the DNA binding nickase domain is selected from Cas9-DI0A, Cas9-H840A, and Casl2a/b nickase.

98. The system of claim 92, wherein the reverse transcriptase domain is selected from the group consisting of Moloney Murine Leukemia Virus (M-MLV) reverse transcriptase domain, transcription xenopolymerase (RTX), avian myeloblastosis virus reverse transcriptase (AMV-RT), and Eubacterium rectale maturase RT.

99. The system of claim 98, wherein the reverse transcriptase domain is a M-MLV reverse transcriptase domain.

100. The system of claim 98, wherein the M-MLV reverse transcriptase domain comprises one or more mutations selected from the group consisting of D200N, T306K, W313F, T330P, and L603W.

101. The system of claim 92, wherein the exogenous nucleic acid is a minicircle, a plasmid, a mRNA, or a linear DNA

102. The system of claim 100, wherein exogenous nucleic acid is a minicircle.

103. The system of claim 102, wherein the minicircle does not comprise a sequence of a bacterial origin.

104. The system of claim 92, wherein the integration enzyme is selected from the group consisting of Dre, Vika, Bxb1, φC31, RDF, FLP, φBT1, RI, R2, R3, R4, RS, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, LI, Tol2 Tel, Tc3, Mariner Rimar 1, Mariner mos-I, and Minos.

105. The system of claim 104, wherein the integration enzyme is Bxb1.

106. The system of claim 104, wherein the integration recognition sequence is an attB sequence, an attP sequence, a Vox sequence, or a FRT sequence.

107. The system of claim 106, wherein the integration recognition sequence is an attB sequence and the integration cognate is an attP sequence.

108. The system of claim 106, wherein the exogenous nucleic acid encodes:
a reporter gene;
a degradation tag for programmable knockdown of proteins in the presence of small molecules;
a T-cell receptor (TCR), a chimeric antigen receptor (CAR), an interleukin, a cytokine, or an immune checkpoint gene and the mammalian cell is a T-cell or natural killer (NK) cell;
a beta hemoglobin (HBB) gene and the mammalian cell is a hematopoietic stem cell (HSC);
a metabolic gene; or
a gene involved in an inherited disease or syndrome.

109. The system of claim 92, wherein the exogenous nucleic acid is between 1000 bp and 36,000 bp in length.

110. The system of claim 92, wherein the exogenous nucleic acid is more than 36,000 bp in length.

111. The system of claim 92, wherein the exogenous nucleic acid is less than 1000 bp in length.

112. The system of claim 108, wherein the inherited disease is cystic fibrosis, familial hypercholesterolemia, adenosine deaminase (ADA) deficiency, X-linked SCID (X-SCID), Wiskott-Aldrich syndrome (WAS), hemochromatosis, Tay-Sachs, fragile X syndrome, Huntington's disease, Marfan syndrome, phenylketonuria, or muscular dystrophy.

113. The system of claim 92, further comprising a nicking gRNA.

114. The system of claim 92, wherein a)-d) are introduced into the mammalian cell as an adeno-associated virus (AAV) or an adenovirus (AdV).

115. The system of claim 92, wherein the nucleic acid encoding at least two gRNAs comprises a nucleic acid encoding two gRNAs.


SEQUENCE LISTING (431 sequences)

SEQ ID NO: 1 - DNA, 34 bp, synthetic construct
  misc_feature 1..34: note = Description of Artificial Sequence: Synthetic oligonucleotide
  misc_feature 1..34: note = Lox71
  source 1..34: mol_type = other DNA
ataacttcgtataatgtatgctatacgaacggta

SEQ ID NO: 2 - DNA, 34 bp, synthetic construct
  misc_feature 1..34: note = Description of Artificial Sequence: Synthetic oligonucleotide
  misc_feature 1..34: note = Lox66
  source 1..34: mol_type = other DNA
taccgttcgtataatgtatgctatacgaagttat

SEQ ID NO: 3 - DNA, 46 bp, synthetic construct
  misc_feature 1..46: note = Description of Artificial Sequence: Synthetic oligonucleotide
  misc_feature 1..46: note = AttB
  source 1..46: mol_type = other DNA
ggccggcttgtcgacgacggcggtctccgtcgtcaggatcatccgg

SEQ ID NO: 4 - DNA, 46 bp, synthetic construct
  misc_feature 1..46: note = Description of Artificial Sequence: Synthetic oligonucleotide
  misc_feature 1..46: note = AttP
  source 1..46: mol_type = other DNA
ccggatgatcctgacgacggagaccgccgtcgtcgacaagccggcc

SEQ ID NO: 5 - DNA, 38 bp, synthetic construct
  misc_feature 1..38: note = Description of Artificial Sequence: Synthetic oligonucleotide
  misc_feature 1..38: note = AttB-TT
  source 1..38: mol_type = other DNA
ggcttgtcgacgacggcgttctccgtcgtcaggatcat

SEQ ID NO: 6 - DNA, 52 bp, synthetic construct
  misc_feature 1..52: note = Description of Artificial Sequence: Synthetic oligonucleotide
  misc_feature 1..52: note = AttP-TT
  source 1..52: mol_type = other DNA
gtggtttgtctggtcaaccaccgcgttctcagtggtgtacggtacaaaccca

SEQ ID NO: 7 - DNA, 38 bp, synthetic construct
  misc_feature 1..38: note = Description of Artificial Sequence: Synthetic oligonucleotide
  misc_feature 1..38: note = AttB-AA
  source 1..38: mol_type = other DNA
ggcttgtcgacgacggcgaactccgtcgtcaggatcat

SEQ ID NO: 8 - DNA, 52 bp, synthetic construct
  misc_feature 1..52: note = Description of Artificial Sequence: Synthetic oligonucleotide
  misc_feature 1..52: note = AttP-AA
  source 1..52: mol_type = other DNA
gtggtttgtctggtcaaccaccgcgaactcagtggtgtacggtacaaaccca

SEQ ID NO: 9 - DNA, 38 bp, synthetic construct
  misc_feature 1..38: note = Description of Artificial Sequence: Synthetic oligonucleotide
  misc_feature 1..38: note = AttB-CC
  source 1..38: mol_type = other DNA
ggcttgtcgacgacggcgccctccgtcgtcaggatcat

SEQ ID NO: 10 - DNA, 52 bp, synthetic construct
  misc_feature 1..52: note = Description of Artificial Sequence: Synthetic oligonucleotide
  misc_feature 1..52: note = AttP-CC
  source 1..52: mol_type = other DNA
gtggtttgtctggtcaaccaccgcgccctcagtggtgtacggtacaaaccca

SEQ ID NO: 11 - DNA, 38 bp, synthetic construct
  misc_feature 1..38: note = Description of Artificial Sequence: Synthetic oligonucleotide
  misc_feature 1..38: note = AttB-GG
  source 1..38: mol_type = other DNA
ggcttgtcgacgacggcgggctccgtcgtcaggatcat

SEQ ID NO: 12 - DNA, 52 bp, synthetic construct
  misc_feature 1..52: note = Description of Artificial Sequence: Synthetic oligonucleotide
  misc_feature 1..52: note = AttP-GG
  source 1..52: mol_type = other DNA
gtggtttgtctggtcaaccaccgcgggctcagtggtgtacggtacaaaccca

SEQ ID NO: 13 - DNA, 38 bp, synthetic construct
  misc_feature 1..38: note = Description of Artificial Sequence: Synthetic oligonucleotide
  misc_feature 1..38: note = AttB-TG
  source 1..38: mol_type = other DNA
ggcttgtcgacgacggcgtgctccgtcgtcaggatcat

SEQ ID NO: 14 - DNA, 52 bp, synthetic construct
  misc_feature 1..52: note = Description of Artificial Sequence: Synthetic oligonucleotide
  misc_feature 1..52: note = AttP-TG
  source 1..52: mol_type = other DNA
gtggtttgtctggtcaaccaccgcgtgctcagtggtgtacggtacaaaccca

SEQ ID NO: 15 - DNA, 38 bp, synthetic construct
  misc_feature 1..38: note = Description of Artificial Sequence: Synthetic oligonucleotide
  misc_feature 1..38: note = AttB-GT
  source 1..38: mol_type = other DNA
ggcttgtcgacgacggcggtctccgtcgtcaggatcat

SEQ ID NO: 16 - DNA, 52 bp, synthetic construct
  misc_feature 1..52: note = Description of Artificial Sequence: Synthetic oligonucleotide
  misc_feature 1..52: note = AttP-GT
  source 1..52: mol_type = other DNA
gtggtttgtctggtcaaccaccgcggtctcagtggtgtacggtacaaaccca

SEQ ID NO: 17 - DNA, 38 bp, synthetic construct
  misc_feature 1..38: note = Description of Artificial Sequence: Synthetic oligonucleotide
  misc_feature 1..38: note = AttB-CT
  source 1..38: mol_type = other DNA
ggcttgtcgacgacggcgctctccgtcgtcaggatcat

SEQ ID NO: 18 - DNA, 52 bp, synthetic construct
  misc_feature 1..52: note = Description of Artificial Sequence: Synthetic oligonucleotide
  misc_feature 1..52: note = AttP-CT
  source 1..52: mol_type = other DNA
gtggtttgtctggtcaaccaccgcgctctcagtggtgtacggtacaaaccca

SEQ ID NO: 19 - DNA, 38 bp, synthetic construct
  misc_feature 1..38: note = Description of Artificial Sequence: Synthetic oligonucleotide
  misc_feature 1..38: note = AttB-CA
  source 1..38: mol_type = other DNA
ggcttgtcgacgacggcgcactccgtcgtcaggatcat

SEQ ID NO: 20 - DNA, 52 bp, synthetic construct
  misc_feature 1..52: note = Description of Artificial Sequence: Synthetic oligonucleotide
  misc_feature 1..52: note = AttP-CA
  source 1..52: mol_type = other DNA
gtggtttgtctggtcaaccaccgcgcactcagtggtgtacggtacaaaccca

SEQ ID NO: 21 - DNA, 38 bp, synthetic construct
  misc_feature 1..38: note = Description of Artificial Sequence: Synthetic oligonucleotide
  misc_feature 1..38: note = AttB-TC
  source 1..38: mol_type = other DNA
ggcttgtcgacgacggcgtcctccgtcgtcaggatcat

SEQ ID NO: 22 - DNA, 52 bp, synthetic construct
  misc_feature 1..52: note = Description of Artificial Sequence: Synthetic oligonucleotide
  misc_feature 1..52: note = AttP-TC
  source 1..52: mol_type = other DNA
gtggtttgtctggtcaaccaccgcgtcctcagtggtgtacggtacaaaccca

SEQ ID NO: 23 - DNA, 38 bp, synthetic construct
  misc_feature 1..38: note = Description of Artificial Sequence: Synthetic oligonucleotide
  misc_feature 1..38: note = AttB-GA
  source 1..38: mol_type = other DNA
ggcttgtcgacgacggcggactccgtcgtcaggatcat

SEQ ID NO: 24 - DNA, 52 bp, synthetic construct
  misc_feature 1..52: note = Description of Artificial Sequence: Synthetic oligonucleotide
  misc_feature 1..52: note = AttP-GA
  source 1..52: mol_type = other DNA
gtggtttgtctggtcaaccaccgcggactcagtggtgtacggtacaaaccca

SEQ ID NO: 25 - DNA, 38 bp, synthetic construct
  misc_feature 1..38: note = Description of Artificial Sequence: Synthetic oligonucleotide
  misc_feature 1..38: note = AttB-AG
  source 1..38: mol_type = other DNA
ggcttgtcgacgacggcgagctccgtcgtcaggatcat

SEQ ID NO: 26 - DNA, 52 bp, synthetic construct
  misc_feature 1..52: note = Description of Artificial Sequence: Synthetic oligonucleotide
  misc_feature 1..52: note = AttP-AG
  source 1..52: mol_type = other DNA
gtggtttgtctggtcaaccaccgcgagctcagtggtgtacggtacaaaccca

SEQ ID NO: 27 - DNA, 38 bp, synthetic construct
  misc_feature 1..38: note = Description of Artificial Sequence: Synthetic oligonucleotide
  misc_feature 1..38: note = AttB-AC
  source 1..38: mol_type = other DNA
ggcttgtcgacgacggcgacctccgtcgtcaggatcat

SEQ ID NO: 28 - DNA, 52 bp, synthetic construct
  misc_feature 1..52: note = Description of Artificial Sequence: Synthetic oligonucleotide
  misc_feature 1..52: note = AttP-AC
  source 1..52: mol_type = other DNA
gtggtttgtctggtcaaccaccgcgacctcagtggtgtacggtacaaaccca

SEQ ID NO: 29 - DNA, 38 bp, synthetic construct
  misc_feature 1..38: note = Description of Artificial Sequence: Synthetic oligonucleotide
  misc_feature 1..38: note = AttB-AT
  source 1..38: mol_type = other DNA
ggcttgtcgacgacggcgatctccgtcgtcaggatcat

SEQ ID NO: 30 - DNA, 52 bp, synthetic construct
  misc_feature 1..52: note = Description of Artificial Sequence: Synthetic oligonucleotide
  misc_feature 1..52: note = AttP-AT
  source 1..52: mol_type = other DNA
gtggtttgtctggtcaaccaccgcgatctcagtggtgtacggtacaaaccca

SEQ ID NO: 31 - DNA, 38 bp, synthetic construct
  misc_feature 1..38: note = Description of Artificial Sequence: Synthetic oligonucleotide
  misc_feature 1..38: note = AttB-GC
  source 1..38: mol_type = other DNA
ggcttgtcgacgacggcggcctccgtcgtcaggatcat

SEQ ID NO: 32 - DNA, 52 bp, synthetic construct
  misc_feature 1..52: note = Description of Artificial Sequence: Synthetic oligonucleotide
  misc_feature 1..52: note = AttP-GC
  source 1..52: mol_type = other DNA
gtggtttgtctggtcaaccaccgcggcctcagtggtgtacggtacaaaccca

SEQ ID NO: 33 - DNA, 38 bp, synthetic construct
  misc_feature 1..38: note = Description of Artificial Sequence: Synthetic oligonucleotide
  misc_feature 1..38: note = AttB-CG
  source 1..38: mol_type = other DNA
ggcttgtcgacgacggcgcgctccgtcgtcaggatcat

SEQ ID NO: 34 - DNA, 52 bp, synthetic construct
  misc_feature 1..52: note = Description of Artificial Sequence: Synthetic oligonucleotide
  misc_feature 1..52: note = AttB-CG
  source 1..52: mol_type = other DNA
gtggtttgtctggtcaaccaccgcgcgctcagtggtgtacggtacaaaccca

SEQ ID NO: 35 - DNA, 38 bp, synthetic construct
  misc_feature 1..38: note = Description of Artificial Sequence: Synthetic oligonucleotide
  misc_feature 1..38: note = AttB-TA
  source 1..38: mol_type = other DNA
ggcttgtcgacgacggcgtactccgtcgtcaggatcat

SEQ ID NO: 36 - DNA, 52 bp, synthetic construct
  misc_feature 1..52: note = Description of Artificial Sequence: Synthetic oligonucleotide
  misc_feature 1..52: note = AttP-TA
  source 1..52: mol_type = other DNA
gtggtttgtctggtcaaccaccgcgtactcagtggtgtacggtacaaaccca

SEQ ID NO: 37 - DNA, 45 bp, synthetic construct
  misc_feature 1..45: note = Description of Artificial Sequence: Synthetic oligonucleotide
  misc_feature 1..45: note = C-31-B
  source 1..45: mol_type = other DNA
tgcgggtgccagggcgtgcccttgggctccccgggcgcgtactcc

SEQ ID NO: 38 - DNA, 42 bp, synthetic construct
  misc_feature 1..42: note = Description of Artificial Sequence: Synthetic oligonucleotide
  misc_feature 1..42: note = C31-P
  source 1..42: mol_type = other DNA
gtgccccaactggggtaacctttgagttctctcagttggggg

SEQ ID NO: 39 - DNA, 57 bp, synthetic construct
  misc_feature 1..57: note = Description of Artificial Sequence: Synthetic oligonucleotide
  misc_feature 1..57: note = R4-B
  source 1..57: mol_type = other DNA
gcgcccaagttgcccatgaccatgccgaagcagtggtagaagggcaccggcagacac

SEQ ID NO: 40 - DNA, 70 bp, synthetic construct
  misc_feature 1..70: note = Description of Artificial Sequence: Synthetic oligonucleotide
  misc_feature 1..70: note = R4-P
  source 1..70: mol_type = other DNA
aggcatgttccccaaagcgataccacttgaagcagtggtactgcttgtgggtacactctgcgggtgatga

SEQ ID NO: 41 - DNA, 60 bp, synthetic construct
  misc_feature 1..60: note = Description of Artificial Sequence: Synthetic oligonucleotide
  misc_feature 1..60: note = BT1-B
  source 1..60: mol_type = other DNA
gtccttgaccaggtttttgacgaaagtgatccagatgatccagctccacaccccgaacgc

SEQ ID NO: 42 - DNA, 63 bp, synthetic construct
  misc_feature 1..63: note = Description of Artificial Sequence: Synthetic oligonucleotide
  misc_feature 1..63: note = BT1-P
  source 1..63: mol_type = other DNA
ggtgctgggttgttgtctctggacagtgatccatgggaaactactcagcaccaccaatgttcc

SEQ ID NO: 43 - DNA, 50 bp, synthetic construct
  misc_feature 1..50: note = Description of Artificial Sequence: Synthetic oligonucleotide
  misc_feature 1..50: note = Bxb-B
  source 1..50: mol_type = other DNA
tcggccggcttgtcgacgacggcggtctccgtcgtcaggatcatccgggc

SEQ ID NO: 44 - DNA, 58 bp, synthetic construct
  misc_feature 1..58: note = Description of Artificial Sequence: Synthetic oligonucleotide
  misc_feature 1..58: note = Bxb-P
  source 1..58: mol_type = other DNA
gtcgtggtttgtctggtcaaccaccgcggtctcagtggtgtacggtacaaaccccgac

SEQ ID NO: 45 - DNA, 46 bp, synthetic construct
  misc_feature 1..46: note = Description of Artificial Sequence: Synthetic oligonucleotide
  misc_feature 1..46: note = TG1-B
  source 1..46: mol_type = other DNA
gatcagctccgcgggcaagaccttctccttcacggggtggaaggtc

SEQ ID NO: 46 - DNA, 67 bp, synthetic construct
  misc_feature 1..67: note = Description of Artificial Sequence: Synthetic oligonucleotide
  misc_feature 1..67: note = TG1-P
  source 1..67: mol_type = other DNA
tcaaccccgttccagcccaacagtgttagtctttgctcttacccagttgggcgggatagcctgcccg

SEQ ID NO: 47 - DNA, 57 bp, synthetic construct
  misc_feature 1..57: note = Description of Artificial Sequence: Synthetic oligonucleotide
  misc_feature 1..57: note = C1-B
  source 1..57: mol_type = other DNA
aacgattttcaaaggatcactgaatcaaaagtattgctcatccacgcgaaatttttc

SEQ ID NO: 48 - DNA, 57 bp, synthetic construct
  misc_feature 1..57: note = Description of Artificial Sequence: Synthetic oligonucleotide
  misc_feature 1..57: note = C1-P
  source 1..57: mol_type = other DNA
aatattttaggtatatgattttgtttattagtgtaaataacactatgtacctaaaat

SEQ ID NO: 49 - DNA, 53 bp, synthetic construct
  misc_feature 1..53: note = Description of Artificial Sequence: Synthetic oligonucleotide
  misc_feature 1..53: note = C370-B
  source 1..53: mol_type = other DNA
tgtaaaggagactgataatggcatgtacaactatactcgtcggtaaaaaggca

SEQ ID NO: 50 - DNA, 52 bp, synthetic construct
  misc_feature 1..52: note = Description of Artificial Sequence: Synthetic oligonucleotide
  misc_feature 1..52: note = C370-P
  source 1..52: mol_type = other DNA
taaaaaaatacagcgtttttcatgtacaactatactagttgtagtgcctaaa

SEQ ID NO: 51 - DNA, 56 bp, synthetic construct
  misc_feature 1..56: note = Description of Artificial Sequence: Synthetic oligonucleotide
  misc_feature 1..56: note = K38-B
  source 1..56: mol_type = other DNA
gagcgccggatcagggagtggacggcctgggagcgctacacgctgtggctgcggtc

SEQ ID NO: 52 - DNA, 56 bp, synthetic construct
  misc_feature 1..56: note = Description of Artificial Sequence: Synthetic oligonucleotide
  misc_feature 1..56: note = K38-P
  source 1..56: mol_type = other DNA
ccctaatacgcaagtcgataactctcctgggagcgttgacaacttgcgcaccctga

SEQ ID NO: 53 - DNA, 68 bp, synthetic construct
  misc_feature 1..68: note = Description of Artificial Sequence: Synthetic oligonucleotide
  misc_feature 1..68: note = RB-B
  source 1..68: mol_type = other DNA
tctcgtggtggtggaaggtgttggtgcggggttggccgtggtcgaggtggggtggtggtagccattcg

SEQ ID NO: 54 - DNA, 69 bp, synthetic construct
  misc_feature 1..69: note = Description of Artificial Sequence: Synthetic oligonucleotide
  misc_feature 1..69: note = RV-P
  source 1..69: mol_type = other DNA
gcacaggtgtagtgtatctcacaggtccacggttggccgtggactgctgaagaacattccacgccagga

SEQ ID NO: 55 - DNA, 65 bp, synthetic construct
  misc_feature 1..65: note = Description of Artificial Sequence: Synthetic oligonucleotide
  misc_feature 1..65: note = SPBC-B
  source 1..65: mol_type = other DNA
agtgcagcatgtcattaatatcagtacagataaagctgtatctcctgtgaacacaatgggtgcca

SEQ ID NO: 56 - DNA, 55 bp, synthetic construct
  misc_feature 1..55: note = Description of Artificial Sequence: Synthetic oligonucleotide
  misc_feature 1..55: note = SPBC-P
  source 1..55: mol_type = other DNA
aaagtagtaagtatcttaaaaaacagataaagctgtatattaagatacttactac

SEQ ID NO: 57 - DNA, 54 bp, synthetic construct
  misc_feature 1..54: note = Description of Artificial Sequence: Synthetic oligonucleotide
  misc_feature 1..54: note = TP901-B
  source 1..54: mol_type = other DNA
tgataattgccaacacaattaacatctcaatcaaggtaaatgctttttcgtttt

SEQ ID NO: 58 - DNA, 54 bp, synthetic construct
  misc_feature 1..54: note = Description of Artificial Sequence: Synthetic oligonucleotide
  misc_feature 1..54: note = TP901-P
  source 1..54: mol_type = other DNA
aattgcgagtttttatttcgtttatttcaattaaggtaactaaaaaactccttt

SEQ ID NO: 59 - DNA, 68 bp, synthetic construct
  misc_feature 1..68: note = Description of Artificial Sequence: Synthetic oligonucleotide
  misc_feature 1..68: note = Wbeta-B
  source 1..68: mol_type = other DNA
aaggtagcgtcaacgataggtgtaactgtcgtgtttgtaacggtacttccaacagctggcgtttcagt

SEQ ID NO: 60 - DNA, 68 bp, synthetic construct
  misc_feature 1..68: note = Description of Artificial Sequence: Synthetic oligonucleotide
  misc_feature 1..68: note = Wbeta-P
  source 1..68: mol_type = other DNA
tagttttaaagttggttattagttactgtgatatttatcacggtacccaataaccaatgaatatttga

SEQ ID NO: 61 - DNA, 57 bp, synthetic construct
  misc_feature 1..57: note = Description of Artificial Sequence: Synthetic oligonucleotide
  misc_feature 1..57: note = A118-B
  source 1..57: mol_type = other DNA
tgtaactttttcggatcaagctatgaaggacgcaaagagggaactaaacacttaatt

SEQ ID NO: 62 - DNA, 57 bp, synthetic construct
  misc_feature 1..57: note = Description of Artificial Sequence: Synthetic oligonucleotide
  misc_feature 1..57: note = A118-P
  source 1..57: mol_type = other DNA
ttgtttagttcctcgttttctctcgttggaagaagaagaaacgagaaactaaaatta

SEQ ID NO: 63 - DNA, 63 bp, synthetic construct
  misc_feature 1..63: note = Description of Artificial Sequence: Synthetic oligonucleotide
  misc_feature 1..63: note = BL3-B
  source 1..63: mol_type = other DNA
caacctgttgacatgtttccacagacaactcacgtggaggtagtcacggcttttacgttagtt

SEQ ID NO: 64 - DNA, 61 bp, synthetic construct
  misc_feature 1..61: note = Description of Artificial Sequence: Synthetic oligonucleotide
  misc_feature 1..61: note = BL3-P
  source 1..61: mol_type = other DNA
gagaatactgttgaacaatgaaaaactaggcatgtagaagttgtttgtgcactaactttaa

SEQ ID NO: 65 - DNA, 120 bp, synthetic construct
  misc_feature 1..120: note = Description of Artificial Sequence: Synthetic polynucleotide
  misc_feature 1..120: note = MR11-B
  source 1..120: mol_type = other DNA
acaggtcaacacatcgcagttatcgaacaatcttcgaaaatgtatggaggcacttgtatcaatataggatgtataccttcgaagacacttgtacatgatggattagaaggcaaatccttt

SEQ ID NO: 66 - DNA, 120 bp, synthetic construct
  misc_feature 1..120: note = Description of Artificial Sequence: Synthetic polynucleotide
  misc_feature 1..120: note = MR11-P
  source 1..120: mol_type = other DNA
caaaataaaaaacattgatttttattaacttcttttgtgcggaactacgaacagttcattaatacgaagtgtacaaacttccatacaaaaataaccacgacaattaagacgtggtttcta

SEQ ID NO: 67 - DNA, 17 bp, synthetic construct
  misc_feature 1..17: note = Description of Artificial Sequence: Synthetic oligonucleotide
  misc_feature 1..17: note = AttL
  source 1..17: mol_type = other DNA
attatttctcaccctga

SEQ ID NO: 68 - DNA, 17 bp, synthetic construct
  misc_feature 1..17: note = Description of Artificial Sequence: Synthetic oligonucleotide
  misc_feature 1..17: note = AttR
  source 1..17: mol_type = other DNA
atcatctcccacccgga

SEQ ID NO: 69 - DNA, 34 bp, synthetic construct
  misc_feature 1..34: note = Description of Artificial Sequence: Synthetic oligonucleotide
  misc_feature 1..34: note = Vox
  source 1..34: mol_type = other DNA
aataggtctgagaacgcccattctcagacgtatt

SEQ ID NO: 70 - DNA, 34 bp, synthetic construct
  misc_feature 1..34: note = Description of Artificial Sequence: Synthetic oligonucleotide
  misc_feature 1..34: note = FRT
  source 1..34: mol_type = other DNA
gaagttcctatactttctagagaataggaacttc

SEQ ID NO: 71 - DNA, 5881 bp, synthetic construct
  misc_feature 1..5881: note = Description of Artificial Sequence: Synthetic polynucleotide
  misc_feature 1..5881: note = Cre Recombinase Expression Plasmid
  source 1..5881: mol_type = other DNA
ggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggggggggggggggggggggggggggggggggggcgggggggggcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgagccgccaccatggccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatggaccggtggaacaaaaacttatttctgaagaagatctgtgatagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 72 - DNA, 4915 bp, synthetic construct
  misc_feature 1..4915: note = Description of Artificial Sequence: Synthetic polynucleotide
  misc_feature 1..4915: note = GFP-Lox66-Cre expression plasmid
  source 1..4915: mol_type = other DNA
agctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctccatgtcatctacaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaactcgagatccactagagtgtggcggccgcattcttataatcagcatcatgatgtggtaccacatcatgatgctgattacccccaactgagagaactcaaaggttaccccagttggggcgggcccacaaataaagcaatagcatcacaaatttcacaaataaagcatttttttcactgcattctagttgtggtttgtccaaactcatcgagctcgagatctggcgaaggcgatgggggtcttgaaggcgtgctggtactccacgatgcccagctcggtgttgctgtgcagctcctccacgcggcggaaggcgaacatggggcccccgttctgcaggatgctggggtggatggcgctcttgaagtgcatgtggctgtccaccacgaagctgtagtagccgccgtcgcgcaggctgaaggtgcgggcgaagctgcccaccagcacgttatcgcccatggggtgcaggtgctccacggtggcgttgctgcggatgatcttgtcggtgaagatcacgctgtcctcggggaagccggtgcccaccaccttgaagtcgccgatcacgcggccggcctcgtagcggtagctgaagctcacgtgcagcacgccgccgtcctcgtacttctcgatgcgggtgttggtgtagccgccgttgttgatggcgtgcaggaaggggttctcgtagccgctggggtaggtgccgaagtggtagaagccgtagcccatcacgtggctcagcaggtaggggctgaaggtcagggcgcctttggtgctcttcatcttgttggtcatgcggccctgctcgggggtgccctctccgccgcccaccagctcgaactccacgccgttcagggtgccggtgatgcggcactcgatcttcatggcgggcatggtggcgaccggtagcgctagcggcttcggataacttcgtatagcatacattatacgaacggtaagcgctaccgccggcatacccaagtgaagttgctcgcagcttatagtcgcgcccggggagcccaagggcacgccctggcaccgcggccgctgagtctcgaccatcatcatcatcatcattgagtttatctgggataacagggtaatgtcatctagggataacagggtatgtcatctgggataacagggtaatgtatctagggataacagggtaatgtcatctgggataacagggtaatgtcatctagggataacagggtatgtcatctgggataacagggtaatgtatctagggataacagggtaatgtcatctgggataacagggtaatgtcatctagggataacagggtatgtcatctgggataacagggtaatgtatctagggataacagggtaatgtcatctgggataacagggtaatgtcatctagggataacagggtatgtcatctgggataacagggtaatgtatctagggataacagggtaatgtcatctgggataacagggtaatgtcatctagggataacagggtatgtcatctgggataacagggtaatgtatctagggataacagggtaatgtcatctgggataacagggtaatgtcatctagggataacagggtatgtcatctgggataacagggtaatgtatctagggataacagggtaatgtcatctgggataacagggtaatgtcatctagggataacagggtaaatgtcatctagggataacagggtaatgtcatctagggataacagggtaatgtcatctgggataacagggtaatgtcatctagggataacagggtaatgtatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataattggtaacgaatcagacaattgacggcttgacggagtagcatagggtttgcagaatccctgcttcgtccatttgacaggcacattatgcatgccgcttcgccttcgcgcgcgaattgatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagctggagaccgtttggcccccctcgagcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatca

SEQ ID NO: 73 - DNA, 10815 bp, synthetic construct
  misc_feature 1..10815: note = Description of Artificial Sequence: Synthetic polynucleotide
  misc_feature 1..10815: note = pCMV-PE2-P2A-Cre
  source 1..10815: mol_type = other DNA
acgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtcggaagcggagctactaacttcagcctgctgaagcaggctggcgacgtggaggagaaccctggacctaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaatttaaacccgctgatcagcctcgactgtgccttctagttgccagccatctgttgtttgcccctcccccgtgccttccttgaccctggaaggtgccactcccactgtcctttcctaataaaatgagaaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatat

SEQ ID NO: 74 - DNA, 20 bp, synthetic construct
  misc_feature 1..20: note = Description of Artificial Sequence: Synthetic oligonucleotide
  misc_feature 1..20: note = +90ngRNA guide sequence
  source 1..20: mol_type = other DNA
gtcaaccagtatcccggtgc

SEQ ID NO: 75 - DNA, 96 bp, synthetic construct
  misc_feature 1..96: note = Description of Artificial Sequence: Synthetic oligonucleotide
  misc_feature 1..96: note = +90ngRNA
  source 1..96: mol_type = other DNA
gtcaaccagtatcccggtgcgttttagagctagaaatagcaagttaaaataaggctagtccgttatcaacttgaaaaagtggcaccgagtcggtgc

SEQ ID NO: 76 - DNA, 4968 bp, synthetic construct
  misc_feature 1..4968: note = Description of Artificial Sequence: Synthetic polynucleotide
  misc_feature 1..4968: note = GFP minicircle template (before cleavage)
  source 1..4968: mol_type = other DNA
tgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatacattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactataagctgcgagcaacttcacttgggtatgccggcggtagcgcttaccgttcgtataatgtatgctatacgaagttatccgaagccgctagcggtggtttgtctggtcaaccaccgcggtctcagtggtgtacggtacaaacccagctaccggtcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgagtttggacaaaccacaactagaatgcagtgaaaaaaatgctttatttgtgaaatttgtgatgctattgctttatttgtgggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagct

SEQ ID NO: 77 - DNA, 4855 bp, synthetic construct
  misc_feature 1..4855: note = Description of Artificial Sequence: Synthetic polynucleotide
  misc_feature 1..4855: note = GLuc minicircle template
  source 1..4855: mol_type = other DNA
tgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatacattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactataagctgcgagcaacttcacttgggtatgccggcggtagcgcttaccgttcgtataatgtatgctatacgaagttatccgaagccgctagcggtggtttgtctggtcaaccaccgcggtctcagtggtgtacggtacaaacccactaccggtcgccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaagcggagctcgatgagtttggacaaaccacaactagaatgcagtgaaaaaaatgctttatttgtgaaatttgtgatgctattgctttatttgtgggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagct

SEQ ID NO: 78 - DNA, 38 bp, synthetic construct
  misc_feature 1..38: note = Description of Artificial Sequence: Synthetic oligonucleotide
  misc_feature 1..38: note = pseudo-attP
  source 1..38: mol_type = other DNA
ccccaactggggtaacctttgagttctctcagttgggg

SEQ ID NO: 79 - DNA, 194 bp, synthetic construct
  misc_feature 1..194: note = Description of Artificial Sequence: Synthetic polynucleotide
  misc_feature 1..194: note = Albumin-pegRNA-SERPIN
  source 1..194: mol_type = other DNA
gactgaaacttcacagaatagttttagagctagaaatagcaagttaaaataaggctagtccgttatcaacttgaaaaagtggcaccgagtcggtgcttgggatagttatgaattcaatcttcaaccctatccggatgatcctgacgacggagaccgccgtcgtcgacaagccggcctctgtgaagtttcagtca

SEQ ID NO: 80 - DNA, 189 bp, synthetic construct
  misc_feature 1..189: note = Description of Artificial Sequence: Synthetic polynucleotide
  misc_feature 1..189: note = Albumin-pegRNA-CPS1
  source 1..189: mol_type = other DNA
gactgaaacttcacagaatagttttagagctagaaatagcaagttaaaataaggctagtccgttatcaacttgaaaaagtggcaccgagtcggtgcttgggatagttatgaattcaatcttcaaccctatccggatgatcctgacgacggagaccgccgtcgtcgacaagccggcctctgtgaagtttc

SEQ ID NO: 81 - DNA, 177 bp, synthetic construct
  misc_feature 1..177: note = Description of Artificial Sequence: Synthetic polynucleotide
  misc_feature 1..177: note = 34bp lox71 pegRNA
  source 1..177: mol_type = other DNA
ggcccagactgagcacgtgagttttagagctagaaatagcaagttaaaataaggctagtccgttatcaacttgaaaaagtggcaccgagtcggtgctggaggaagcagggcttcctttcctctgccatcataccgttcgtatagcatacattatacgaagttatcgtgctcagtctg

SEQ ID NO: 82 - DNA, 177 bp, synthetic construct
  misc_feature 1..177: note = Description of Artificial Sequence: Synthetic polynucleotide
  misc_feature 1..177: note = 34bp lox66 pegRNA
  source 1..177: mol_type = other DNA
ggcccagactgagcacgtgagttttagagctagaaatagcaagttaaaataaggctagtccgttatcaacttgaaaaagtggcaccgagtcggtgctggaggaagcagggcttcctttcctctgccatcaataacttcgtatagcatacattatacgaacggtacgtgctcagtctg

SEQ ID NO: 83 - DNA, 20 bp, synthetic construct
  misc_feature 1..20: note = Description of Artificial Sequence: Synthetic oligonucleotide
  misc_feature 1..20: note = gRNA2
  source 1..20: mol_type = other DNA
ggcccagactgagcacgtga

SEQ ID NO: 84 - DNA, 184 bp, synthetic construct
  misc_feature 1..184: note = Description of Artificial Sequence: Synthetic polynucleotide
  source 1..184: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggccggatgatcctgacgacggagaccgccgtcgtcgacaagccggcctgagctgcgagaa

SEQ ID NO: 85 - DNA, 179 bp, synthetic construct
  misc_feature 1..179: note = Description of Artificial Sequence: Synthetic polynucleotide
  source 1..179: mol_type = other DNA
gctattctcgcagctcaccagttttagagctagaaatagcaagttaaaataaggctagtccgttatcaacttgaaaaagtggcaccgagtcggtgcgagtcggtgcgacgagcgcggcgatatcatcatccatggcacaattaacatctcaatcaaggtaaatgcttgagctgcgagaa

SEQ ID NO: 86 - DNA, 179 bp, synthetic construct
  misc_feature 1..179: note = Description of Artificial Sequence: Synthetic polynucleotide
  source 1..179: mol_type = other DNA
gctattctcgcagctcaccagttttagagctagaaatagcaagttaaaataaggctagtccgttatcaacttgaaaaagtggcaccgagtcggtgcgagtcggtgcgacgagcgcggcgatatcatcatccatggagcatttaccttgattgagatgttaattgtgtgagctgcgagaa

SEQ ID NO: 87 - DNA, 182 bp, synthetic construct
  misc_feature 1..182: note = Description of Artificial Sequence: Synthetic polynucleotide
  source 1..182: mol_type = other DNA
gctattctcgcagctcaccagttttagagctagaaatagcaagttaaaataaggctagtccgttatcaacttgaaaaagtggcaccgagtcggtgcgagtcggtgcgacgagcgcggcgatatcatcatccatggcaggtttttgacgaaagtgatccagatgatccagtgagctgcgagaa

SEQ ID NO: 88 - DNA, 182 bp, synthetic construct
  misc_feature 1..182: note = Description of Artificial Sequence: Synthetic polynucleotide
  source 1..182: mol_type = other DNA
gctattctcgcagctcaccagttttagagctagaaatagcaagttaaaataaggctagtccgttatcaacttgaaaaagtggcaccgagtcggtgcgagtcggtgcgacgagcgcggcgatatcatcatccatggctggatcatctggatcactttcgtcaaaaacctgtgagctgcgagaa

SEQ ID NO: 89 - DNA, 96 bp, synthetic construct
  misc_feature 1..96: note = Description of Artificial Sequence: Synthetic oligonucleotide
  source 1..96: mol_type = other DNA
gaagccggccttgcacatgcgttttagagctagaaatagcaagttaaaataaggctagtccgttatcaacttgaaaaagtggcaccgagtcggtgc

SEQ ID NO: 90 - DNA, 164 bp, synthetic construct
  misc_feature 1..164: note = Description of Artificial Sequence: Synthetic polynucleotide
  source 1..164: mol_type = other DNA
gaagccggccttgcacatgcgttttagagctagaaatagcaagttaaaataaggctagtccgttatcaacttgaaaaagtggcaccgagtcggtgcatatcatcatccatggtaccgttcgtatagcatacattatacgaagttattgagctgcgagaatagcc

SEQ ID NO: 91 - DNA, 172 bp, synthetic construct
  misc_feature 1..172: note = Description of Artificial Sequence: Synthetic polynucleotide
  source 1..172: mol_type = other DNA
gaagccggccttgcacatgcgttttagagctagaaatagcaagttaaaataaggctagtccgttatcaacttgaaaaagtggcaccgagtcggtgcgacgagcgcggcgatatcatcatccatggtaccgttcgtatagcatacattatacgaagttattgagctgcgagaa

SEQ ID NO: 92 - DNA, 189 bp, synthetic construct
  misc_feature 1..189: note = Description of Artificial Sequence: Synthetic polynucleotide
  source 1..189: mol_type = other DNA
gctattctcgcagctcaccagttttagagctagaaatagcaagttaaaataaggctagtccgttatcaacttgaaaaagtggcaccgagtcggtgctcgacgacgagcgcggcgatatcatcatccatggccggatgatcctgacgacggagaccgccgtcgtcgacaagccggcctgagctgcgagaa

SEQ ID NO: 93 - DNA, 181 bp, synthetic construct
  misc_feature 1..181: note = Description of Artificial Sequence: Synthetic polynucleotide
  source 1..181: mol_type = other DNA
gctattctcgcagctcaccagttttagagctagaaatagcaagttaaaataaggctagtccgttatcaacttgaaaaagtggcaccgagtcggtgcgagcgcggcgatatcatcatccatggccggatgatcctgacgacggagaccgccgtcgtcgacaagccggcctgagctgcgagaa

SEQ ID NO: 94 - DNA, 178 bp, synthetic construct
  misc_feature 1..178: note = Description of Artificial Sequence: Synthetic polynucleotide
  source 1..178: mol_type = other DNA
gctattctcgcagctcaccagttttagagctagaaatagcaagttaaaataaggctagtccgttatcaacttgaaaaagtggcaccgagtcggtgccgcggcgatatcatcatccatggccggatgatcctgacgacggagaccgccgtcgtcgacaagccggcctgagctgcgagaa

SEQ ID NO: 95 - DNA, 175 bp, synthetic construct
  misc_feature 1..175: note = Description of Artificial Sequence: Synthetic polynucleotide
  source 1..175: mol_type = other DNA
gctattctcgcagctcaccagttttagagctagaaatagcaagttaaaataaggctagtccgttatcaacttgaaaaagtggcaccgagtcggtgcggcgatatcatcatccatggccggatgatcctgacgacggagaccgccgtcgtcgacaagccggcctgagctgcgagaa

SEQ ID NO: 96 - DNA, 171 bp, synthetic construct
  misc_feature 1..171: note = Description of Artificial Sequence: Synthetic polynucleotide
  source 1..171: mol_type = other DNA
gctattctcgcagctcaccagttttagagctagaaatagcaagttaaaataaggctagtccgttatcaacttgaaaaagtggcaccgagtcggtgcatatcatcatccatggccggatgatcctgacgacggagaccgccgtcgtcgacaagccggcctgagctgcgagaa

SEQ ID NO: 97 - DNA, 194 bp, synthetic construct
  misc_feature 1..194: note = Description of Artificial Sequence: Synthetic polynucleotide
  source 1..194: mol_type = other DNA
gctattctcgcagctcaccagttttagagctagaaatagcaagttaaaataaggctagtccgttatcaacttgaaaaagtggcaccgagtcggtgctcgacgacgagcgcggcgatatcatcatccatggccggatgatcctgacgacggagaccgccgtcgtcgacaagccggcctgagctgcgagaatagcc

SEQ ID NO: 98 - DNA, 189 bp, synthetic construct
  misc_feature 1..189: note = Description of Artificial Sequence: Synthetic polynucleotide
  source 1..189: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggccggatgatcctgacgacggagaccgccgtcgtcgacaagccggcctgagctgcgagaatagcc

SEQ ID NO: 99 - DNA, 176 bp, synthetic construct
  misc_feature 1..176: note = Description of Artificial Sequence: Synthetic polynucleotide
  source 1..176: mol_type = other DNA
gctattctcgcagctcaccagttttagagctagaaatagcaagttaaaataaggctagtccgttatcaacttgaaaaagtggcaccgagtcggtgcatatcatcatccatggccggatgatcctgacgacggagaccgccgtcgtcgacaagccggcctgagctgcgagaatagcc

SEQ ID NO: 100 - DNA, 194 bp, synthetic construct
  misc_feature 1..194: note = Description of Artificial Sequence: Synthetic polynucleotide
  source 1..194: mol_type = other DNA
gctgtctccgccgcccgccagttttagagctagaaatagcaagttaaaataaggctagtccgttatcaacttgaaaaagtggcaccgagtcggtgcctgcccatccgcggcggcacgggggtcgcagtcgccatgccggatgatcctgacgacggagaccgccgtcgtcgacaagccggcccgggcggcggaga

SEQ ID NO: 101 - DNA, 189 bp, synthetic construct
  misc_feature 1..189: note = Description of Artificial Sequence: Synthetic polynucleotide
  source 1..189: mol_type = other DNA
gctgtctccgccgcccgccagttttagagctagaaatagcaagttaaaataaggctagtccgttatcaacttgaaaaagtggcaccgagtcggtgccatccgcggcggcacgggggtcgcagtcgccatgccggatgatcctgacgacggagaccgccgtcgtcgacaagccggcccgggcggcggaga

SEQ ID NO: 102 - DNA, 184 bp, synthetic construct
  misc_feature 1..184: note = Description of Artificial Sequence: Synthetic polynucleotide
  source 1..184: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgccggatgatcctgacgacggagaccgccgtcgtcgacaagccggcccgggcggcggaga

SEQ ID NO: 103 - DNA, 179 bp, synthetic construct
  misc_feature 1..179: note = Description of Artificial Sequence: Synthetic polynucleotide
  source 1..179: mol_type = other DNA
gctgtctccgccgcccgccagttttagagctagaaatagcaagttaaaataaggctagtccgttatcaacttgaaaaagtggcaccgagtcggtgcggcacgggggtcgcagtcgccatgccggatgatcctgacgacggagaccgccgtcgtcgacaagccggcccgggcggcggaga

SEQ ID NO: 104 - DNA, 174 bp, synthetic construct
  misc_feature 1..174: note = Description of Artificial Sequence: Synthetic polynucleotide
  source 1..174: mol_type = other DNA
gctgtctccgccgcccgccagttttagagctagaaatagcaagttaaaataaggctagtccgttatcaacttgaaaaagtggcaccgagtcggtgcgggggtcgcagtcgccatgccggatgatcctgacgacggagaccgccgtcgtcgacaagccggcccgggcggcggaga

SEQ ID NO: 105 - DNA, 199 bp, synthetic construct
  misc_feature 1..199: note = Description of Artificial Sequence: Synthetic polynucleotide
  source 1..199: mol_type = other DNA
gctgtctccgccgcccgccagttttagagctagaaatagcaagttaaaataaggctagtccgttatcaacttgaaaaagtggcaccgagtcggtgcctgcccatccgcggcggcacgggggtcgcagtcgccatgccggatgatcctgacgacggagaccgccgtcgtcgacaagccggcccgggcggcggagacagcg

SEQ ID NO: 106 - DNA, 194 bp, synthetic construct
  misc_feature 1..194: note = Description of Artificial Sequence: Synthetic polynucleotide
  source 1..194: mol_type = other DNA
gctgtctccgccgcccgccagttttagagctagaaatagcaagttaaaataaggctagtccgttatcaacttgaaaaagtggcaccgagtcggtgccatccgcggcggcacgggggtcgcagtcgccatgccggatgatcctgacgacggagaccgccgtcgtcgacaagccggcccgggcggcggagacagcg

SEQ ID NO: 107 - DNA, 189 bp, synthetic construct
  misc_feature 1..189: note = Description of Artificial Sequence: Synthetic polynucleotide
  source 1..189: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgccggatgatcctgacgacggagaccgccgtcgtcgacaagccggcccgggcggcggagacagcg

SEQ ID NO: 108 - DNA, 184 bp, synthetic construct
  misc_feature 1..184: note = Description of Artificial Sequence: Synthetic polynucleotide
  source 1..184: mol_type = other DNA
gctgtctccgccgcccgccagttttagagctagaaatagcaagttaaaataaggctagtccgttatcaacttgaaaaagtggcaccgagtcggtgcggcacgggggtcgcagtcgccatgccggatgatcctgacgacggagaccgccgtcgtcgacaagccggcccgggcggcggagacagcg

SEQ ID NO: 109 - DNA, 179 bp, synthetic construct
  misc_feature 1..179: note = Description of Artificial Sequence: Synthetic polynucleotide
  source 1..179: mol_type = other DNA
gctgtctccgccgcccgccagttttagagctagaaatagcaagttaaaataaggctagtccgttatcaacttgaaaaagtggcaccgagtcggtgcgggggtcgcagtcgccatgccggatgatcctgacgacggagaccgccgtcgtcgacaagccggcccgggcggcggagacagcg

SEQ ID NO: 110 - DNA, 96 bp, synthetic construct
  misc_feature 1..96: note = Description of Artificial Sequence: Synthetic oligonucleotide
  source 1..96: mol_type = other DNA
gcgtggtggggccgccagcggttttagagctagaaatagcaagttaaaataaggctagtccgttatcaacttgaaaaagtggcaccgagtcggtgc

SEQ ID NO: 111 - DNA, 180 bp, synthetic construct
  misc_feature 1..180: note = Description of Artificial Sequence: Synthetic polynucleotide
  source 1..180: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggggatgatcctgacgacggagaccgccgtcgtcgacaagccggtgagctgcgagaa

SEQ ID NO: 112 - DNA, 178 bp, synthetic construct
  misc_feature 1..178: note = Description of Artificial Sequence: Synthetic polynucleotide
  source 1..178: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggatgatcctgacgacggagaccgccgtcgtcgacaagccgtgagctgcgagaa

SEQ ID NO: 113 - DNA, 176 bp, synthetic construct
  misc_feature 1..176: note = Description of Artificial Sequence: Synthetic polynucleotide
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggatgatcctgacgacggagaccgccgtcgtcgacaagcctgagctgcgagaa

SEQ ID NO: 114 - DNA, 174 bp, synthetic construct
  misc_feature 1..174: note = Description of Artificial Sequence: Synthetic polynucleotide
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgatcctgacgacggagaccgccgtcgtcgacaagctgagctgcgagaa

SEQ ID NO: 115 - DNA, 182 bp, synthetic construct
  misc_feature 1..182: note = Description of Artificial Sequence: Synthetic polynucleotide
  source 1..182: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgcggatgatcctgacgacggagaccgccgtcgtcgacaagccggccgggcggcggaga

SEQ ID NO: 116 - DNA, 180 bp, synthetic construct
  misc_feature 1..180: note = Description of Artificial Sequence: Synthetic polynucleotide
  source 1..180: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgggatgatcctgacgacggagaccgccgtcgtcgacaagccggcgggcggcggaga

SEQ ID NO: 117 - DNA, 178 bp, synthetic construct
  misc_feature 1..178: note = Description of Artificial Sequence: Synthetic polynucleotide
  source 1..178: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatggatgatcctgacgacggagaccgccgtcgtcgacaagccgcgggcggcggaga

SEQ ID NO: 118 - DNA, 176 bp, synthetic construct
  misc_feature 1..176: note = Description of Artificial Sequence: Synthetic polynucleotide
  source 1..176: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgatgatcctgacgacggagaccgccgtcgtcgacaagcccgggcggcggaga

SEQ ID NO: 119 - DNA, 189 bp, synthetic construct
  misc_feature 1..189: note = Description of Artificial Sequence: Synthetic polynucleotide
  source 1..189: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccccggatgatcctgacgacggagaccgccgtcgtcgacaagccggcctcctccaggcaatacgcg

SEQ ID NO: 120 - DNA, 184 bp, synthetic construct
  misc_feature 1..184: note = Description of Artificial Sequence: Synthetic polynucleotide
  source 1..184: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccccggatgatcctgacgacggagaccgccgtcgtcgacaagccggcctcctccaggcaat

SEQ ID NO: 121 - DNA, 182 bp, synthetic construct
  misc_feature 1..182: note = Description of Artificial Sequence: Synthetic polynucleotide
  source 1..182: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgcccggatgatcctgacgacggagaccgccgtcgtcgacaagccggctcctccaggcaat

SEQ ID NO: 122 - DNA, 180 bp, synthetic construct
  misc_feature 1..180: note = Description of Artificial Sequence: Synthetic polynucleotide
  source 1..180: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccggatgatcctgacgacggagaccgccgtcgtcgacaagccggtcctccaggcaat

SEQ ID NO: 123 - DNA, 178 bp, synthetic construct
  misc_feature 1..178: note = Description of Artificial Sequence: Synthetic polynucleotide
  source 1..178: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccgatgatcctgacgacggagaccgccgtcgtcgacaagccgtcctccaggcaat

SEQ ID NO: 124 - DNA, 176 bp, synthetic construct
  misc_feature 1..176: note = Description of Artificial Sequence: Synthetic polynucleotide
  source 1..176: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccatgatcctgacgacggagaccgccgtcgtcgacaagcctcctccaggcaat

SEQ ID NO: 125 - DNA, 97 bp, synthetic construct
  misc_feature 1..97: note = Description of Artificial Sequence: Synthetic oligonucleotide
  source 1..97: mol_type = other DNA
gagccgagcacgaggggatacgttttagagctagaaatagcaagttaaaataaggctagtccgttatcaacttgaaaaagtggcaccgagtcggtgc

SEQ ID NO: 126 - DNA, 167 bp, synthetic construct
  misc_feature 1..167: note = Description of Artificial Sequence: Synthetic polynucleotide
  source 1..167: mol_type = other DNA
gctattctcgcagctcaccagttttagagctagaaatagcaagttaaaataaggctagtccgttatcaacttgaaaaagtggcaccgagtcggtgcggcgatatcatcatccatggatgatcctgacgacggagaccgccgtcgtcgacaagcctgagctgcgagaa

SEQ ID NO: 127 - DNA, 162 bp, synthetic construct
  misc_feature 1..162: note = Description of Artificial Sequence: Synthetic polynucleotide
  source 1..162: mol_type = other DNA
gctattctcgcagctcaccagttttagagctagaaatagcaagttaaaataaggctagtccgttatcaacttgaaaaagtggcaccgagtcggtgctatcatcatccatggatgatcctgacgacggagaccgccgtcgtcgacaagcctgagctgcgagaa

SEQ ID NO: 128 - DNA, 157 bp, synthetic construct
  misc_feature 1..157: note = Description of Artificial Sequence: Synthetic polynucleotide
  source 1..157: mol_type = other DNA
gctattctcgcagctcaccagttttagagctagaaatagcaagttaaaataaggctagtccgttatcaacttgaaaaagtggcaccgagtcggtgctcatccatggatgatcctgacgacggagaccgccgtcgtcgacaagcctgagctgcgagaa

SEQ ID NO: 129 - DNA, 163 bp, synthetic construct
  misc_feature 1..163: note = Description of Artificial Sequence: Synthetic polynucleotide
  source 1..163: mol_type = other DNA
gctattctcgcagctcaccagttttagagctagaaatagcaagttaaaataaggctagtccgttatcaacttgaaaaagtggcaccgagtcggtgcggcgatatcatcatccatggatgatcctgacgacggagaccgccgtcgtcgacaagcctgagctgcg

SEQ ID NO: 130 - DNA, 158 bp, synthetic construct
  misc_feature 1..158: note = Description of Artificial Sequence: Synthetic polynucleotide
  source 1..158: mol_type = other DNA
gctattctcgcagctcaccagttttagagctagaaatagcaagttaaaataaggctagtccgttatcaacttgaaaaagtggcaccgagtcggtgctatcatcatccatggatgatcctgacgacggagaccgccgtcgtcgacaagcctgagctgcg

SEQ ID NO: 131 - DNA, 153 bp, synthetic construct
  misc_feature 1..153: note = Description of Artificial Sequence: Synthetic polynucleotide
  source 1..153: mol_type = other DNA
gctattctcgcagctcaccagttttagagctagaaatagcaagttaaaataaggctagtccgttatcaacttgaaaaagtggcaccgagtcggtgctcatccatggatgatcctgacgacggagaccgccgtcgtcgacaagcctgagctgcg

SEQ ID NO: 132 - DNA, 167 bp, synthetic construct
  misc_feature 1..167: note = Description of Artificial Sequence: Synthetic polynucleotide
  source 1..167: mol_type = other DNA
gctgtctccgccgcccgccagttttagagctagaaatagcaagttaaaataaggctagtccgttatcaacttgaaaaagtggcaccgagtcggtgccgggggtcgcagtcgccatgatgatcctgacgacggagaccgccgtcgtcgacaagcccgggcggcggaga

SEQ ID NO: 133 - DNA, 162 bp, synthetic construct
  misc_feature 1..162: note = Description of Artificial Sequence: Synthetic polynucleotide
  source 1..162: mol_type = other DNA
gctgtctccgccgcccgccagttttagagctagaaatagcaagttaaaataaggctagtccgttatcaacttgaaaaagtggcaccgagtcggtgcgtcgcagtcgccatgatgatcctgacgacggagaccgccgtcgtcgacaagcccgggcggcggaga

SEQ ID NO: 134 - DNA, 157 bp, synthetic construct
  misc_feature 1..157: note = Description of Artificial Sequence: Synthetic polynucleotide
  source 1..157: mol_type = other DNA
gctgtctccgccgcccgccagttttagagctagaaatagcaagttaaaataaggctagtccgttatcaacttgaaaaagtggcaccgagtcggtgcagtcgccatgatgatcctgacgacggagaccgccgtcgtcgacaagcccgggcggcggaga

SEQ ID NO: 135 - DNA, 163 bp, synthetic construct
  misc_feature 1..163: note = Description of Artificial Sequence: Synthetic polynucleotide
  source 1..163: mol_type = other DNA
gctgtctccgccgcccgccagttttagagctagaaatagcaagttaaaataaggctagtccgttatcaacttgaaaaagtggcaccgagtcggtgccgggggtcgcagtcgccatgatgatcctgacgacggagaccgccgtcgtcgacaagcccgggcggcg

SEQ ID NO: 136 - DNA, 158 bp, synthetic construct
  misc_feature 1..158: note = Description of Artificial Sequence: Synthetic polynucleotide
  source 1..158: mol_type = other DNA
gctgtctccgccgcccgccagttttagagctagaaatagcaagttaaaataaggctagtccgttatcaacttgaaaaagtggcaccgagtcggtgcgtcgcagtcgccatgatgatcctgacgacggagaccgccgtcgtcgacaagcccgggcggcg

SEQ ID NO: 137 - DNA, 153 bp, synthetic construct
  misc_feature 1..153: note = Description of Artificial Sequence: Synthetic polynucleotide
  source 1..153: mol_type = other DNA
gctgtctccgccgcccgccagttttagagctagaaatagcaagttaaaataaggctagtccgttatcaacttgaaaaagtggcaccgagtcggtgcagtcgccatgatgatcctgacgacggagaccgccgtcgtcgacaagcccgggcggcg

SEQ ID NO: 138 - DNA, 180 bp, synthetic construct
  misc_feature 1..180: note = Description of Artificial Sequence: Synthetic polynucleotide
  source 1..180: mol_type = other DNA
gagaagcggcgtccggggctagttttagagctagaaatagcaagttaaaataaggctagtccgttatcaacttgaaaaagtggcaccgagtcggtgctctttgtccagagtcacagccataccggatgatcctgacgacggagaccgccgtcgtcgacaagccggccccccggacgccgc

SEQ ID NO: 139 - DNA, 179 bp, synthetic construct
  misc_feature 1..179: note = Description of Artificial Sequence: Synthetic polynucleotide
  source 1..179: mol_type = other DNA
gggcacggggccatgtacaagttttagagctagaaatagcaagttaaaataaggctagtccgttatcaacttgaaaaagtggcaccgagtcggtgcggcgtcggcagcccgatcccgttgccggatgatcctgacgacggagaccgccgtcgtcgacaagccggcctacatggccccgt

SEQ ID NO: 140 - DNA, 185 bp, synthetic construct
  misc_feature 1..185: note = Description of Artificial Sequence: Synthetic polynucleotide
  source 1..185: mol_type = other DNA
gtgtcaggtggggcggggctagttttagagctagaaatagcaagttaaaataaggctagtccgttatcaacttgaaaaagtggcaccgagtcggtgcgctggctcctcccctggcaccataccggatgatcctgacgacggagaccgccgtcgtcgacaagccggccccccgccccacctgacac

SEQ ID NO: 141 - DNA, 184 bp, synthetic construct
  misc_feature 1..184: note = Description of Artificial Sequence: Synthetic polynucleotide
  source 1..184: mol_type = other DNA
gagtgggtcagacgagcaggagttttagagctagaaatagcaagttaaaataaggctagtccgttatcaacttgaaaaagtggcaccgagtcggtgcgatggagggctgcatgggggaggagtcgccggatgatcctgacgacggagaccgccgtcgtcgacaagccggcctgctcgtctgacc

SEQ ID NO: 142 - DNA, 97 bp, synthetic construct
  misc_feature 1..97: note = Description of Artificial Sequence: Synthetic oligonucleotide
  source 1..97: mol_type = other DNA
gcagccacccgctctcggcccgttttagagctagaaatagcaagttaaaataaggctagtccgttatcaacttgaaaaagtggcaccgagtcggtgc

SEQ ID NO: 143 - DNA, 97 bp, synthetic construct
  misc_feature 1..97: note = Description of Artificial Sequence: Synthetic oligonucleotide
  source 1..97: mol_type = other DNA
gtgtagtcaggccgctcacccgttttagagctagaaatagcaagttaaaataaggctagtccgttatcaacttgaaaaagtggcaccgagtcggtgc

SEQ ID NO: 144 - DNA, 97 bp, synthetic construct
  misc_feature 1..97: note = Description of Artificial Sequence: Synthetic oligonucleotide
  source 1..97: mol_type = other DNA
gctgacaagtctacggaacctgttttagagctagaaatagcaagttaaaataaggctagtccgttatcaacttgaaaaagtggcaccgagtcggtgc

SEQ ID NO: 145 - DNA, 96 bp, synthetic construct
  misc_feature 1..96: note = Description of Artificial Sequence: Synthetic oligonucleotide
  source 1..96: mol_type = other DNA
gctcctccagcgccttgaccgttttagagctagaaatagcaagttaaaataaggctagtccgttatcaacttgaaaaagtggcaccgagtcggtgc

SEQ ID NO: 146 - DNA, 20 bp, synthetic construct
  misc_feature 1..20: note = Description of Artificial Sequence: Synthetic oligonucleotide
  source 1..20: mol_type = other DNA
gctattctcgcagctcacca

SEQ ID NO: 147 - DNA, 20 bp, synthetic construct
  misc_feature 1..20: note = Description of Artificial Sequence: Synthetic oligonucleotide
  source 1..20: mol_type = other DNA
agaagcggcgtccggggcta

SEQ ID NO: 148 - DNA, 20 bp, synthetic construct
  misc_feature 1..20: note = Description of Artificial Sequence: Synthetic oligonucleotide
  source 1..20: mol_type = other DNA
gggcacggggccatgtacaa

SEQ ID NO: 149 - DNA, 20 bp, synthetic construct
  misc_feature 1..20: note = Description of Artificial Sequence: Synthetic oligonucleotide
  source 1..20: mol_type = other DNA
gcgtattgcctggaggatgg

SEQ ID NO: 150 - DNA, 20 bp, synthetic construct
  misc_feature 1..20: note = Description of Artificial Sequence: Synthetic oligonucleotide
  source 1..20: mol_type = other DNA
tgtcaggtggggcggggcta

SEQ ID NO: 151 - DNA, 20 bp, synthetic construct
  misc_feature 1..20: note = Description of Artificial Sequence: Synthetic oligonucleotide
  source 1..20: mol_type = other DNA
agtgggtcagacgagcagga

SEQ ID NO: 152 - DNA, 20 bp, synthetic construct
  misc_feature 1..20: note = Description of Artificial Sequence: Synthetic oligonucleotide
  source 1..20: mol_type = other DNA
gctgtctccgccgcccgcca

SEQ ID NO: 153 - DNA, 96 bp, synthetic construct
  misc_feature 1..96: note = Description of Artificial Sequence: Synthetic oligonucleotide
  source 1..96: mol_type = other DNA
gctattctcgcagctcaccagttttagagctagaaatagcaagttaaaataaggctagtccgttatcaacttgaaaaagtggcaccgagtcggtgc

SEQ ID NO: 154 - DNA, 184 bp, synthetic construct
  misc_feature 1..184: note = Description of Artificial Sequence: Synthetic polynucleotide
  misc_difference 148..149: note = CG, GC, AT, TA, GG, TT, GA, AG, CC, TC, CT, AA, TG, GT, CA, or AC
  source 1..184: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggccggatgatcctgacgacggagnncgccgtcgtcgacaagccggcctgagctgcgagaa

SEQ ID NO: 155 - DNA, 183 bp, synthetic construct
  misc_feature 1..183: note = Description of Artificial Sequence: Synthetic polynucleotide
  source 1..183: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgccggatgatcctgacgacggagaccgccgtcgtcgacaagccggcctgagctgcgagaa

SEQ ID NO: 156 - DNA, 183 bp, synthetic construct
  misc_feature 1..183: note = Description of Artificial Sequence: Synthetic polynucleotide
  source 1..183: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgccggatgatcctgacgacggagagcgccgtcgtcgacaagccggcctgagctgcgagaa

SEQ ID NO: 157 - DNA, 189 bp, synthetic construct
  misc_feature 1..189: note = Description of Artificial Sequence: Synthetic polynucleotide
  source 1..189: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccccggatgatcctgacgacggagtccgccgtcgtcgacaagccggcctcctccaggcaatacgcg

SEQ ID NO: 158 - DNA, 189 bp, synthetic construct
  misc_feature 1..189: note = Description of Artificial Sequence: Synthetic polynucleotide
  source 1..189: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgccggatgatcctgacgacggagctcgccgtcgtcgacaagccggcccgggcggcggagacagcg

SEQ ID NO: 159 - DNA, 20 bp, synthetic construct
  misc_feature 1..20: note = Description of Artificial Sequence: Synthetic oligonucleotide
  source 1..20: mol_type = other DNA
gtcacctccaatgactaggg

SEQ ID NO: 160 - DNA, 20 bp, synthetic construct
  misc_feature 1..20: note = Description of Artificial Sequence: Synthetic oligonucleotide
  source 1..20: mol_type = other DNA
gggcaaccacaaacccacga

SEQ ID NO: 161 - DNA, 194 bp, synthetic construct
  misc_feature 1..194: note = Description of Artificial Sequence: Synthetic polynucleotide
  source 1..194: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggctatgccggatgatcctgacgacggagtccgccgtcgtcgacaagccggccctagctgagctgcgagaa

SEQ ID NO: 162 - DNA, 189 bp, synthetic construct
  misc_feature 1..189: note = Description of Artificial Sequence: Synthetic polynucleotide
  source 1..189: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgccggatgatcctgacgacggagtccgccgtcgtcgacaagccggccctatgagctgcgagaa

SEQ ID NO: 163 - DNA, 184 bp, synthetic construct
  misc_feature 1..184: note = Description of Artificial Sequence: Synthetic polynucleotide
  source 1..184: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggccggatgatcctgacgacggagtccgccgtcgtcgacaagccggcctgagctgcgagaa

SEQ ID NO: 164 - DNA, 179 bp, synthetic construct
  misc_feature 1..179: note = Description of Artificial Sequence: Synthetic polynucleotide
  source 1..179: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggggatgatcctgacgacggagtccgccgtcgtcgacaagccgtgagctgcgagaa

SEQ ID NO: 165 - DNA, 174 bp, synthetic construct
  misc_feature 1..174: note = Description of Artificial Sequence: Synthetic polynucleotide
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgatcctgacgacggagtccgccgtcgtcgacaagctgagctgcgagaa

SEQ ID NO: 166 - DNA, 169 bp, synthetic construct
  misc_feature 1..169: note = Description of Artificial Sequence: Synthetic polynucleotide
  source 1..169: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggatcctgacgacggagtccgccgtcgtcgacatgagctgcgagaa

SEQ ID NO: 167 - DNA, 164 bp, synthetic construct
  misc_feature 1..164: note = Description of Artificial Sequence: Synthetic polynucleotide
  source 1..164: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcctgacgacggagtccgccgtcgtcgtgagctgcgagaa

SEQ ID NO: 168 - DNA, 159 bp, synthetic construct
  misc_feature 1..159: note = Description of Artificial Sequence: Synthetic polynucleotide
  source 1..159: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgacgacggagtccgccgtcgtgagctgcgagaa

SEQ ID NO: 169 - DNA, 154 bp, synthetic construct
  misc_feature 1..154: note = Description of Artificial Sequence: Synthetic polynucleotide
  source 1..154: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggacgacggagtccgccgtgagctgcgagaa

SEQ ID NO: 170 - DNA, 149 bp, synthetic construct
  misc_feature 1..149: note = Description of Artificial Sequence: Synthetic polynucleotide
  source 1..149: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggacggagtccgtgagctgcgagaa

SEQ ID NO: 171 - DNA, 144 bp, synthetic construct
  misc_feature 1..144: note = Description of Artificial Sequence: Synthetic polynucleotide
  source 1..144: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcggagttgagctgcgagaa

SEQ ID NO: 172 - DNA, 182 bp, synthetic construct
  misc_feature 1..182: note = Description of Artificial Sequence: Synthetic polynucleotide
  source 1..182: mol_type = other DNA
gaagccggccttgcacatgcgttttagagctagaaatagcaagttaaaataaggctagtccgttatcaacttgaaaaagtggcaccgagtcggtgctcgacgacgagcgcggcgatatcatcatccatggtaccgttcgtatagcatacattatacgaagttattgagctgcgagaatagcc

SEQ ID NO: 173 - DNA, 177 bp, synthetic construct
  misc_feature 1..177: note = Description of Artificial Sequence: Synthetic polynucleotide
  source 1..177: mol_type = other DNA
gaagccggccttgcacatgcgttttagagctagaaatagcaagttaaaataaggctagtccgttatcaacttgaaaaagtggcaccgagtcggtgcgacgagcgcggcgatatcatcatccatggtaccgttcgtatagcatacattatacgaagttattgagctgcgagaatagcc

SEQ ID NO: 174 - DNA, 177 bp, synthetic construct
  misc_feature 1..177: note = Description of Artificial Sequence: Synthetic polynucleotide
  source 1..177: mol_type = other DNA
gaagccggccttgcacatgcgttttagagctagaaatagcaagttaaaataaggctagtccgttatcaacttgaaaaagtggcaccgagtcggtgctcgacgacgagcgcggcgatatcatcatccatggtaccgttcgtatagcatacattatacgaagttattgagctgcgagaa

SEQ ID NO: 175 - DNA, 159 bp, synthetic construct
  misc_feature 1..159: note = Description of Artificial Sequence: Synthetic polynucleotide
  source 1..159: mol_type = other DNA
gaagccggccttgcacatgcgttttagagctagaaatagcaagttaaaataaggctagtccgttatcaacttgaaaaagtggcaccgagtcggtgcatatcatcatccatggtaccgttcgtatagcatacattatacgaagttattgagctgcgagaa

SEQ ID NO: 176 - DNA, 96 bp, synthetic construct
  misc_feature 1..96: note = Description of Artificial Sequence: Synthetic oligonucleotide
  source 1..96: mol_type = other DNA
ccccacgatggaggggaagagttttagagctagaaatagcaagttaaaataaggctagtccgttatcaacttgaaaaagtggcaccgagtcggtgc

SEQ ID NO: 177 - DNA, 96 bp, synthetic construct
  misc_feature 1..96: note = Description of Artificial Sequence: Synthetic oligonucleotide
  source 1..96: mol_type = other DNA
ccttctcctggagccgcgacgttttagagctagaaatagcaagttaaaataaggctagtccgttatcaacttgaaaaagtggcaccgagtcggtgc

SEQ ID NO: 178 - DNA, 52 bp, synthetic construct; identical to SEQ ID NO: 16
  misc_feature 1..52: note = Description of Artificial Sequence: Synthetic oligonucleotide
  source 1..52: mol_type = other DNA

SEQ ID NO: 179 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgagaccgcggtggttgaccagacaaaccac

SEQ ID NO: 180 - DNA, 52 bp, synthetic construct; identical to SEQ ID NO: 34
  misc_feature 1..52: note = Description of Artificial Sequence: Synthetic oligonucleotide
  source 1..52: mol_type = other DNA

SEQ ID NO: 181 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgagcgcgcggtggttgaccagacaaaccac

SEQ ID NO: 182 - DNA, 52 bp, synthetic construct; identical to SEQ ID NO: 32
  misc_feature 1..52: note = Description of Artificial Sequence: Synthetic oligonucleotide
  source 1..52: mol_type = other DNA

SEQ ID NO: 183 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgaggccgcggtggttgaccagacaaaccac

SEQ ID NO: 184 - DNA, 52 bp, synthetic construct; identical to SEQ ID NO: 30
  misc_feature 1..52: note = Description of Artificial Sequence: Synthetic oligonucleotide
  source 1..52: mol_type = other DNA

SEQ ID NO: 185 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgagatcgcggtggttgaccagacaaaccac

SEQ ID NO: 186 - DNA, 52 bp, synthetic construct; identical to SEQ ID NO: 36
  misc_feature 1..52: note = Description of Artificial Sequence: Synthetic oligonucleotide
  source 1..52: mol_type = other DNA

SEQ ID NO: 187 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgagtacgcggtggttgaccagacaaaccac

SEQ ID NO: 188 - DNA, 52 bp, synthetic construct; identical to SEQ ID NO: 12
  misc_feature 1..52: note = Description of Artificial Sequence: Synthetic oligonucleotide
  source 1..52: mol_type = other DNA

SEQ ID NO: 189 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgagcccgcggtggttgaccagacaaaccac

SEQ ID NO: 190 - DNA, 52 bp, synthetic construct; identical to SEQ ID NO: 6
  misc_feature 1..52: note = Description of Artificial Sequence: Synthetic oligonucleotide
  source 1..52: mol_type = other DNA

SEQ ID NO: 191 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgagaacgcggtggttgaccagacaaaccac

SEQ ID NO: 192 - DNA, 52 bp, synthetic construct; identical to SEQ ID NO: 24
  misc_feature 1..52: note = Description of Artificial Sequence: Synthetic oligonucleotide
  source 1..52: mol_type = other DNA

SEQ ID NO: 193 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgagtccgcggtggttgaccagacaaaccac

SEQ ID NO: 194 - DNA, 52 bp, synthetic construct; identical to SEQ ID NO: 26
  misc_feature 1..52: note = Description of Artificial Sequence: Synthetic oligonucleotide
  source 1..52: mol_type = other DNA

SEQ ID NO: 195 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgagctcgcggtggttgaccagacaaaccac

SEQ ID NO: 196 - DNA, 52 bp, synthetic construct; identical to SEQ ID NO: 10
  misc_feature 1..52: note = Description of Artificial Sequence: Synthetic oligonucleotide
  source 1..52: mol_type = other DNA

SEQ ID NO: 197 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgagggcgcggtggttgaccagacaaaccac

SEQ ID NO: 198 - DNA, 52 bp, synthetic construct; identical to SEQ ID NO: 22
  misc_feature 1..52: note = Description of Artificial Sequence: Synthetic oligonucleotide
  source 1..52: mol_type = other DNA

SEQ ID NO: 199 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgaggacgcggtggttgaccagacaaaccac

SEQ ID NO: 200 - DNA, 52 bp, synthetic construct; identical to SEQ ID NO: 18
  misc_feature 1..52: note = Description of Artificial Sequence: Synthetic oligonucleotide
  source 1..52: mol_type = other DNA

SEQ ID NO: 201 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgagagcgcggtggttgaccagacaaaccac

SEQ ID NO: 202 - DNA, 52 bp, synthetic construct; identical to SEQ ID NO: 8
  misc_feature 1..52: note = Description of Artificial Sequence: Synthetic oligonucleotide
  source 1..52: mol_type = other DNA

SEQ ID NO: 203 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgagttcgcggtggttgaccagacaaaccac

SEQ ID NO: 204 - DNA, 52 bp, synthetic construct; identical to SEQ ID NO: 20
  misc_feature 1..52: note = Description of Artificial Sequence: Synthetic oligonucleotide
  source 1..52: mol_type = other DNA

SEQ ID NO: 205 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgagtgcgcggtggttgaccagacaaaccac

SEQ ID NO: 206 - DNA, 52 bp, synthetic construct; identical to SEQ ID NO: 28
  misc_feature 1..52: note = Description of Artificial Sequence: Synthetic oligonucleotide
  source 1..52: mol_type = other DNA

SEQ ID NO: 207 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgaggtcgcggtggttgaccagacaaaccac

SEQ ID NO: 208 - DNA, 52 bp, synthetic construct; identical to SEQ ID NO: 14
  misc_feature 1..52: note = Description of Artificial Sequence: Synthetic oligonucleotide
  source 1..52: mol_type = other DNA

SEQ ID NO: 209 - DNA, 52 bp, synthetic construct
  misc_feature 1..52: note = Description of Artificial Sequence: Synthetic oligonucleotide
  source 1..52: mol_type = other DNA
tgggtttgtaccgtacaccactgagcacgcggtggttgaccagacaaaccac

SEQ ID NO: 210 - DNA, 46 bp, synthetic construct; identical to SEQ ID NO: 3
  misc_feature 1..46: note = Description of Artificial Sequence: Synthetic oligonucleotide
  source 1..46: mol_type = other DNA

SEQ ID NO: 211 - DNA, 46 bp, synthetic construct; identical to SEQ ID NO: 4
  misc_feature 1..46: note = Description of Artificial Sequence: Synthetic oligonucleotide
  source 1..46: mol_type = other DNA

SEQ ID NO: 212 - DNA, 46 bp, synthetic construct
  misc_feature 1..46: note = Description of Artificial Sequence: Synthetic oligonucleotide
  source 1..46: mol_type = other DNA
ggccggcttgtcgacgacggcgaactccgtcgtcaggatcatccgg

SEQ ID NO: 213 - DNA, 46 bp, synthetic construct
  misc_feature 1..46: note = Description of Artificial Sequence: Synthetic oligonucleotide
  source 1..46: mol_type = other DNA
ccggatgatcctgacgacggagttcgccgtcgtcgacaagccggcc

SEQ ID NO: 214 - DNA, 46 bp, synthetic construct
  misc_feature 1..46: note = Description of Artificial Sequence: Synthetic oligonucleotide
  source 1..46: mol_type = other DNA
ggccggcttgtcgacgacggcggactccgtcgtcaggatcatccgg

SEQ ID NO: 215 - DNA, 46 bp, synthetic construct
  misc_feature 1..46: note = Description of Artificial Sequence: Synthetic oligonucleotide
  source 1..46: mol_type = other DNA
ccggatgatcctgacgacggagtccgccgtcgtcgacaagccggcc

SEQ ID NO: 216 - DNA, 46 bp, synthetic construct
  misc_feature 1..46: note = Description of Artificial Sequence: Synthetic oligonucleotide
  source 1..46: mol_type = other DNA
ggccggcttgtcgacgacggcgcactccgtcgtcaggatcatccgg

SEQ ID NO: 217 - DNA, 46 bp, synthetic construct
  misc_feature 1..46: note = Description of Artificial Sequence: Synthetic oligonucleotide
  source 1..46: mol_type = other DNA
ccggatgatcctgacgacggagtgcgccgtcgtcgacaagccggcc

SEQ ID NO: 218 - DNA, 46 bp, synthetic construct
  misc_feature 1..46: note = Description of Artificial Sequence: Synthetic oligonucleotide
  source 1..46: mol_type = other DNA
ggccggcttgtcgacgacggcgtactccgtcgtcaggatcatccgg

SEQ ID NO: 219 - DNA, 46 bp, synthetic construct
  misc_feature 1..46: note = Description of Artificial Sequence: Synthetic oligonucleotide
  source 1..46: mol_type = other DNA
ccggatgatcctgacgacggagtacgccgtcgtcgacaagccggcc

SEQ ID NO: 220 - DNA, 46 bp, synthetic construct
  misc_feature 1..46: note = Description of Artificial Sequence: Synthetic oligonucleotide
  source 1..46: mol_type = other DNA
ggccggcttgtcgacgacggcgagctccgtcgtcaggatcatccgg

SEQ ID NO: 221 - DNA, 46 bp, synthetic construct
  misc_feature 1..46: note = Description of Artificial Sequence: Synthetic oligonucleotide
  source 1..46: mol_type = other DNA
ccggatgatcctgacgacggagctcgccgtcgtcgacaagccggcc

SEQ ID NO: 222 - DNA, 46 bp, synthetic construct
  misc_feature 1..46: note = Description of Artificial Sequence: Synthetic oligonucleotide
  source 1..46: mol_type = other DNA
ggccggcttgtcgacgacggcgggctccgtcgtcaggatcatccgg

SEQ ID NO: 223 - DNA, 46 bp, synthetic construct
  misc_feature 1..46: note = Description of Artificial Sequence: Synthetic oligonucleotide
  source 1..46: mol_type = other DNA
ccggatgatcctgacgacggagcccgccgtcgtcgacaagccggcc

SEQ ID NO: 224 - DNA, 46 bp, synthetic construct
  misc_feature 1..46: note = Description of Artificial Sequence: Synthetic oligonucleotide
  source 1..46: mol_type = other DNA
ggccggcttgtcgacgacggcgcgctccgtcgtcaggatcatccgg

SEQ ID NO: 225 - DNA, 46 bp, synthetic construct
  misc_feature 1..46: note = Description of Artificial Sequence: Synthetic oligonucleotide
  source 1..46: mol_type = other DNA
ccggatgatcctgacgacggagcgcgccgtcgtcgacaagccggcc

SEQ ID NO: 226 - DNA, 46 bp, synthetic construct
  misc_feature 1..46: note = Description of Artificial Sequence: Synthetic oligonucleotide
  source 1..46: mol_type = other DNA
ggccggcttgtcgacgacggcgtgctccgtcgtcaggatcatccgg

SEQ ID NO: 227 - DNA, 46 bp, synthetic construct
  misc_feature 1..46: note = Description of Artificial Sequence: Synthetic oligonucleotide
  source 1..46: mol_type = other DNA
ccggatgatcctgacgacggagcacgccgtcgtcgacaagccggcc

SEQ ID NO: 228 - DNA, 46 bp, synthetic construct
  misc_feature 1..46: note = Description of Artificial Sequence: Synthetic oligonucleotide
  source 1..46: mol_type = other DNA
ggccggcttgtcgacgacggcgacctccgtcgtcaggatcatccgg

SEQ ID NO: 229 - DNA, 46 bp, synthetic construct
  misc_feature 1..46: note = Description of Artificial Sequence: Synthetic oligonucleotide
  source 1..46: mol_type = other DNA
ccggatgatcctgacgacggaggtcgccgtcgtcgacaagccggcc

SEQ ID NO: 230 - DNA, 46 bp, synthetic construct
  misc_feature 1..46: note = Description of Artificial Sequence: Synthetic oligonucleotide
  source 1..46: mol_type = other DNA
ggccggcttgtcgacgacggcggcctccgtcgtcaggatcatccgg

SEQ ID NO: 231 - DNA, 46 bp, synthetic construct
  misc_feature 1..46: note = Description of Artificial Sequence: Synthetic oligonucleotide
  source 1..46: mol_type = other DNA
ccggatgatcctgacgacggaggccgccgtcgtcgacaagccggcc

SEQ ID NO: 232 - DNA, 46 bp, synthetic construct
  misc_feature 1..46: note = Description of Artificial Sequence: Synthetic oligonucleotide
  source 1..46: mol_type = other DNA
ggccggcttgtcgacgacggcgccctccgtcgtcaggatcatccgg

SEQ ID NO: 233 - DNA, 46 bp, synthetic construct
  misc_feature 1..46: note = Description of Artificial Sequence: Synthetic oligonucleotide
  source 1..46: mol_type = other DNA
ccggatgatcctgacgacggagggcgccgtcgtcgacaagccggcc

SEQ ID NO: 234 - DNA, 46 bp, synthetic construct
  misc_feature 1..46: note = Description of Artificial Sequence: Synthetic oligonucleotide
  source 1..46: mol_type = other DNA
ggccggcttgtcgacgacggcgtcctccgtcgtcaggatcatccgg

SEQ ID NO: 235 - DNA, 46 bp, synthetic construct
  misc_feature 1..46: note = Description of Artificial Sequence: Synthetic oligonucleotide
  source 1..46: mol_type = other DNA
ccggatgatcctgacgacggaggacgccgtcgtcgacaagccggcc

SEQ ID NO: 236 - DNA, 46 bp, synthetic construct
  misc_feature 1..46: note = Description of Artificial Sequence: Synthetic oligonucleotide
  source 1..46: mol_type = other DNA
ggccggcttgtcgacgacggcgatctccgtcgtcaggatcatccgg

SEQ ID NO: 237 - DNA, 46 bp, synthetic construct
  misc_feature 1..46: note = Description of Artificial Sequence: Synthetic oligonucleotide
  source 1..46: mol_type = other DNA
ccggatgatcctgacgacggagatcgccgtcgtcgacaagccggcc

SEQ ID NO: 238 - DNA, 46 bp, synthetic construct
  misc_feature 1..46: note = Description of Artificial Sequence: Synthetic oligonucleotide
  source 1..46: mol_type = other DNA
ggccggcttgtcgacgacggcgctctccgtcgtcaggatcatccgg

SEQ ID NO: 239 - DNA, 46 bp, synthetic construct
  misc_feature 1..46: note = Description of Artificial Sequence: Synthetic oligonucleotide
  source 1..46: mol_type = other DNA
ccggatgatcctgacgacggagagcgccgtcgtcgacaagccggcc

SEQ ID NO: 240 - DNA, 46 bp, synthetic construct
  misc_feature 1..46: note = Description of Artificial Sequence: Synthetic oligonucleotide
  source 1..46: mol_type = other DNA
ggccggcttgtcgacgacggcgttctccgtcgtcaggatcatccgg

SEQ ID NO: 241 - DNA, 46 bp, synthetic construct
  misc_feature 1..46: note = Description of Artificial Sequence: Synthetic oligonucleotide
  source 1..46: mol_type = other DNA
ccggatgatcctgacgacggagaacgccgtcgtcgacaagccggcc

SEQ ID NO: 242 - DNA, 38 bp, synthetic construct; identical to SEQ ID NO: 15
  misc_feature 1..38: note = Description of Artificial Sequence: Synthetic oligonucleotide
  source 1..38: mol_type = other DNA

SEQ ID NO: 243 - DNA, 38 bp, synthetic construct
  misc_feature 1..38: note = Description of Artificial Sequence: Synthetic oligonucleotide
  source 1..38: mol_type = other DNA
atgatcctgacgacggagaccgccgtcgtcgacaagcc

SEQ ID NO: 244 - DNA, 38 bp, synthetic construct; identical to SEQ ID NO: 7
  misc_feature 1..38: note = Description of Artificial Sequence: Synthetic oligonucleotide
  source 1..38: mol_type = other DNA

SEQ ID NO: 245 - DNA, 38 bp, synthetic construct
  misc_feature 1..38: note = Description of Artificial Sequence: Synthetic oligonucleotide
  source 1..38: mol_type = other DNA
atgatcctgacgacggagttcgccgtcgtcgacaagcc

SEQ ID NO: 246 - DNA, 38 bp, synthetic construct; identical to SEQ ID NO: 23
  misc_feature 1..38: note = Description of Artificial Sequence: Synthetic oligonucleotide
  source 1..38: mol_type = other DNA

SEQ ID NO: 247 - DNA, 38 bp, synthetic construct
  misc_feature 1..38: note = Description of Artificial Sequence: Synthetic oligonucleotide
  source 1..38: mol_type = other DNA
atgatcctgacgacggagtccgccgtcgtcgacaagcc

SEQ ID NO: 248 - DNA, 38 bp, synthetic construct; identical to SEQ ID NO: 19
  misc_feature 1..38: note = Description of Artificial Sequence: Synthetic oligonucleotide
  source 1..38: mol_type = other DNA

SEQ ID NO: 249 - DNA, 38 bp, synthetic construct
  misc_feature 1..38: note = Description of Artificial Sequence: Synthetic oligonucleotide
  source 1..38: mol_type = other DNA
atgatcctgacgacggagtgcgccgtcgtcgacaagcc

SEQ ID NO: 250 - DNA, 38 bp, synthetic construct; identical to SEQ ID NO: 35
  misc_feature 1..38: note = Description of Artificial Sequence: Synthetic oligonucleotide
  source 1..38: mol_type = other DNA

SEQ ID NO: 251 - DNA, 38 bp, synthetic construct
  misc_feature 1..38: note = Description of Artificial Sequence: Synthetic oligonucleotide
  source 1..38: mol_type = other DNA
atgatcctgacgacggagtacgccgtcgtcgacaagcc

SEQ ID NO: 252 - DNA, 38 bp, synthetic construct; identical to SEQ ID NO: 25
  misc_feature 1..38: note = Description of Artificial Sequence: Synthetic oligonucleotide
  source 1..38: mol_type = other DNA

SEQ ID NO: 253 - DNA, 38 bp, synthetic construct
  misc_feature 1..38: note = Description of Artificial Sequence: Synthetic oligonucleotide
  source 1..38: mol_type = other DNA
atgatcctgacgacggagctcgccgtcgtcgacaagcc

SEQ ID NO: 254 - DNA, 38 bp, synthetic construct; identical to SEQ ID NO: 11
  misc_feature 1..38: note = Description of Artificial Sequence: Synthetic oligonucleotide
  source 1..38: mol_type = other DNA

SEQ ID NO: 255 - DNA, 38 bp, synthetic construct
  misc_feature 1..38: note = Description of Artificial Sequence: Synthetic oligonucleotide
  source 1..38: mol_type = other DNA
atgatcctgacgacggagcccgccgtcgtcgacaagcc

SEQ ID NO: 256 - DNA, 38 bp, synthetic construct; identical to SEQ ID NO: 33
  misc_feature 1..38: note = Description of Artificial Sequence: Synthetic oligonucleotide
  source 1..38: mol_type = other DNA

SEQ ID NO: 257 - DNA, 38 bp, synthetic construct
  misc_feature 1..38: note = Description of Artificial Sequence: Synthetic oligonucleotide
  source 1..38: mol_type = other DNA
atgatcctgacgacggagcgcgccgtcgtcgacaagcc

SEQ ID NO: 258 - DNA, 38 bp, synthetic construct; identical to SEQ ID NO: 13
  misc_feature 1..38: note = Description of Artificial Sequence: Synthetic oligonucleotide
  source 1..38: mol_type = other DNA

SEQ ID NO: 259 - DNA, 38 bp, synthetic construct
  misc_feature 1..38: note = Description of Artificial Sequence: Synthetic oligonucleotide
  source 1..38: mol_type = other DNA
atgatcctgacgacggagcacgccgtcgtcgacaagcc

SEQ ID NO: 260 - DNA, 38 bp, synthetic construct; identical to SEQ ID NO: 27
  misc_feature 1..38: note = Description of Artificial Sequence: Synthetic oligonucleotide
  source 1..38: mol_type = other DNA

SEQ ID NO: 261 - DNA, 38 bp, synthetic construct
  misc_feature 1..38: note = Description of Artificial Sequence: Synthetic oligonucleotide
  source 1..38: mol_type = other DNA
atgatcctgacgacggaggtcgccgtcgtcgacaagcc

SEQ ID NO: 262 - DNA, 38 bp, synthetic construct; identical to SEQ ID NO: 31
  misc_feature 1..38: note = Description of Artificial Sequence: Synthetic oligonucleotide
  source 1..38: mol_type = other DNA

SEQ ID NO: 263 - DNA, 38 bp, synthetic construct
  misc_feature 1..38: note = Description of Artificial Sequence: Synthetic oligonucleotide
  source 1..38: mol_type = other DNA
atgatcctgacgacggaggccgccgtcgtcgacaagcc

SEQ ID NO: 264 - DNA, 38 bp, synthetic construct; identical to SEQ ID NO: 9
  misc_feature 1..38: note = Description of Artificial Sequence: Synthetic oligonucleotide
  source 1..38: mol_type = other DNA

SEQ ID NO: 265 - DNA, 38 bp, synthetic construct
  misc_feature 1..38: note = Description of Artificial Sequence: Synthetic oligonucleotide
  source 1..38: mol_type = other DNA
atgatcctgacgacggagggcgccgtcgtcgacaagcc

SEQ ID NO: 266 - DNA, 38 bp, synthetic construct; identical to SEQ ID NO: 21
  misc_feature 1..38: note = Description of Artificial Sequence: Synthetic oligonucleotide
  source 1..38: mol_type = other DNA

SEQ ID NO: 267 - DNA, 38 bp, synthetic construct
  misc_feature 1..38: note = Description of Artificial Sequence: Synthetic oligonucleotide
  source 1..38: mol_type = other DNA
atgatcctgacgacggaggacgccgtcgtcgacaagcc

SEQ ID NO: 268 - DNA, 38 bp, synthetic construct; identical to SEQ ID NO: 29
  misc_feature 1..38: note = Description of Artificial Sequence: Synthetic oligonucleotide
  source 1..38: mol_type = other DNA

SEQ ID NO: 269 - DNA, 38 bp, synthetic construct
  misc_feature 1..38: note = Description of Artificial Sequence: Synthetic oligonucleotide
  source 1..38: mol_type = other DNA
atgatcctgacgacggagatcgccgtcgtcgacaagcc

SEQ ID NO: 270 - DNA, 38 bp, synthetic construct; identical to SEQ ID NO: 17
  misc_feature 1..38: note = Description of Artificial Sequence: Synthetic oligonucleotide
  source 1..38: mol_type = other DNA

SEQ ID NO: 271 - DNA, 38 bp, synthetic construct
  misc_feature 1..38: note = Description of Artificial Sequence: Synthetic oligonucleotide
  source 1..38: mol_type = other DNA
atgatcctgacgacggagagcgccgtcgtcgacaagcc

SEQ ID NO: 272 - DNA, 38 bp, synthetic construct; identical to SEQ ID NO: 5
  misc_feature 1..38: note = Description of Artificial Sequence: Synthetic oligonucleotide
  source 1..38: mol_type = other DNA

SEQ ID NO: 273 - DNA, 38 bp, synthetic construct
  misc_feature 1..38: note = Description of Artificial Sequence: Synthetic oligonucleotide
  source 1..38: mol_type = other DNA
atgatcctgacgacggagaacgccgtcgtcgacaagcc

SEQ ID NO: 274 - DNA, 34 bp, synthetic construct; identical to SEQ ID NO: 2
  misc_feature 1..34: note = Description of Artificial Sequence: Synthetic oligonucleotide
  source 1..34: mol_type = other DNA

SEQ ID NO: 275 - DNA, 34 bp, synthetic construct
  misc_feature 1..34: note = Description of Artificial Sequence: Synthetic oligonucleotide
  source 1..34: mol_type = other DNA
ataacttcgtatagcatacattatacgaacggta

SEQ ID NO: 276 - DNA, 34 bp, synthetic construct; identical to SEQ ID NO: 1
  misc_feature 1..34: note = Description of Artificial Sequence: Synthetic oligonucleotide
  source 1..34: mol_type = other DNA

SEQ ID NO: 277 - DNA, 34 bp, synthetic construct
  misc_feature 1..34: note = Description of Artificial Sequence: Synthetic oligonucleotide
  source 1..34: mol_type = other DNA
taccgttcgtatagcatacattatacgaagttat

SEQ ID NO: 278 - DNA, 27 bp, synthetic construct
  misc_feature 1..27: note = Description of Artificial Sequence: Synthetic oligonucleotide
  source 1..27: mol_type = other DNA
tttaccttgattgagatgttaattgtg

SEQ ID NO: 279 - DNA, 27 bp, synthetic construct
  misc_feature 1..27: note = Description of Artificial Sequence: Synthetic oligonucleotide
  source 1..27: mol_type = other DNA
cacaattaacatctcaatcaaggtaaa

SEQ ID NO: 280 - DNA, 50 bp, synthetic construct
  misc_feature 1..50: note = Description of Artificial Sequence: Synthetic oligonucleotide
  source 1..50: mol_type = other DNA
gcgagtttttatttcgtttatttcaattaaggtaactaaaaaactccttt

SEQ ID NO: 281 - DNA, 50 bp, synthetic construct
  misc_feature 1..50: note = Description of Artificial Sequence: Synthetic oligonucleotide
  source 1..50: mol_type = other DNA
aaaggagttttttagttaccttaattgaaataaacgaaataaaaactcgc

SEQ ID NO: 282 - DNA, 34 bp, synthetic construct
  misc_feature 1..34: note = Description of Artificial Sequence: Synthetic oligonucleotide
  source 1..34: mol_type = other DNA
ctggatcatctggatcactttcgtcaaaaacctg

SEQ ID NO: 283 - DNA, 34 bp, synthetic construct
  misc_feature 1..34: note = Description of Artificial Sequence: Synthetic oligonucleotide
  source 1..34: mol_type = other DNA
caggtttttgacgaaagtgatccagatgatccag

SEQ ID NO: 284 - DNA, 57 bp, synthetic construct
  misc_feature 1..57: note = Description of Artificial Sequence: Synthetic oligonucleotide
  source 1..57: mol_type = other DNA
ttcgggtgctgggttgttgtctctggacagtgatccatgggaaactactcagcacca

SEQ ID NO: 285 - DNA, 57 bp, synthetic construct
  misc_feature 1..57: note = Description of Artificial Sequence: Synthetic oligonucleotide
  source 1..57: mol_type = other DNA
tggtgctgagtagtttcccatggatcactgtccagagacaacaacccagcacccgaa

SEQ ID NO: 286 - DNA, 24 bp, synthetic construct
  misc_feature 1..24: note = Description of Artificial Sequence: Synthetic oligonucleotide
  source 1..24: mol_type = other DNA
aaaagtgtgggctgcaggatctga

SEQ ID NO: 287 - DNA, 22 bp, synthetic construct
  misc_feature 1..22: note = Description of Artificial Sequence: Synthetic oligonucleotide
  source 1..22: mol_type = other DNA
ggagctggcagctgtcaatgcc

SEQ ID NO: 288 - DNA, 21 bp, synthetic construct
  misc_feature 1..21: note = Description of Artificial Sequence: Synthetic oligonucleotide
  source 1..21: mol_type = other DNA
agtcaatgccgctctcgtgga

SEQ ID NO: 289 - DNA, 21 bp, synthetic construct
  misc_feature 1..21: note = Description of Artificial Sequence: Synthetic oligonucleotide
  source 1..21: mol_type = other DNA
cagcgggctcagctgatagca

SEQ ID NO: 290 - DNA, 21 bp, synthetic construct
  misc_feature 1..21: note = Description of Artificial Sequence: Synthetic oligonucleotide
  source 1..21: mol_type = other DNA
cggatggctaaccaagcggcc

SEQ ID NO: 291 - DNA, 17 bp, synthetic construct
  misc_feature 1..17: note = Description of Artificial Sequence: Synthetic primer
  source 1..17: mol_type = other DNA
cccggcttcctttgtcc

SEQ ID NO: 292 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 293 - DNA, 17 bp, synthetic construct
  misc_feature 1..17: note = Description of Artificial Sequence: Synthetic primer
  source 1..17: mol_type = other DNA
cccggcttcctttgtcc

SEQ ID NO: 294 - DNA, 22 bp, synthetic construct
  misc_feature 1..22: note = Description of Artificial Sequence: Synthetic primer
  source 1..22: mol_type = other DNA
aaccacaactagaatgcagtga

SEQ ID NO: 295 - DNA, 17 bp, synthetic construct
  misc_feature 1..17: note = Description of Artificial Sequence: Synthetic primer
  source 1..17: mol_type = other DNA
cccggcttcctttgtcc

SEQ ID NO: 296 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 297 - DNA, 17 bp, synthetic construct
  misc_feature 1..17: note = Description of Artificial Sequence: Synthetic primer
  source 1..17: mol_type = other DNA
cccggcttcctttgtcc

SEQ ID NO: 298 - DNA, 22 bp, synthetic construct
  misc_feature 1..22: note = Description of Artificial Sequence: Synthetic primer
  source 1..22: mol_type = other DNA
aaccacaactagaatgcagtga

SEQ ID NO: 299 - DNA, 17 bp, synthetic construct
  misc_feature 1..17: note = Description of Artificial Sequence: Synthetic primer
  source 1..17: mol_type = other DNA
cccggcttcctttgtcc

SEQ ID NO: 300 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 301 - DNA, 21 bp, synthetic construct
  misc_feature 1..21: note = Description of Artificial Sequence: Synthetic primer
  source 1..21: mol_type = other DNA
tccttatcacggtcccgctcg

SEQ ID NO: 302 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 303 - DNA, 17 bp, synthetic construct
  misc_feature 1..17: note = Description of Artificial Sequence: Synthetic primer
  source 1..17: mol_type = other DNA
cgtcgacaacggtagtg

SEQ ID NO: 304 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 305 - DNA, 18 bp, synthetic construct
  misc_feature 1..18: note = Description of Artificial Sequence: Synthetic primer
  source 1..18: mol_type = other DNA
tcgcgtgattctcggaac

SEQ ID NO: 306 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 307 - DNA, 20 bp, synthetic construct
  misc_feature 1..20: note = Description of Artificial Sequence: Synthetic primer
  source 1..20: mol_type = other DNA
gggcggtaagtggttagttt

SEQ ID NO: 308 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 309 - DNA, 17 bp, synthetic construct
  misc_feature 1..17: note = Description of Artificial Sequence: Synthetic primer
  source 1..17: mol_type = other DNA
aagaggcggagccagta

SEQ ID NO: 310 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 311 - DNA, 19 bp, synthetic construct
  misc_feature 1..19: note = Description of Artificial Sequence: Synthetic primer
  source 1..19: mol_type = other DNA
ctcccttctcccggtgccc

SEQ ID NO: 312 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 313 - DNA, 17 bp, synthetic construct
  misc_feature 1..17: note = Description of Artificial Sequence: Synthetic primer
  source 1..17: mol_type = other DNA
cccggcttcctttgtcc

SEQ ID NO: 314 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 315 - DNA, 20 bp, synthetic construct
  misc_feature 1..20: note = Description of Artificial Sequence: Synthetic primer
  source 1..20: mol_type = other DNA
gggcggtaagtggttagttt

SEQ ID NO: 316 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 317 - DNA, 17 bp, synthetic construct
  misc_feature 1..17: note = Description of Artificial Sequence: Synthetic primer
  source 1..17: mol_type = other DNA
cgtcgacaacggtagtg

SEQ ID NO: 318 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 319 - DNA, 17 bp, synthetic construct
  misc_feature 1..17: note = Description of Artificial Sequence: Synthetic primer
  source 1..17: mol_type = other DNA
aagaggcggagccagta

SEQ ID NO: 320 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 321 - DNA, 19 bp, synthetic construct
  misc_feature 1..19: note = Description of Artificial Sequence: Synthetic primer
  source 1..19: mol_type = other DNA
ctcccttctcccggtgccc

SEQ ID NO: 322 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 323 - DNA, 21 bp, synthetic construct
  misc_feature 1..21: note = Description of Artificial Sequence: Synthetic primer
  source 1..21: mol_type = other DNA
tccttatcacggtcccgctcg

SEQ ID NO: 324 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 325 - DNA, 17 bp, synthetic construct
  misc_feature 1..17: note = Description of Artificial Sequence: Synthetic primer
  source 1..17: mol_type = other DNA
cccggcttcctttgtcc

SEQ ID NO: 326 - DNA, 18 bp, synthetic construct
  misc_feature 1..18: note = Description of Artificial Sequence: Synthetic primer
  source 1..18: mol_type = other DNA
ggcctgccagcaggagga

SEQ ID NO: 327 - DNA, 17 bp, synthetic construct
  misc_feature 1..17: note = Description of Artificial Sequence: Synthetic primer
  source 1..17: mol_type = other DNA
cccggcttcctttgtcc

SEQ ID NO: 328 - DNA, 25 bp, synthetic construct
  misc_feature 1..25: note = Description of Artificial Sequence: Synthetic primer
  source 1..25: mol_type = other DNA
ggtgtgcagtcacattggtaaagcc

SEQ ID NO: 329 - DNA, 17 bp, synthetic construct
  misc_feature 1..17: note = Description of Artificial Sequence: Synthetic primer
  source 1..17: mol_type = other DNA
cccggcttcctttgtcc

SEQ ID NO: 330 - DNA, 22 bp, synthetic construct
  misc_feature 1..22: note = Description of Artificial Sequence: Synthetic primer
  source 1..22: mol_type = other DNA
gatgggtctagtccagctaaag

SEQ ID NO: 331 - DNA, 17 bp, synthetic construct
  misc_feature 1..17: note = Description of Artificial Sequence: Synthetic primer
  source 1..17: mol_type = other DNA
cccggcttcctttgtcc

SEQ ID NO: 332 - DNA, 18 bp, synthetic construct
  misc_feature 1..18: note = Description of Artificial Sequence: Synthetic primer
  source 1..18: mol_type = other DNA
gagagacaaggctgcaca

SEQ ID NO: 333 - DNA, 26 bp, synthetic construct
  misc_feature 1..26: note = Description of Artificial Sequence: Synthetic primer
  source 1..26: mol_type = other DNA
ccaggtgagagtcagggtagtgttca

SEQ ID NO: 334 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 335 - DNA, 23 bp, synthetic construct
  misc_feature 1..23: note = Description of Artificial Sequence: Synthetic primer
  source 1..23: mol_type = other DNA
agggacctttgcctgtgtgagtc

SEQ ID NO: 336 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 337 - DNA, 21 bp, synthetic construct
  misc_feature 1..21: note = Description of Artificial Sequence: Synthetic primer
  source 1..21: mol_type = other DNA
tcagctctgtgctgaggcgaa

SEQ ID NO: 338 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 339 - DNA, 32 bp, synthetic construct
  misc_feature 1..32: note = Description of Artificial Sequence: Synthetic primer
  source 1..32: mol_type = other DNA
aagccatctcccagaatatctgcttagaaatg

SEQ ID NO: 340 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 341 - DNA, 25 bp, synthetic construct
  misc_feature 1..25: note = Description of Artificial Sequence: Synthetic primer
  source 1..25: mol_type = other DNA
gagaggagcaacagtgagcatgatg

SEQ ID NO: 342 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 343 - DNA, 32 bp, synthetic construct; identical to SEQ ID NO: 339
  misc_feature 1..32: note = Description of Artificial Sequence: Synthetic primer
  source 1..32: mol_type = other DNA

SEQ ID NO: 344 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 345 - DNA, 25 bp, synthetic construct
  misc_feature 1..25: note = Description of Artificial Sequence: Synthetic primer
  source 1..25: mol_type = other DNA
gagaggagcaacagtgagcatgatg

SEQ ID NO: 346 - DNA, 17 bp, synthetic construct
  misc_feature 1..17: note = Description of Artificial Sequence: Synthetic primer
  source 1..17: mol_type = other DNA
gaactccacgccgttca

SEQ ID NO: 347 - DNA, 17 bp, synthetic construct
  misc_feature 1..17: note = Description of Artificial Sequence: Synthetic primer
  source 1..17: mol_type = other DNA
cccggcttcctttgtcc

SEQ ID NO: 348 - DNA, 22 bp, synthetic construct
  misc_feature 1..22: note = Description of Artificial Sequence: Synthetic primer
  source 1..22: mol_type = other DNA
ggctatgaactaatgaccccgt

SEQ ID NO: 349 - DNA, 17 bp, synthetic construct
  misc_feature 1..17: note = Description of Artificial Sequence: Synthetic primer
  source 1..17: mol_type = other DNA
cccggcttcctttgtcc

SEQ ID NO: 350 - DNA, 18 bp, synthetic construct
  misc_feature 1..18: note = Description of Artificial Sequence: Synthetic primer
  source 1..18: mol_type = other DNA
ggcctgccagcaggagga

SEQ ID NO: 351 - DNA, 17 bp, synthetic construct
  misc_feature 1..17: note = Description of Artificial Sequence: Synthetic primer
  source 1..17: mol_type = other DNA
cccggcttcctttgtcc

SEQ ID NO: 352 - DNA, 25 bp, synthetic construct
  misc_feature 1..25: note = Description of Artificial Sequence: Synthetic primer
  source 1..25: mol_type = other DNA
ggtgtgcagtcacattggtaaagcc

SEQ ID NO: 353 - DNA, 52 bp, synthetic construct
  misc_feature 1..52: note = Description of Artificial Sequence: Synthetic oligonucleotide
  source 1..52: mol_type = other DNA
acactctttccctacacgacgctcttccgatctccgacctcggctcacagcg

SEQ ID NO: 354 - DNA, 53 bp, synthetic construct
  misc_feature 1..53: note = Description of Artificial Sequence: Synthetic oligonucleotide
  source 1..53: mol_type = other DNA
acactctttccctacacgacgctcttccgatctaccgacctcggctcacagcg

SEQ ID NO: 355 - DNA, 54 bp, synthetic construct
  misc_feature 1..54: note = Description of Artificial Sequence: Synthetic oligonucleotide
  source 1..54: mol_type = other DNA
acactctttccctacacgacgctcttccgatctgaccgacctcggctcacagcg

SEQ ID NO: 356 - DNA, 55 bp, synthetic construct
  misc_feature 1..55: note = Description of Artificial Sequence: Synthetic oligonucleotide
  source 1..55: mol_type = other DNA
acactctttccctacacgacgctcttccgatcttgaccgacctcggctcacagcg

SEQ ID NO: 357 - DNA, 56 bp, synthetic construct
  misc_feature 1..56: note = Description of Artificial Sequence: Synthetic oligonucleotide
  source 1..56: mol_type = other DNA
acactctttccctacacgacgctcttccgatctctgaccgacctcggctcacagcg

SEQ ID NO: 358 - DNA, 57 bp, synthetic construct
  misc_feature 1..57: note = Description of Artificial Sequence: Synthetic oligonucleotide
  source 1..57: mol_type = other DNA
acactctttccctacacgacgctcttccgatctactgaccgacctcggctcacagcg

SEQ ID NO: 359 - DNA, 58 bp, synthetic construct
  misc_feature 1..58: note = Description of Artificial Sequence: Synthetic oligonucleotide
  source 1..58: mol_type = other DNA
acactctttccctacacgacgctcttccgatcttactgaccgacctcggctcacagcg

SEQ ID NO: 360 - DNA, 59 bp, synthetic construct
  misc_feature 1..59: note = Description of Artificial Sequence: Synthetic oligonucleotide
  source 1..59: mol_type = other DNA
acactctttccctacacgacgctcttccgatctgtactgaccgacctcggctcacagcg

SEQ ID NO: 361 - DNA, 51 bp, synthetic construct
  misc_feature 1..51: note = Description of Artificial Sequence: Synthetic oligonucleotide
  source 1..51: mol_type = other DNA
gtgactggagttcagacgtgtgctcttccgatctccacccagccagctccc

SEQ ID NO: 362 - DNA, 51 bp, synthetic construct
  misc_feature 1..51: note = Description of Artificial Sequence: Synthetic oligonucleotide
  source 1..51: mol_type = other DNA
acactctttccctacacgacgctcttccgatctccggtggcgcattgccac

SEQ ID NO: 363 - DNA, 52 bp, synthetic construct
  misc_feature 1..52: note = Description of Artificial Sequence: Synthetic oligonucleotide
  source 1..52: mol_type = other DNA
acactctttccctacacgacgctcttccgatctaccggtggcgcattgccac

SEQ ID NO: 364 - DNA, 53 bp, synthetic construct
  misc_feature 1..53: note = Description of Artificial Sequence: Synthetic oligonucleotide
  source 1..53: mol_type = other DNA
acactctttccctacacgacgctcttccgatctgaccggtggcgcattgccac

SEQ ID NO: 365 - DNA, 54 bp, synthetic construct
  misc_feature 1..54: note = Description of Artificial Sequence: Synthetic oligonucleotide
  source 1..54: mol_type = other DNA
acactctttccctacacgacgctcttccgatcttgaccggtggcgcattgccac

SEQ ID NO: 366 - DNA, 55 bp, synthetic construct
  misc_feature 1..55: note = Description of Artificial Sequence: Synthetic oligonucleotide
  source 1..55: mol_type = other DNA
acactctttccctacacgacgctcttccgatctctgaccggtggcgcattgccac

SEQ ID NO: 367 - DNA, 56 bp, synthetic construct
  misc_feature 1..56: note = Description of Artificial Sequence: Synthetic oligonucleotide
  source 1..56: mol_type = other DNA
acactctttccctacacgacgctcttccgatctactgaccggtggcgcattgccac

SEQ ID NO: 368 - DNA, 57 bp, synthetic construct
  misc_feature 1..57: note = Description of Artificial Sequence: Synthetic oligonucleotide
  source 1..57: mol_type = other DNA
acactctttccctacacgacgctcttccgatcttactgaccggtggcgcattgccac

SEQ ID NO: 369 - DNA, 58 bp, synthetic construct
  misc_feature 1..58: note = Description of Artificial Sequence: Synthetic oligonucleotide
  source 1..58: mol_type = other DNA
acactctttccctacacgacgctcttccgatctgtactgaccggtggcgcattgccac

SEQ ID NO: 370 - DNA, 54 bp, synthetic construct
  misc_feature 1..54: note = Description of Artificial Sequence: Synthetic oligonucleotide
  source 1..54: mol_type = other DNA
gtgactggagttcagacgtgtgctcttccgatctcagagtccagcttgggccca

SEQ ID NO: 371 - DNA, 20 bp, synthetic construct
  misc_feature 1..20: note = Description of Artificial Sequence: Synthetic oligonucleotide
  source 1..20: mol_type = other DNA
gatattttcccagctcacca

SEQ ID NO: 372 - DNA, 20 bp, synthetic construct
  misc_feature 1..20: note = Description of Artificial Sequence: Synthetic oligonucleotide
  source 1..20: mol_type = other DNA
tctattctcccagctcccca

SEQ ID NO: 373 - DNA, 40 bp, synthetic construct
  misc_feature 1..40: note = Description of Artificial Sequence: Synthetic oligonucleotide
  source 1..40: mol_type = other DNA
agcggcttctgtctctgtgagtgagctggcggtctccgtc

SEQ ID NO: 374 - DNA, 43 bp, synthetic construct
  misc_feature 1..43: note = Description of Artificial Sequence: Synthetic oligonucleotide
  source 1..43: mol_type = other DNA
gactagcccacgctccggttctgagccgcgacggcggtctccg

SEQ ID NO: 375 - DNA, 41 bp, synthetic construct
  misc_feature 1..41: note = Description of Artificial Sequence: Synthetic oligonucleotide
  source 1..41: mol_type = other DNA
cccagggtcccatgcgctccccggccctgacggcggtctcc

SEQ ID NO: 376 - protein, 2560 aa, synthetic construct
  REGION 1..2560: note = Description of Artificial Sequence: Synthetic polypeptide
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGSETPGTSESATPESSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDGTGGGGVTVKFKYKGEELEVDISKIKKVWRVGKMISFTYDDNGKTGRGAVSEKDAPKELLQMLEKSGKKSGGSKRTADGSEFEPKKKRKVGGGGSPKKKRKVYPYDVPDYAGSRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 377 - DNA, 7680 bp, synthetic construct
  misc_feature 1..7680: note = Description of Artificial Sequence: Synthetic polynucleotide
  source 1..7680: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagctctggtagcgagacacccggtaccagtgaaagcgccacgccagaaagcagtgggagtgagactccgggtacatctgaatcagcgacaccggaatcaagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggcccccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatgggacaggtggcggtggtgtcaccgtcaagttcaagtacaagggtgaggaacttgaagttgatattagcaaaatcaagaaggtttggcgcgttggtaaaatgatatcttttacttatgacgacaacggcaagacaggtagaggggcagtgtctgagaaagacgcccccaaggagctgttgcaaatgttggaaaagtctgggaaaaagtctggcggctcaaaaagaaccgccgacggcagcgaattcgagcccaagaagaagaggaaagtcggaggtggcgggagcccaaaaaagaaaagaaaagtgtatccctatgatgtccccgattatgccggttcaagagccctggtcgtgattagactgagccgagtgacagacgccaccacaagtcccgagagacagctggaatcatgccagcagctctgtgctcagcggggttgggatgtggtcggcgtggcagaggatctggacgtgagcggggccgtcgatccattcgacagaaagaggaggcccaacctggcaagatggctcgctttcgaggaacagccctttgatgtgatcgtcgcctacagagtggaccggctgacccgctcaattcgacatctccagcagctggtgcattgggctgaggaccacaagaaactggtggtcagcgcaacagaagcccacttcgatactaccacaccttttgccgctgtggtcatcgcactgatgggcactgtggcccagatggagctcgaagctatcaaggagcgaaacaggagcgcagcccatttcaatattagggccggtaaatacagaggctccctgcccccttggggatatctccctaccagggtggatggggagtggagactggtgccagaccccgtccagagagagcggattctggaagtgtaccacagagtggtcgataaccacgaaccactccatctggtggcacacgacctgaatagacgcggcgtgctctctccaaaggattattttgctcagctgcagggaagagagccacagggaagagaatggagtgctactgcactgaagagatctatgatcagtgaggctatgctgggttacgcaacactcaatggcaaaactgtccgggacgatgacggagcccctctggtgagggctgagcctattctcaccagagagcagctcgaagctctgcgggcagaactggtcaagactagtcgcgccaaacctgccgtgagcaccccaagcctgctcctgagggtgctgttctgcgccgtctgtggagagccagcatacaagtttgccggcggagggcgcaaacatccccgctatcgatgcaggagcatggggttccctaagcactgtggaaacgggacagtggccatggctgagtgggacgccttttgcgaggaacaggtgctggatctcctgggtgacgctgagcggctggaaaaagtgtgggtggcaggatctgactccgctgtggagctggcagaagtcaatgccgagctcgtggatctgacttccctcatcggatctcctgcatatagagctgggtccccacagagagaagctctggacgcacgaattgctgcactcgctgctagacaggaggaactggagggcctggaggccaggccctctggatgggagtggcgagaaaccggacagaggtttggggattggtggagggagcaggacaccgcagccaagaacacatggctgagatccatgaatgtccggctcacattcgacgtgcgcggtggcctgactcgaaccatcgattttggcgacctgcaggagtatgaacagcacctgagactggggtccgtggtcgaaagactgcacactgggatgtcc

SEQ ID NO: 378 - protein, 1367 aa, synthetic construct
  REGION 1..1367: note = Description of Artificial Sequence: Synthetic polypeptide
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 379 - protein, 576 aa, synthetic construct
  REGION 1..576: note = Description of Artificial Sequence: Synthetic polypeptide
LNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDGTGGGGVTVKFKYKGEELEVDISKIKKVWRVGKMISFTYDDNGKTGRGAVSEKDAPKELLQMLEKSGKKSGGSKRTADGS

SEQ ID NO: 380 - protein, 500 aa, synthetic construct
  REGION 1..500: note = Description of Artificial Sequence: Synthetic polypeptide
SRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 381 - DNA, 11344 bp, synthetic construct
  misc_feature 1..11344: note = Description of Artificial Sequence: Synthetic polynucleotide
  source 1..11344: mol_type = other DNA
ccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtcggaagcggagctactaacttcagcctgctgaagcaggctggcgacgtggaggagaaccctggacctccaaaaaagaaaagaaaagtgtatccctatgatgtccccgattatgccggttcaagagccctggtcgtgattagactgagccgagtgacagacgccaccacaagtcccgagagacagctggaatcatgccagcagctctgtgctcagcggggttgggatgtggtcggcgtggcagaggatctggacgtgagcggggccgtcgatccattcgacagaaagaggaggcccaacctggcaagatggctcgctttcgaggaacagccctttgatgtgatcgtcgcctacagagtggaccggctgacccgctcaattcgacatctccagcagctggtgcattgggctgaggaccacaagaaactggtggtcagcgcaacagaagcccacttcgatactaccacaccttttgccgctgtggtcatcgcactgatgggcactgtggcccagatggagctcgaagctatcaaggagcgaaacaggagcgcagcccatttcaatattagggccggtaaatacagaggctccctgcccccttggggatatctccctaccagggtggatggggagtggagactggtgccagaccccgtccagagagagcggattctggaagtgtaccacagagtggtcgataaccacgaaccactccatctggtggcacacgacctgaatagacgcggcgtgctctctccaaaggattattttgctcagctgcagggaagagagccacagggaagagaatggagtgctactgcactgaagagatctatgatcagtgaggctatgctgggttacgcaacactcaatggcaaaactgtccgggacgatgacggagcccctctggtgagggctgagcctattctcaccagagagcagctcgaagctctgcgggcagaactggtcaagactagtcgcgccaaacctgccgtgagcaccccaagcctgctcctgagggtgctgttctgcgccgtctgtggagagccagcatacaagtttgccggcggagggcgcaaacatccccgctatcgatgcaggagcatggggttccctaagcactgtggaaacgggacagtggccatggctgagtgggacgccttttgcgaggaacaggtgctggatctcctgggtgacgctgagcggctggaaaaagtgtgggtggcaggatctgactccgctgtggagctggcagaagtcaatgccgagctcgtggatctgacttccctcatcggatctcctgcatatagagctgggtccccacagagagaagctctggacgcacgaattgctgcactcgctgctagacaggaggaactggagggcctggaggccaggccctctggatgggagtggcgagaaaccggacagaggtttggggattggtggagggagcaggacaccgcagccaagaacacatggctgagatccatgaatgtccggctcacattcgacgtgcgcggtggcctgactcgaaccatcgattttggcgacctgcaggagtatgaacagcacctgagactggggtccgtggtcgaaagactgcacactgggatgtcctaggtttaaacccgctgatcagcctcgactgtgccttctagttgccagccatctgttgtttgcccctcccccgtgccttccttgaccctggaaggtgccactcccactgtcctttcctaataaaatgagaaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttcc

SEQ ID NO: 382 - DNA, 9753 bp, synthetic construct
  misc_feature 1..9753: note = Description of Artificial Sequence: Synthetic polynucleotide
  source 1..9753: mol_type = other DNA
ccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgagaaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttcc

SEQ ID NO: 383 - DNA, 11433 bp, synthetic construct
  misc_feature 1..11433: note = Description of Artificial Sequence: Synthetic polynucleotide
  source 1..11433: mol_type = other DNA
ccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgcccgcggctaagagggtgaagcttgacggtggaaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatatggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaaaaaagaaccgctgactctcaacattccacacctccaaaaaccaagcgaaaagtggaattcgagcccaagaagaagaggaaagtcggaagcggagctactaacttcagcctgctgaagcaggctggcgacgtggaggagaaccctggacctccaaaaaagaaaagaaaagtgtatccctatgatgtccccgattatgccggttcaagagccctggtcgtgattagactgagccgagtgacagacgccaccacaagtcccgagagacagctggaatcatgccagcagctctgtgctcagcggggttgggatgtggtcggcgtggcagaggatctggacgtgagcggggccgtcgatccattcgacagaaagaggaggcccaacctggcaagatggctcgctttcgaggaacagccctttgatgtgatcgtcgcctacagagtggaccggctgacccgctcaattcgacatctccagcagctggtgcattgggctgaggaccacaagaaactggtggtcagcgcaacagaagcccacttcgatactaccacaccttttgccgctgtggtcatcgcactgatgggcactgtggcccagatggagctcgaagctatcaaggagcgaaacaggagcgcagcccatttcaatattagggccggtaaatacagaggctccctgcccccttggggatatctccctaccagggtggatggggagtggagactggtgccagaccccgtccagagagagcggattctggaagtgtaccacagagtggtcgataaccacgaaccactccatctggtggcacacgacctgaatagacgcggcgtgctctctccaaaggattattttgctcagctgcagggaagagagccacagggaagagaatggagtgctactgcactgaagagatctatgatcagtgaggctatgctgggttacgcaacactcaatggcaaaactgtccgggacgatgacggagcccctctggtgagggctgagcctattctcaccagagagcagctcgaagctctgcgggcagaactggtcaagactagtcgcgccaaacctgccgtgagcaccccaagcctgctcctgagggtgctgttctgcgccgtctgtggagagccagcatacaagtttgccggcggagggcgcaaacatccccgctatcgatgcaggagcatggggttccctaagcactgtggaaacgggacagtggccatggctgagtgggacgccttttgcgaggaacaggtgctggatctcctgggtgacgctgagcggctggaaaaagtgtgggtggcaggatctgactccgctgtggagctggcagaagtcaatgccgagctcgtggatctgacttccctcatcggatctcctgcatatagagctgggtccccacagagagaagctctggacgcacgaattgctgcactcgctgctagacaggaggaactggagggcctggaggccaggccctctggatgggagtggcgagaaaccggacagaggtttggggattggtggagggagcaggacaccgcagccaagaacacatggctgagatccatgaatgtccggctcacattcgacgtgcgcggtggcctgactcgaaccatcgattttggcgacctgcaggagtatgaacagcacctgagactggggtccgtggtcgaaagactgcacactgggatgtcctaggtttaaacccgctgatcagcctcgactgtgccttctagttgccagccatctgttgtttgcccctcccccgtgccttccttgaccctggaaggtgccactcccactgtcctttcctaataaaatgagaaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttcc

SEQ ID NO: 384 - DNA, 11056 bp, synthetic construct
  misc_feature 1..11056: note = Description of Artificial Sequence: Synthetic polynucleotide
  source 1..11056: mol_type = other DNA
ccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagctctggtagcgagacacccggtaccagtgaaagcgccacgccagaaagcagtgggagtgagactccgggtacatctgaatcagcgacaccggaatcaagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggcccccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatgggacaggtggcggtggtgtcaccgtcaagttcaagtacaagggtgaggaacttgaagttgatattagcaaaatcaagaaggtttggcgcgttggtaaaatgatatcttttacttatgacgacaacggcaagacaggtagaggggcagtgtctgagaaagacgcccccaaggagctgttgcaaatgttggaaaagtctgggaaaaagtctggcggctcaaaaagaaccgccgacggcagcgaattcgagcccaagaagaagaggaaagtcggaggtggcgggagcccaaaaaagaaaagaaaagtgtatccctatgatgtccccgattatgccggttcaagagccctggtcgtgattagactgagccgagtgacagacgccaccacaagtcccgagagacagctggaatcatgccagcagctctgtgctcagcggggttgggatgtggtcggcgtggcagaggatctggacgtgagcggggccgtcgatccattcgacagaaagaggaggcccaacctggcaagatggctcgctttcgaggaacagccctttgatgtgatcgtcgcctacagagtggaccggctgacccgctcaattcgacatctccagcagctggtgcattgggctgaggaccacaagaaactggtggtcagcgcaacagaagcccacttcgatactaccacaccttttgccgctgtggtcatcgcactgatgggcactgtggcccagatggagctcgaagctatcaaggagcgaaacaggagcgcagcccatttcaatattagggccggtaaatacagaggctccctgcccccttggggatatctccctaccagggtggatggggagtggagactggtgccagaccccgtccagagagagcggattctggaagtgtaccacagagtggtcgataaccacgaaccactccatctggtggcacacgacctgaatagacgcggcgtgctctctccaaaggattattttgctcagctgcagggaagagagccacagggaagagaatggagtgctactgcactgaagagatctatgatcagtgaggctatgctgggttacgcaacactcaatggcaaaactgtccgggacgatgacggagcccctctggtgagggctgagcctattctcaccagagagcagctcgaagctctgcgggcagaactggtcaagactagtcgcgccaaacctgccgtgagcaccccaagcctgctcctgagggtgctgttctgcgccgtctgtggagagccagcatacaagtttgccggcggagggcgcaaacatccccgctatcgatgcaggagcatggggttccctaagcactgtggaaacgggacagtggccatggctgagtgggacgccttttgcgaggaacaggtgctggatctcctgggtgacgctgagcggctggaaaaagtgtgggtggcaggatctgactccgctgtggagctggcagaagtcaatgccgagctcgtggatctgacttccctcatcggatctcctgcatatagagctgggtccccacagagagaagctctggacgcacgaattgctgcactcgctgctagacaggaggaactggagggcctggaggccaggccctctggatgggagtggcgagaaaccggacagaggtttggggattggtggagggagcaggacaccgcagccaagaacacatggctgagatccatgaatgtccggctcacattcgacgtgcgcggtggcctgactcgaaccatcgattttggcgacctgcaggagtatgaacagcacctgagactggggtccgtggtcgaaagactgcacactgggatgtcctaggtttaaacccgctgatcagcctcgactgtgccttctagttgccagccatctgttgtttgcccctcccccgtgccttccttgaccctggaaggtgccactcccactgtcctttcctaataaaatgagaaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttcc

SEQ ID NO: 385 - DNA, 2367 bp, synthetic construct
  misc_feature 1..2367: note = Description of Artificial Sequence: Synthetic polynucleotide
  source 1..2367: mol_type = other DNA
tgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgctagctgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctattctcgcagctcaccagttttagagctagaaatagcaagttaaaataaggctagtccgttatcaacttgaaaaagtggcaccgagtcggtgcgacgagcgcggcgatatcatcatccatggccggatgatcctgacgacggagaccgccgtcgtcgacaagccggcctgagctgcgagaatttttttaagcttgggccgctcgaggtacctctctacatatgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgc

SEQ ID NO: 386 - DNA, 2280 bp, synthetic construct
  misc_feature 1..2280: note = Description of Artificial Sequence: Synthetic polynucleotide
  source 1..2280: mol_type = other DNA
ctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgctagctgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaagccggccttgcacatgcgttttagagctagaaatagcaagttaaaataaggctagtccgttatcaacttgaaaaagtggcaccgagtcggtgcgtttttttaagcttgggccgctcgaggtacctctctacatatgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgc

SEQ ID NO: 387 - DNA, 6386 bp, synthetic construct
  misc_feature 1..6386: note = Description of Artificial Sequence: Synthetic polynucleotide
  source 1..6386: mol_type = other DNA
tgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccrggsggggsggggsggggsgrggggsggggsggggsgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactatagggggtcgacccgccaccatgccaaaaaagaaaagaaaagtgtatccctatgatgtccccgattatgccggttcaagagccctggtcgtgattagactgagccgagtgacagacgccaccacaagtcccgagagacagctggaatcatgccagcagctctgtgctcagcggggttgggatgtggtcggcgtggcagaggatctggacgtgagcggggccgtcgatccattcgacagaaagaggaggcccaacctggcaagatggctcgctttcgaggaacagccctttgatgtgatcgtcgcctacagagtggaccggctgacccgctcaattcgacatctccagcagctggtgcattgggctgaggaccacaagaaactggtggtcagcgcaacagaagcccacttcgatactaccacaccttttgccgctgtggtcatcgcactgatgggcactgtggcccagatggagctcgaagctatcaaggagcgaaacaggagcgcagcccatttcaatattagggccggtaaatacagaggctccctgcccccttggggatatctccctaccagggtggatggggagtggagactggtgccagaccccgtccagagagagcggattctggaagtgtaccacagagtggtcgataaccacgaaccactccatctggtggcacacgacctgaatagacgcggcgtgctctctccaaaggattattttgctcagctgcagggaagagagccacagggaagagaatggagtgctactgcactgaagagatctatgatcagtgaggctatgctgggttacgcaacactcaatggcaaaactgtccgggacgatgacggagcccctctggtgagggctgagcctattctcaccagagagcagctcgaagctctgcgggcagaactggtcaagactagtcgcgccaaacctgccgtgagcaccccaagcctgctcctgagggtgctgttctgcgccgtctgtggagagccagcatacaagtttgccggcggagggcgcaaacatccccgctatcgatgcaggagcatggggttccctaagcactgtggaaacgggacagtggccatggctgagtgggacgccttttgcgaggaacaggtgctggatctcctgggtgacgctgagcggctggaaaaagtgtgggtggcaggatctgactccgctgtggagctggcagaagtcaatgccgagctcgtggatctgacttccctcatcggatctcctgcatatagagctgggtccccacagagagaagctctggacgcacgaattgctgcactcgctgctagacaggaggaactggagggcctggaggccaggccctctggatgggagtggcgagaaaccggacagaggtttggggattggtggagggagcaggacaccgcagccaagaacacatggctgagatccatgaatgtccggctcacattcgacgtgcgcggtggcctgactcgaaccatcgattttggcgacctgcaggagtatgaacagcacctgagactggggtccgtggtcgaaagactgcacactgggatgtcctaggtcagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacrmktymsrtkssmcwttymggsgaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcag

SEQ ID NO: 388 - DNA, 6317 bp, synthetic construct
  misc_feature 1..6317: note = Description of Artificial Sequence: Synthetic polynucleotide
  source 1..6317: mol_type = other DNA
gattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccrggsggggsggggsggggsgrggggsggggsggggsgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactatagggggtcgacccgccaccatgacagcgccaaagaaaaagaggaaggtcatgaccaagaaagtggccatctatactagagtgagcacaacgaatcaggccgaggaggggttctctattgacgagcaaatcgatcgtctgaccaagtacgcggaagcaatgggctggcaagtcagcgacacttacaccgatgctgggttctccggcgccaaactggaaaggcctgccatgcagcggctgattaacgacattgagaacaaggcctttgatacagtgctcgtatacaagctcgacaggctcagccgatctgtgcgggacacgctttacctcgtaaaggatgttttcactaagaataaaatcgacttcattagcctgaacgaatccattgacaccagctcagctatgggctctctgttcctgaccatcctgagcgctatcaatgagtttgagagggagaatataaaggagcgcatgacaatgggaaagctgggtagagcgaagtccgggaaatctatgatgtggaccaagaccgcttttggatactaccacaataggaagacgggcattctggagatcgtgcccttgcaggcaaccatcgttgagcagatcttcaccgactacctgagcggaatatctctcacgaagttgcgagataagctgaatgagagcggacacattggcaaggatattccttggtcatatagaaccctccgccaaactctggataatccggtgtactgcggttacatcaagttcaaagacagcctcttcgagggaatgcataaacctatcattccatacgagacatacctgaaagtccaaaaggaactcgaagagcgccagcaacagacttacgaacggaataataatcccaggcctttccaggccaaatatatgctgtccggcatggcaagatgcggatactgcggggcaccactcaagattgtgcttggccataaacggaaggatggaagcagaaccatgaaatatcactgcgcaaaccgctttccaaggaaaacgaaggggattaccgtgtacaatgacaacaaaaaatgtgatagcggaacctacgatctgtccaacttggaaaacaccgtcattgacaatttaattggatttcaggaaaataatgacagccttctgaagattatcaacgggaacaatcagccgattctggacacttcatctttcaaaaaacagatctctcagattgataagaaaattcagaaaaattccgatttatacctcaatgatttcataacgatggatgagctgaaggaccggaccgacagtttgcaggccgagaagaaactgctgaaagcaaagatctccgagaacaagttcaatgacagtaccgatgtcttcgagttggtgaagacccagctgggtagtatcccaatcaacgagttgagctatgacaataagaagaagattgttaataacctggtgagcaaagtggacgtgaccgctgataacgtggatattatcttcaagttccagctggcctgagtcagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacrmktymsrtkssmcwttymggsgaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccg

SEQ ID NO: 389 - DNA, 6638 bp, synthetic construct
  misc_feature 1..6638: note = Description of Artificial Sequence: Synthetic polynucleotide
  source 1..6638: mol_type = other DNA
gattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccrggsggggsggggsggggsgrggggsggggsggggsgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactatagggggtcgacccgccaccatgcccaagaagaaacggaaagtgatgagcccctttatcgccccggacgtgcccgagcacctcctggacactgtgcgcgtctttctgtacgcccgtcagagtaaaggacggtcagatggatctgacgtgtccaccgaagcacagctcgctgccggacgggcccttgttgcctcaagaaacgcacaagggggagctagatgggtggtggcgggcgaattcgtggatgtgggcagatcagggtgggacccgaatgtgacacgcgccgacttcgaaagaatgatgggcgaggtgcgcgccggtgagggagacgtagtggtggttaatgaactgagtcgccttacgaggaagggcgcccacgacgctctggagatcgataacgaactcaaaaaacacggtgtgcggttcatgagcgtgctggaaccattcctggataccagcaccccaatcggtgtcgcgatctttgccctgattgccgcgctcgctaaacaggattcagaccttaaagctgagcggctgaagggggctaaagatgagatcgctgccttggggggtgtgcacagctcatctgcgccattcggcatgagggcggtcagaaagaaagtggataacctggtcatatctgttctggagcctgatgaggacaacccggaccacgttgagcttgtggaacggatggctaagatgtctttcgaaggcgtcagcgataacgcaattgccacaacatttgagaaggagaaaatcccctctccggggatggctgagagacgagccacggagaagaggcttgcttctattaaggcacggaggctcaatggcgccgaaaagccgatcatgtggcgggcgcagacagttagatggattcttaaccatcccgcgattggtggattcgcattcgagcgggtgaaacacggaaaagcccacatcaacgtgatacgaagagatcccggcggcaaaccccttacccctcacactggtatcctgtctggatccaagtggttggaactccaggagaagagaagcgggaaaaatctctccgaccgcaaaccaggtgccgaagtggaacctacgctgctttccgggtggagatttctgggatgtcggatatgcggtgggtcaatgggccagtcccaagggggccgtaagaggaatggggacttggctgagggcaattacatgtgtgcaaacccaaaggggcacggcggtctgagcgtcaagaggtctgagcttgatgaattcgtggcatcaaaagtctgggccaggttgcgcacggctgacatggaggatgaacatgaccaagcatggattgcagctgcagctgaacggtttgctttgcagcacgacctggcgggggtagctgacgagcgacgggagcaacaagctcacctggataacgttcggagatcaataaaagatctccaggcggataggaaggcaggtctctacgtgggacgcgaagaactggagacctggcgcagtaccgtcctgcaatataggagctacgaggctgagtgtactactaggttggctgagctggatgaaaaaatgaatggatccacccgggtgccttcagaatggtttagcggcgaggacccaaccgcggaaggaggcatatgggcgagctgggatgtctatgagcgccgggagtttctcagcttttttttggactccgtaatggttgacaggggcagacatcctgaaaccaagaaatatataccattgaaagaccgggtgaccttaaagtgggcggagctgttaaaggaagaggatgaagcaagcgaggccacagaacgggagctggcagctctttaggtcagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacrmktymsrtkssmcwttymggsgaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccg

SEQ ID NO: 390 - DNA, 9530 bp, synthetic construct
  misc_feature 1..9530: note = Description of Artificial Sequence: Synthetic polynucleotide
  source 1..9530: mol_type = other DNA
taatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatacattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactataagctgcgagcaacttcacttgggtatgccggcggtagcgctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatcgtggttcgcaccgactcggtgccacagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagtaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggactgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgggcccgccccaactggggtaacctttgagttctctcagttggggg

SEQ ID NO: 391 - DNA, 5722 bp, synthetic construct
  misc_feature 1..5722: note = Description of Artificial Sequence: Synthetic polynucleotide
  source 1..5722: mol_type = other DNA
tgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatacattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctattctcgcagctcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgaaccatggatgatgatatcgccgcgctcgtcgtcgacaacggctccggcatgtgcaaggccggcttcgcgggcgacgatgccccccgggccgtcttcccctccatcgtggggcgccccaggcaccaggtaggggagctggctgggtggggcagccccgggagcgggcgggaggcaagggcgctttctctgcacaggagcctcccggtttccggggtgggggctgcgcccgtgctcagggcttcttgtcctttccttcccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagct

SEQ ID NO: 392 - DNA, 15424 bp, synthetic construct
  misc_feature 1..15424: note = Description of Artificial Sequence: Synthetic polynucleotide
  source 1..15424: mol_type = other DNA
tcgacggtatcgataagcttgatatcgaattcctgcagcccgggggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttaggctgccgctgtgaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaaatgtttttttgttattttattttgtgtttatgcagaaacccgcagacatgtttgagagaaaaatggtgtctttttctgtggtggttccggagcttacctgcctttatctgcatgagcatgactacgatgtgctttcttttttgcgcgaggctttgcctgattttttgagcagcaccttgcattttatatcgccgcccatgcaacaaagcttacatcggggctacgctggttagcatagctccgagtatgcgtgtcataatcagtgtgggttcttttgtcaaggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactccattttcgtcaaatcttataataagatgagcactttgaactgttccagatattggagccaaactgcctttaacagccaaaactgaaactgtagcaagtatttgactgccacattttgttaagaccaaagtgagtttagcatctttctctgcatttagtctacagttaggagatggagctggtgtggtccacaaagttagcttatcattatttttgtttcctactgtaatggcacctgtgctgtcaaaactaaggccagttcctagtttaggaaccatagccttgtttgaatcaaattctaggccatggccaatttttgttttgaggggatttgtgtttggtgcattaggtgaaccaaattcaagcccatctcctgcattaatggctatggctgtagcgtcaaacatcaaccccttggcagtgcttaggttaacctcaagctttttggaattgtttgaagctgtaaacaagtaaaggcctttgttgtagttaatatccaagttgtgggctgagtttataaaaagagggccctgtcctagtcttagatttagttggttttgagcatcaaacggataactaacatcaagtataaggcgtctgttttgagaatcaatccttagtcctcctgctacattaagttgcatattgccttgtgaatcaaaacccaaggctccagtaactttagtttgcaaggaagtattattaatagtcacacctggaccagttgctacggtcaaagtgtttaggtcgtctgttacatgcaaaggagccccgtactttagtcctagttttccattttgtgtataaatgggctctttcaagtcaatgcccaagctaccagtggcagtagttagagggggtgaggcagtgatagtaagggtactgctatcggtggtggtgagggggcctgatgtttgcagggctagctttccttctgacactgtgaggggtccttgggtggcaatgctaagtttggagtcgtgcacggttagcggggcctgtgattgcatggtgagtgtgttgcccgcgaccattagaggtgcggcggcagccacagttagggcttctgaggtaactgtgaggggtgcagatatttccaggtttatgtttgacttggtttttttgagaggtgggctcacagtggttacattttgggaggtaaggttgccggcctcgtccagagagaggccgttgcccattttgagcgcaagcatgccattggaggtaactagaggttcggataggcgcaaagagagtaccccagggggactctcttgaaacccattgggggatacaaagggaggagtaagaaaaggcacagttggaggaccggtttccgtgtcatatggatacacggggttgaaggtatcttcagacggtcttgcgcgcttcatctgcaacaacatgaagatagtgggtgcggatggacaggaacaggaggaaactgacattccatttagattgtggagaaagtttgcagccaggaggaagctgcaataccagagctgggaggagggcaaggaggtgctgctgaataaactggacagaaatttgctaactgattttaagtaagtgatgctttattatttttttttattagttaaagggaataagatccccgggtactctagttaattaactagaggatcttgatgtaatccaaggttaggacagttgcaaatcacagtgagaacacagggtcccctgtcccgctcaactagcagggggcgctgggtaaactcccgaatcaggctacgggcaagctctccctgggcggtaagccggacgccgtgcgccgggccctcgatatgatcctcgggcaattcaaagtagcaaaactcaccggagtcgcgggcaaagcacttgtggcggcgacagtggaccaggtgtttcaggcgcagttgctctgcctctccacttaacattcagtcgtagccgtccgccgagtcctttaccgcgtcaaagttaggaataaattgatccggatagtggccgggaggtcccgagaaggggttaaagtagaccgatggcacaaactcctcaataaattgcagagttccaatgcctccagagcgcggctcagaggacgaggtctgcagagttaggattgcctgacgaggcgtgaatgaaggacggccggcgccgccgatctgaaatgtcccgtccggacggagaccaagcgaggagctcaccgactcgtcgttgagctgaatacctcgccctctgattgtcaggtgagttataccctgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttccaagagaattcggtttcggtgggcgcggattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacgacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcatttggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcggcgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacggtagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctg

SEQ ID NO: 393 - DNA, 3849 bp, synthetic construct
  misc_feature 1..3849: note = Description of Artificial Sequence: Synthetic polynucleotide
  source 1..3849: mol_type = other DNA
cggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcagtggtttgtctggtcaaccaccgcggtctcagtggtgtacggtacaaacccagctaccggtcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgagtttggacaaaccacaactagaatgcagtgaaaaaaatgctttatttgtgaaatttgtgatgctattgctttatttgtgggcccgggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcc

SEQ ID NO: 394 - DNA, 7336 bp, synthetic construct
  misc_feature 1..7336: note = Description of Artificial Sequence: Synthetic polynucleotide
  source 1..7336: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgcc

SEQ ID NO: 395 - DNA, 969 bp, synthetic construct
  misc_feature 1..969: note = Description of Artificial Sequence: Synthetic polynucleotide
  source 1..969: mol_type = other DNA
ccccaactggggtaacctttgggctccccgggcgcgactataagctgcgagcaacttcacttgggtatgccggcggtagcgcttaccgttcgtataatgtatgctatacgaagttatccgaagccgctagcggtggtttgtctggtcaaccaccgcggtctcagtggtgtacggtacaaacccagctaccggtcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgagtttggacaaaccacaactagaatgcagtgaaaaaaatgctttatttgtgaaatttgtgatgctattgctttatttgtgggcccg

SEQ ID NO: 396 - DNA, 4769 bp, synthetic construct
  misc_feature 1..4769: note = Description of Artificial Sequence: Synthetic polynucleotide
  source 1..4769: mol_type = other DNA
tgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatacattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgaggtggtttgtctggtcaaccaccgcggtctcagtggtgtacggtacaaacccaccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagct

SEQ ID NO: 397 - DNA, 797 bp, synthetic construct
  misc_feature 1..797: note = Description of Artificial Sequence: Synthetic polynucleotide
  source 1..797: mol_type = other DNA
ccccaactggggtaacctttgggctccccgggcgcgatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggtggtttgtctggtcaaccaccgcgagctcagtggtgtacggtacaaaccca

SEQ ID NO: 398 - DNA, 815 bp, synthetic construct
  misc_feature 1..815: note = Description of Artificial Sequence: Synthetic polynucleotide
  source 1..815: mol_type = other DNA
ccccaactggggtaacctttgggctccccgggcgcggccgccaccatggtgtccaagggtgaggaactttttaccggagtggtgccgatactggtagagctggatggcgacgtaaacgggcacaagttcagtgtacggggagagggcgagggcgacgctacgaatgggaaattgactttgaaatttatttgcaccacgggcaaattgccggtcccgtggccaactttggttacgaccttgacctatggcgttcagtgtttctcacggtacccagaccacatgaaacagcatgacttttttaagtcagcgatgccggagggatatgtgcaagaacggactatctcatttaaagatgatggcacatataagacaagagcggaagtcaaattcgaaggggacaccctcgtcaatcgaatagaactcaagggaatagacttcaaagaagatggtaatatactggggcacaaactcgaatacaatttcaacagtcataacgtctacatcactgccgacaaacaaaaaaatgggatcaaagcgaacttcaaaatccgacataatgtcgaggatgggagcgtccaactggcagaccattaccagcaaaatactccaataggtgatggtccagtgcttttgccagataatcattatcttagctatcagagcaagttgagtaaggatccgaatgaaaagcgagatcacatggtcttgctggagtttgttacggcggctggtatcacacttggtatggatgaattgtacaagggtggtttgtctggtcaaccaccgcggactcagtggtgtacggtacaaaccca

SEQ ID NO: 399 - DNA, 1660 bp, synthetic construct
  misc_feature 1..1660: note = Description of Artificial Sequence: Synthetic polynucleotide
  source 1..1660: mol_type = other DNA
ccccaactggggtaacctttgggctccccgggcgcggccaccatgaaatgggttactttcatatctctgttgtttttgttttcctctagttccagggccatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcgagctcgaggtggtttgtctggtcaaccaccgcggtctcagtggtgtacggtacaaaccca

SEQ ID NO: 400 - DNA, 4906 bp, synthetic construct
  misc_feature 1..4906: note = Description of Artificial Sequence: Synthetic polynucleotide
  source 1..4906: mol_type = other DNA
ccccaactggggtaacctttgggctccccgggcgcggccaccatgaaatgggttactttcatatctctgttgtttttgttttcctctagttccagggccatgacgaggattttgacagctttcaaagtggtgaggacactgaagactggttttggctttaccaatgtgactgcacaccaaaaatggaaattttcaagacctggcatcaggctcctttctgtcaaggcacagacagcacacattgtcctggaagatggaactaagatgaaaggttactcctttggccatccatcctctgttgctggtgaagtggtttttaatactggcctgggagggtacccagaagctattactgaccctgcctacaaaggacagattctcacaatggccaaccctattattgggaatggtggagctcctgatactactgctctggatgaactgggacttagcaaatatttggagtctaatggaatcaaggtttcaggtttgctggtgctggattatagtaaagactacaaccactggctggctaccaagagtttagggcaatggctacaggaagaaaaggttcctgcaatttatggagtggacacaagaatgctgactaaaataattcgggataagggtaccatgcttgggaagattgaatttgaaggtcagcctgtggattttgtggatccaaataaacagaatttgattgctgaggtttcaaccaaggatgtcaaagtgtacggcaaaggaaaccccacaaaagtggtagctgtagactgtgggattaaaaacaatgtaatccgcctgctagtaaagcgaggagctgaagtgcacttagttccctggaaccatgatttcaccaagatggagtatgatgggattttgatcgcgggaggaccggggaacccagctcttgcagaaccactaattcagaatgtcagaaagattttggagagtgatcgcaaggagccattgtttggaatcagtacaggaaacttaataacaggattggctgctggtgccaaaacctacaagatgtccatggccaacagagggcagaatcagcctgttttgaatatcacaaacaaacaggctttcattactgctcagaatcatggctatgccttggacaacaccctccctgctggctggaaaccactttttgtgaatgtcaacgatcaaacaaatgaggggattatgcatgagagcaaacccttcttcgctgtgcagttccacccagaggtcaccccggggccaatagacactgagtacctgtttgattcctttttctcactgataaagaaaggaaaagctaccaccattacatcagtcttaccgaagccagcactagttgcatctcgggttgaggtttccaaagtccttattctaggatcaggaggtctgtccattggtcaggctggagaatttgattactcaggatctcaagctgtaaaagccatgaaggaagaaaatgtcaaaactgttctgatgaacccaaacattgcatcagtccagaccaatgaggtgggcttaaagcaagcggatactgtctactttcttcccatcacccctcagtttgtcacagaggtcatcaaggcagaacagccagatgggttaattctgggcatgggtggccagacagctctgaactgtggagtggaactattcaagagaggtgtgctcaaggaatatggtgtgaaagtcctgggaacttcagttgagtccattatggctacggaagacaggcagctgttttcagataaactaaatgagatcaatgaaaagattgctccaagttttgcagtggaatcgattgaggatgcactgaaggcagcagacaccattggctacccagtgatgatccgttccgcctatgcactgggtgggttaggctcaggcatctgtcccaacagagagactttgatggacctcagcacaaaggcctttgctatgaccaaccaaattctggtggagaagtcagtgacaggttggaaagaaatagaatatgaagtggttcgagatgctgatgacaattgtgtcactgtctgtaacatggaaaatgttgatgccatgggtgttcacacaggtgactcagttgttgtggctcctgcccagacactctccaatgccgagtttcagatgttgagacgtacttcaatcaatgttgttcgccacttgggcattgtgggtgaatgcaacattcagtttgcccttcatcctacctcaatggaatactgcatcattgaagtgaatgccagactgtcccgaagctctgctctggcctcaaaagccactggctacccattggcattcattgctgcaaagattgccctaggaatcccacttccagaaattaagaacgtcgtatccgggaagacatcagcctgttttgaacctagcctggattacatggtcaccaagattccccgctgggatcttgaccgttttcatggaacatctagccgaattggtagctctatgaaaagtgtaggagaggtcatggctattggtcgtacctttgaggagagtttccagaaagctttacggatgtgccacccatctatagaaggtttcactccccgtctcccaatgaacaaagaatggccatctaatttagatcttagaaaagagttgtctgaaccaagcagcacgcgtatctatgccattgccaaggccattgatgacaacatgtcccttgatgagattgagaagctcacatacattgacaagtggtttttgtataagatgcgtgatattttaaacatggaaaagacactgaaaggcctcaacagtgagtccatgacagaagaaaccctgaaaagggcaaaggagattgggttctcagataagcagatttcaaaatgccttgggctcactgaggcccagacaagggagctgaggttaaagaaaaacatccacccttgggttaaacagattgatacactggctgcagaatacccatcagtaacaaactatctctatgttacctacaatggtcaggagcatgatgtcaattttgatgaccatggaatgatggtgctaggctgtggtccatatcacattggcagcagtgtggaatttgattggtgtgctgtctctagtatccgcacactgcgtcaacttggcaagaagacggtggtggtgaattgcaatcctgagactgtgagcacagactttgatgagtgtgacaaactgtactttgaagagttgtccttggagagaatcctagacatctaccatcaggaggcatgtggtggctgcatcatatcagttggaggccagattccaaacaacctggcagttcctctatacaagaatggtgtcaagatcatgggcacaagccccctgcagatcgacagggctgaggatcgctccatcttctcagctgtcttggatgagctgaaggtggctcaggcaccttggaaagctgttaatactttgaatgaagcactggaatttgcaaagtctgtggactacccctgcttgttgaggccttcctatgttttgagtgggtctgctatgaatgtggtattctctgaggatgagatgaaaaaattcctagaagaggcgactagagtttctcaggagcacccagtggtgctgacaaaatttgttgaaggggcccgagaagtagaaatggacgctgttggcaaagatggaagggttatctctcatgccatctctgaacatgttgaagatgcaggtgtccactcgggagatgccactctgatgctgcccacacaaaccatcagccaaggggccattgaaaaggtgaaggatgctacccggaagattgcaaaggcttttgccatctctggtccattcaacgtccaatttcttgtcaaaggaaatgatgtcttggtgattgagtgtaacttgagagcttctcgatccttcccctttgtttccaagactcttggggttgacttcattgatgtggccaccaaggtgatgattggagagaatgttgatgagaaacatcttccaacattggaccatcccataattcctgctgactatgttgcaattaaggctcccatgttttcctggccccggttgagggatgctgaccccattctgagatgtgagatggcttccactggagaggtggcttgctttggtgaaggtattcatacagccttcctaaaggcaatgctttccacaggatttaagataccccagaaaggcatcctgataggcatccagcaatcattccggccaagattccttggtgtggctgaacaattacacaatgaaggtttcaagctgtttgccacggaagccacatcagactggctcaacgccaacaatgtccctgccaccccagtggcatggccgtctcaagaaggacagaatcccagcctctcttccatcagaaaattgattagagatggcagcattgacctagtgattaaccttcccaacaacaacactaaatttgtccatgataattatgtgattcggaggacagctgttgatagtggaatccctctcctcactaattttcaggtgaccaaactttttgctgaagctgtgcagaaatctcgcaaggtggactccaagagtcttttccactacaggcagtacagtgctggaaaagcagcataggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcgagctcgaggtggtttgtctggtcaaccaccgcggtctcagtggtgtacggtacaaaccca

SEQ ID NO: 401 - DNA, 4882 bp, synthetic construct
  misc_feature 1..4882: note = Description of Artificial Sequence: Synthetic polynucleotide
  source 1..4882: mol_type = other DNA
ccccaactggggtaacctttgggctccccgggcgcgactataagctgcgagcaacttcacttgggtatgccggcggtagcgcttaccgttcgtataatgtatgctatacgaagttatccgaagccgctagcggtggtttgtctggtcaaccaccgcggtctcagtggtgtacggtacaaacccacccgagagaccatgcagaggtcgcctctggaaaaggccagcgttgtctccaaacttttctttagctggactagacccatccttcgtaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagacgtattgactgagaaggagatcttcgagtcctgcgtttgcaagcttatggccaataagacaagaatcctggttacaagtaagatggagcacctgaagaaggccgataagattctgatcctgcacgagggatcttcatacttctacggcactttcagcgagcttcagaacttgcaacctgatttctctagcaagcttatgggctgcgactcctttgatcagttctctgccgagcgtcgcaactccattctgaccgaaacactgcataggttttccctcgagggcgacgcaccagtgtcttggactgagactaagaagcagagcttcaagcaaaccggcgaattcggtgagaagagaaagaacagtatcctgaaccccattaattcaattcggaagttcagtatcgttcagaaaacgcctcttcagatgaacgggattgaggaagactcagacgaaccgcttgaaaggcgactctcattggttcctgacagtgaacaaggggaagctattctcccccggatttcagtaatttccacaggtccgactctgcaagcccggagaagacaatccgtgttgaatcttatgacccattccgtgaatcaggggcaaaatatccatagaaagactactgcctctacgaggaaggtatcccttgcaccccaagccaatctgacggagctcgacatctactctcgccgcctgtcccaggagacaggactggagattagcgaggagatcaatgaagaggatctgaaagaatgtttcttcgacgacatggaatccatccctgccgtcacgacgtggaatacctatttgcgttacatcacggtacataaaagtctgatattcgtcctgatctggtgtcttgtgatcttcctcgctgaagtcgcagccagcctggtcgttctttggctgctcgggaatacccccttgcaggataagggaaactccacccactctcggaacaatagttacgccgtcatcattacttccacttcctcatactacgtattctatatatatgtcggggtcgctgatacactgctggccatgggcttctttcgcggcctgccgctcgtccacacgctgataactgtctccaagatcttgcatcataagatgctgcactcagtgctgcaggctccaatgagtacactgaatactcttaaggctggcggcatcctgaaccgctttagtaaggacatcgccatacttgacgatctcttgcccctgacaatcttcgattttattcaactccttttgatcgttatcggggcgatcgctgtggttgctgtgttgcagccatatatattcgtagctactgttcccgtcatcgtcgcgttcatcatgctccgtgcctactttctgcagacgtcccaacagctgaagcagctcgagagcgagggacggtcccccatatttacgcacttggtaactagtctgaaggggctgtggactctgagagcatttggtcgacaaccatatttcgagaccctctttcataaggccctcaacctgcacaccgcgaattggtttctgtatttgagtacgttgcggtggtttcagatgcgcatcgagatgatattcgtgatattctttatcgcagtcacatttatcagcatcctgactacgggcgagggagagggtcgcgtgggcatcatactcacgctcgctatgaacattatgagcaccctgcaatgggccgtgaatagctctatcgacgttgacagtcttatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagacccgctgatcagcctcgactgtgccttctagttgccagccatctgttgtttgcccctcccccgtgccttccttgaccctggaaggtgccactcccactgtcctttcctaataaaatgagaaaattgcatcgcattgtctgagtaggtgtcattctattctggggggtggggtggggcaggacagcaagggggaggattgggaagacaatagcaggcatgctggggatgcggtgggctctatgg

SEQ ID NO: 402 - DNA, 1594 bp, synthetic construct
  misc_feature 1..1594: note = Description of Artificial Sequence: Synthetic polynucleotide
  source 1..1594: mol_type = other DNA
ccccaactggggtaacctttgggctccccgggcgcggttccggatccggagagggcaggggatctctccttacttgtggcgacgtggaggagaaccccggccccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggctatggtcaagagaaaggattccagaggccgggccaagcggtccggatccggagccaccaacttcagcctgctgaagcaggccggcgacgtggaggagaaccccggccccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgcgggtggtttgtctggtcaaccaccgcggtctcagtggtgtacggtacaaaccca

SEQ ID NO: 403 - DNA, 19 bp, synthetic construct
  misc_feature 1..19: note = Description of Artificial Sequence: Synthetic oligonucleotide
  source 1..19: mol_type = other DNA
ttgagcgggcccccaccgt

SEQ ID NO: 404 - DNA, 393 bp, synthetic construct
  misc_feature 1..393: note = Description of Artificial Sequence: Synthetic polynucleotide
  source 1..393: mol_type = other DNA
atgactcactatcaggccttgcttttggacacggaccgggtccagttcggaccggtggtagccctgaacccggctacgctgctcccactgcctgaggaagggctgcaacacaactgccttgatgggacaggtggcggtggtgtcaccgtcaagttcaagtacaagggtgaggaacttgaagttgatattagcaaaatcaagaaggtttggcgcgttggtaaaatgatatcttttacttatgacgacaacggcaagacaggtagaggggcagtgtctgagaaagacgcccccaaggagctgttgcaaatgttggaaaagtctgggaaaaagtctggcggctcaaaaagaaccgccgacggcagcgaattcgagcccaagaagaagaggaaagtc

SEQ ID NO: 405 - DNA, 11 bp, synthetic construct
  misc_feature 1..11: note = Description of Artificial Sequence: Synthetic probe
  source 1..11: mol_type = other DNA
cgacgacggcg

SEQ ID NO: 406 - DNA, 16 bp, synthetic construct
  misc_feature 1..16: note = Description of Artificial Sequence: Synthetic probe
  source 1..16: mol_type = other DNA
tttatttgtgggcccg

SEQ ID NO: 407 - DNA, 15 bp, synthetic construct
  misc_feature 1..15: note = Description of Artificial Sequence: Synthetic probe
  source 1..15: mol_type = other DNA
tcgagtgccgcatca

SEQ ID NO: 408 - DNA, 17 bp, synthetic construct
  misc_feature 1..17: note = Description of Artificial Sequence: Synthetic probe
  source 1..17: mol_type = other DNA
aaagtggtgaggacact

SEQ ID NO: 409 - DNA, 15 bp, synthetic construct
  misc_feature 1..15: note = Description of Artificial Sequence: Synthetic probe
  source 1..15: mol_type = other DNA
aacccacccgagaga

SEQ ID NO: 410 - DNA, 66 bp, synthetic construct
  misc_feature 1..66: note = Description of Artificial Sequence: Synthetic oligonucleotide
  source 1..66: mol_type = other DNA
ggaagcggagctactaacttcagcctgctgaagcaggctggcgacgtggaggagaaccctggacct

SEQ ID NO: 411 - DNA, 45 bp, synthetic construct
  misc_feature 1..45: note = Description of Artificial Sequence: Synthetic oligonucleotide
  source 1..45: mol_type = other DNA
gggggaggaggttctggaggcggaggctccggaggcggagggtca

SEQ ID NO: 412 - DNA, 15 bp, synthetic construct
  misc_feature 1..15: note = Description of Artificial Sequence: Synthetic oligonucleotide
  source 1..15: mol_type = other DNA
ggaggtggcgggagc

SEQ ID NO: 413 - DNA, 15 bp, synthetic construct
  misc_feature 1..15: note = Description of Artificial Sequence: Synthetic oligonucleotide
  source 1..15: mol_type = other DNA
cccgcaccagcgcct

SEQ ID NO: 414 - DNA, 45 bp, synthetic construct
  misc_feature 1..45: note = Description of Artificial Sequence: Synthetic oligonucleotide
  source 1..45: mol_type = other DNA
gaggcagctgccaaggaagccgctgccaaggaggcggccgcaaag

SEQ ID NO: 415 - DNA, 48 bp, synthetic construct
  misc_feature 1..48: note = Description of Artificial Sequence: Synthetic oligonucleotide
  source 1..48: mol_type = other DNA
agtgggagcgagacccctgggactagcgagtcagctacacccgaaagc

SEQ ID NO: 416 - DNA, 54 bp, synthetic construct
  misc_feature 1..54: note = Description of Artificial Sequence: Synthetic oligonucleotide
  source 1..54: mol_type = other DNA
ggggggtcaggtggatccggcggaagtggcggatccggtggatctggcggcagt

SEQ ID NO: 417 - DNA, 15 bp, synthetic construct
  misc_feature 1..15: note = Description of Artificial Sequence: Synthetic oligonucleotide
  source 1..15: mol_type = other DNA
gaagctgctgctaag

SEQ ID NO: 418 - protein, 22 aa, synthetic construct
  REGION 1..22: note = Description of Artificial Sequence: Synthetic peptide
GSGATNFSLLKQAGDVEENPGP

SEQ ID NO: 419 - protein, 15 aa, synthetic construct
  REGION 1..15: note = Description of Artificial Sequence: Synthetic peptide
GGGGSGGGGSGGGGS

SEQ ID NO: 420 - protein, 5 aa, synthetic construct
  REGION 1..5: note = Description of Artificial Sequence: Synthetic peptide
GGGGS

SEQ ID NO: 421 - protein, 5 aa, synthetic construct
  REGION 1..5: note = Description of Artificial Sequence: Synthetic peptide
PAPAP

SEQ ID NO: 422 - protein, 15 aa, synthetic construct
  REGION 1..15: note = Description of Artificial Sequence: Synthetic peptide
EAAAKEAAAKEAAAK

SEQ ID NO: 423 - protein, 16 aa, synthetic construct
  REGION 1..16: note = Description of Artificial Sequence: Synthetic peptide
SGSETPGTSESATPES

SEQ ID NO: 424 - protein, 18 aa, synthetic construct
  REGION 1..18: note = Description of Artificial Sequence: Synthetic peptide
GGSGGSGGSGGSGGSGGS

SEQ ID NO: 425 - protein, 5 aa, synthetic construct
  REGION 1..5: note = Description of Artificial Sequence: Synthetic peptide
EAAAK

SEQ ID NO: 426 - protein, 16 aa, synthetic construct
  REGION 1..16: note = Description of Artificial Sequence: Synthetic peptide
GLSGQPPRSPSSGSSG

SEQ ID NO: 427 - protein, 17 aa, synthetic construct
  REGION 1..17: note = Description of Artificial Sequence: Synthetic peptide
GGLSGQPPRSPSSGSSG

SEQ ID NO: 428 - RNA, 88 nt, synthetic construct
  misc_feature 1..88: note = Description of Artificial Sequence: Synthetic oligonucleotide
  source 1..88: mol_type = other RNA
gacgagcgcggcgatatcatcatccatggccggatgatcctgacgacggagaccgccgtcgtcgacaagccggcctgagctgcgagaa

SEQ ID NO: 429 - RNA, 20 nt, synthetic construct
  misc_feature 1..20: note = Description of Artificial Sequence: Synthetic oligonucleotide
  source 1..20: mol_type = other RNA
gaagccggccttgcacatgc

SEQ ID NO: 430 - DNA, 95 bp, Homo sapiens
  source 1..95: mol_type = unassigned DNA
gcgcgcccggctattctcgcagctcaccatggatgatgatatcgccgcgctcgtcgtcgacaacggctccggcatgtgcaaggccggcttcgcgg

SEQ ID NO: 431 - RNA, 20 nt, synthetic construct
  misc_feature 1..20: note = Description of Artificial Sequence: Synthetic oligonucleotide
  source 1..20: mol_type = other RNA
accactcgacgctcttatcg